SEQUENCE LISTING (58 sequences)

SEQ ID NO: 1 - RNA, 36 nt, synthetic construct
  source 1..36: mol_type = other RNA
ggtggatgaaactcagtttagcagccgaaaggctgc

SEQ ID NO: 2 - RNA, 29 nt, synthetic construct
  source 1..29: mol_type = other RNA
gaaactcagtttagcagccgaaaggctgc

SEQ ID NO: 3 - RNA, 23 nt, synthetic construct
  source 1..23: mol_type = other RNA
gaaactcagtttagccgaaaggc

SEQ ID NO: 4 - DNA, 30 bp, synthetic construct
  misc_feature 1: note = DNA fragment
  misc_feature 2..30: note = RNA fragment
  source 1..30: mol_type = other DNA
tgaaactcagtttagcagccgaaaggctgc

SEQ ID NO: 5 - RNA, 31 nt, synthetic construct
  source 1..31: mol_type = other RNA
atgaaactcagtttagcagccgaaaggctgc

SEQ ID NO: 6 - RNA, 32 nt, synthetic construct
  source 1..32: mol_type = other RNA
gatgaaactcagtttagcagccgaaaggctgc

SEQ ID NO: 7 - RNA, 33 nt, synthetic construct
  source 1..33: mol_type = other RNA
ggatgaaactcagtttagcagccgaaaggctgc

SEQ ID NO: 8 - DNA, 34 bp, synthetic construct
  misc_feature 1: note = DNA fragment
  misc_feature 2..34: note = RNA fragment
  source 1..34: mol_type = other DNA
tggatgaaactcagtttagcagccgaaaggctgc

SEQ ID NO: 9 - RNA, 16 nt, synthetic construct
  source 1..16: mol_type = other RNA
gatgaaactcagttta

SEQ ID NO: 10 - RNA, 36 nt, synthetic construct
  source 1..36: mol_type = other RNA
accatcaaggtgaagttcaagcagccgaaaggctgc

SEQ ID NO: 11 - DNA, 32 bp, synthetic construct
  misc_feature 1: note = DNA fragment
  misc_feature 2..32: note = RNA fragment
  source 1..32: mol_type = other DNA
tcaaggtgaagttcaagcagccgaaaggctgc

SEQ ID NO: 12 - DNA, 16 bp, synthetic construct
  misc_feature 1: note = DNA fragment
  misc_feature 2..16: note = RNA fragment
  source 1..16: mol_type = other DNA
tcaaggtgaagttcaa

SEQ ID NO: 13 - RNA, 36 nt, synthetic construct
  source 1..36: mol_type = other RNA
agttgaattatcagcttaaagcagccgaaaggctgc

SEQ ID NO: 14 - RNA, 16 nt, synthetic construct
  source 1..16: mol_type = other RNA
gaattatcagcttaaa

SEQ ID NO: 15 - RNA, 16 nt, synthetic construct
  source 1..16: mol_type = other RNA
gcagccgaaaggctgc

SEQ ID NO: 16 - RNA, 22 nt, synthetic construct
  source 1..22: mol_type = other RNA
taaactgagtttcatccaccgg

SEQ ID NO: 17 - RNA, 22 nt, synthetic construct
  source 1..22: mol_type = other RNA
taaactgagtttcatccactgg

SEQ ID NO: 18 - RNA, 22 nt, synthetic construct
  source 1..22: mol_type = other RNA
ttgaacttcaccttgatggtgg

SEQ ID NO: 19 - RNA, 22 nt, synthetic construct
  source 1..22: mol_type = other RNA
tttaagctgataattcaactgg

SEQ ID NO: 20 - RNA, 36 nt, synthetic construct; identical to SEQ ID NO: 1
  modified_base 1^2: mod_base = OTHER; note = linked via phosphorothioate
  modified_base 1: mod_base = gm
  modified_base 2: mod_base = gm
  modified_base 3: mod_base = OTHER; note = 2'-fluoro-uridine
  modified_base 4: mod_base = gm
  modified_base 5: mod_base = OTHER; note = 2'-fluoro-guanosine
  modified_base 6: mod_base = OTHER; note = 2'-O-methyl-adenosine
  modified_base 7: mod_base = um
  modified_base 8: mod_base = OTHER; note = 2'-fluoro-guanosine
  modified_base 9: mod_base = OTHER; note = 2'-O-methyl-adenosine
  modified_base 10: mod_base = OTHER; note = 2'-fluoro-adenosine
  modified_base 11: mod_base = OTHER; note = 2'-O-methyl-adenosine
  modified_base 12: mod_base = OTHER; note = 2'-fluoro-cytidine
  modified_base 13: mod_base = OTHER; note = 2'-fluoro-uridine
  modified_base 14: mod_base = cm
  modified_base 15: mod_base = OTHER; note = 2'-fluoro-adenosine
  modified_base 16: mod_base = gm
  modified_base 17: mod_base = OTHER; note = 2'-fluoro-uridine
  modified_base 18: mod_base = um
  modified_base 19: mod_base = um
  modified_base 20: mod_base = OTHER; note = 2'-O-methyl-adenosine
  modified_base 21: mod_base = gm
  modified_base 22: mod_base = cm
  modified_base 23: mod_base = OTHER; note = 2'-O-methyl-adenosine
  modified_base 24: mod_base = gm
  modified_base 25: mod_base = cm
  modified_base 26: mod_base = cm
  modified_base 27: mod_base = gm
  modified_base 28: mod_base = OTHER; note = docosanamidodiethoxymethanol-adenosine
  modified_base 29: mod_base = OTHER; note = 2'-O-methyl-adenosine
  modified_base 30: mod_base = OTHER; note = 2'-O-methyl-adenosine
  modified_base 31: mod_base = gm
  modified_base 32: mod_base = gm
  modified_base 33: mod_base = cm
  modified_base 34: mod_base = um
  modified_base 35: mod_base = gm
  modified_base 36: mod_base = cm
  source 1..36: mol_type = other RNA

SEQ ID NO: 21 - RNA, 29 nt, synthetic construct
  modified_base 1^2: mod_base = OTHER; note = linked via phosphorothioate
  modified_base 1: mod_base = OTHER; note = 2'-O,4'-C-methyleneguanosine
  modified_base 2: mod_base = OTHER; note = 2'-O-methyl-adenosine
  modified_base 3: mod_base = OTHER; note = 2'-fluoro-adenosine
  modified_base 4: mod_base = OTHER; note = 2'-O-methyl-adenosine
  modified_base 5: mod_base = OTHER; note = 2'-fluoro-cytidine
  modified_base 6: mod_base = OTHER; note = 2'-fluoro-uridine
  modified_base 7: mod_base = cm
  modified_base 8: mod_base = OTHER; note = 2'-fluoro-adenosine
  modified_base 9: mod_base = gm
  modified_base 10: mod_base = OTHER; note = 2'-fluoro-uridine
  modified_base 11: mod_base = um
  modified_base 12: mod_base = um
  modified_base 13: mod_base = OTHER; note = 2'-O-methyl-adenosine
  modified_base 14: mod_base = gm
  modified_base 15: mod_base = cm
  modified_base 16: mod_base = OTHER; note = 2'-O-methyl-adenosine
  modified_base 17: mod_base = gm
  modified_base 18: mod_base = cm
  modified_base 19: mod_base = cm
  modified_base 20: mod_base = gm
  modified_base 21: mod_base = OTHER; note = docosanamidodiethoxymethanol-adenosine
  modified_base 22: mod_base = OTHER; note = 2'-O-methyl-adenosine
  modified_base 23: mod_base = OTHER; note = 2'-O-methyl-adenosine
  modified_base 24: mod_base = gm
  modified_base 25: mod_base = gm
  modified_base 26: mod_base = cm
  modified_base 27: mod_base = um
  modified_base 28: mod_base = gm
  modified_base 29: mod_base = cm
  source 1..29: mol_type = other RNA
gaaactcagtttagcagccgaaaggctgc

SEQ ID NO: 22 - RNA, 23 nt, synthetic construct
  modified_base 1^2: mod_base = OTHER; note = linked via phosphorothioate
  modified_base 1: mod_base = OTHER; note = 2'-O,4'-C-methyleneguanosine
  modified_base 2: mod_base = OTHER; note = 2'-O-methyl-adenosine
  modified_base 3: mod_base = OTHER; note = 2'-fluoro-adenosine
  modified_base 4: mod_base = OTHER; note = 2'-O-methyl-adenosine
  modified_base 5: mod_base = OTHER; note = 2'-fluoro-cytidine
  modified_base 6: mod_base = OTHER; note = 2'-fluoro-uridine
  modified_base 7: mod_base = cm
  modified_base 8: mod_base = OTHER; note = 2'-fluoro-adenosine
  modified_base 9: mod_base = gm
  modified_base 10: mod_base = OTHER; note = 2'-fluoro-uridine
  modified_base 11: mod_base = um
  modified_base 12: mod_base = um
  modified_base 13: mod_base = OTHER; note = 2'-O-methyl-adenosine
  modified_base 14: mod_base = OTHER; note = 2'-O,4'-C-methyleneguanosine
  modified_base 15: mod_base = cm
  modified_base 16: mod_base = cm
  modified_base 17: mod_base = gm
  modified_base 18: mod_base = OTHER; note = docosanamidodiethoxymethanol-adenosine
  modified_base 19: mod_base = OTHER; note = 2'-O-methyl-adenosine
  modified_base 20: mod_base = OTHER; note = 2'-O-methyl-adenosine
  modified_base 21: mod_base = gm
  modified_base 22: mod_base = gm
  modified_base 23: mod_base = OTHER; note = 2'-O,4'-C-methylenecytidine
  source 1..23: mol_type = other RNA
gaaactcagtttagccgaaaggc

SEQ ID NO: 23 - RNA, 23 nt, synthetic construct
  modified_base 1^2: mod_base = OTHER; note = linked via phosphorothioate
  modified_base 1: mod_base = OTHER; note = 2'-O,4'-C-methyleneguanosine
  modified_base 2: mod_base = OTHER; note = 2'-fluoro-adenosine
  modified_base 3: mod_base = OTHER; note = 2'-fluoro-adenosine
  modified_base 4: mod_base = OTHER; note = 2'-fluoro-adenosine
  modified_base 5: mod_base = cm
  modified_base 6: mod_base = um
  modified_base 7: mod_base = cm
  modified_base 8: mod_base = OTHER; note = 2'-O-methyl-adenosine
  modified_base 9: mod_base = gm
  modified_base 10: mod_base = um
  modified_base 11: mod_base = um
  modified_base 12: mod_base = um
  modified_base 13: mod_base = OTHER; note = 2'-O-methyl-adenosine
  modified_base 14: mod_base = OTHER; note = 2'-O,4'-C-methyleneguanosine
  modified_base 15: mod_base = cm
  modified_base 16: mod_base = cm
  modified_base 17: mod_base = gm
  modified_base 18: mod_base = OTHER; note = docosanamidodiethoxymethanol-adenosine
  modified_base 19: mod_base = OTHER; note = 2'-O-methyl-adenosine
  modified_base 20: mod_base = OTHER; note = 2'-O-methyl-adenosine
  modified_base 21: mod_base = gm
  modified_base 22: mod_base = gm
  modified_base 23: mod_base = OTHER; note = 2'-O,4'-C-methylenecytidine
  source 1..23: mol_type = other RNA
gaaactcagtttagccgaaaggc

SEQ ID NO: 24 - RNA, 23 nt, synthetic construct
  modified_base 1^2: mod_base = OTHER; note = linked via phosphorothioate
  modified_base 1: mod_base = OTHER; note = 2'-O,4'-C-methyleneguanosine
  modified_base 2: mod_base = OTHER; note = 2'-fluoro-adenosine
  modified_base 3: mod_base = OTHER; note = 2'-fluoro-adenosine
  modified_base 4: mod_base = OTHER; note = 2'-fluoro-adenosine
  modified_base 5: mod_base = cm
  modified_base 6: mod_base = um
  modified_base 7: mod_base = cm
  modified_base 8: mod_base = OTHER; note = 2'-O-methyl-adenosine
  modified_base 9: mod_base = gm
  modified_base 10: mod_base = um
  modified_base 11: mod_base = um
  modified_base 12: mod_base = um
  modified_base 13: mod_base = OTHER; note = 2'-O-methyl-adenosine
  modified_base 14: mod_base = OTHER; note = 2'-O,4'-C-methyleneguanosine
  modified_base 15: mod_base = cm
  modified_base 16: mod_base = cm
  modified_base 17: mod_base = gm
  modified_base 18: mod_base = OTHER; note = hexadecanamidodiethoxymethanol-adenosine
  modified_base 19: mod_base = OTHER; note = 2'-O-methyl-adenosine
  modified_base 20: mod_base = OTHER; note = 2'-O-methyl-adenosine
  modified_base 21: mod_base = gm
  modified_base 22: mod_base = gm
  modified_base 23: mod_base = OTHER; note = 2'-O,4'-C-methylenecytidine
  source 1..23: mol_type = other RNA
gaaactcagtttagccgaaaggc

SEQ ID NO: 25 - RNA, 29 nt, synthetic construct
  modified_base 1^2: mod_base = OTHER; note = linked via phosphorothioate
  modified_base 1: mod_base = OTHER; note = 2'-O,4'-C-methyleneguanosine
  modified_base 2: mod_base = OTHER; note = 2'-fluoro-adenosine
  modified_base 3: mod_base = OTHER; note = 2'-fluoro-adenosine
  modified_base 4: mod_base = OTHER; note = 2'-fluoro-adenosine
  modified_base 5: mod_base = cm
  modified_base 6: mod_base = um
  modified_base 7: mod_base = cm
  modified_base 8: mod_base = OTHER; note = 2'-O-methyl-adenosine
  modified_base 9: mod_base = gm
  modified_base 10: mod_base = um
  modified_base 11: mod_base = um
  modified_base 12: mod_base = um
  modified_base 13: mod_base = OTHER; note = 2'-O-methyl-adenosine
  modified_base 14: mod_base = gm
  modified_base 15: mod_base = cm
  modified_base 16: mod_base = OTHER; note = 2'-O-methyl-adenosine
  modified_base 17: mod_base = gm
  modified_base 18: mod_base = cm
  modified_base 19: mod_base = cm
  modified_base 20: mod_base = gm
  modified_base 21: mod_base = OTHER; note = hexadecanamidodiethoxymethanol-adenosine
  modified_base 22: mod_base = OTHER; note = 2'-O-methyl-adenosine
  modified_base 23: mod_base = OTHER; note = 2'-O-methyl-adenosine
  modified_base 24: mod_base = gm
  modified_base 25: mod_base = gm
  modified_base 26: mod_base = cm
  modified_base 27: mod_base = um
  modified_base 28: mod_base = gm
  modified_base 29: mod_base = cm
  source 1..29: mol_type = other RNA
gaaactcagtttagcagccgaaaggctgc

SEQ ID NO: 26 - DNA, 30 bp, synthetic construct; identical to SEQ ID NO: 4
  modified_base 1^2: mod_base = OTHER; note = linked via phosphorothioate
  misc_feature 1: note = DNA fragment
  misc_feature 2..30: note = RNA fragment
  modified_base 1: mod_base = OTHER; note = 2'-O,4'-C-methylenedeoxythymidine
  modified_base 2: mod_base = OTHER; note = 2'-fluoro-guanosine
  modified_base 3: mod_base = OTHER; note = 2'-fluoro-adenosine
  modified_base 4: mod_base = OTHER; note = 2'-fluoro-adenosine
  modified_base 5: mod_base = OTHER; note = 2'-fluoro-adenosine
  modified_base 6: mod_base = cm
  modified_base 7: mod_base = um
  modified_base 8: mod_base = cm
  modified_base 9: mod_base = OTHER; note = 2'-O-methyl-adenosine
  modified_base 10: mod_base = gm
  modified_base 11: mod_base = um
  modified_base 12: mod_base = um
  modified_base 13: mod_base = um
  modified_base 14: mod_base = OTHER; note = 2'-O-methyl-adenosine
  modified_base 15: mod_base = gm
  modified_base 16: mod_base = cm
  modified_base 17: mod_base = OTHER; note = 2'-O-methyl-adenosine
  modified_base 18: mod_base = gm
  modified_base 19: mod_base = cm
  modified_base 20: mod_base = cm
  modified_base 21: mod_base = gm
  modified_base 22: mod_base = OTHER; note = hexadecanamidodiethoxymethanol-adenosine
  modified_base 23: mod_base = OTHER; note = 2'-O-methyl-adenosine
  modified_base 24: mod_base = OTHER; note = 2'-O-methyl-adenosine
  modified_base 25: mod_base = gm
  modified_base 26: mod_base = gm
  modified_base 27: mod_base = cm
  modified_base 28: mod_base = um
  modified_base 29: mod_base = gm
  modified_base 30: mod_base = cm
  source 1..30: mol_type = other DNA

SEQ ID NO: 27 - RNA, 31 nt, synthetic construct; identical to SEQ ID NO: 5
  modified_base 1^2: mod_base = OTHER; note = linked via phosphorothioate
  modified_base 1: mod_base = OTHER; note = 2'-O,4'-C-methyleneadenosine
  modified_base 2: mod_base = um
  modified_base 3: mod_base = OTHER; note = 2'-fluoro-guanosine
  modified_base 4: mod_base = OTHER; note = 2'-fluoro-adenosine
  modified_base 5: mod_base = OTHER; note = 2'-fluoro-adenosine
  modified_base 6: mod_base = OTHER; note = 2'-fluoro-adenosine
  modified_base 7: mod_base = cm
  modified_base 8: mod_base = um
  modified_base 9: mod_base = cm
  modified_base 10: mod_base = OTHER; note = 2'-O-methyl-adenosine
  modified_base 11: mod_base = gm
  modified_base 12: mod_base = um
  modified_base 13: mod_base = um
  modified_base 14: mod_base = um
  modified_base 15: mod_base = OTHER; note = 2'-O-methyl-adenosine
  modified_base 16: mod_base = gm
  modified_base 17: mod_base = cm
  modified_base 18: mod_base = OTHER; note = 2'-O-methyl-adenosine
  modified_base 19: mod_base = gm
  modified_base 20: mod_base = cm
  modified_base 21: mod_base = cm
  modified_base 22: mod_base = gm
  modified_base 23: mod_base = OTHER; note = hexadecanamidodiethoxymethanol-adenosine
  modified_base 24: mod_base = OTHER; note = 2'-O-methyl-adenosine
  modified_base 25: mod_base = OTHER; note = 2'-O-methyl-adenosine
  modified_base 26: mod_base = gm
  modified_base 27: mod_base = gm
  modified_base 28: mod_base = cm
  modified_base 29: mod_base = um
  modified_base 30: mod_base = gm
  modified_base 31: mod_base = cm
  source 1..31: mol_type = other RNA

SEQ ID NO: 28 - RNA, 32 nt, synthetic construct; identical to SEQ ID NO: 6
  modified_base 1^2: mod_base = OTHER; note = linked via phosphorothioate
  modified_base 1: mod_base = OTHER; note = 2'-O,4'-C-methyleneguanosine
  modified_base 2: mod_base = OTHER; note = 2'-O-methyl-adenosine
  modified_base 3: mod_base = um
  modified_base 4: mod_base = OTHER; note = 2'-fluoro-guanosine
  modified_base 5: mod_base = OTHER; note = 2'-fluoro-adenosine
  modified_base 6: mod_base = OTHER; note = 2'-fluoro-adenosine
  modified_base 7: mod_base = OTHER; note = 2'-fluoro-adenosine
  modified_base 8: mod_base = cm
  modified_base 9: mod_base = um
  modified_base 10: mod_base = cm
  modified_base 11: mod_base = OTHER; note = 2'-O-methyl-adenosine
  modified_base 12: mod_base = gm
  modified_base 13: mod_base = um
  modified_base 14: mod_base = um
  modified_base 15: mod_base = um
  modified_base 16: mod_base = OTHER; note = 2'-O-methyl-adenosine
  modified_base 17: mod_base = gm
  modified_base 18: mod_base = cm
  modified_base 19: mod_base = OTHER; note = 2'-O-methyl-adenosine
  modified_base 20: mod_base = gm
  modified_base 21: mod_base = cm
  modified_base 22: mod_base = cm
  modified_base 23: mod_base = gm
  modified_base 24: mod_base = OTHER; note = hexadecanamidodiethoxymethanol-adenosine
  modified_base 25: mod_base = OTHER; note = 2'-O-methyl-adenosine
  modified_base 26: mod_base = OTHER; note = 2'-O-methyl-adenosine
  modified_base 27: mod_base = gm
  modified_base 28: mod_base = gm
  modified_base 29: mod_base = cm
  modified_base 30: mod_base = um
  modified_base 31: mod_base = gm
  modified_base 32: mod_base = cm
  source 1..32: mol_type = other RNA

SEQ ID NO: 29 - RNA, 33 nt, synthetic construct; identical to SEQ ID NO: 7
  modified_base 1^2: mod_base = OTHER; note = linked via phosphorothioate
  modified_base 1: mod_base = OTHER; note = 2'-O,4'-C-methyleneguanosine
  modified_base 2: mod_base = gm
  modified_base 3: mod_base = OTHER; note = 2'-O-methyl-adenosine
  modified_base 4: mod_base = um
  modified_base 5: mod_base = OTHER; note = 2'-fluoro-guanosine
  modified_base 6: mod_base = OTHER; note = 2'-fluoro-adenosine
  modified_base 7: mod_base = OTHER; note = 2'-fluoro-adenosine
  modified_base 8: mod_base = OTHER; note = 2'-fluoro-adenosine
  modified_base 9: mod_base = cm
  modified_base 10: mod_base = um
  modified_base 11: mod_base = cm
  modified_base 12: mod_base = OTHER; note = 2'-O-methyl-adenosine
  modified_base 13: mod_base = gm
  modified_base 14: mod_base = um
  modified_base 15: mod_base = um
  modified_base 16: mod_base = um
  modified_base 17: mod_base = OTHER; note = 2'-O-methyl-adenosine
  modified_base 18: mod_base = gm
  modified_base 19: mod_base = cm
  modified_base 20: mod_base = OTHER; note = 2'-O-methyl-adenosine
  modified_base 21: mod_base = gm
  modified_base 22: mod_base = cm
  modified_base 23: mod_base = cm
  modified_base 24: mod_base = gm
  modified_base 25: mod_base = OTHER; note = hexadecanamidodiethoxymethanol-adenosine
  modified_base 26: mod_base = OTHER; note = 2'-O-methyl-adenosine
  modified_base 27: mod_base = OTHER; note = 2'-O-methyl-adenosine
  modified_base 28: mod_base = gm
  modified_base 29: mod_base = gm
  modified_base 30: mod_base = cm
  modified_base 31: mod_base = um
  modified_base 32: mod_base = gm
  modified_base 33: mod_base = cm
  source 1..33: mol_type = other RNA

SEQ ID NO: 30 - DNA, 34 bp, synthetic construct; identical to SEQ ID NO: 8
  modified_base 1^2: mod_base = OTHER; note = linked via phosphorothioate
  misc_feature 1: note = DNA fragment
  misc_feature 2..34: note = RNA fragment
  modified_base 1: mod_base = OTHER; note = 2'-O,4'-C-methylenedeoxythymidine
  modified_base 2: mod_base = gm
  modified_base 3: mod_base = gm
  modified_base 4: mod_base = OTHER; note = 2'-O-methyl-adenosine
  modified_base 5: mod_base = um
  modified_base 6: mod_base = OTHER; note = 2'-fluoro-guanosine
  modified_base 7: mod_base = OTHER; note = 2'-fluoro-adenosine
  modified_base 8: mod_base = OTHER; note = 2'-fluoro-adenosine
  modified_base 9: mod_base = OTHER; note = 2'-fluoro-adenosine
  modified_base 10: mod_base = cm
  modified_base 11: mod_base = um
  modified_base 12: mod_base = cm
  modified_base 13: mod_base = OTHER; note = 2'-O-methyl-adenosine
  modified_base 14: mod_base = gm
  modified_base 15: mod_base = um
  modified_base 16: mod_base = um
  modified_base 17: mod_base = um
  modified_base 18: mod_base = OTHER; note = 2'-O-methyl-adenosine
  modified_base 19: mod_base = gm
  modified_base 20: mod_base = cm
  modified_base 21: mod_base = OTHER; note = 2'-O-methyl-adenosine
  modified_base 22: mod_base = gm
  modified_base 23: mod_base = cm
  modified_base 24: mod_base = cm
  modified_base 25: mod_base = gm
  modified_base 26: mod_base = OTHER; note = hexadecanamidodiethoxymethanol-adenosine
  modified_base 27: mod_base = OTHER; note = 2'-O-methyl-adenosine
  modified_base 28: mod_base = OTHER; note = 2'-O-methyl-adenosine
  modified_base 29: mod_base = gm
  modified_base 30: mod_base = gm
  modified_base 31: mod_base = cm
  modified_base 32: mod_base = um
  modified_base 33: mod_base = gm
  modified_base 34: mod_base = cm
  source 1..34: mol_type = other DNA

SEQ ID NO: 31 - RNA, 32 nt, synthetic construct; identical to SEQ ID NO: 6
  modified_base 1^2: mod_base = OTHER; note = linked via phosphorothioate
  modified_base 1: mod_base = OTHER; note = hexadecanamidodiethoxymethanol-guanosine
  modified_base 2: mod_base = OTHER; note = 2'-O-methyl-adenosine
  modified_base 3: mod_base = um
  modified_base 4: mod_base = OTHER; note = 2'-fluoro-guanosine
  modified_base 5: mod_base = OTHER; note = 2'-fluoro-adenosine
  modified_base 6: mod_base = OTHER; note = 2'-fluoro-adenosine
  modified_base 7: mod_base = OTHER; note = 2'-fluoro-adenosine
  modified_base 8: mod_base = cm
  modified_base 9: mod_base = um
  modified_base 10: mod_base = cm
  modified_base 11: mod_base = OTHER; note = 2'-O-methyl-adenosine
  modified_base 12: mod_base = gm
  modified_base 13: mod_base = um
  modified_base 14: mod_base = um
  modified_base 15: mod_base = um
  modified_base 16: mod_base = OTHER; note = 2'-O-methyl-adenosine
  modified_base 17: mod_base = gm
  modified_base 18: mod_base = cm
  modified_base 19: mod_base = OTHER; note = 2'-O-methyl-adenosine
  modified_base 20: mod_base = gm
  modified_base 21: mod_base = cm
  modified_base 22: mod_base = cm
  modified_base 23: mod_base = gm
  modified_base 24: mod_base = OTHER; note = 2'-O-methyl-adenosine
  modified_base 25: mod_base = OTHER; note = 2'-O-methyl-adenosine
  modified_base 26: mod_base = OTHER; note = 2'-O-methyl-adenosine
  modified_base 27: mod_base = gm
  modified_base 28: mod_base = gm
  modified_base 29: mod_base = cm
  modified_base 30: mod_base = um
  modified_base 31: mod_base = gm
  modified_base 32: mod_base = cm
  source 1..32: mol_type = other RNA

SEQ ID NO: 32 - RNA, 16 nt, synthetic construct
  modified_base 1^2: mod_base = OTHER; note = linked via phosphorothioate
  modified_base 1: mod_base = OTHER; note = 2'-O,4'-C-methyleneguanosine
  modified_base 2: mod_base = OTHER; note = 2'-O-methyl-adenosine
  modified_base 3: mod_base = um
  modified_base 4: mod_base = OTHER; note = 2'-fluoro-guanosine
  modified_base 5: mod_base = OTHER; note = 2'-fluoro-adenosine
  modified_base 6: mod_base = OTHER; note = 2'-fluoro-adenosine
  modified_base 7: mod_base = OTHER; note = 2'-fluoro-adenosine
  modified_base 8: mod_base = cm
  modified_base 9: mod_base = um
  modified_base 10: mod_base = cm
  modified_base 11: mod_base = OTHER; note = 2'-O-methyl-adenosine
  modified_base 12: mod_base = gm
  modified_base 13: mod_base = um
  modified_base 14..16: mod_base = OTHER; note = linked via phosphorothioate
  modified_base 14: mod_base = um
  modified_base 15: mod_base = um
  modified_base 16: mod_base = OTHER; note = hexadecanamidodiethoxymethanol-adenosine
  source 1..16: mol_type = other RNA
gatgaaactcagttta

SEQ ID NO: 33 - RNA, 36 nt, synthetic construct; identical to SEQ ID NO: 10
  modified_base 1^2: mod_base = OTHER; note = linked via phosphorothioate
  modified_base 1: mod_base = OTHER; note = 2'-O-methyl-adenosine
  modified_base 2: mod_base = cm
  modified_base 3: mod_base = cm
  modified_base 4: mod_base = OTHER; note = 2'-O-methyl-adenosine
  modified_base 5: mod_base = um
  modified_base 6: mod_base = cm
  modified_base 7: mod_base = OTHER; note = 2'-O-methyl-adenosine
  modified_base 8: mod_base = OTHER; note = 2'-fluoro-adenosine
  modified_base 9: mod_base = OTHER; note = 2'-fluoro-guanosine
  modified_base 10: mod_base = OTHER; note = 2'-fluoro-guanosine
  modified_base 11: mod_base = OTHER; note = 2'-fluoro-uridine
  modified_base 12: mod_base = gm
  modified_base 13: mod_base = OTHER; note = 2'-O-methyl-adenosine
  modified_base 14: mod_base = OTHER; note = 2'-O-methyl-adenosine
  modified_base 15: mod_base = gm
  modified_base 16: mod_base = um
  modified_base 17: mod_base = um
  modified_base 18: mod_base = cm
  modified_base 19: mod_base = OTHER; note = 2'-O-methyl-adenosine
  modified_base 20: mod_base = OTHER; note = 2'-O-methyl-adenosine
  modified_base 21: mod_base = gm
  modified_base 22: mod_base = cm
  modified_base 23: mod_base = OTHER; note = 2'-O-methyl-adenosine
  modified_base 24: mod_base = gm
  modified_base 25: mod_base = cm
  modified_base 26: mod_base = cm
  modified_base 27: mod_base = gm
  modified_base 28: mod_base = OTHER; note = hexadecanamidodiethoxymethanol-adenosine
  modified_base 29: mod_base = OTHER; note = 2'-O-methyl-adenosine
  modified_base 30: mod_base = OTHER; note = 2'-O-methyl-adenosine
  modified_base 31: mod_base = gm
  modified_base 32: mod_base = gm
  modified_base 33: mod_base = cm
  modified_base 34: mod_base = um
  modified_base 35: mod_base = gm
  modified_base 36: mod_base = cm
  source 1..36: mol_type = other RNA

SEQ ID NO: 34 - DNA, 32 bp, synthetic construct; identical to SEQ ID NO: 11
  modified_base 1^2: mod_base = OTHER; note = linked via phosphorothioate
  misc_feature 1: note = DNA fragment
  misc_feature 2..32: note = RNA fragment
  modified_base 1: mod_base = OTHER; note = 2'-O,4'-C-methylenedeoxythymidine
  modified_base 2: mod_base = cm
  modified_base 3: mod_base = OTHER; note = 2'-O-methyl-adenosine
  modified_base 4: mod_base = OTHER; note = 2'-fluoro-adenosine
  modified_base 5: mod_base = OTHER; note = 2'-fluoro-guanosine
  modified_base 6: mod_base = OTHER; note = 2'-fluoro-guanosine
  modified_base 7: mod_base = OTHER; note = 2'-fluoro-uridine
  modified_base 8: mod_base = gm
  modified_base 9: mod_base = OTHER; note = 2'-O-methyl-adenosine
  modified_base 10: mod_base = OTHER; note = 2'-O-methyl-adenosine
  modified_base 11: mod_base = gm
  modified_base 12: mod_base = um
  modified_base 13: mod_base = um
  modified_base 14: mod_base = cm
  modified_base 15: mod_base = OTHER; note = 2'-O-methyl-adenosine
  modified_base 16: mod_base = OTHER; note = 2'-O-methyl-adenosine
  modified_base 17: mod_base = gm
  modified_base 18: mod_base = cm
  modified_base 19: mod_base = OTHER; note = 2'-O-methyl-adenosine
  modified_base 20: mod_base = gm
  modified_base 21: mod_base = cm
  modified_base 22: mod_base = cm
  modified_base 23: mod_base = gm
  modified_base 24: mod_base = OTHER; note = hexadecanamidodiethoxymethanol-adenosine
  modified_base 25: mod_base = OTHER; note = 2'-O-methyl-adenosine
  modified_base 26: mod_base = OTHER; note = 2'-O-methyl-adenosine
  modified_base 27: mod_base = gm
  modified_base 28: mod_base = gm
  modified_base 29: mod_base = cm
  modified_base 30: mod_base = um
  modified_base 31: mod_base = gm
  modified_base 32: mod_base = cm
  source 1..32: mol_type = other DNA

SEQ ID NO: 35 - DNA, 16 bp, synthetic construct
  modified_base 1^2: mod_base = OTHER; note = linked via phosphorothioate
  misc_feature 1: note = DNA fragment
  misc_feature 2..16: note = RNA fragment
  modified_base 1: mod_base = OTHER; note = 2'-O,4'-C-methylenedeoxythymidine
  modified_base 2: mod_base = cm
  modified_base 3: mod_base = OTHER; note = 2'-O-methyl-adenosine
  modified_base 4: mod_base = OTHER; note = 2'-fluoro-adenosine
  modified_base 5: mod_base = OTHER; note = 2'-fluoro-guanosine
  modified_base 6: mod_base = OTHER; note = 2'-fluoro-guanosine
  modified_base 7: mod_base = OTHER; note = 2'-fluoro-uridine
  modified_base 8: mod_base = gm
  modified_base 9: mod_base = OTHER; note = 2'-O-methyl-adenosine
  modified_base 10: mod_base = OTHER; note = 2'-O-methyl-adenosine
  modified_base 11: mod_base = gm
  modified_base 12: mod_base = um
  modified_base 13: mod_base = um
  modified_base 14..16: mod_base = OTHER; note = linked via phosphorothioate
  modified_base 14: mod_base = cm
  modified_base 15: mod_base = OTHER; note = 2'-O-methyl-adenosine
  modified_base 16: mod_base = OTHER; note = hexadecanamidodiethoxymethanol-adenosine
  source 1..16: mol_type = other DNA
tcaaggtgaagttcaa

SEQ ID NO: 36 - RNA, 36 nt, synthetic construct; identical to SEQ ID NO: 13
  modified_base 1^2: mod_base = OTHER; note = linked via phosphorothioate
  modified_base 1: mod_base = OTHER; note = 2'-O-methyl-adenosine
  modified_base 2: mod_base = gm
  modified_base 3: mod_base = um
  modified_base 4: mod_base = um
  modified_base 5: mod_base = gm
  modified_base 6: mod_base = OTHER; note = 2'-O-methyl-adenosine
  modified_base 7: mod_base = OTHER; note = 2'-O-methyl-adenosine
  modified_base 8: mod_base = OTHER; note = 2'-fluoro-uridine
  modified_base 9: mod_base = OTHER; note = 2'-fluoro-uridine
  modified_base 10: mod_base = OTHER; note = 2'-fluoro-adenosine
  modified_base 11: mod_base = OTHER; note = 2'-fluoro-uridine
  modified_base 12: mod_base = cm
  modified_base 13: mod_base = OTHER; note = 2'-O-methyl-adenosine
  modified_base 14: mod_base = gm
  modified_base 15: mod_base = cm
  modified_base 16: mod_base = um
  modified_base 17: mod_base = um
  modified_base 18: mod_base = OTHER; note = 2'-O-methyl-adenosine
  modified_base 19: mod_base = OTHER; note = 2'-O-methyl-adenosine
  modified_base 20: mod_base = OTHER; note = 2'-O-methyl-adenosine
  modified_base 21: mod_base = gm
  modified_base 22: mod_base = cm
  modified_base 23: mod_base = OTHER; note = 2'-O-methyl-adenosine
  modified_base 24: mod_base = gm
  modified_base 25: mod_base = cm
  modified_base 26: mod_base = cm
  modified_base 27: mod_base = gm
  modified_base 28: mod_base = OTHER; note = hexadecanamidodiethoxymethanol-adenosine
  modified_base 29: mod_base = OTHER; note = 2'-O-methyl-adenosine
  modified_base 30: mod_base = OTHER; note = 2'-O-methyl-adenosine
  modified_base 31: mod_base = gm
  modified_base 32: mod_base = gm
  modified_base 33: mod_base = cm
  modified_base 34: mod_base = um
  modified_base 35: mod_base = gm
  modified_base 36: mod_base = cm
  source 1..36: mol_type = other RNA

SEQ ID NO: 37 - RNA, 16 nt, synthetic construct
  modified_base 1^2: mod_base = OTHER; note = linked via phosphorothioate
  modified_base 1: mod_base = OTHER; note = 2'-O,4'-C-methyleneguanosine
  modified_base 2: mod_base = OTHER; note = 2'-O-methyl-adenosine
  modified_base 3: mod_base = OTHER; note = 2'-O-methyl-adenosine
  modified_base 4: mod_base = OTHER; note = 2'-fluoro-uridine
  modified_base 5: mod_base = OTHER; note = 2'-fluoro-uridine
  modified_base 6: mod_base = OTHER; note = 2'-fluoro-adenosine
  modified_base 7: mod_base = OTHER; note = 2'-fluoro-uridine
  modified_base 8: mod_base = cm
  modified_base 9: mod_base = OTHER; note = 2'-O-methyl-adenosine
  modified_base 10: mod_base = gm
  modified_base 11: mod_base = cm
  modified_base 12: mod_base = um
  modified_base 13: mod_base = um
  modified_base 14..16: mod_base = OTHER; note = linked via phosphorothioate
  modified_base 14: mod_base = OTHER; note = 2'-O-methyl-adenosine
  modified_base 15: mod_base = OTHER; note = 2'-O-methyl-adenosine
  modified_base 16: mod_base = OTHER; note = hexadecanamidodiethoxymethanol-adenosine
  source 1..16: mol_type = other RNA
gaattatcagcttaaa

SEQ ID NO: 38 - skipped

SEQ ID NO: 39 - RNA, 22 nt, synthetic construct
  modified_base 1..3: mod_base = OTHER; note = linked via phosphorothioate
  modified_base 1: mod_base = OTHER; note = 4'-O-monomethylphosphonate-2'-O-methyluridine
  modified_base 2: mod_base = OTHER; note = 2'-fluoro-adenosine
  modified_base 3: mod_base = OTHER; note = 2'-fluoro-adenosine
  modified_base 4: mod_base = OTHER; note = 2'-fluoro-adenosine
  modified_base 5: mod_base = OTHER; note = 2'-fluoro-cytidine
  modified_base 6: mod_base = um
  modified_base 7: mod_base = OTHER; note = 2'-fluoro-guanosine
  modified_base 8: mod_base = OTHER; note = 2'-O-methyl-adenosine
  modified_base 9: mod_base = gm
  modified_base 10: mod_base = OTHER; note = 2'-fluoro-uridine
  modified_base 11: mod_base = um
  modified_base 12: mod_base = um
  modified_base 13: mod_base = cm
  modified_base 14: mod_base = OTHER; note = 2'-fluoro-adenosine
  modified_base 15: mod_base = um
  modified_base 16: mod_base = OTHER; note = 2'-fluoro-cytidine
  modified_base 17: mod_base = cm
  modified_base 18: mod_base = OTHER; note = 2'-O-methyl-adenosine
  modified_base 19: mod_base = OTHER; note = 2'-fluoro-cytidine
  modified_base 20..22: mod_base = OTHER; note = linked via phosphorothioate
  modified_base 20: mod_base = cm
  modified_base 21: mod_base = gm
  modified_base 22: mod_base = gm
  source 1..22: mol_type = other RNA
taaactgagtttcatccaccgg

SEQ ID NO: 40 - RNA, 22 nt, synthetic construct
  modified_base 1..4: mod_base = OTHER; note = linked via phosphorothioate
  modified_base 1: mod_base = OTHER; note = 4'-O-monomethylphosphonate-2'-O-methyluridine
  modified_base 2: mod_base = OTHER; note = 2'-fluoro-adenosine
  modified_base 3: mod_base = OTHER; note = 2'-fluoro-adenosine
  modified_base 4: mod_base = OTHER; note = 2'-fluoro-adenosine
  modified_base 5: mod_base = OTHER; note = 2'-fluoro-cytidine
  modified_base 6: mod_base = um
  modified_base 7: mod_base = OTHER; note = 2'-fluoro-guanosine
  modified_base 8: mod_base = OTHER; note = 2'-O-methyl-adenosine
  modified_base 9: mod_base = gm
  modified_base 10: mod_base = OTHER; note = 2'-fluoro-uridine
  modified_base 11: mod_base = um
  modified_base 12: mod_base = um
  modified_base 13..15: mod_base = OTHER; note = linked via phosphorothioate
  modified_base 13: mod_base = cm
  modified_base 14: mod_base = OTHER; note = 2'-fluoro-adenosine
  modified_base 15: mod_base = um
  modified_base 16: mod_base = OTHER; note = 2'-fluoro-cytidine
  modified_base 17: mod_base = cm
  modified_base 18: mod_base = OTHER; note = 2'-O-methyl-adenosine
  modified_base 19: mod_base = OTHER; note = 2'-fluoro-cytidine
  modified_base 20..22: mod_base = OTHER; note = linked via phosphorothioate
  modified_base 20: mod_base = cm
  modified_base 21: mod_base = gm
  modified_base 22: mod_base = gm
  source 1..22: mol_type = other RNA
taaactgagtttcatccaccgg

SEQ ID NO: 41 - RNA, 22 nt, synthetic construct
  modified_base 1..4: mod_base = OTHER; note = linked via phosphorothioate
  modified_base 1: mod_base = OTHER; note = 4'-O-monomethylphosphonate-2'-O-methyluridine
  modified_base 2: mod_base = OTHER; note = 2'-fluoro-adenosine
  modified_base 3: mod_base = OTHER; note = 2'-fluoro-adenosine
  modified_base 4: mod_base = OTHER; note = 2'-fluoro-adenosine
  modified_base 5: mod_base = OTHER; note = 2'-fluoro-cytidine
  modified_base 6: mod_base = um
  modified_base 7: mod_base = OTHER; note = 2'-fluoro-guanosine
  modified_base 8: mod_base = OTHER; note = 2'-O-methyl-adenosine
  modified_base 9: mod_base = gm
  modified_base 10: mod_base = OTHER; note = 2'-fluoro-uridine
  modified_base 11: mod_base = um
  modified_base 12: mod_base = um
  modified_base 13..15: mod_base = OTHER; note = linked via phosphorothioate
  modified_base 13: mod_base = cm
  modified_base 14: mod_base = OTHER; note = 2'-fluoro-adenosine
  modified_base 15: mod_base = um
  modified_base 16: mod_base = cm
  modified_base 17: mod_base = cm
  modified_base 18: mod_base = OTHER; note = 2'-O-methyl-adenosine
  modified_base 19: mod_base = cm
  modified_base 20..22: mod_base = OTHER; note = linked via phosphorothioate
  modified_base 20: mod_base = cm
  modified_base 21: mod_base = gm
  modified_base 22: mod_base = gm
  source 1..22: mol_type = other RNA
taaactgagtttcatccaccgg

SEQ ID NO: 42 - RNA, 22 nt, synthetic construct
  modified_base 1..4: mod_base = OTHER; note = linked via phosphorothioate
  modified_base 1: mod_base = OTHER; note = 4'-O-monomethylphosphonate-2'-O-methyluridine
  modified_base 2: mod_base = OTHER; note = 2'-fluoro-adenosine
  modified_base 3: mod_base = OTHER; note = 2'-fluoro-adenosine
  modified_base 4: mod_base = OTHER; note = 2'-fluoro-adenosine
  modified_base 5: mod_base = OTHER; note = 2'-fluoro-cytidine
  modified_base 6: mod_base = um
  modified_base 7: mod_base = OTHER; note = 2'-fluoro-guanosine
  modified_base 8: mod_base = OTHER; note = 2'-O-methyl-adenosine
  modified_base 9: mod_base = gm
  modified_base 10: mod_base = OTHER; note = 2'-fluoro-uridine
  modified_base 11: mod_base = um
  modified_base 12: mod_base = um
  modified_base 13..22: mod_base = OTHER; note = linked via phosphorothioate
  modified_base 13: mod_base = cm
  modified_base 14: mod_base = OTHER; note = 2'-fluoro-adenosine
  modified_base 15: mod_base = um
  modified_base 16: mod_base = cm
  modified_base 17: mod_base = cm
  modified_base 18: mod_base = OTHER; note = 2'-O-methyl-adenosine
  modified_base 19: mod_base = cm
  modified_base 20: mod_base = cm
  modified_base 21: mod_base = gm
  modified_base 22: mod_base = gm
  source 1..22: mol_type = other RNA
taaactgagtttcatccaccgg

SEQ ID NO: 43 - RNA, 22 nt, synthetic construct
  modified_base 1..4: mod_base = OTHER; note = linked via phosphorothioate
  modified_base 1: mod_base = OTHER; note = 4'-O-monomethylphosphonate-2'-O-methyluridine
  modified_base 2: mod_base = OTHER; note = 2'-fluoro-adenosine
  modified_base 3: mod_base = OTHER; note = 2'-fluoro-adenosine
  modified_base 4: mod_base = OTHER; note = 2'-fluoro-adenosine
  modified_base 5: mod_base = OTHER; note = 2'-fluoro-cytidine
  modified_base 6: mod_base = um
  modified_base 7: mod_base = OTHER; note = 2'-fluoro-guanosine
  modified_base 8: mod_base = OTHER; note = 2'-O-methyl-adenosine
  modified_base 9: mod_base = gm
  modified_base 10: mod_base = OTHER; note = 2'-fluoro-uridine
  modified_base 11: mod_base = um
  modified_base 12: mod_base = um
  modified_base 13..15: mod_base = OTHER; note = linked via phosphorothioate
  modified_base 13: mod_base = cm
  modified_base 14: mod_base = OTHER; note = 2'-fluoro-adenosine
  modified_base 15: mod_base = um
  modified_base 16: mod_base = cm
  modified_base 17: mod_base = cm
  modified_base 18: mod_base = OTHER; note = 2'-O-methyl-adenosine
  modified_base 19: mod_base = cm
  modified_base 20..22: mod_base = OTHER; note = linked via phosphorothioate
  modified_base 20: mod_base = cm
  modified_base 21: mod_base = gm
  modified_base 22: mod_base = gm
  source 1..22: mol_type = other RNA
taaactgagtttcatccaccgg

SEQ ID NO: 44 - RNA, 22 nt, synthetic construct
  modified_base 1..4: mod_base = OTHER; note = linked via phosphorothioate
  modified_base 1: mod_base = OTHER; note = 4'-O-monomethylphosphonate-2'-O-methyluridine
  modified_base 2: mod_base = OTHER; note = 2'-fluoro-adenosine
  modified_base 3: mod_base = OTHER; note = 2'-fluoro-adenosine
  modified_base 4: mod_base = OTHER; note = 2'-fluoro-adenosine
  modified_base 5: mod_base = OTHER; note = 2'-fluoro-cytidine
  modified_base 6: mod_base = um
  modified_base 7: mod_base = OTHER; note = 2'-fluoro-guanosine
  modified_base 8: mod_base = OTHER; note = 2'-O-methyl-adenosine
  modified_base 9: mod_base = gm
  modified_base 10: mod_base = OTHER; note = 2'-fluoro-uridine
  modified_base 11: mod_base = um
  modified_base 12: mod_base = um
  modified_base 13..15: mod_base = OTHER; note = linked via phosphorothioate
  modified_base 13: mod_base = cm
  modified_base 14: mod_base = OTHER; note = 2'-fluoro-adenosine
  modified_base 15: mod_base = um
  modified_base 16: mod_base = cm
  modified_base 17: mod_base = cm
  modified_base 18: mod_base = OTHER; note = 2'-O-methyl-adenosine
  modified_base 19: mod_base = OTHER; note = 2'-fluoro-cytidine
  modified_base 20..22: mod_base = OTHER; note = linked via phosphorothioate
  modified_base 20: mod_base = cm
  modified_base 21: mod_base = gm
  modified_base 22: mod_base = gm
  source 1..22: mol_type = other RNA
taaactgagtttcatccaccgg

SEQ ID NO: 45 - RNA, 22 nt, synthetic construct
  modified_base 1..4: mod_base = OTHER; note = linked via phosphorothioate
  modified_base 1: mod_base = OTHER; note = 4'-O-monomethylphosphonate-2'-O-methyluridine
  modified_base 2: mod_base = OTHER; note = 2'-fluoro-adenosine
  modified_base 3: mod_base = OTHER; note = 2'-fluoro-adenosine
  modified_base 4: mod_base = OTHER; note = 2'-fluoro-adenosine
  modified_base 5: mod_base = OTHER; note = 2'-fluoro-cytidine
  modified_base 6: mod_base = um
  modified_base 7: mod_base = OTHER; note = 2'-fluoro-guanosine
  modified_base 8: mod_base = OTHER; note = 2'-O-methyl-adenosine
  modified_base 9: mod_base = gm
  modified_base 10: mod_base = OTHER; note = 2'-fluoro-uridine
  modified_base 11: mod_base = um
  modified_base 12: mod_base = um
  modified_base 13..15: mod_base = OTHER; note = linked via phosphorothioate
  modified_base 13: mod_base = cm
  modified_base 14: mod_base = OTHER; note = 2'-fluoro-adenosine
  modified_base 15: mod_base = um
  modified_base 16: mod_base = OTHER; note = 2'-fluoro-cytidine
  modified_base 17: mod_base = cm
  modified_base 18: mod_base = OTHER; note = 2'-O-methyl-adenosine
  modified_base 19: mod_base = cm
  modified_base 20..22: mod_base = OTHER; note = linked via phosphorothioate
  modified_base 20: mod_base = cm
  modified_base 21: mod_base = gm
  modified_base 22: mod_base = gm
  source 1..22: mol_type = other RNA
taaactgagtttcatccaccgg

SEQ ID NO: 46 - RNA, 22 nt, synthetic construct
  modified_base 1..4: mod_base = OTHER; note = linked via phosphorothioate
  modified_base 1: mod_base = OTHER; note = 4'-O-monomethylphosphonate-2'-O-methyluridine
  modified_base 2: mod_base = OTHER; note = 2'-fluoro-adenosine
  modified_base 3: mod_base = OTHER; note = 2'-fluoro-adenosine
  modified_base 4: mod_base = OTHER; note = 2'-fluoro-adenosine
  modified_base 5: mod_base = OTHER; note = 2'-fluoro-cytidine
  modified_base 6: mod_base = um
  modified_base 7: mod_base = OTHER; note = 2'-fluoro-guanosine
  modified_base 8: mod_base = OTHER; note = 2'-O-methyl-adenosine
  modified_base 9: mod_base = gm
  modified_base 10: mod_base = OTHER; note = 2'-fluoro-uridine
  modified_base 11: mod_base = um
  modified_base 12: mod_base = um
  modified_base 13..15: mod_base = OTHER; note = linked via phosphorothioate
  modified_base 13: mod_base = cm
  modified_base 14: mod_base = OTHER; note = 2'-fluoro-adenosine
  modified_base 15: mod_base = um
  modified_base 16: mod_base = OTHER; note = 2'-fluoro-cytidine
  modified_base 17: mod_base = cm
  modified_base 18: mod_base = OTHER; note = 2'-O-methyl-adenosine
  modified_base 19: mod_base = OTHER; note = 2'-fluoro-cytidine
  modified_base 20..22: mod_base = OTHER; note = linked via phosphorothioate
  modified_base 20: mod_base = cm
  modified_base 21: mod_base = gm
  modified_base 22: mod_base = gm
  source 1..22: mol_type = other RNA
taaactgagtttcatccaccgg

SEQ ID NO: 47 - RNA, 22 nt, synthetic construct
  modified_base 1..4: mod_base = OTHER; note = linked via phosphorothioate
  modified_base 1: mod_base = OTHER; note = 4'-O-monomethylphosphonate-2'-O-methyluridine
  modified_base 2: mod_base = OTHER; note = 2'-fluoro-adenosine
  modified_base 3: mod_base = OTHER; note = 2'-fluoro-adenosine
  modified_base 4: mod_base = OTHER; note = 2'-fluoro-adenosine
  modified_base 5: mod_base = OTHER; note = 2'-fluoro-cytidine
  modified_base 6: mod_base = um
  modified_base 7: mod_base = OTHER; note = 2'-fluoro-guanosine
  modified_base 8: mod_base = OTHER; note = 2'-O-methyl-adenosine
  modified_base 9: mod_base = gm
  modified_base 10: mod_base = OTHER; note = 2'-fluoro-uridine
  modified_base 11: mod_base = um
  modified_base 12: mod_base = um
  modified_base 13..22: mod_base = OTHER; note = linked via phosphorothioate
  modified_base 13: mod_base = cm
  modified_base 14: mod_base = OTHER; note = 2'-fluoro-adenosine
  modified_base 15: mod_base = um
  modified_base 16: mod_base = OTHER; note = 2'-fluoro-cytidine
  modified_base 17: mod_base = cm
  modified_base 18: mod_base = OTHER; note = 2'-O-methyl-adenosine
  modified_base 19: mod_base = OTHER; note = 2'-fluoro-cytidine
  modified_base 20: mod_base = cm
  modified_base 21: mod_base = gm
  modified_base 22: mod_base = gm
  source 1..22: mol_type = other RNA
taaactgagtttcatccaccgg

SEQ ID NO: 48 - RNA, 22 nt, synthetic construct
  modified_base 1..4: mod_base = OTHER; note = linked via phosphorothioate
  modified_base 1: mod_base = OTHER; note = 4'-O-monomethylphosphonate-2'-O-methyluridine
  modified_base 2: mod_base = OTHER; note = 2'-fluoro-adenosine
  modified_base 3: mod_base = OTHER; note = 2'-fluoro-adenosine
  modified_base 4: mod_base = OTHER; note = 2'-fluoro-adenosine
  modified_base 5: mod_base = OTHER; note = 2'-fluoro-cytidine
  modified_base 6: mod_base = um
  modified_base 7: mod_base = OTHER; note = 2'-fluoro-guanosine
  modified_base 8: mod_base = OTHER; note = 2'-O-methyl-adenosine
  modified_base 9: mod_base = gm
  modified_base 10: mod_base = OTHER; note = 2'-fluoro-uridine
  modified_base 11: mod_base = um
  modified_base 12: mod_base = um
  modified_base 13..15: mod_base = OTHER; note = linked via phosphorothioate
  modified_base 13: mod_base = cm
  modified_base 14: mod_base = OTHER; note = 2'-fluoro-adenosine
  modified_base 15: mod_base = um
  modified_base 16: mod_base = cm
  modified_base 17: mod_base = cm
  modified_base 18..22: mod_base = OTHER; note = linked via phosphorothioate
  modified_base 18: mod_base = OTHER; note = 2'-O-methyl-adenosine
  modified_base 19: mod_base = OTHER; note = 2'-fluoro-cytidine
  modified_base 20: mod_base = cm
  modified_base 21: mod_base = gm
  modified_base 22: mod_base = gm
  source 1..22: mol_type = other RNA
taaactgagtttcatccaccgg

SEQ ID NO: 49 - RNA, 22 nt, synthetic construct
  modified_base 1..4: mod_base = OTHER; note = linked via phosphorothioate
  modified_base 1: mod_base = OTHER; note = 4'-O-monomethylphosphonate-2'-O-methyluridine
  modified_base 2: mod_base = OTHER; note = 2'-fluoro-adenosine
  modified_base 3: mod_base = OTHER; note = 2'-fluoro-adenosine
  modified_base 4: mod_base = OTHER; note = 2'-fluoro-adenosine
  modified_base 5: mod_base = OTHER; note = 2'-fluoro-cytidine
  modified_base 6: mod_base = um
  modified_base 7: mod_base = OTHER; note = 2'-fluoro-guanosine
  modified_base 8: mod_base = OTHER; note = 2'-O-methyl-adenosine
  modified_base 9: mod_base = gm
  modified_base 10: mod_base = OTHER; note = 2'-fluoro-uridine
  modified_base 11: mod_base = um
  modified_base 12: mod_base = um
  modified_base 13..15: mod_base = OTHER; note = linked via phosphorothioate
  modified_base 13: mod_base = cm
  modified_base 14: mod_base = OTHER; note = 2'-fluoro-adenosine
  modified_base 15: mod_base = um
  modified_base 16: mod_base = cm
  modified_base 17: mod_base = cm
  modified_base 18..19: mod_base = OTHER; note = linked via phosphorothioate
  modified_base 18: mod_base = OTHER; note = 2'-O-methyl-adenosine
  modified_base 19: mod_base = OTHER; note = 2'-fluoro-cytidine
  modified_base 20..22: mod_base = OTHER; note = linked via phosphorothioate
  modified_base 20: mod_base = cm
  modified_base 21: mod_base = gm
  modified_base 22: mod_base = gm
  source 1..22: mol_type = other RNA
taaactgagtttcatccaccgg

SEQ ID NO: 50 - RNA, 22 nt, synthetic construct
  modified_base 1..4: mod_base = OTHER; note = linked via phosphorothioate
  modified_base 1: mod_base = OTHER; note = 4'-O-monomethylphosphonate-2'-O-methyluridine
  modified_base 2: mod_base = OTHER; note = 2'-fluoro-adenosine
  modified_base 3: mod_base = OTHER; note = 2'-fluoro-adenosine
  modified_base 4: mod_base = OTHER; note = 2'-fluoro-adenosine
  modified_base 5: mod_base = OTHER; note = 2'-fluoro-cytidine
  modified_base 6: mod_base = um
  modified_base 7: mod_base = OTHER; note = 2'-fluoro-guanosine
  modified_base 8: mod_base = OTHER; note = 2'-O-methyl-adenosine
  modified_base 9: mod_base = gm
  modified_base 10: mod_base = OTHER; note = 2'-fluoro-uridine
  modified_base 11: mod_base = um
  modified_base 12: mod_base = um
  modified_base 13: mod_base = cm
  modified_base 14: mod_base = OTHER; note = 2'-fluoro-adenosine
  modified_base 15: mod_base = um
  modified_base 16: mod_base = cm
  modified_base 17: mod_base = cm
  modified_base 18: mod_base = OTHER; note = 2'-O-methyl-adenosine
  modified_base 19: mod_base = OTHER; note = 2'-fluoro-cytidine
  modified_base 20..22: mod_base = OTHER; note = linked via phosphorothioate
  modified_base 20: mod_base = cm
  modified_base 21: mod_base = gm
  modified_base 22: mod_base = gm
  source 1..22: mol_type = other RNA
taaactgagtttcatccaccgg

SEQ ID NO: 51 - RNA, 22 nt, synthetic construct
  modified_base 1..4: mod_base = OTHER; note = linked via phosphorothioate
  modified_base 1: mod_base = OTHER; note = 4'-O-monomethylphosphonate-2'-O-methyluridine
  modified_base 2: mod_base = OTHER; note = 2'-fluoro-adenosine
  modified_base 3: mod_base = OTHER; note = 2'-fluoro-adenosine
  modified_base 4: mod_base = OTHER; note = 2'-fluoro-adenosine
  modified_base 5: mod_base = OTHER; note = 2'-fluoro-cytidine
  modified_base 6: mod_base = um
  modified_base 7: mod_base = OTHER; note = 2'-fluoro-guanosine
  modified_base 8: mod_base = OTHER; note = 2'-O-methyl-adenosine
  modified_base 9: mod_base = gm
  modified_base 10: mod_base = OTHER; note = 2'-fluoro-uridine
  modified_base 11: mod_base = um
  modified_base 12: mod_base = um
  modified_base 13: mod_base = cm
  modified_base 14: mod_base = OTHER; note = 2'-fluoro-adenosine
  modified_base 15: mod_base = um
  modified_base 16: mod_base = cm
  modified_base 17: mod_base = cm
  modified_base 18: mod_base = OTHER; note = 2'-O-methyl-adenosine
  modified_base 19: mod_base = cm
  modified_base 20..22: mod_base = OTHER; note = linked via phosphorothioate
  modified_base 20: mod_base = um
  modified_base 21: mod_base = gm
  modified_base 22: mod_base = gm
  source 1..22: mol_type = other RNA
taaactgagtttcatccactgg

SEQ ID NO: 52 - RNA, 22 nt, synthetic construct
  modified_base 1..4: mod_base = OTHER; note = linked via phosphorothioate
  modified_base 1: mod_base = OTHER; note = 4'-O-monomethylphosphonate-2'-O-methyluridine
  modified_base 2: mod_base = OTHER; note = 2'-fluoro-adenosine
  modified_base 3: mod_base = OTHER; note = 2'-fluoro-adenosine
  modified_base 4: mod_base = OTHER; note = 2'-fluoro-adenosine
  modified_base 5: mod_base = OTHER; note = 2'-fluoro-cytidine
  modified_base 6: mod_base = um
  modified_base 7: mod_base = OTHER; note = 2'-fluoro-guanosine
  modified_base 8: mod_base = OTHER; note = 2'-O-methyl-adenosine
  modified_base 9: mod_base = gm
  modified_base 10: mod_base = OTHER; note = 2'-fluoro-uridine
  modified_base 11: mod_base = um
  modified_base 12: mod_base = um
  modified_base 13: mod_base = cm
  modified_base 14: mod_base = OTHER; note = 2'-fluoro-adenosine
  modified_base 15: mod_base = um
  modified_base 16: mod_base = cm
  modified_base 17: mod_base = cm
  modified_base 18: mod_base = OTHER; note = 2'-O-methyl-adenosine
  modified_base 19: mod_base = OTHER; note = 2'-fluoro-cytidine
  modified_base 20..22: mod_base = OTHER; note = linked via phosphorothioate
  modified_base 20: mod_base = um
  modified_base 21: mod_base = gm
  modified_base 22: mod_base = gm
  source 1..22: mol_type = other RNA
taaactgagtttcatccactgg

SEQ ID NO: 53 - RNA, 22 nt, synthetic construct
  modified_base 1..4: mod_base = OTHER; note = linked via phosphorothioate
  modified_base 1: mod_base = OTHER; note = 4'-O-monomethylphosphonate-2'-O-methyluridine
  modified_base 2: mod_base = OTHER; note = 2'-fluoro-adenosine
  modified_base 3: mod_base = OTHER; note = 2'-fluoro-adenosine
  modified_base 4: mod_base = OTHER; note = 2'-fluoro-adenosine
  modified_base 5: mod_base = OTHER; note = 2'-fluoro-cytidine
  modified_base 6: mod_base = um
  modified_base 7: mod_base = OTHER; note = 2'-fluoro-guanosine
  modified_base 8: mod_base = OTHER; note = 2'-O-methyl-adenosine
  modified_base 9: mod_base = gm
  modified_base 10: mod_base = OTHER; note = 2'-fluoro-uridine
  modified_base 11: mod_base = um
  modified_base 12: mod_base = um
  modified_base 13: mod_base = cm
  modified_base 14: mod_base = OTHER; note = 2'-fluoro-adenosine
  modified_base 15: mod_base = um
  modified_base 16: mod_base = cm
  modified_base 17: mod_base = cm
  modified_base 18: mod_base = OTHER; note = 2'-O-methyl-adenosine
  modified_base 19..22: mod_base = OTHER; note = linked via phosphorothioate
  modified_base 19: mod_base = OTHER; note = 2'-fluoro-cytidine
  modified_base 20: mod_base = um
  modified_base 21: mod_base = gm
  modified_base 22: mod_base = gm
  source 1..22: mol_type = other RNA
taaactgagtttcatccactgg

SEQ ID NO: 54 - RNA, 22 nt, synthetic construct
  modified_base 1..4: mod_base = OTHER; note = linked via phosphorothioate
  modified_base 1: mod_base = OTHER; note = 4'-O-monomethylphosphonate-2'-O-methyluridine
  modified_base 2: mod_base = OTHER; note = 2'-fluoro-uridine
  modified_base 3: mod_base = OTHER; note = 2'-fluoro-guanosine
  modified_base 4: mod_base = OTHER; note = 2'-fluoro-adenosine
  modified_base 5: mod_base = OTHER; note = 2'-fluoro-adenosine
  modified_base 6: mod_base = cm
  modified_base 7: mod_base = OTHER; note = 2'-fluoro-uridine
  modified_base 8: mod_base = um
  modified_base 9: mod_base = cm
  modified_base 10: mod_base = OTHER; note = 2'-fluoro-adenosine
  modified_base 11: mod_base = cm
  modified_base 12: mod_base = cm
  modified_base 13: mod_base = um
  modified_base 14: mod_base = OTHER; note = 2'-fluoro-uridine
  modified_base 15: mod_base = gm
  modified_base 16: mod_base = OTHER; note = 2'-O-methyl-adenosine
  modified_base 17: mod_base = um
  modified_base 18: mod_base = gm
  modified_base 19: mod_base = gm
  modified_base 20..22: mod_base = OTHER; note = linked via phosphorothioate
  modified_base 20: mod_base = um
  modified_base 21: mod_base = gm
  modified_base 22: mod_base = gm
  source 1..22: mol_type = other RNA
ttgaacttcaccttgatggtgg

SEQ ID NO: 55 - RNA, 22 nt, synthetic construct
  modified_base 1..4: mod_base = OTHER; note = linked via phosphorothioate
  modified_base 1: mod_base = OTHER; note = 4'-O-monomethylphosphonate-2'-O-methyluridine
  modified_base 2: mod_base = OTHER; note = 2'-fluoro-uridine
  modified_base 3: mod_base = OTHER; note = 2'-fluoro-guanosine
  modified_base 4: mod_base = OTHER; note = 2'-fluoro-adenosine
  modified_base 5: mod_base = OTHER; note = 2'-fluoro-adenosine
  modified_base 6: mod_base = cm
  modified_base 7: mod_base = OTHER; note = 2'-fluoro-uridine
  modified_base 8: mod_base = um
  modified_base 9: mod_base = cm
  modified_base 10: mod_base = OTHER; note = 2'-fluoro-adenosine
  modified_base 11: mod_base = cm
  modified_base 12: mod_base = cm
  modified_base 13: mod_base = um
  modified_base 14: mod_base = OTHER; note = 2'-fluoro-uridine
  modified_base 15: mod_base = gm
  modified_base 16: mod_base = OTHER; note = 2'-O-methyl-adenosine
  modified_base 17: mod_base = um
  modified_base 18: mod_base = gm
  modified_base 19: mod_base = OTHER; note = 2'-fluoro-guanosine
  modified_base 20..22: mod_base = OTHER; note = linked via phosphorothioate
  modified_base 20: mod_base = um
  modified_base 21: mod_base = gm
  modified_base 22: mod_base = gm
  source 1..22: mol_type = other RNA
ttgaacttcaccttgatggtgg

SEQ ID NO: 56 - RNA, 22 nt, synthetic construct
  modified_base 1..4: mod_base = OTHER; note = linked via phosphorothioate
  modified_base 1: mod_base = OTHER; note = 4'-O-monomethylphosphonate-2'-O-methyluridine
  modified_base 2: mod_base = OTHER; note = 2'-fluoro-uridine
  modified_base 3: mod_base = OTHER; note = 2'-fluoro-guanosine
  modified_base 4: mod_base = OTHER; note = 2'-fluoro-adenosine
  modified_base 5: mod_base = OTHER; note = 2'-fluoro-adenosine
  modified_base 6: mod_base = cm
  modified_base 7: mod_base = OTHER; note = 2'-fluoro-uridine
  modified_base 8: mod_base = um
  modified_base 9: mod_base = cm
  modified_base 10: mod_base = OTHER; note = 2'-fluoro-adenosine
  modified_base 11: mod_base = cm
  modified_base 12: mod_base = cm
  modified_base 13: mod_base = um
  modified_base 14: mod_base = OTHER; note = 2'-fluoro-uridine
  modified_base 15: mod_base = gm
  modified_base 16: mod_base = OTHER; note = 2'-O-methyl-adenosine
  modified_base 17: mod_base = um
  modified_base 18: mod_base = gm
  modified_base 19..22: mod_base = OTHER; note = linked via phosphorothioate
  modified_base 19: mod_base = OTHER; note = 2'-fluoro-guanosine
  modified_base 20: mod_base = um
  modified_base 21: mod_base = gm
  modified_base 22: mod_base = gm
  source 1..22: mol_type = other RNA
ttgaacttcaccttgatggtgg

SEQ ID NO: 57 - RNA, 22 nt, synthetic construct
  modified_base 1..4: mod_base = OTHER; note = linked via phosphorothioate
  modified_base 1: mod_base = OTHER; note = 4'-O-monomethylphosphonate-2'-O-methyluridine
  modified_base 2: mod_base = OTHER; note = 2'-fluoro-uridine
  modified_base 3: mod_base = OTHER; note = 2'-fluoro-uridine
  modified_base 4: mod_base = OTHER; note = 2'-fluoro-adenosine
  modified_base 5: mod_base = OTHER; note = 2'-fluoro-adenosine
  modified_base 6: mod_base = gm
  modified_base 7: mod_base = OTHER; note = 2'-fluoro-cytidine
  modified_base 8: mod_base = um
  modified_base 9: mod_base = gm
  modified_base 10: mod_base = OTHER; note = 2'-fluoro-adenosine
  modified_base 11: mod_base = um
  modified_base 12: mod_base = OTHER; note = 2'-O-methyl-adenosine
  modified_base 13: mod_base = OTHER; note = 2'-O-methyl-adenosine
  modified_base 14: mod_base = OTHER; note = 2'-fluoro-uridine
  modified_base 15: mod_base = um
  modified_base 16: mod_base = cm
  modified_base 17: mod_base = OTHER; note = 2'-O-methyl-adenosine
  modified_base 18: mod_base = OTHER; note = 2'-O-methyl-adenosine
  modified_base 19: mod_base = cm
  modified_base 20..22: mod_base = OTHER; note = linked via phosphorothioate
  modified_base 20: mod_base = um
  modified_base 21: mod_base = gm
  modified_base 22: mod_base = gm
  source 1..22: mol_type = other RNA
tttaagctgataattcaactgg

SEQ ID NO: 58 - RNA, 22 nt, synthetic construct
  modified_base 1..4: mod_base = OTHER; note = linked via phosphorothioate
  modified_base 1: mod_base = OTHER; note = 4'-O-monomethylphosphonate-2'-O-methyluridine
  modified_base 2: mod_base = OTHER; note = 2'-fluoro-uridine
  modified_base 3: mod_base = OTHER; note = 2'-fluoro-uridine
  modified_base 4: mod_base = OTHER; note = 2'-fluoro-adenosine
  modified_base 5: mod_base = OTHER; note = 2'-fluoro-adenosine
  modified_base 6: mod_base = gm
  modified_base 7: mod_base = OTHER; note = 2'-fluoro-cytidine
  modified_base 8: mod_base = um
  modified_base 9: mod_base = gm
  modified_base 10: mod_base = OTHER; note = 2'-fluoro-adenosine
  modified_base 11: mod_base = um
  modified_base 12: mod_base = OTHER; note = 2'-O-methyl-adenosine
  modified_base 13: mod_base = OTHER; note = 2'-O-methyl-adenosine
  modified_base 14: mod_base = OTHER; note = 2'-fluoro-uridine
  modified_base 15: mod_base = um
  modified_base 16: mod_base = cm
  modified_base 17: mod_base = OTHER; note = 2'-O-methyl-adenosine
  modified_base 18: mod_base = OTHER; note = 2'-O-methyl-adenosine
  modified_base 19: mod_base = OTHER; note = 2'-fluoro-cytidine
  modified_base 20..22: mod_base = OTHER; note = linked via phosphorothioate
  modified_base 20: mod_base = um
  modified_base 21: mod_base = gm
  modified_base 22: mod_base = gm
  source 1..22: mol_type = other RNA
tttaagctgataattcaactgg

(19) United States
(12) Patent Application Publication
(10) Pub. No.: US 2026/0049309 A1
(43) Pub. Date: Feb. 19, 2026

(54) REGULATION OF ACTIVITY OF RNAI MOLECULES

(71) Applicant: NOVO NORDISK A/S, Bagsvaerd (DE)

(72) Inventors: Bob Dale Brown, Littleton, MA (US); Marc Abrams, Natick, MA (US); Martin Lee Koser, Westford, MA (US)

(21) Appl. No.: 19/103,843

(22) PCT Filed: Aug. 15, 2023
(86) PCT No.: PCT/US23/72194

§ 371 Date: Feb. 14, 2025

Related U.S. Application Data
(60) Provisional application No. 63/398,094, filed on Aug. 15, 2022.

(51) Int. Cl. C12N 15/113; A61K 31/711; A61K 31/712; A61K 31/713

(52) U.S. Cl. CPC C12N 15/113; A61K 31/711; A61K 31/712; A61K 31/713; C12N 2310/11; C12N 2310/14; C12N 2310/312; C12N 2310/315; C12N 2310/321; C12N 2310/322; C12N 2310/3515

(57) ABSTRACT

Double-stranded oligonucleotides are provided herein that provide increased inhibition or reduction of expression of target genes in extra-hepatic tissue compared to hepatocytes. Also provided are compositions including the same and uses thereof, particularly uses relating to treating diseases, disorders and/or conditions associated with an RNAi trigger induced decrease in target gene expression.

### CROSS REFERENCE TO RELATED APPLICATIONS

This application claims the benefit of and relies on the filing date of U.S. Provisional Application No. 63/398,094, filed 15 Aug. 2022, the entire disclosure of each application is incorporated herein by reference.

### REFERENCE TO AN ELECTRONIC SEQUENCE LISTING

The contents of the electronic sequence listing (DICN_022_01WO_SeqList_ST26.xml; Size: 521,339 bytes; and Date of Creation: Aug. 11, 2023) are herein incorporated by reference in its entirety.

### TECHNICAL FIELD

The disclosure relates to oligonucleotides useful in the inhibition of target genes in a variety of tissues. In some embodiments, the present disclosure relates to oligonucleotide-lipid conjugates, methods to prepare them, their chemical configuration, and methods to modulate (e.g., inhibit or reduce) the expression of a target gene using the conjugated nucleic acids and oligonucleotides according to the description provided herein. The disclosure also provides pharmaceutically acceptable compositions comprising the conjugates of the present description and methods of using said compositions in the treatment of various diseases or disorders.

### BACKGROUND OF THE DISCLOSURE

Regulation of gene expression by modified nucleic acids shows great potential as both a research tool in the laboratory and a therapeutic approach in the clinic. Several classes of oligonucleotide or nucleic acid-based therapeutics have been under the clinical investigation, including antisense oligonucleotides (ASO), short interfering RNA (siRNA), double-stranded nucleic acids (dsNA), aptamers, ribozymes, exon-skipping and splice-altering oligonucleotides, immunomodulatory oligonucleotides, mRNAs, and CRISPR. Chemical modifications in the relevant molecules to allow functionality in various tissues, organs and/or cell types play a key role in overcoming challenges of oligonucleotide therapeutics, including improving nuclease stability, RNA-binding affinity, and pharmacokinetics. Various chemical modification strategies for oligonucleotides have been developed in the past three decades including modification of the sugars, nucleobases, and phosphodiester backbone to improve and optimize performance and therapeutic efficacy (Deleavey and Darma, CHEM. BIOL. 2012, 19 (8): 937-54; Wan and Seth, J. MED. CHEM. 2016, 59 (21): 9645-67; and Egli and Manoharan, ACC. CHEM. RES. 2019, 54 (4): 1036-47).

Dicer processed RNAi technologies utilize short double-stranded RNA (dsRNA) of approximately 21 base pair length with a two nucleotide (nt) 3′-overhang for the silencing of genes. These dsRNAs are generally called small interfering RNA (siRNA). siRNA 12 to 22 nucleotides in length are the active agent in RNAi. The siRNA duplex serves as a guide for mRNA degradation. Upon siRNA incorporation into the RNA-induced silencing complex (RISC) the complex interacts with a specific mRNA and ultimately suppresses the mRNA signal. The sense strand or passenger strand of siRNA is typically cleaved at the 9th nucleotide downstream from the 5′-end of the sense strand by Argonaute 2 (Ago2) endonuclease. The activated RISC complex containing the antisense strand or guide strand binds to the target mRNA through Watson-Crick base pairing causing degradation or translational blocking of the targeted RNA.

However, the in vivo use of RNAi or siRNA molecules as pharmaceuticals has remained difficult due to obstacles encountered such as low biostability and unacceptable toxicity possibly caused by off-target effects. Various types of chemical modifications to improve the pharmacokinetics and to overcome bio-instability problems have been investigated over the years to improve the stability and specificity of the RNAi duplexes. In some cases, the chemical modification in siRNAs has improved the serum stability of siRNAs. However, often RNAi activity was lost, but the careful placement of some specific modified residues enables enhanced siRNA biostability without loss of siRNA potency. Some of these modifications have reduced siRNA side effects, such as the induction of recipient immune responses and inherent off-targeting effects and have even enhanced siRNA potency. Various chemically modified siRNAs have been investigated, among them were bridged nucleic acids (BNA's) such as 2′,4′-methylene bridged nucleic acid 2′,4′-BNAs, also known as locked nucleic acid or LNA's. Some of these modified siRNAs showed promising effects.

Therapeutic gene silencing mediated by RNAi oligonucleotide-based therapeutics comprising siRNAs or double-stranded nucleic acids (dsNAs) offer the potential for considerable expansion of the druggable target space and the possibility for treating orphan diseases that may be therapeutically unapproachable by other drug modalities (e.g., antibodies and/or small molecules). RNAi oligonucleotide-based therapeutics that inhibit or reduce expression of specific target genes in the liver have been developed and are currently in clinical use (Sehgal et al., (2013) JOURNAL OF HEPATOLOGY 59:1354-59). Technological hurdles remain for the development and clinical use of RNAi oligonucleotides in extrahepatic cells, tissues, and organs. Thus, an ongoing need exists in the art for the successful development of new and effective RNAi oligonucleotides to modulate the expression of a target genes in extrahepatic cells, tissues, and/or organs. This is complicated by the variant nature of the cell types in extrahepatic as well as concerns about circulatory patterns and cell membrane constituents such as receptor types.

Over the past decade, synthetic RNAi triggers such as double stranded RNAs have become ubiquitous tools in biological research, and extensive basic and clinical development efforts have recently culminated in the FDA approval of ONPATTRO™, the first RNAi drug. Despite a burgeoning drug development pipeline and an extensive compendium of excipients targeting ligands and delivery techniques, the difficulty of delivering RNAi agents to specific populations of disease related cells and or tissues, particularly outside the liver continues to limit the potential of RNAi therapy. Repeated attempts over the past several years to develop useful, active, and persistent RNAi agents and structures for use based on known liver delivery technology have not convincingly demonstrated the intended effects outside the liver. Thus, new dsRNA's with variant structures have been developed to overcome the limitations in the field.

### BRIEF SUMMARY OF THE DISCLOSURE

The present disclosure is based, in part, on the discovery of double-stranded oligonucleotides capable of reducing or inhibiting expression of a target gene in extra-hepatic tissues while having reduced inhibition in hepatocytes. As demonstrated herein, double-stranded oligonucleotides having an antisense strand comprising a 3′ overhang of at least 4 nucleotides wherein the overhang comprises at least one 2′-F modified nucleotide showed improved efficacy and duration in in extra-hepatic tissues, including skeletal muscle, adipose tissue, and adrenal tissue, relative to hepatocytes. Specifically, target gene expression was reduced in extra-hepatic tissue by the double-stranded oligonucleotides at a higher amount than reduction of expression of the same target gene in hepatocytes, e.g., reduction by 95% in extra-hepatic tissue compared to reduction by 20% in hepatocytes.

Accordingly, in some aspects, the disclosure provides a double-stranded oligonucleotide comprising an antisense strand of 15-30 nucleotides in length and a sense strand of about 9-50 nucleotides in length, wherein (i) the antisense and sense strands form a duplex region of about 9-26 base pairs, (ii) the antisense strand comprises an orientation of 5′ to 3′, (iii) the antisense strand comprises a 3′ overhang of at least four nucleotides, (iv) the antisense strand comprises a region of complementarity to an mRNA target sequence in a target mRNA, (v) the 3′ overhang comprises a sequence motif that reduces inhibition of the target mRNA by the double-stranded oligonucleotide in a cell of the liver, and (vi) the sequence motif comprises at least one 2′-F modified nucleotide, provided the at least one 2′-F modified nucleotide is not one of the two 3′terminal nucleotides of the antisense strand.

In some embodiments, inhibition of the target mRNA is reduced compared to inhibition of the target mRNA by a double-stranded oligonucleotide not having the sequence motif. In some embodiments, the cell of the liver is a hepatocyte.

In some or any of the foregoing or related embodiments, the double-stranded oligonucleotides described herein reduce target mRNA expression in an extra-hepatic tissue by 50-100% and reduce target mRNA expression in a hepatocyte by 5-45%. In some or any of the foregoing or related embodiments, the double-stranded oligonucleotides described herein reduce target mRNA expression in an extra-hepatic tissue by 40-90% and reduce target mRNA expression in a hepatocyte by 5-25%.

In some or any of the foregoing or related embodiments, the sequence motif comprises:

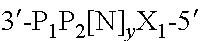

wherein:
P₁ and P₂ are each independently a purine or a pyrimidine, and do not comprise a 2′-F modification;
X₁ is any nucleotide located immediately adjacent to the duplex, and comprises at least one phosphorothioate linkage to an adjacent nucleotide;
N is any nucleotide and y is an integer selected from 1-6, specifying N₁-N₆, wherein:
   (a) when y is 1, N₁ comprises a 2′-F modification;
   (b) when y is 2, one or more of N₁ and N₂ comprise a 2′-F modification;
   (c) when y is 3, one or more of N₁-N₃ comprise a 2′-F modification;
   (d) when y is 4, one or more of N₁-N₄ comprise a 2′-F modification;
   (e) when y is 5, one or more of N₁-N₅ comprise a 2′-F modification; or
   (f) when y is 6, one or more of N₁-N₆ comprise a 2′-F modification.

In other aspects, the disclosure provides a double-stranded oligonucleotide for inhibiting a target mRNA in a cell of an extra-hepatic tissue comprising an antisense strand of 15-30 nucleotides in length and a sense strand of about 9-50 nucleotides in length, wherein (i) the antisense and sense strands form a duplex region of about 9-26 base pairs, (ii) the antisense strand comprises an orientation of 5′ to 3′, (iii) the antisense strand comprises a 3′ overhang of at least 4 nucleotides, (iv) the antisense strand comprises a region of complementarity to a mRNA target sequence in a target mRNA, and (v) the antisense strand comprises at least one 2′-F modified nucleotide in the 3′ overhang, provided the at least one 2′-F modified nucleotide is not one of the two 3′ terminal nucleotides of the antisense strand.

In further aspects, the disclosure provides a double-stranded oligonucleotide for increasing inhibition of a target mRNA in a cell of an extra-hepatic tissue relative to inhibition of the target mRNA in a cell of liver tissue, comprising an antisense strand of 15-30 nucleotides in length and a sense strand of about 9-50 nucleotides in length, wherein (i) the antisense and sense strands form a duplex region of about 9-26 base pairs, (ii) the antisense strand comprises an orientation of 5′ to 3′, (iii) the antisense strand comprises a 3′ overhang of at least 4 nucleotides, (iv) the antisense strand comprises a region of complementarity to a mRNA target sequence in a target mRNA, and (v) the antisense strand comprises at least one 2′-F modified nucleotide in the 3′ overhang, provided the at least one 2′-F modified nucleotide is not one of the two 3′ terminal nucleotides of the antisense strand.

In some embodiments, the extra-hepatic tissue is selected from skeletal muscle, adipose tissue, adrenal tissue, and any combination thereof. In some embodiments, the cell of the cell of the extra-hepatic tissue is selected from a cardiomyocyte, an immune cell, a liver non-parenchymal cell, a cell of skeletal muscle, a cell of adipose tissue, a cell of adrenal tissue, and any combination thereof. In some embodiments,

In some or any of the foregoing or related embodiments, the 3′ overhang comprises a sequence motif of: 3′-P₁P₂[N]_{y}X₁-5′

wherein
P₁ and P₂ are each independently a purine or a pyrimidine, and do not comprise a 2′-F modification;
X₁ is any nucleotide located immediately adjacent to the duplex, and comprises at least one phosphorothioate linkage to an adjacent nucleotide;
N is any nucleotide and y is an integer selected from 1-6, specifying N₁-N₆, wherein:
   (a) when y is 1, N₁ comprises a 2′-F modification;
   (b) when y is 2, one or more of N₁ and N₂ comprise a 2′-F modification;
   (c) when y is 3, one or more of N₁-N₃ comprise a 2′-F modification;
   (d) when y is 4, one or more of N₁-N₄ comprise a 2′-F modification;
   (e) when y is 5, one or more of N₁-N₅ comprise a 2′-F modification; or
   (f) when y is 6, one or more of N₁-N₆ comprise a 2′-F modification.

In some or any of the foregoing or related embodiments, when y is 3, N₂ comprises the 2′-F modification. In some embodiments, X₁ does not comprise a 2′-F modification. In some embodiments, N₁, N₃, and N₄ each comprise a 2′-OMe modification.

In some or any of the foregoing or related embodiments, when y is 6, N₂ comprises the 2′-F modification. In some embodiments, X₁ comprises a 2′-F modification. In some embodiments, N₁, N₃, N₄. N₅, and N₆ each comprise a 2′-OMe modification.

In some or any of the foregoing or related embodiments, when y is 6, N₂ and N₅ each comprise the 2′-F modification. In some embodiments, X₁ comprises a 2′-F modification. In some embodiments, N₁, N₃, N₄ and No each comprise a 2′-OMe modification.

In some or any of the foregoing or related embodiments, P₁ and P₂ are each independently a purine. In some embodiments, P₁ and P₂ are each independently selected from adenosine and guanine. In some embodiments, P₁ and P₂ are each guanine.

In some or any of the foregoing or related embodiments, the double-stranded oligonucleotide reduces expression of the target mRNA in an extra-hepatic cell, provided the double-stranded oligonucleotide does not reduce expression of the mRNA target in a cell of the liver.

In some or any of the foregoing or related embodiments, the antisense strand is 20-22 nucleotides, and the 2′-F modified nucleotide is at position 19. In other embodiments, the antisense strand is 20-22 nucleotides, and the 2′-F modified nucleotide is at position 16. In some embodiments, the antisense strand is 20-22 nucleotides, and the 2′-F modified nucleotide is at position 16 and position 19.

In some or any of the foregoing or related embodiments, the antisense strand is 20-22 nucleotides, the 3′ overhang is 4-9 nucleotides, and the 2′-F modified nucleotide is at position 19. In other embodiments, the antisense strand is 20-22 nucleotides, the 3′ overhang is 6-9 nucleotides, and the 2′-F modified nucleotide is at position 16. In some embodiments, the antisense strand is 20-22 nucleotides, the 3′ overhang is 6-9 nucleotides, and the 2′-F modified nucleotide is at position 16 and position 19.

In some or any of the foregoing or related embodiments, the antisense strand is 22 nucleotides.

In some or any of the foregoing or related embodiments:
(i) the sense strand is 29 nucleotides, the antisense strand is 22 nucleotides, and the duplex region is 13 nucleotides;
(ii) the sense strand is 23 nucleotides, the antisense strand is 22 nucleotides, and the duplex region is 13 nucleotides;
(iii) the sense strand is 30 nucleotides, the antisense strand is 22 nucleotides, and the duplex region is 14 nucleotides;
(iv) the sense strand is 31 nucleotides, the antisense strand is 22 nucleotides, and the duplex region is 15 nucleotides;
(v) the sense strand is 32 nucleotides, the antisense strand is 22 nucleotides, and the duplex region is 16 nucleotides; or
(vi) the sense strand is 16 nucleotides, the antisense strand is 22 nucleotides, and the duplex region is 16 nucleotides.

In some or any of the foregoing or related embodiments, the 2′-F modified nucleotide comprises a phosphorothioate linkage. In some embodiments, the nucleotides adjacent to the 2′-F modified nucleotide do not have phosphorothioate linkages.

In some or any of the foregoing or related embodiments, the sense strand comprises at least one lipid moiety conjugated to a nucleotide on the sense strand. In some embodiments, the lipid moiety is selected from:

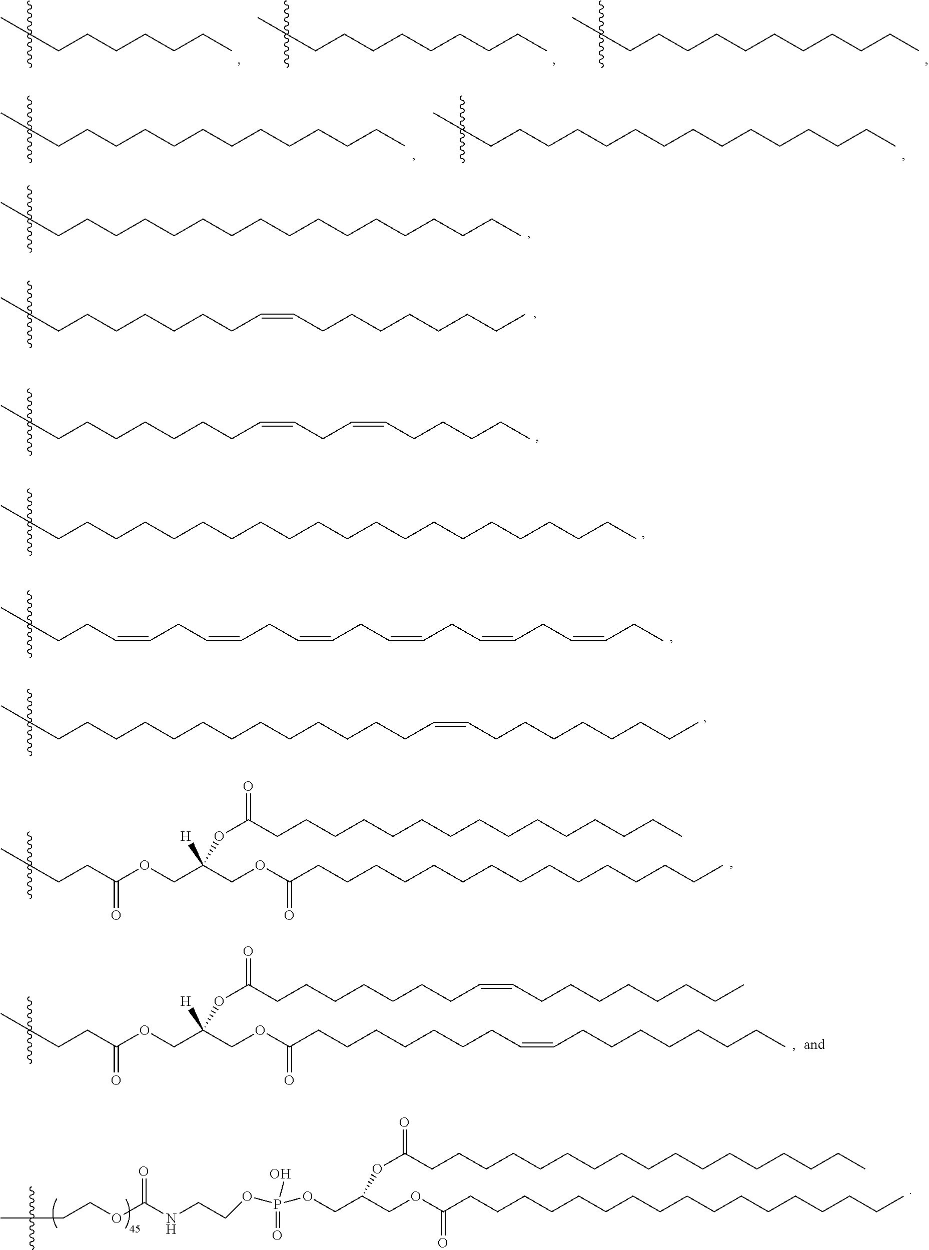

In some embodiments, the lipid moiety is a hydrocarbon chain. In some embodiments, the hydrocarbon chain is a C8-C30 hydrocarbon chain. In some embodiments, the hydrocarbon chain is a C16 hydrocarbon chain. In some embodiments, the C16 hydrocarbon chain is represented by

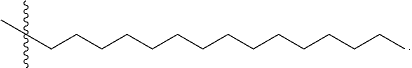 [SMILES: *CCCCCCCCCCCCCCC]

In some embodiments, the hydrocarbon chain is a C22 hydrocarbon chain. In some embodiments, the C22 hydrocarbon chain is represented by

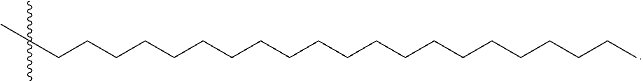 [SMILES: *CCCCCCCCCCCCCCCCCCCCC]

In some embodiments, the lipid moiety is conjugated to the 5′terminal nucleotide of the sense strand. In some embodiments, the sense strand comprises a stem-loop, and wherein the lipid moiety is conjugated to a nucleotide of the stem-loop. In some embodiments, the lipid moiety is conjugated to the 2′ carbon of the ribose ring of the nucleotide.

In some or any of the foregoing or related embodiments, the sense strand comprises a stem-loop. In some embodiments, the stem-loop comprises a nucleotide sequence represented by the formula: 5′-S1-L-S2-3′, wherein S1 is complementary to S2, and wherein L forms a loop between S1 and S2. In some embodiments, S1 and S2 are each independently 1-20 nucleotides in length, optionally wherein S1 and S2 are the same length. In some embodiments, L is a triloop or a tetraloop. In some embodiments, the tetraloop comprises the sequence 5′-GAAA-3′. In some embodiments, the stem-loop comprises the sequence 5′-GCAGCCGAAAGGCUGC-3′ (SEQ ID NO: 15).

In some or any of the foregoing or related embodiments, the double-stranded oligonucleotide comprises a blunt end. In some embodiments, the blunt end comprises the 3′ end of the sense strand and the 5′ end of the antisense strand.

In some or any of the foregoing or related embodiments, the double-stranded oligonucleotide comprises an overhang at the 5′ end of the antisense strand. In some embodiments, the overhang at the 5′ end of the antisense strand is 2-6 nucleotides in length.

In some aspects, the disclosure provides a double-stranded oligonucleotide comprising an antisense strand of 15-30 nucleotides in length and a sense strand of about 9-50 nucleotides in length, wherein (i) the antisense and sense strands form a duplex region of about 9-26 base pairs, (ii) the antisense strand comprises an orientation of 5′ to 3′, (iii) the antisense strand comprises a 3′ overhang of at least four nucleotides and a 5′ overhang of at least 2 nucleotides, (iv) the antisense strand comprises a region of complementarity to an mRNA target sequence in a target mRNA, (v) the 3′ overhang comprises a sequence motif that reduces inhibition of the target mRNA by the double-stranded oligonucleotide in a cell of the liver, and (vi) the sequence motif comprises at least one 2′-F modified nucleotide, provided the at least one 2′-F modified nucleotide is not one of the two 3′terminal nucleotides of the antisense strand. In some embodiments, the antisense strand is 22 nucleotides, wherein the 3′ overhang is 4-9 nucleotides, and wherein the 2′-F nucleotide is located at position 19. In some embodiments, the antisense strand is 22 nucleotides, wherein the 3′ overhang is 6-9 nucleotides, and wherein the 2′-F nucleotide is located at position 16. In some embodiments, the antisense strand is 22 nucleotides, wherein the 3′ overhang is 6-9 nucleotides, and wherein the 2′-F nucleotide is located at position 16 and position 19. In some embodiments, the sense strand comprises a lipid moiety conjugated to a nucleotide of the sense strand.

In some or any of the foregoing or related embodiments, the region of complementarity is fully complementary to the mRNA target sequence. In other embodiments, the region of complementarity is partially complementary to the mRNA target sequence. In some embodiments, the region of complementarity comprises no more than four mismatches to the mRNA target sequence.

In some or any of the foregoing or related embodiments, the sense strand comprises at least one modified nucleotide. In some embodiments, the antisense strand comprises at least one modified nucleotide in addition to the 2′-F modified nucleotide. In some embodiments, the modified nucleotide comprises a 2′-modification. In some embodiments, the 2′-modification is a modification selected from 2′-aminoethyl, 2′-fluoro, 2′-O-methyl, 2′-O-methoxyethyl, and 2′-deoxy-2′-fluoro-β-d-arabinonucleic acid. In some embodiments, the sense strand comprises a 2′-fluoro modification at each of nucleotides forming a base pair with nucleotides at positions 10-13 of the antisense strand. In some embodiments, the sense strand comprises a 2′-fluoro modification at each of nucleotides forming a base pair with nucleotides at positions 10-12 of the antisense strand. In some embodiments, the sense strand comprises 16-32 nucleotides, wherein nucleotides at each of positions 3, 5, 6, 8, and 10 comprise a 2′-fluoro modification. In some embodiments, the sense strand comprises 16-32 nucleotides, wherein nucleotides at each of positions 4-7 comprise a 2′-fluoro modification. In some embodiments, the antisense strand comprises 22 nucleotides, and wherein each of positions 2-5, 7, 10, and 13 comprise a 2′-fluoro modification. In some embodiments, the remaining nucleotides comprise a 2′-O-methyl modification, provided the nucleotide of the sense strand conjugated to the at least one lipid moiety does not comprise a 2′-O-methyl modification.

In some or any of the foregoing or related embodiments, the double-stranded oligonucleotide comprises at least one modified internucleotide linkage. In some embodiments, the at least one modified internucleotide linkage is a phosphorothioate linkage. In some embodiments, the antisense strand comprises a phosphorothioate linkage (i) between positions 1 and 2, and between positions 2 and 3; or (ii) between positions 1 and 2, between positions 2 and 3, and between positions 3 and 4. In some embodiments, the antisense strand is 22 nucleotides in length, and wherein the antisense strand comprises a phosphorothioate linkage between positions 20 and 21 and between positions 21 and 22. In some embodiments, the sense strand comprises a phosphorothioate linkage between positions 1 and 2.

In some or any of the foregoing or related embodiments, the antisense strand comprises a phosphorylated nucleotide at the 5′ terminus, wherein the phosphorylated nucleotide is selected from uridine and adenosine. In some embodiments, the phosphorylated nucleotide is uridine. In some embodiments, the 4′-carbon of the sugar of the 5′-nucleotide of the antisense strand comprises a phosphate analog. In some embodiments, the phosphate analog is oxymethyl phosphonate, vinyl phosphonate or malonyl phosphonate. In some embodiments, the phosphorylated nucleotide is 4′-O-monomethylphosphonate-2′-O-methyl uridine.

In some or any of the foregoing or related embodiments, the sense strand comprises at least one Tm-increasing nucleotide. In some embodiments, the sense strand comprises up to four Tm-increasing nucleotides. In some embodiments, the 5′ terminal nucleotide of the sense strand is a Tm-increasing nucleotide. In some embodiments, the sense strand comprises a stem-loop, and wherein the stem comprises at least one pair of Tm-increasing nucleotides. In some embodiments, the Tm-increasing nucleotide is a bicyclic nucleotide. In some embodiments, the Tm-increasing nucleotide is a locked nucleic acid.

In some or any of the foregoing or related embodiments, the double-stranded oligonucleotide is a Dicer substrate.

In some aspects, the disclosure provides a pharmaceutical composition comprising a double-stranded oligonucleotide described herein, and a pharmaceutically acceptable carrier, delivery agent, or excipient.

In other aspects, the disclosure provides a method of inhibiting target mRNA expression in a cell of an extra-hepatic tissue in a subject, comprising administering to the subject a double-stranded oligonucleotide or pharmaceutical composition described herein, thereby inhibiting target mRNA expression in the cell of the extra-hepatic tissue. In some embodiments, the extra-hepatic tissue is selected from skeletal muscle, adipose tissue, adrenal tissue, and any combination thereof. In some embodiments, the cell of the cell of the extra-hepatic tissue is selected from a cardiomyocyte, an immune cell, a liver non-parenchymal cell, a cell of skeletal muscle, a cell of adipose tissue, a cell of adrenal tissue, and any combination thereof. In some embodiments, reduction of the target mRNA in the cell of the extra-hepatic tissue is increased compared to reduction in a cell of the liver, optionally wherein reduction of the target mRNA is increased by at least 10%. In some embodiments, reduction of the target mRNA is increased by at least 15%, at least 20%, at least 25%, at least 30%, at least 35%, at least 40%, at least 45% or at least 50%. In some embodiments, the cell of the liver is a hepatocyte.

In some aspects, the disclosure provides use of a double-stranded oligonucleotide described herein for inhibiting target mRNA expression in a cell of an extra-hepatic tissue. In other aspects, the disclosure provides a double-stranded oligonucleotide described herein for use as a medicament for inhibiting target mRNA expression in a cell of an extra-hepatic tissue.

### BRIEF DESCRIPTION OF FIGURES

The patent or application file contains at least one drawing executed in color. Copies of this patent or patent application publication with color drawing(s) will be provided by the Office upon request and payment of the necessary fee.

FIG. 1 provides schematics of RNAi oligonucleotide-lipid conjugates targeting Aldh2 mRNA having the structures of Compounds 1-4.

FIGS. 2A-2D provide graphs measuring percent (%) murine Aldh2 mRNA remaining in liver (FIG. 2A), skeletal muscle (FIG. 2B), adipose tissue (FIG. 2C), and adrenal tissue (FIG. 2D) 14 days after subcutaneous injection in control mice (group A administered PBS) or mice administered Compounds 1˜4 (respectively groups B-E).

FIGS. 2E-2H provide graphs measuring percent (%) murine Aldh2 mRNA remaining in liver (FIG. 2E), skeletal muscle (FIG. 2F), adipose tissue (FIG. 2G), and adrenal tissue (FIG. 2H) 28 days after subcutaneous injection in control mice (group A administered PBS) or mice administered Compounds 1˜4 (respectively groups B-E).

FIGS. 21-2L provide graphs measuring percent (%) murine Aldh2 mRNA remaining in liver (FIG. 2I), skeletal muscle (FIG. 2J), adipose tissue (FIG. 2K), and adrenal tissue (FIG. 2L) 14 and 35 days after subcutaneous injection in control mice (group A administered PBS) or mice administered Compounds 1˜4 (respectively groups B-E).

FIG. 3 provides schematics of RNAi oligonucleotide-lipid conjugates targeting Aldh2 mRNA having the structures of Compounds 1, and 5-9.

FIGS. 4A-4D provide graphs measuring percent (%) murine Aldh2 mRNA remaining in liver (FIG. 4A), skeletal muscle (FIG. 4B), adipose tissue (FIG. 4C), and adrenal tissue (FIG. 4D) 14 days after subcutaneous injection in control mice (group A administered PBS) or mice administered Compounds 1, and 5-9 (respectively groups B-G).

FIGS. 5A-5D provide graphs measuring percent (%) murine Aldh2 mRNA remaining in liver (FIG. 5A), skeletal muscle (FIG. 5B), adipose tissue (FIG. 5C), and adrenal tissue (FIG. 5D) 35 days after subcutaneous injection in control mice (group A administered PBS) or mice administered Compounds 1, and 5-9 (respectively groups B-G).

FIGS. 6A-6D provide graphs measuring percent (%) murine Aldh2 mRNA remaining in liver (FIG. 6A), skeletal muscle (FIG. 6B), adipose tissue (FIG. 6C), and adrenal tissue (FIG. 6D) 14 and 35 days after subcutaneous injection in control mice (group A administered PBS) or mice administered Compounds 1, and 5-9 (respectively groups B-G).

FIG. 7 provides schematics of RNAi oligonucleotide-lipid conjugates targeting Aldh2 mRNA having the structures of Compounds 10 and 11.

FIGS. 8A-8D provide graphs measuring percent (%) murine Aldh2 mRNA remaining in liver (FIG. 8A), skeletal muscle (FIG. 8B), adipose tissue (FIG. 8C), and adrenal tissue (FIG. 8D) 14 days after subcutaneous injection in control mice (administered PBS) or mice administered Compounds 10 and 11 (respectively groups B and E).

FIG. 9 provides schematics of RNAi oligonucleotide-lipid conjugates targeting Aldh2 mRNA having the structures of Compounds 11-15.

FIGS. 10A-10D provide graphs measuring percent (%) murine Aldh2 mRNA remaining in liver (FIG. 10A), skeletal muscle (FIG. 10B), adipose tissue (FIG. 10C), and adrenal tissue (FIG. 10D) 14 days after subcutaneous injection in control mice (administered PBS) or mice administered Compounds 12, 13, 11, 14, and 15 (respectively groups C-G).

FIG. 11 provides schematics of RNAi oligonucleotide-lipid conjugates targeting Aldh2 mRNA having the structures of Compounds 16-22.

FIGS. 12A-12D provide graphs measuring percent (%) murine Aldh2 mRNA remaining in liver (FIG. 12A), skeletal muscle (FIG. 12B), adipose tissue (FIG. 12C), and adrenal tissue (FIG. 12D) 14 days after subcutaneous injection in control mice (group A administered PBS) or mice administered Compounds 16-22 (respectively groups B, C, G-K).

FIG. 13 provides schematics of RNAi oligonucleotide-lipid conjugates targeting Aldh2 mRNA having the structures of Compounds 12 and 23-28.

FIGS. 14A-14D provide graphs measuring percent (%) murine Aldh2 mRNA remaining in liver (FIG. 14A), skeletal muscle (FIG. 14B), adipose tissue (FIG. 14C), and adrenal tissue (FIG. 14D) 14 days after subcutaneous injection in control mice (group A administered PBS) or mice administered Compounds 12 and 23-28 (respectively groups B-J).

FIG. 15 provides schematics of RNAi oligonucleotide-lipid conjugates targeting SLC25A1 mRNA having the structures of Compounds 29-34.

FIGS. 16A-16D provide graphs measuring percent (%) murine SLC25A1 mRNA remaining in liver (FIG. 16A), skeletal muscle (FIG. 16B), adipose tissue (FIG. 16C), and adrenal tissue (FIG. 16D) 14 days after subcutaneous injection in control mice (group A administered PBS) or mice administered Compounds 29-34 (respectively groups B-D, F, H, and I).

FIG. 17 provides schematics of RNAi oligonucleotide-lipid conjugates targeting STAT3 mRNA having the structures of Compounds 35-37.

FIGS. 18A-18D provide graphs measuring percent (%) murine STAT3 mRNA remaining in liver (FIG. 18A), skeletal muscle (FIG. 18B), adipose tissue (FIG. 18C), and adrenal tissue (FIG. 18D) 14 days after subcutaneous injection in control mice (group A administered PBS) or mice administered Compounds 35-37 (respectively groups B, H, and I).

### DETAILED DESCRIPTION

In some aspects, the disclosure provides double-stranded oligonucleotides (e.g., RNAi oligonucleotide-lipid conjugates) that reduce expression of a target gene. In other aspects, the disclosure provides methods of treating a disease or disorder associated with expression of a target gene. In other aspects, the disclosure provides methods of treating a disease or disorder associated with expression of a target gene using the double-stranded oligonucleotides, or pharmaceutically acceptable compositions thereof, described herein. In other aspects, the disclosure provides methods of using the double-stranded oligonucleotides described herein in the manufacture of a medicament for treating a disease or disorder associated with expression of a target gene.

### Double-Stranded Oligonucleotides

The disclosure provides, inter alia, double-stranded oligonucleotides (e.g., RNAi oligonucleotide-lipid conjugates) that reduce expression of a target gene. In some embodiments, a double-stranded oligonucleotide provided by the disclosure is targeted to an mRNA encoding the target gene. Messenger RNA (mRNA) that encodes a target gene and is targeted by a double-stranded oligonucleotide of the disclosure is referred to herein as “target mRNA”.

mRNA Target Sequences

In some embodiments, the double-stranded oligonucleotide is targeted to a target sequence comprising a target mRNA. In some embodiments, the double-stranded oligonucleotide is targeted to a target sequence within a target mRNA. In some embodiments, the double-stranded oligonucleotide, or a portion, fragment, or strand thereof (e.g., an antisense strand or a guide strand of a double-stranded oligonucleotide) binds or anneals to a target sequence comprising a target mRNA, thereby reducing target gene expression. In some embodiments, the double-stranded oligonucleotide is targeted to a target sequence comprising target mRNA for the purpose of reducing expression of a target gene in vivo. In some embodiments, the amount or extent of reduction of target gene expression by a double-stranded oligonucleotide targeted to a specific target sequence correlates with the potency of the double-stranded oligonucleotide. In some embodiments, the amount or extent of reduction of target gene expression by a double-stranded oligonucleotide targeted to a specific target sequence correlates with the amount or extent of therapeutic benefit in a subject or patient having a disease, disorder or condition associated with target gene expression treated with the double-stranded oligonucleotide.

Through examination of the nucleotide sequence of mRNAs encoding target genes, including mRNAs of multiple different species (e.g., human, cynomolgus monkey, mouse, and rat) and as a result of in vitro and in vivo testing, it has been discovered that certain nucleotide sequences and certain systemic modifications to those oligonucleotides are more amenable than others to RNAi oligonucleotide-mediated reduction and are thus useful as part of oligonucleotides that are otherwise targeted to specific gene target sequences. In some embodiments, a sense strand of a double-stranded oligonucleotide, or a portion or fragment thereof, described herein, comprises a nucleotide sequence that is similar (e.g., having no more than 4 mismatches) or is identical to a target sequence comprising a target mRNA. In some embodiments, a portion or region of the sense strand of a double-stranded oligonucleotide described herein comprises a target sequence comprising a target mRNA.

In some embodiments, the target mRNA is expressed in hepatic tissue, hepatic non-parenchymal cells, cardiomyocytes, immune cells, skeletal muscle, adipose tissue, adrenal tissue, or any combination thereof. In some embodiments, the target mRNA is expressed in the hepatic tissue, adipose tissue, adrenal tissue, or skeletal muscle tissue. In some embodiments, the target mRNA is expressed in hepatic tissue. In some embodiments, the target mRNA is expressed in a hepatocyte. In some embodiments, the target mRNA is expressed in adipose tissue. In some embodiments, the target mRNA is expressed in skeletal muscle tissue. In some embodiments, the target mRNA is expressed in adrenal tissue. In some embodiments, the target mRNA is expressed in hepatic non-parenchymal cells. In some embodiments, the target mRNA is expressed in cardiomyocytes. In some embodiments, the target mRNA is expressed in immune cells.

### RNAi Oligonucleotide Targeting Sequences

In some embodiments, the double-stranded oligonucleotides provided by the disclosure comprise a targeting sequence. As used herein, the term “targeting sequence” refers to a nucleotide sequence having a region of complementarity to a specific nucleotide sequence comprising an mRNA. In some embodiments, the double-stranded oligonucleotides provided by the disclosure comprise a gene targeting sequence having a region of complementarity to a nucleotide sequence comprising a target sequence of a target mRNA.

The targeting sequence imparts the double-stranded oligonucleotide with the ability to specifically target an mRNA by binding or annealing to a target sequence comprising a target mRNA by complementary (Watson-Crick) base pairing. In some embodiments, the double-stranded oligonucleotides herein (or a strand thereof, e.g., an antisense strand or a guide strand of a double-stranded oligonucleotide) comprise a targeting sequence having a region of complementarity that binds or anneals to a target sequence comprising a target mRNA by complementary (Watson-Crick) base pairing. In some embodiments, the double-stranded oligonucleotides herein (or a strand thereof, e.g., an antisense strand or a guide strand of a double-stranded oligonucleotide) comprise a targeting sequence having a region of complementarity that binds or anneals to a target sequence within a target mRNA by complementary (Watson-Crick) base pairing. The targeting sequence is generally of suitable length and base content to enable binding or annealing of the double-stranded oligonucleotide (or a strand thereof) to a specific target mRNA for purposes of inhibiting target gene expression. In some embodiments, the targeting sequence is at least about 12, at least about 13, at least about 14, at least about 15, at least about 16, at least about 17, at least about 18, at least about 19, at least about 20, at least about 21, at least about 22, at least about 23, at least about 24, at least about 25, at least about 26, at least about 27, at least about 28, at least about 29 or at least about 30 nucleotides in length. In some embodiments, the targeting sequence is at least 12, at least 13, at least 14, at least 15, at least 16, at least 17, at least 18, at least 19 or at least 20 nucleotides. In some embodiments, the targeting sequence is about 12 to about 30 (e.g., 12 to 30, 12 to 22, 15 to 25, 17 to 21, 18 to 27, 19 to 27, or 15 to 30) nucleotides in length. In some embodiments, the targeting sequence is about 12, 13, 14, 15, 16, 17, 18, 19, 20, 21, 22, 23, 24, 25, 26, 27, 28, 29 or 30 nucleotides in length. In some embodiments, the targeting sequence is 18 nucleotides in length. In some embodiments, the targeting sequence is 19 nucleotides in length. In some embodiments, the targeting sequence is 20 nucleotides in length. In some embodiments, the targeting sequence is 21 nucleotides in length. In some embodiments, the targeting sequence is 22 nucleotides in length. In some embodiments, the targeting sequence is 23 nucleotides in length. In some embodiments, the targeting sequence is 24 nucleotides in length.

In some embodiments, the double-stranded oligonucleotides herein comprise a targeting sequence that is fully complementary to a target sequence comprising a target mRNA. In some embodiments, the double-stranded oligonucleotides herein comprise a targeting sequence that is fully complementary to a target sequence within a target mRNA. In some embodiments, the targeting sequence is partially complementary to a target sequence comprising a target mRNA. In some embodiments, the targeting sequence is partially complementary to a target sequence within a target mRNA. In some embodiments, the targeting sequence comprises a region of contiguous nucleotides comprising the antisense strand.

In some embodiments, the double-stranded oligonucleotides herein comprise a targeting sequence that is complementary to a contiguous sequence of nucleotides comprising a target mRNA, wherein the contiguous sequence of nucleotides is about 12 to about 30 nucleotides in length (e.g., 12 to 30, 12 to 28, 12 to 26, 12 to 24, 12 to 20, 12 to 18, 12 to 16, 14 to 22, 16 to 20, 18 to 20 or 18 to 19 nucleotides in length). In some embodiments, the double-stranded oligonucleotides comprise a targeting sequence that is complementary to a contiguous sequence of nucleotides comprising a target mRNA, wherein the contiguous sequence of nucleotides is 10, 11, 12, 13, 14, 15, 16, 17, 18, 19 or 20 nucleotides in length. In some embodiments, the double-stranded oligonucleotides comprise a targeting sequence that is complementary to a contiguous sequence of nucleotides comprising a target mRNA, wherein the contiguous sequence of nucleotides is 15 nucleotides in length. In some embodiments, the double-stranded oligonucleotides comprise a targeting sequence that is complementary to a contiguous sequence of nucleotides comprising a target mRNA, wherein the contiguous sequence of nucleotides is 19 nucleotides in length.

In some embodiments, the double-stranded oligonucleotide comprises a targeting sequence that is complementary to a contiguous sequence of nucleotides comprising a target mRNA, wherein the contiguous sequence of nucleotides is 15 nucleotides in length. In some embodiments, the double-stranded oligonucleotide comprises a targeting sequence that is complementary to a contiguous sequence of nucleotides comprising a target mRNA, wherein the contiguous sequence of nucleotides is 19 nucleotides in length.

In some embodiments, a targeting sequence of a double-stranded oligonucleotide herein is fully complementary (e.g., having no mismatches) to a target sequence comprising a target mRNA and comprises the entire length of an antisense strand. In some embodiments, a targeting sequence of a double-stranded oligonucleotide herein is fully complementary (e.g., having no mismatches) to a target sequence comprising a target mRNA and comprises a portion of the entire length of an antisense strand. In some embodiments, a targeting sequence of a double-stranded oligonucleotide herein is fully complementary (e.g., having no mismatches) to a target sequence comprising a target mRNA and comprises 10 to 20 nucleotides of the antisense strand. In some embodiments, a targeting sequence of a double-stranded oligonucleotide herein is fully complementary (e.g., having no mismatches) to a target sequence comprising a target mRNA and comprises 15 to 19 nucleotides of the antisense strand. In some embodiments, a targeting sequence of a double-stranded oligonucleotide herein is fully complementary (e.g., having no mismatches) to a target sequence comprising a target mRNA and comprises 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, or 22 nucleotides of the antisense strand. In some embodiments, a targeting sequence of a double-stranded oligonucleotide herein is fully complementary (e.g., having no mismatches) to a target sequence comprising a target mRNA and comprises 19 nucleotides of the antisense strand.

In some embodiments, a targeting sequence of a double-stranded oligonucleotide herein is partially complementary (e.g., having no more than 4 mismatches) to a target sequence comprising a target mRNA and comprises the entire length of an antisense strand. In some embodiments, a targeting sequence of a double-stranded oligonucleotide herein is partially complementary (e.g., having no more than 4 mismatches) to a target sequence comprising a target mRNA and comprises a portion of the entire length of an antisense strand. In some embodiments, a targeting sequence of a double-stranded oligonucleotide herein is partially complementary (e.g., having no more than 4 mismatches) to a target sequence comprising a target mRNA and comprises 10 to 20 nucleotides of the antisense strand. In some embodiments, a targeting sequence of a double-stranded oligonucleotide herein is partially complementary (e.g., having no more than 4 mismatches) to a target sequence comprising a target mRNA and comprises 15 to 19 nucleotides of the antisense strand. In some embodiments, a targeting sequence of a double-stranded oligonucleotide herein is partially complementary (e.g., having no more than 4 mismatches) to a target sequence comprising a target mRNA and comprises 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, or 22 nucleotides of the antisense strand. In some embodiments, a targeting sequence of a double-stranded oligonucleotide herein is partially complementary (e.g., having no more than 4 mismatches) to a target sequence comprising a target mRNA and comprises 19 nucleotides of the antisense strand.

In some embodiments, a double-stranded oligonucleotide herein comprises a targeting sequence having one or more base pair (bp) mismatches with the corresponding target sequence comprising a target mRNA. In some embodiments, the targeting sequence has a 1 bp mismatch, a 2 bp mismatch, a 3 bp mismatch, a 4 bp mismatch, or a 5 bp mismatch with the corresponding target sequence comprising a target mRNA provided that the ability of the targeting sequence to bind or anneal to the target sequence under appropriate hybridization conditions and/or the ability of the double-stranded oligonucleotide to inhibit or reduce target gene expression is maintained (e.g., under physiological conditions). Alternatively, in some embodiments, the targeting sequence comprises no more than 1, no more than 2, no more than 3, no more than 4, or no more than 5 bp mismatches with the corresponding target sequence comprising a target mRNA provided that the ability of the targeting sequence to bind or anneal to the target sequence under appropriate hybridization conditions and/or the ability of the double-stranded oligonucleotide to inhibit or reduce target gene expression is maintained. In some embodiments, the double-stranded oligonucleotide comprises a targeting sequence having 1 mismatch with the corresponding target sequence. In some embodiments, the double-stranded oligonucleotide comprises a targeting sequence having 2 mismatches with the corresponding target sequence. In some embodiments, the double-stranded oligonucleotide comprises a targeting sequence having 3 mismatches with the corresponding target sequence. In some embodiments, the double-stranded oligonucleotide comprises a targeting sequence having 4 mismatches with the corresponding target sequence. In some embodiments, the double-stranded oligonucleotide comprises a targeting sequence having 5 mismatches with the corresponding target sequence. In some embodiments, the double-stranded oligonucleotide comprises a targeting sequence having more than one mismatch (e.g., 2, 3, 4, 5 or more mismatches) with the corresponding target sequence, wherein at least 2 (e.g., all) of the mismatches are positioned consecutively (e.g., 2, 3, 4, 5 or more mismatches in a row), or wherein the mismatches are interspersed in any position throughout the targeting sequence. In some embodiments, the double-stranded oligonucleotide comprises a targeting sequence having more than one mismatch (e.g., 2, 3, 4, 5 or more mismatches) with the corresponding target sequence, wherein at least 2 (e.g., all) of the mismatches are positioned consecutively (e.g., 2, 3, 4, 5 or more mismatches in a row), or wherein at least one or more non-mismatched base pair is located between the mismatches, or a combination thereof.

### Types of Oligonucleotides

A variety of RNAi oligonucleotide types and/or structures are useful for reducing target gene expression in the methods herein. Any of the RNAi oligonucleotide types described herein or elsewhere are contemplated for use as a framework to incorporate a targeting sequence herein for the purposes of inhibiting or reducing corresponding target gene expression.

In some embodiments, the double-stranded oligonucleotides herein inhibit target gene expression by engaging with RNA interference (RNAi) pathways upstream or downstream of Dicer involvement. For example, RNAi oligonucleotides have been developed with each strand having sizes of about 19-25 nucleotides with at least one 3′ overhang of 1 to 5 nucleotides (see, e.g., U.S. Pat. No. 8,372,968). Longer oligonucleotides also have been developed that are processed by Dicer to generate active RNAi products (see, e.g., U.S. Pat. No. 8,883,996). Further work produced extended double-stranded oligonucleotides where at least one end of at least one strand is extended beyond a duplex targeting region, including structures where one of the strands includes a thermodynamically stabilizing tetraloop structure (see, e.g., U.S. Pat. Nos. 8,513,207 and 8,927,705, as well as Intl. Patent Application Publication No. WO 2010/033225). Such structures may include single-stranded extensions (on one or both sides of the molecule) as well as double-stranded extensions.

In some embodiments, the RNAi oligonucleotides herein engage with the RNAi pathway downstream of the involvement of Dicer (e.g., Dicer cleavage). In some embodiments, the oligonucleotides described herein are Dicer substrates. In some embodiments, upon endogenous Dicer processing, double-stranded nucleic acids of 19-23 nucleotides in length capable of reducing expression of a target mRNA are produced.

In some embodiments, the RNAi oligonucleotides conjugates disclosed herein comprise sense and antisense strands that are both in the range of about 16 to 34 (e.g., 16 to 26, 20 to 34 or 30-34) nucleotides in length.

### Antisense Strands

In some embodiments, an antisense strand of a double-stranded oligonucleotide is referred to as a “guide strand.” For example, an antisense strand that engages with RNA-induced silencing complex (RISC) and binds to an Argonaute protein such as Ago2, or engages with or binds to one or more similar factors, and directs silencing of a target gene, the antisense strand is referred to as a guide strand. In some embodiments, a sense strand complementary to a guide strand is referred to as a “passenger strand.”

In some embodiments, a double-stranded oligonucleotide herein comprises an antisense strand of up to about 50 nucleotides in length (e.g., up to 50, up to 40, up to 35, up to 30, up to 27, up to 25, up to 21, up to 19, up to 17, up to 15, or up to 8 nucleotides in length). In some embodiments, a double-stranded oligonucleotide herein comprises an antisense strand of at least about 12 nucleotides in length (e.g., at least 12, at least 15, at least 19, at least 21, at least 22, at least 25, at least 27, at least 30, at least 35 or at least 38 nucleotides in length). In some embodiments, a herein comprises an antisense strand in a range of about 8 to about 40 (e.g., 8 to 40, 8 to 36, 8 to 32, 8 to 28, 15 to 40, 15 to 36, 15 to 32, 15 to 30, 15 to 28, 17 to 22, 17 to 25, 19 to 27, 19 to 30, 20 to 40, 22 to 40, 25 to 40 or 32 to 40) nucleotides in length. In some embodiments, a double-stranded oligonucleotide herein comprises an antisense strand of 15 to 30 nucleotides in length. In some embodiments, an antisense strand of any one of the double-stranded oligonucleotide disclosed herein is of 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides in length. In some embodiments, a double-stranded oligonucleotide comprises an antisense strand of 19-23 nucleotides in length. In some embodiments, a double-stranded oligonucleotide comprises an antisense strand of 19 nucleotides in length. In some embodiments, a double-stranded oligonucleotide comprises an antisense strand of 20 nucleotides in length. In some embodiments, a double-stranded oligonucleotide comprises an antisense strand of 21 nucleotides in length. In some embodiments, a double-stranded oligonucleotide comprises an antisense strand of 22 nucleotides in length. In some embodiments, a double-stranded oligonucleotide comprises an antisense strand of 23 nucleotides in length.

### Sense Strands

In some embodiments, a double-stranded oligonucleotide disclosed herein comprises a sense strand (or passenger strand) of up to about 50 nucleotides in length (e.g., up to 50, up to 40, up to 36, up to 30, up to 27, up to 25, up to 21, up to 19, up to 17 or up to 12 nucleotides in length). In some embodiments, a double-stranded oligonucleotide herein comprises a sense strand of at least about 12 nucleotides in length (e.g., at least 12, at least 15, at least 19, at least 21, at least 25, at least 27, at least 30, at least 36 or at least 38 nucleotides in length). In some embodiments, a double-stranded oligonucleotide herein comprises a sense strand in a range of about 12 to about 50 (e.g., 12 to 50, 12 to 40, 12 to 36, 12 to 32, 12 to 28, 15 to 40, 15 to 36, 15 to 32, 15 to 28, 17 to 21, 17 to 25, 19 to 27, 19 to 30, 20 to 40, 22 to 40, 25 to 40 or 32 to 40) nucleotides in length. In some embodiments, a double-stranded oligonucleotide herein comprises a sense strand 15 to 50 nucleotides in length. In some embodiments, a double-stranded oligonucleotide herein comprises a sense strand 18 to 38 nucleotides in length. In some embodiments, a double-stranded oligonucleotide herein comprises a sense strand of 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, a double-stranded oligonucleotide herein comprises a sense strand of 12-21 nucleotides in length. In some embodiments, a double-stranded oligonucleotide herein comprises a sense strand of 12 nucleotides in length. In some embodiments, a double-stranded oligonucleotide herein comprises a sense strand of 13 nucleotides in length. In some embodiments, a double-stranded oligonucleotide herein comprises a sense strand of 14 nucleotides in length. In some embodiments, a double-stranded oligonucleotide herein comprises a sense strand of 15 nucleotides in length. In some embodiments, a double-stranded oligonucleotide herein comprises a sense strand of 16 nucleotides in length. In some embodiments, a double-stranded oligonucleotide herein comprises a sense strand of 17 nucleotides in length. In some embodiments, a double-stranded oligonucleotide herein comprises a sense strand of 18 nucleotides in length. In some embodiments, a double-stranded oligonucleotide herein comprises a sense strand of 19 nucleotides in length. In some embodiments, a double-stranded oligonucleotide herein comprises a sense strand of 20 nucleotides in length. In some embodiments, a double-stranded oligonucleotide herein comprises a sense strand of 21 nucleotides in length. In some embodiments, a double-stranded oligonucleotide herein comprises a sense strand of 22 nucleotides in length. In some embodiments, a double-stranded oligonucleotide herein comprises a sense strand of 23 nucleotides in length. In some embodiments, a double-stranded oligonucleotide herein comprises a sense strand of 24 nucleotides in length. In some embodiments, a double-stranded oligonucleotide herein comprises a sense strand of 25 nucleotides in length. In some embodiments, a double-stranded oligonucleotide herein comprises a sense strand of 26 nucleotides in length. In some embodiments, a double-stranded oligonucleotide herein comprises a sense strand of 27 nucleotides in length. In some embodiments, a double-stranded oligonucleotide herein comprises a sense strand of 28 nucleotides in length. In some embodiments, a double-stranded oligonucleotide herein comprises a sense strand of 29 nucleotides in length. In some embodiments, a double-stranded oligonucleotide herein comprises a sense strand of 30 nucleotides in length. In some embodiments, a double-stranded oligonucleotide herein comprises a sense strand of 31 nucleotides in length. In some embodiments, a double-stranded oligonucleotide herein comprises a sense strand of 32 nucleotides in length. In some embodiments, a double-stranded oligonucleotide herein comprises a sense strand of 33 nucleotides in length. In some embodiments, a double-stranded oligonucleotide herein comprises a sense strand of 34 nucleotides in length. In some embodiments, a double-stranded oligonucleotide herein comprises a sense strand of 35 nucleotides in length. In some embodiments, a double-stranded oligonucleotide herein comprises a sense strand of 36 nucleotides in length. In some embodiments, a double-stranded oligonucleotide herein comprises a sense strand of 37 nucleotides in length. In some embodiments, a double-stranded oligonucleotide herein comprises a sense strand of 38 nucleotides in length.

In some embodiments, a sense strand comprises a blunt end at its 3′ end. In some embodiments, the blunt end comprises the 3′ end of the sense strand.

In some embodiments, a sense strand comprises a stem-loop structure at its 3′ end. In some embodiments, a sense strand comprises a stem-loop structure at its 5′ end. In some embodiments, the stem-loop is formed by intrastrand base pairing. In some embodiments, a sense strand comprises a stem-loop structure at its 5′ end. In some embodiments, a stem is a duplex of 1, 2, 3, 4, 5, 6, 7, 8, 9, 10, 11, 12, 13 or 14 nucleotides in length. In some embodiments, the stem of the stem-loop comprises a duplex of 1 nucleotide in length. In some embodiments, the stem of the stem-loop comprises a duplex of 2 nucleotides in length. In some embodiments, the stem of the stem-loop comprises a duplex of 3 nucleotides in length. In some embodiments, the stem of the stem-loop comprises a duplex of 4 nucleotides in length. In some embodiments, the stem of the stem-loop comprises a duplex of 5 nucleotides in length. In some embodiments, the stem of the stem-loop comprises a duplex of 6 nucleotides in length. In some embodiments, the stem of the stem-loop comprises a duplex of 7 nucleotides in length. In some embodiments, the stem of the stem-loop comprises a duplex of 8 nucleotides in length. In some embodiments, the stem of the stem-loop comprises a duplex of 9 nucleotides in length. In some embodiments, the stem of the stem-loop comprises a duplex of 10 nucleotides in length. In some embodiments, the stem of the stem-loop comprises a duplex of 11 nucleotides in length. In some embodiments, the stem of the stem-loop comprises a duplex of 12 nucleotides in length. In some embodiments, the stem of the stem-loop comprises a duplex of 13 nucleotides in length. In some embodiments, the stem of the stem-loop comprises a duplex of 14 nucleotides in length.

In some embodiments, a stem-loop provides the double-stranded oligonucleotide protection against degradation (e.g., enzymatic degradation), facilitates or improves targeting and/or delivery to a target cell, tissue, or organ, or both. For example, in some embodiments, the loop of a stem-loop provides nucleotides comprising one or more modifications that facilitate, improve, or increase targeting to a target mRNA (e.g., a target mRNA expressed in extra-hepatic tissue), inhibition of target gene expression, and/or delivery to a target cell, tissue, or organ, or a combination thereof. In some embodiments, the stem-loop itself or modification(s) to the stem-loop do not substantially affect the inherent gene expression inhibition activity of the double-stranded oligonucleotide, but facilitates, improves, or increases stability (e.g., provides protection against degradation) and/or delivery of the double-stranded oligonucleotide to a target cell, tissue, or organ. In certain embodiments, a double-stranded oligonucleotide herein comprises a sense strand comprising (e.g., at its 3′ end) a stem-loop set forth as: S1-L-S2, in which S1 is complementary to S2, and in which L forms a single-stranded loop between S1 and S2 of up to about 10 nucleotides in length (e.g., 3, 4, 5, 6, 7, 8, 9 or 10 nucleotides in length). In some embodiments, the loop (L) is 3 nucleotides in length. In some embodiments, the loop (L) is 4 nucleotides in length.

In some embodiments, a double-stranded oligonucleotide herein comprises a sense strand comprising (e.g., at its 3′ end) a stem-loop set forth as: S1-L-S2, in which S1 is complementary to S2, and in which L forms a single-stranded loop between S1 and S2 of 3 nucleotides in length, where L is 4 nucleotides in length. In some embodiments, a double-stranded oligonucleotide herein comprises a sense strand comprising (e.g., at its 3′ end) a stem-loop set forth as: S1-L-S2, in which S1 is complementary to S2, and in which L forms a single-stranded loop between S1 and S2 of 6 nucleotides in length, where L is 4 nucleotides in length.

In some embodiments, the tetraloop comprises the sequence 5′-GAAA-3′. In some embodiments, the stem loop comprises the sequence 5′-GCAGCCGAAAGGCUGC-3′ (SEQ ID NO: 15).

In some embodiments, a loop (L) of a stem-loop having the structure S1-L-S2 as described above is a triloop. In some embodiments, the triloop comprises ribonucleotides, deoxyribonucleotides, modified nucleotides, delivery ligands, and combinations thereof.

In some embodiments, a loop (L) of a stem-loop having the structure S1-L-S2 as described above is a tetraloop (e.g., within a nicked tetraloop structure). In some embodiments, the tetraloop comprises ribonucleotides, deoxyribonucleotides, modified nucleotides, delivery ligands, and combinations thereof.

In some embodiments, a loop (L) of a stem-loop having the structure S1-L-S2 as described above is a tetraloop as described in U.S. Pat. No. 10,131,912, incorporated herein by reference (e.g., within a nicked tetraloop structure).

### Duplex Length

In some embodiments, a duplex formed between a sense and antisense strand is at least 10 (e.g., at least 11, at least 12, at least 13, at least 14, at least 15, or at least 16) nucleotides in length. In some embodiments, a duplex formed between a sense and antisense strand is at least 12 (e.g., at least 15, at least 16, at least 17, at least 18, at least 19, at least 20, or at least 21) nucleotides in length. In some embodiments, a duplex formed between a sense and antisense strand is in the range of 12-30 nucleotides in length (e.g., 12 to 30, 12 to 27, 12 to 22, 15 to 25, 18 to 30, 18 to 22, 18 to 25, 18 to 27, 18 to 30, 19 to 30 or 21 to 30 nucleotides in length). In some embodiments, a duplex formed between a sense and antisense strand is 12, 13, 14, 15, 16, 17, 18, 19, 29, 21, 22, 23, 24, 25, 26, 27, 28, 29 or 30 nucleotides in length. In some embodiments, a duplex formed between a sense and antisense strand is 10-18 base pairs in length. In some embodiments, a duplex formed between a sense and antisense strand is 15-30 base pairs in length. In some embodiments, a duplex formed between a sense and antisense strand is 17-21 base pairs in length. In some embodiments, a duplex formed between a sense and antisense strand is 12 base pairs in length. In some embodiments, a duplex formed between a sense and antisense strand is 13 base pairs in length. In some embodiments, a duplex formed between a sense and antisense strand is 14 base pairs in length. In some embodiments, a duplex formed between a sense and antisense strand is 15 base pairs in length. In some embodiments, a duplex formed between a sense and antisense strand is 16 base pairs in length. In some embodiments, a duplex formed between a sense and antisense strand is 17 base pairs in length. In some embodiments, a duplex formed between a sense and antisense strand is 18 base pairs in length. In some embodiments, a duplex formed between a sense and antisense strand is 19 base pairs in length. In some embodiments, a duplex formed between a sense and antisense strand is 20 base pairs in length. In some embodiments, a duplex formed between a sense and antisense strand is 21 base pairs in length. In some embodiments, a duplex formed between a sense and antisense strand does not span the entire length of the sense strand and/or antisense strand. In some embodiments, a duplex between a sense and antisense strand spans the entire length of either the sense or antisense strands. In some embodiments, a duplex between a sense and antisense strand spans the entire length of both the sense strand and the antisense strand.

### Oligonucleotide Ends

In some embodiments, a double-stranded oligonucleotide disclosed herein comprises sense and antisense strands, such that there is a 3′-overhang on either the sense strand or the antisense strand, or both the sense and antisense strand. In some embodiments, a double-stranded oligonucleotide herein has one 5′end that is thermodynamically less stable compared to the other 5′ end. In some embodiments, an asymmetric double-stranded oligonucleotide conjugate is provided that includes a blunt end at the 3′end of a sense strand and overhang at the 3′ end of the antisense strand.

In some embodiments, the 3′-overhang is about one (1) to ten (10) nucleotides in length (e.g., about 1, 2, 3, 4, 5, 6, 7, 8, 9, or about 10 nucleotides in length). In some embodiments, the 3′ overhang is about one (1) to nine (9), one (1) to eight (8), one (1) to seven (7), one (1) to six (6), one (1) to five (5), one (1) to four (4), one (1) to three (3), or about one (1) to two (2) nucleotides in length. In some embodiments, the 3′-overhang is four (4) nucleotides in length. In some embodiments, the 3′-overhang is five (5) nucleotides in length. In some embodiments, the 3′-overhang is six (6) nucleotides in length. In some embodiments, the 3′-overhang is seven (7) nucleotides in length. In some embodiments, the 3′-overhang is eight (8) nucleotides in length. In some embodiments, the 3′-overhang is nine (9) nucleotides in length. In some embodiments, the 3′-overhang is ten (10) nucleotides in length.

In some embodiments, an overhang is a 3′ overhang comprising a length of between four and nine nucleotides, optionally four to nine, four to eight, four to seven, four to six, four to five, five to nine, five to eight, five to seven, five to six, six to nine, six to eight, six to seven, seven to nine, or seven to eight nucleotides. In some embodiments, the overhang is a 5′ overhang comprising a length of between one and four nucleotides, optionally one to four, one to three, one to two, two to four, two to three, or one, two, three, or four nucleotides.

In some embodiments, an oligonucleotide herein comprises a sense strand and an antisense strand, wherein the 5′ terminus of either or both strands comprise a 5′-overhang comprising one or more nucleotides. In some embodiments, an oligonucleotide herein comprises a sense strand and an antisense strand, wherein the sense strand comprises a 5′-overhang comprising one or more nucleotides. In some embodiments, an oligonucleotide herein comprises a sense strand and an antisense strand, wherein the antisense strand comprises a 5′-overhang comprising one or more nucleotides. In some embodiments, an oligonucleotide herein comprises a sense strand and an antisense strand, wherein both the sense strand and the antisense strand comprises a 5′-overhang comprising one or more nucleotides.

In some embodiments, the 5′-overhang is about one (1) to ten (10) nucleotides in length (e.g., about 1, 2, 3, 4, 5, 6, 7, 8, 9, or about 10 nucleotides in length). In some embodiments, the 5′ overhang is about one (1) to ten (10), one (1) to nine (9), one (1) to eight (8), one (1) to seven (7), one (1) to six (6), one (1) to five (5), one (1) to four (4), one (1) to three (3), or about one (1) to two (2) nucleotides in length. In some embodiments, the 5′-overhang is (1) nucleotide in length. In some embodiments, the 5′-overhang is two (2) nucleotides in length. In some embodiments, the 5′-overhang is three (3) nucleotides in length. In some embodiments, the 5′-overhang is four (4) nucleotides in length. In some embodiments, the 5′-overhang is five (5) nucleotides in length. In some embodiments, the 5′-overhang is six (6) nucleotides in length. In some embodiments, the 5′-overhang is seven (7) nucleotides in length. In some embodiments, the 5′-overhang is eight (8) nucleotides in length. In some embodiments, the 5′-overhang is nine (9) nucleotides in length. In some embodiments, the 5′-overhang is ten (10) nucleotides in length.

In some embodiments, one or more (e.g., 2, 3, or 4) terminal nucleotides of the 3′ end or 5′ end of a sense and/or antisense strand are modified. For example, in some embodiments, one or two terminal nucleotides of the 3′ end of the antisense strand are modified. In some embodiments, the last nucleotide at the 3′ end of an antisense strand is modified, e.g., comprises 2′ modification, e.g., a 2′-O-methoxyethyl. In some embodiments, the last one or two terminal nucleotides at the 3′ end of an antisense strand are complementary with the target. In some embodiments, the last one or two nucleotides at the 3′ end of the antisense strand are not complementary with the target.

In some embodiments, a double-stranded oligonucleotide disclosed herein comprises a stem-loop structure at the 3′ end of the sense strand and comprises four to nine terminal overhang nucleotides at the 3′ end of the antisense strand. In some embodiments, a double-stranded oligonucleotide herein comprises a nicked tetraloop structure, wherein the 3′ end of the sense strand comprises a stem-tetraloop structure and comprises four to nine terminal overhang nucleotides at the 3′ end of the antisense strand.

In some embodiments, the two terminal nucleotides of the overhang are purines. In some embodiments, the two terminal nucleotides of the overhang are pyrimidines. In some embodiments, two terminal nucleotides of the overhang are purines and pyrimidines. In some embodiments, the two terminal nucleotides of the overhang are selected from AA, GG, AG, and GA. In some embodiments, the overhang is AA. In some embodiments, the overhang is AG. In some embodiments, the overhang is GA. In some embodiments, the two terminal overhang nucleotides are GG. Typically, one or both of the two terminal GG nucleotides of the antisense strand are not complementary with the target.

In some embodiments, one or more (e.g., 2, 3, 4, 5, 6) modified internucleotide linkages are provided between terminal nucleotides of the 3′ end or 5′ end of a sense and/or antisense strand. In some embodiments, modified internucleotide linkages are provided between overhang nucleotides at the 3′ end or 5′ end of a sense and/or antisense strand.

In some embodiments, the terminal nucleotide is a locked nucleic acid. In some embodiments the 5′ end of the sense strand is a locked nucleic acid. In some embodiments, the terminal nucleotide comprises an aliphatic chain (e.g., a C16, C18 or C22 lipid). In some embodiments, the 5′ end of the sense strand comprises an aliphatic chain (e.g., a C16, C18 or C22 lipid). In some embodiments, the 3′ end of the sense strand comprises an aliphatic chain (e.g., a C16, C18 or C22 lipid).

### Oligonucleotide Modifications

In some embodiments, a double-stranded oligonucleotide disclosed herein comprises one or more modifications. Oligonucleotides (e.g., RNAi oligonucleotides) may be modified in various ways to improve or control specificity, stability, delivery, bioavailability, resistance from nuclease degradation, immunogenicity, base-pairing properties, RNA distribution and cellular uptake and other features relevant to therapeutic research use.

In some embodiments, the modification is a modified sugar. In some embodiments, the modification is a 5′-terminal phosphate group. In some embodiments, the modification is a modified internucleoside linkage. In some embodiments, the modification is a modified base. In some embodiments, an oligonucleotide described herein can comprise any one of the modifications described herein or any combination thereof. For example, in some embodiments, an oligonucleotide described herein comprises at least one modified sugar, a 5′-terminal phosphate group, at least one modified internucleoside linkage, and at least one modified base.

The number of modifications on an oligonucleotide (e.g., an RNAi oligonucleotide) and the position of those nucleotide modifications may influence the properties of an oligonucleotide. For example, oligonucleotides may be delivered in vivo by conjugating them to or encompassing them in a lipid nanoparticle (LNP) or similar carrier. However, when an oligonucleotide is not protected by an LNP or similar carrier, it may be advantageous for at least some of the nucleotides to be modified. Accordingly, in some embodiments, all or substantially all of the nucleotides of an oligonucleotides are modified. In some embodiments, more than half of the nucleotides are modified. In some embodiments, less than half of the nucleotides are modified. In some embodiments, the sugar moiety of all nucleotides comprising the oligonucleotide is modified at the 2′ position. In some embodiments, the sugar moiety of all nucleotides comprising the oligonucleotide is modified at the 2′ position, except for the nucleotide conjugated to a lipid (e.g., the 5′-terminal nucleotide of the sense strand). The modifications may be reversible or irreversible. In some embodiments, an oligonucleotide as disclosed herein has a number and type of modified nucleotides sufficient to cause the desired characteristics (e.g., protection from enzymatic degradation, capacity to target a desired cell after in vivo administration, and/or thermodynamic stability).

### Sugar Modifications

In some embodiments, a nucleotide modification in a sugar comprises a 2′-modification. In some embodiments, a 2′-modification is a modification known in the art. In some embodiments, a 2′-modification may be 2′-O-propargyl, 2′-O-propylamin, 2′-amino, 2′-ethyl, 2′-fluoro (2′-F), 2′-aminoethyl (EA), 2′-O-methyl (2′-OMe), 2′-O-methoxyethyl (2′-MOE), 2′-O-[2-(methylamino)-2-oxoethyl](2′-O-NMA) or 2′-deoxy-2′-fluoro-β-d-arabinonucleic acid (2′-FANA). In some embodiments, the modification is 2′-F, 2′-OMe or 2′-MOE. In some embodiments, a modification in a sugar comprises a modification of the sugar ring, which may comprise modification of one or more carbons of the sugar ring. For example, a modification of a sugar of a nucleotide may comprise a 2′-oxygen of a sugar is linked to a1′-carbon or 4′-carbon of the sugar, or a 2′-oxygen is linked to the 1′-carbon or 4′-carbon via an ethylene or methylene bridge. In some embodiments, a modified nucleotide has an acyclic sugar that lacks a 2′-carbon to 3′-carbon bond. In some embodiments, a modified nucleotide has a thiol group, e.g., in the 4′ position of the sugar.

In some embodiments, a double-stranded oligonucleotide described herein comprises at least about 1 modified nucleotide (e.g., at least 1, at least 5, at least 10, at least 15, at least 20, at least 25, at least 30, at least 35, at least 40, at least 45, at least 50, at least 55, at least 60, or more). In some embodiments, the sense strand of the double-stranded oligonucleotide comprises at least about 1 modified nucleotide (e.g., at least 1, at least 5, at least 10, at least 15, at least 20, at least 25, at least 30, at least 35, or more). In some embodiments, the antisense strand of the double-stranded oligonucleotide comprises at least about 1 modified nucleotide (e.g., at least 1, at least 5, at least 10, at least 15, at least 20, or more).

In some embodiments, all the nucleotides of the sense strand of the double-stranded oligonucleotide are modified. In some embodiments, all the nucleotides of the antisense strand of the double-stranded oligonucleotide are modified. In some embodiments, all the nucleotides of the double-stranded oligonucleotide (i.e., both the sense strand and the antisense strand) are modified. In some embodiments, the modified nucleotide comprises a 2′-modification (e.g., a 2′-F or 2′-OMe, 2′-MOE, and 2′-deoxy-2′-fluoro-β-d-arabinonucleic acid).

In some embodiments, the disclosure provides double-stranded oligonucleotides having different modification patterns. In some embodiments, the modified double-stranded oligonucleotides comprise a sense strand sequence having a modification pattern as set forth in the Examples and Sequence Listing and an antisense strand having a modification pattern as set forth in the Examples and Sequence Listing.

In some embodiments, a double-stranded oligonucleotide disclosed herein comprises an antisense strand having nucleotides that are modified with 2′-F. In some embodiments, a double-stranded oligonucleotide disclosed herein comprises an antisense strand comprises nucleotides that are modified with 2′-F and 2′-OMe. In some embodiments, a double-stranded oligonucleotide disclosed herein comprises a sense strand having nucleotides that are modified with 2′-F. In some embodiments, a double-stranded oligonucleotide disclosed herein comprises a sense strand comprising nucleotides that are modified with 2′-F and 2′-OMe. In some embodiments, a double-stranded oligonucleotide disclosed herein comprises a sense strand comprising nucleotides that are modified with 2′-F and 2′-OMe, provided that a nucleotide conjugated to a lipid moiety is not modified with 2′-F or 2′-OMe.

In some embodiments, an oligonucleotide described herein comprises a sense strand with about 10-25%, 10%, 11%, 12%, 13%, 14% 15%, 16%, 17%, 18%, 19% or 20% of the nucleotides of the sense strand comprising a 2′-fluoro modification. In some embodiments, about 11% of the nucleotides of the sense strand comprise a 2-fluoro modification. In some embodiments, about 20% of the nucleotides of the sense strand comprise a 2-fluoro modification. In some embodiments, an oligonucleotide described herein comprises an antisense strand with about 25-35%, 25%, 26%, 27%, 28%, 29%, 30%, 31%, 32%, 33%, 34% or 35% of the nucleotides of the antisense strand comprising a 2′-fluoro modification. In some embodiments, about 32% of the nucleotides of the antisense strand comprise a 2′-fluoro modification. In some embodiments, the oligonucleotide has about 15-25%, 15%, 16%, 17%, 18%, 19%, 20%, 21%, 22%, 23%, 24%, or 25% of its nucleotides comprising a 2′-fluoro modification. In some embodiments, about 19% of the nucleotides in the oligonucleotide comprise a 2′-fluoro modification. In some embodiments, about 26% of the nucleotides in the oligonucleotide comprise a 2′-fluoro modification.

In some embodiments, one or more of positions 3, 5, 6, 8, and 10 of the sense strand is modified with a 2′—F group. In some embodiments, the sugar moiety at each of nucleotides not modified with a 2′—F group or conjugated to a lipid in the sense strand is modified with a 2′-OMe. In some embodiments, the sugar moiety at each of nucleotides at positions 1-2, 4, 7, 9, and 11-29 in the sense strand is modified with a 2′-OMe, provided that a nucleotide conjugated to a lipid moiety is not modified with 2′-F or 2′-OMe.

In some embodiments, one or more of positions 4, 5, 6, and 7 of the sense strand is modified with a 2′—F group. In some embodiments, the sugar moiety at each of nucleotides not modified with a 2′—F group or conjugated to a lipid in the sense strand is modified with a 2′-OMe. In some embodiments, the sugar moiety at each of nucleotides at positions 1-3, 4, and 8-29 in the sense strand is modified with a 2′-OMe, provided that a nucleotide conjugated to a lipid moiety is not modified with 2′-F or 2′-OMe.

In some embodiments, one or more nucleotides of the sense strand forming a base pair with a nucleotide at one or more of positions 10, 11, and 12 of the antisense strand is modified with a 2′F group. In some embodiments, one or more nucleotides of the sense strand forming a base pair with a nucleotide at one or more of positions 10, 11, 12 and 13 of the antisense strand is modified with a 2′F group. In some embodiments, the remaining nucleotides of the sense strand are modified with a 2′-OMe, provided that a nucleotide conjugated to a lipid moiety is not modified with 2′-F or 2′-OMe.

In some embodiments, a double-stranded oligonucleotide provided herein comprises an antisense strand 22 nucleotides in length, with positions 1-22 numbered 5′ to 3′, and a sense strand having a 2′-fluoro modification at each of the nucleotides forming a base pair with nucleotides at one or more of positions 10, 11, and 12 of the antisense strand. In some embodiments, a double-stranded oligonucleotide provided herein comprises an antisense strand 22 nucleotides in length, with positions 1-22 numbered 5′ to 3′, and a sense strand having a 2′-fluoro modification at each of the nucleotides forming a base pair with nucleotides at positions 10, 11, 12, or any combination thereof, of the antisense strand. In some embodiments, a double-stranded oligonucleotide provided herein comprises an antisense strand 22 nucleotides in length, with positions 1-22 numbered 5′ to 3′, and a sense strand having a 2′-fluoro modification at each of the nucleotides forming a base pair with nucleotides at one or more of positions 10, 11, 12, and 13 of the antisense strand. In some embodiments, a double-stranded oligonucleotide provided herein comprises an antisense strand 22 nucleotides in length, with positions 1-22 numbered 5′ to 3′, and a sense strand having a 2′-fluoro modification at each of the nucleotides forming a base pair with nucleotides at positions 10, 11, 12, 13, or any combination thereof, of the antisense strand. In some embodiments, a double-stranded oligonucleotide provided herein comprises an antisense strand 22 nucleotides in length, with positions 1-22 numbered 5′ to 3′, and a sense strand having a 2′-fluoro modification at each of the nucleotides forming a base pair with nucleotides at positions 10, 11, and 12 of the antisense strand. In some embodiments, a double-stranded oligonucleotide provided herein comprises an antisense strand 22 nucleotides in length, with positions 1-22 numbered 5′ to 3′, and a sense strand having a 2′-fluoro modification at each of the nucleotides forming a base pair with nucleotides at positions 10, 11, 12, and 13 of the antisense strand.

In some embodiments, the sense strand comprises at least one 2′-F modified nucleotide wherein the remaining nucleotides not modified with a 2′—F group are modified with a 2′-OMe. In some embodiments, the sense strand comprises at least one 2′-F modified nucleotide wherein the remaining nucleotides not modified with a 2′—F group or conjugated to a lipid are modified with a 2′-OMe. In some embodiments, the remaining nucleotides of the sense strand not modified with a 2′—F group are modified with a 2′-OMe, provided the 5′ terminal nucleotide of the sense strand is an Tm-increasing nucleotide (e.g., LNA). In some embodiments, the sense strand comprises at least one 2′-F modified nucleotide wherein the remaining nucleotides not modified with a 2′—F group, are not a Tm-increasing nucleotide (e.g., LNA), or conjugated to a lipid are modified with a 2′-OMe.

In some embodiments, the antisense strand comprises at least 7 nucleotides that are modified at the 2′-position of the sugar moiety with a 2′-F. In some embodiments, the sugar moiety at positions 2, 3, 4, 5, 7, 10, and optionally up to 3 of the nucleotides at positions 14, 16, and 19 of the antisense strand are modified with a 2′-F. In some embodiments, the sugar moiety at each of the positions at positions 2, 3, 4, 5, 7, 10, 14, and 16 of the antisense strand is modified with the 2′-F. In some embodiments, the sugar moiety at each of the positions at positions 2, 3, 4, 5, 7, 10, 14, and 19 of the antisense strand is modified with the 2′-F. In other embodiments, the sugar moiety at each of the positions at positions 2, 3, 4, 5, 7, 10, 14, 16 and 19 of the antisense strand is modified with the 2′-F. In some embodiments, the sugar moiety at each of the positions at positions 2, 3, 4, 5, 7, 10 and 14 of the antisense strand is modified with the 2′-F.

In some embodiments, a double-stranded oligonucleotide provided herein comprises an antisense strand having the sugar moiety of each of the nucleotides at positions 2, 3, 4, 5, 7, 10, 14, and 16 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl](2′-O-NMA), and 2′-deoxy-2′-fluoro-β-d-arabinonucleic acid (2′-FANA).

In some embodiments, a double-stranded oligonucleotide provided herein comprises an antisense strand having the sugar moiety of each of the nucleotides at positions 2, 3, 4, 5, 7, 10, 14, 16 and 19 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl](2′-O-NMA), and 2′-deoxy-2′-fluoro-β-d-arabinonucleic acid (2′-FANA).

In some embodiments, a double-stranded oligonucleotide provided herein comprises an antisense strand having the sugar moiety of each of the nucleotides at positions 2, 3, 4, 5, 7, 10, 14, and 19 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

In some embodiments, a double-stranded oligonucleotide provided herein comprises an antisense strand having the sugar moiety of each of the nucleotides at positions 2, 3, 4, 5, 7, 10, and 14 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

In some embodiments, a double-stranded oligonucleotide provided herein comprises an antisense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, position 22, or any combination thereof, modified with 2′-F.

In some embodiments, a double-stranded oligonucleotide provided herein comprises an antisense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, position 22, or any combination thereof, modified with 2′-OMe.

In some embodiments, a double-stranded oligonucleotide provided herein comprises an antisense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, position 22, or any combination thereof, modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

In some embodiments, a double-stranded oligonucleotide provided herein comprises a sense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, position 22, position 23, position 24, position 25, position 26, position 27, position 28, position 29, position 30, position 31, position 32, position 33, position 34, position 35, position 36, or any combination thereof, modified with 2′-F.

In some embodiments, a double-stranded oligonucleotide provided herein comprises a sense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, position 22, position 23, position 24, position 25, position 26, position 27, position 28, position 29, position 30, position 31, position 32, position 33, position 34, position 35, position 36, or any combination thereof, modified with 2′-OMe.

In some embodiments, a double-stranded oligonucleotide provided herein comprises a sense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, position 22, position 23, position 24, position 25, position 26, position 27, position 28, position 29, position 30, position 31, position 32, position 33, position 34, position 35, position 36, or any combination thereof, modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

### 5′-Terminal Phosphate

In some embodiments, a double-stranded oligonucleotide described herein comprises a 5′-terminal phosphate. In some embodiments, the 5′-terminal phosphate groups of the double-stranded oligonucleotide enhance the interaction with Ago2. However, oligonucleotides comprising a 5′-phosphate group may be susceptible to degradation via phosphatases or other enzymes, which can limit their bioavailability in vivo. In some embodiments, a double-stranded oligonucleotide herein comprises analogs of 5′ phosphates that are resistant to such degradation. In some embodiments, the phosphate analog is oxymethyl phosphonate, vinyl phosphonate or malonyl phosphonate, or a combination thereof. In some embodiments, the 5′ end of a double-stranded oligonucleotide strand is attached to chemical moiety that mimics the electrostatic and steric properties of a natural 5′-phosphate group (“phosphate mimic”).

In some embodiments, a double-stranded oligonucleotide herein has a phosphate analog at a 4′-carbon position of the sugar (referred to as a “4′-phosphate analog”). See, e.g., Intl. Patent Application Publication No. WO 2018/045317. In some embodiments, a double-stranded oligonucleotide herein comprises a 4′-phosphate analog at a 5′-terminal nucleotide. In some embodiments, a phosphate analog is an oxymethyl phosphonate, in which the oxygen atom of the oxymethyl group is bound to the sugar moiety (e.g., at its 4′-carbon) or analog thereof. In other embodiments, a 4′-phosphate analog is a thiomethyl phosphonate or an aminomethyl phosphonate, in which the sulfur atom of the thiomethyl group or the nitrogen atom of the amino methyl group is bound to the 4′-carbon of the sugar moiety or analog thereof. In some embodiments, a 4′-phosphate analog is an oxymethyl phosphonate. In some embodiments, an oxymethyl phosphonate is represented by the formula —O—CH₂—PO(OH)₂, —O—CH₂—PO(OR)₂, or —O—CH₂—POOH(R), in which R is independently selected from H, CH₃, an alkyl group, CH₂CH₂CN, CH₂OCOC(CH₃)₃, CH₂OCH₂CH₂Si(CH₃)₃ or a protecting group. In some embodiments, the alkyl group is CH₂CH₃. More typically, R is independently selected from H, CH₃ or CH₂CH₃. In some embodiment, R is CH₃. In some embodiments, the 4′-phosphate analog is 5′-methoxyphosphonate-4′-oxy. In some embodiments, the 4′-phosphate analog is 4′-oxymethyl phosphonate.

In some embodiments, a double-stranded oligonucleotide provided herein comprises an antisense strand comprising a 4′-phosphate analog at the 5′-terminal nucleotide, wherein 5′-terminal nucleotide comprises the following structure:

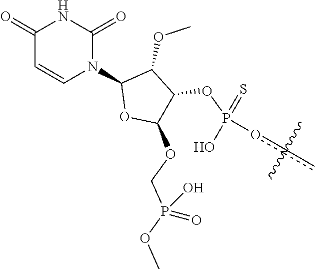

4′-O-monomethylphosphonate-2′-O-methyluridine phosphorothioate
[MePhosphonate-40-mUs, alternatively referred to as “MeMOP”]

### Modified Internucleotide Linkage

In some embodiments, a double-stranded oligonucleotide herein comprises a modified internucleoside linkage. In some embodiments, phosphate modifications or substitutions result in an oligonucleotide that comprises at least about 1 (e.g., at least 1, at least 2, at least 3 or at least 5) modified internucleotide linkage. In some embodiments, any one of the oligonucleotides disclosed herein comprises about 1 to about 10 (e.g., 1 to 10, 2 to 8, 4 to 6, 3 to 10, 5 to 10, 1 to 5, 1 to 3 or 1 to 2) modified internucleotide linkages. In some embodiments, any one of the oligonucleotides disclosed herein comprises 1, 2, 3, 4, 5, 6, 7, 8, 9 or 10 modified internucleotide linkages.

A modified internucleotide linkage may be a phosphorodithioate linkage, a phosphorothioate linkage, a phosphotriester linkage, a thionoalkylphosphonate linkage, a thionalkylphosphotriester linkage, a phosphoramidite linkage, a phosphonate linkage or a boranophosphate linkage. In some embodiments, at least one modified internucleotide linkage of any one of the oligonucleotides as disclosed herein is a phosphorothioate linkage.

In some embodiments, a double-stranded oligonucleotide provided herein has a phosphorothioate linkage between one or more of positions 1 and 2 of the sense strand, positions 1 and 2 of the antisense strand, positions 2 and 3 of the antisense strand, positions 3 and 4 of the antisense strand, positions 20 and 21 of the antisense strand, and positions 21 and 22 of the antisense strand. In some embodiments, the oligonucleotide described herein has a phosphorothioate linkage between each of positions 1 and 2 of the sense strand, positions 1 and 2 of the antisense strand, positions 2 and 3 of the antisense strand, positions 20 and 21 of the antisense strand, and positions 21 and 22 of the antisense strand. In some embodiments, the oligonucleotide described herein has a phosphorothioate linkage between each of positions 1 and 2 of the sense strand, positions 1 and 2 of the antisense strand, positions 2 and 3 of the antisense strand, positions 3 and 4 of the antisense strand, positions 20 and 21 of the antisense strand, and positions 21 and 22 of the antisense strand. In some embodiments, the oligonucleotide described herein has a phosphorothioate linkage between each of positions 1 and 2 of the sense strand, positions 18 and 19 of the sense strand, positions 19 and 20 of the sense strand, positions 1 and 2 of the antisense strand, positions 2 and 3 of the antisense strand, positions 3 and 4 of the antisense strand, positions 20 and 21 of the antisense strand, and positions 21 and 22 of the antisense strand.

In some embodiments, the oligonucleotide described herein has a phosphorothioate linkage between each of positions 1 and 2 of the sense strand, the third to last position and penultimate position of the sense strand, and the penultimate position and ultimate position of the sense strand.

In some embodiments, the oligonucleotide described herein has a phosphorothioate linkage between positions 1 and 2 of the sense strand, positions 1 and 2 of the antisense strand, positions 2 and 3 of the antisense strand, positions 3 and 4 of the antisense strand, positions 13 and 14 of the antisense strand, positions 14 and 15 of the antisense strand, positions 20 and 21 of the antisense strand, and positions 21 and 22 of the antisense strand.

In some embodiments, the oligonucleotide described herein has a phosphorothioate linkage between positions 1 and 2 of the sense strand, positions 1 and 2 of the antisense strand, positions 2 and 3 of the antisense strand, positions 3 and 4 of the antisense strand, positions 16 and 17 of the antisense strand, positions 17 and 18 of the antisense strand, positions 18 and 19 of the antisense strand, positions 19 and 20 of the antisense strand, positions 20 and 21 of the antisense strand, and positions 21 and 22 of the antisense strand.

In some embodiments, the oligonucleotide described herein has a phosphorothioate linkage between positions 1 and 2 of the sense strand, positions 1 and 2 of the antisense strand, positions 2 and 3 of the antisense strand, positions 3 and 4 of the antisense strand, positions 13 and 14 of the antisense strand, positions 14 and 15 of the antisense strand, positions 16 and 17 of the antisense strand, positions 17 and 18 of the antisense strand, positions 18 and 19 of the antisense strand, positions 19 and 20 of the antisense strand, positions 20 and 21 of the antisense strand, and positions 21 and 22 of the antisense strand.

In some embodiments, the oligonucleotide described herein has a phosphorothioate linkage between positions 1 and 2 of the sense strand, positions 1 and 2 of the antisense strand, positions 2 and 3 of the antisense strand, positions 3 and 4 of the antisense strand, positions 12 and 13 of the antisense strand, positions 13 and 14 of the antisense strand, positions 14 and 15 of the antisense strand, positions 15 and 16 of the antisense strand, positions 16 and 17 of the antisense strand, positions 17 and 18 of the antisense strand, positions 18 and 19 of the antisense strand, positions 19 and 20 of the antisense strand, positions 20 and 21 of the antisense strand, and positions 21 and 22 of the antisense strand. In some embodiments, the oligonucleotide comprises a nucleotide at position 14 of a 22 nucleotide antisense strand, wherein the nucleotide is flanked by phosphorothioate linkages (i.e. a phosphorothioate linkage between positions 13 and 14 and between positions 14 and 15). In some embodiments, the flanked nucleotide at position 14 is the ultimate nucleotide of a duplex between the antisense strand and sense strand. In some embodiments, the oligonucleotide comprises a sense and antisense strand, wherein the antisense strand comprises a flanked oligonucleotide at position 14 of a 22 nucleotide antisense strand (i.e. a phosphorothioate linkage between positions 13 and 14 and between positions 14 and 15), wherein the sense and antisense strand form a duplex and the antisense strand comprises an overhang, and wherein the nucleotide at position 14 is within the overhang.

In some embodiments, an oligonucleotide conjugate described herein comprises a peptide nucleic acid (PNA). PNAs are oligonucleotide mimics in which the sugar-phosphate backbone has been replaced by a pseudopeptide skeleton, composed of N-(2-aminoethyl)glycine units. Nucleobases are linked to this skeleton through a two-atom carboxymethyl spacer. In some embodiments, an oligonucleotide conjugate described herein comprises a morpholino oligomer (PMO) comprising an internucleotide linkage backbone of methylene morpholine rings linked through phosphorodiamidate groups.

### Base Modifications

In some embodiments, a double-stranded oligonucleotide herein comprises one or more modified nucleobases. In some embodiments, modified nucleobases (also referred to herein as base analogs) are linked at the 1′ position of a nucleotide sugar moiety. In some embodiments, a modified nucleobase is a nitrogenous base. In some embodiments, a modified nucleobase does not contain nitrogen atom. See, e.g., US Patent Application Publication No. 2008/0274462. In some embodiments, a modified nucleotide comprises a universal base. In some embodiments, a modified nucleotide does not contain a nucleobase (abasic).

In some embodiments, a universal base is a heterocyclic moiety located at the 1′ position of a nucleotide sugar moiety in a modified nucleotide, or the equivalent position in a nucleotide sugar moiety substitution, that, when present in a duplex, can be positioned opposite more than one type of base without substantially altering structure of the duplex. In some embodiments, compared to a reference single-stranded nucleic acid (e.g., oligonucleotide) that is fully complementary to a target nucleic acid, a single-stranded nucleic acid containing a universal base forms a duplex with the target nucleic acid that has a lower Tm than a duplex formed with the complementary nucleic acid. In some embodiments, when compared to a reference single-stranded nucleic acid in which the universal base has been replaced with a base to generate a single mismatch, the single-stranded nucleic acid containing the universal base forms a duplex with the target nucleic acid that has a higher Tm than a duplex formed with the nucleic acid comprising the mismatched base.

Non-limiting examples of universal-binding nucleotides include, but are not limited to, inosine, 1-β-D-ribofuranosyl-5-nitroindole and/or 1-β-D-ribofuranosyl-3-nitropyrrole (see, US Patent Application Publication No. 2007/0254362; Van Aerschot et al. (1995) NUCLEIC ACIDS RES. 23:4363-70; Loakes et al. (1995) NUCLEIC ACIDS RES. 23:2361-66; and Loakes & Brown (1994) NUCLEIC ACIDS RES. 22:4039-43).

### Tm-Increasing Nucleotides

In some embodiments, the oligonucleotide described herein comprises at least one Tm-increasing nucleotide in the sense strand. In some embodiments, the oligonucleotide has one Tm-increasing nucleotide in the sense strand. In some embodiments, the oligonucleotide has up to two Tm-increasing nucleotides in the sense strand. In some embodiments, the oligonucleotide has up to three Tm-increasing nucleotides in the sense strand. In some embodiments, the oligonucleotide has up to four Tm-increasing nucleotides in the sense strand. In some embodiments, the oligonucleotide has up to five Tm-increasing nucleotides in the sense strand. In some embodiments, the oligonucleotide has up to six Tm-increasing nucleotides in the sense strand. In some embodiments, the oligonucleotide has up to seven Tm-increasing nucleotides in the sense strand. In some embodiments, the oligonucleotide has up to eight Tm-increasing nucleotides in the sense strand. In some embodiments, the oligonucleotide has up to nine Tm-increasing nucleotides in the sense strand. In some embodiments, the oligonucleotide has up to ten Tm-increasing nucleotides in the sense strand.

In some embodiments, the oligonucleotide has 1 to 2 Tm-increasing nucleotides in the sense strand. In some embodiments, the oligonucleotide has 1 to 3 Tm-increasing nucleotides in the sense strand. In some embodiments, the oligonucleotide has 1 to 4 Tm-increasing nucleotides in the sense strand. In some embodiments, the oligonucleotide has 1 to 5 Tm-increasing nucleotides in the sense strand.

In some embodiments, an oligonucleotide comprising a stem-loop comprises a Tm-increasing nucleotide in the stem. In some embodiments, an oligonucleotide comprising a stem-loop comprises Tm-increasing nucleotides in at least one base pair of the stem. In some embodiments, an oligonucleotide comprising a stem-loop comprises Tm-increasing nucleotides in one base pair of the stem. In some embodiments, an oligonucleotide comprising a stem-loop comprises Tm-increasing nucleotides in two base pairs of the stem. In some embodiments, an oligonucleotide comprising a stem-loop comprises Tm-increasing nucleotides in three base pairs of the stem. In some embodiments, an oligonucleotide comprising a stem-loop comprises Tm-increasing nucleotides in four base pairs of the stem. In some embodiments, an oligonucleotide comprising a stem-loop comprises Tm-increasing nucleotides in five base pairs of the stem. In some embodiments, an oligonucleotide comprising a stem-loop comprises Tm-increasing nucleotides in six base pairs of the stem.

Tm-increasing nucleotides include, but are not limited to, bicyclic nucleotides, tricyclic nucleotides, a G-clamp, and analogues thereof, hexitol nucleotides, or a modified nucleotide. In some embodiments, the Tm-increasing nucleotide is a bicyclic nucleotide. In some embodiments, the Tm-increasing nucleotide is a locked nucleic acid (LNA).

In some embodiments, the sense strand of the oligonucleotide comprises a Tm-increasing nucleotide at one or more of positions 1, 2, 7, 8, 9, 10, 11, 12, 14, 15, 16, 18, and 19. In some embodiments, the sense strand of the oligonucleotide comprises a Tm-increasing nucleotide at position 1. In some embodiments, the sense strand of the oligonucleotide comprises a Tm-increasing nucleotide at position 2. In some embodiments, the sense strand of the oligonucleotide comprises a Tm-increasing nucleotide at position 9. In some embodiments, the sense strand of the oligonucleotide comprises a Tm-increasing nucleotide at position 10. In some embodiments, the sense strand of the oligonucleotide comprises a Tm-increasing nucleotide at position 11. In some embodiments, the sense strand of the oligonucleotide comprises a Tm-increasing nucleotide at position 12. In some embodiments, the sense strand of the oligonucleotide comprises a Tm-increasing nucleotide at position 14. In some embodiments, the sense strand of the oligonucleotide comprises a Tm-increasing nucleotide at position 15. In some embodiments, the sense strand of the oligonucleotide comprises a Tm-increasing nucleotide at position 16. In some embodiments, the sense strand of the oligonucleotide comprises a Tm-increasing nucleotide at position 18. In some embodiments, the sense strand of the oligonucleotide comprises a Tm-increasing nucleotide at position 19.

In some embodiments, a 29-nucleotide sense strand, with nucleotides numbered 5′ to 3′, comprises a Tm-increasing nucleotide at position 1. In some embodiments, a 30-nucleotide sense strand, with nucleotides numbered 5′ to 3′, comprises a Tm-increasing nucleotide at position 1. In some embodiments, a 31-nucleotide sense strand, with nucleotides numbered 5′ to 3′, comprises a Tm-increasing nucleotide at position 1. In some embodiments, a 32-nucleotide sense strand, with nucleotides numbered 5′ to 3′, comprises a Tm-increasing nucleotide at position 1. In some embodiments, a 33-nucleotide sense strand, with nucleotides numbered 5′ to 3′, comprises a Tm-increasing nucleotide at position 1. In some embodiments, a 34-nucleotide sense strand, with nucleotides numbered 5′ to 3′, comprises a Tm-increasing nucleotide at position 1.

In some embodiments, a 23-nucleotide sense strand, with nucleotides numbered 5′ to 3′, comprises a Tm-increasing nucleotide at one or more of positions 1, 14, and 23. In some embodiments, a 23-nucleotide sense strand, with nucleotides numbered 5′ to 3′, comprises a Tm-increasing nucleotide at position 1. In some embodiments, a 23-nucleotide sense strand, with nucleotides numbered 5′ to 3′, comprises a Tm-increasing nucleotide at position 1 and position 14. In some embodiments, a 23-nucleotide sense strand, with nucleotides numbered 5′ to 3′, comprises a Tm-increasing nucleotide at position 1, position 14, and position 23. In some embodiments, a 12-nucleotide sense strand, with nucleotides numbered 5′ to 3′, comprises a Tm-increasing nucleotide at position 2, position 7, and position 8.

In some embodiments, a 16-nucleotide sense strand, with nucleotides numbered 5′ to 3′, comprises a Tm-increasing nucleotide at position 1. In some embodiments, a 17-nucleotide sense strand, with nucleotides numbered 5′ to 3′, comprises a Tm-increasing nucleotide at position 1. In some embodiments, a 18-nucleotide sense strand, with nucleotides numbered 5′ to 3′, comprises a Tm-increasing nucleotide at position 1. In some embodiments, a 19-nucleotide sense strand, with nucleotides numbered 5′ to 3′, comprises a Tm-increasing nucleotide at position 1. In some embodiments, a 21-nucleotide sense strand, with nucleotides numbered 5′ to 3′, comprises a Tm-increasing nucleotide at position 1.

In some embodiments, the disclosure provides an RNAi oligonucleotide for reducing target gene expression by the RNAi pathway comprising a combination of one or more Tm-increasing nucleotides and one or more nucleotides (e.g., a modified nucleotide) having a lower binding affinity, wherein the duplex region comprising the RNAi oligonucleotide is maintained under physiological conditions and the ability of the RNAi oligonucleotide to inhibit or reduce target gene expression is maintained.

### Bicyclic Nucleotides

Bicyclic nucleotides typically have a sugar moiety with a 4 to 7 membered ring (including but not limited to a furanosyl) comprising a bridge connecting two atoms of the 4 to 7 membered ring to form a second ring, resulting in a bicyclic structure. Such bicyclic nucleotides have various names including BNA's and LNA's for bicyclic nucleic acids and locked nucleic acids, respectively. The synthesis of bicyclic nucleotides and their incorporation into nucleic acid compounds has also been reported in the literature, including, for example, Singh et al., Chem. Commun., 1998, 4, 455-56; Koshkin et al., TETRAHEDRON, 1998, 54, 3607-30; Wahlestedt et al., PROC. NATL. ACAD. SCI. U.S.A., 2000, 97, 5633-38; Kumar et al., BIOORG. MED. CHEM. LETT., 1998, 8, 2219-22; Singh et al., J. ORG. CHEM., 1998, 63, 10035-039; U.S. Pat. Nos. 7,427,672, 7,053,207, 6,794,499, 6,770,748, 6,268,490 and 6,794,499; and published U.S. applications Ser. No. 20/040,219565, 20040014959, 20030207841, 20040192918, 20030224377, 20040143114 and 20030082807; each of which is incorporated by reference herein, in its entirety.

In some embodiments, the Tm-increasing nucleotide is a bicyclic nucleotide that comprises a bicyclic sugar moiety. In certain embodiments, the bicyclic sugar moiety comprises a first ring of 4 to 7 members and a bridge forming a North-type sugar confirmation that connects any two atoms of the first ring of the sugar moiety to form a second ring. In certain embodiments, the bridge connects the 2′-carbon and the 4′-carbon of the first ring to form a second ring.

Typically, the bridge contains 2 to 8 atoms. In certain embodiments, the bridge contains 3 atoms. In certain embodiments, the bridge contains 4 atoms. In certain embodiments, the bridge contains 5 atoms. In certain embodiments, the bridge contains 6 atoms. In certain embodiments, the bridge contains 7 atoms. In certain embodiments, the bridge contains 8 atoms. In certain embodiments, the bridge contains more than 8 atoms.

In certain embodiments, the bicyclic sugar moiety is a substituted furanosyl comprising a bridge that connects the 2′-carbon and the 4′-carbon of the furanosyl to form the second ring. In certain embodiments, the bicyclic nucleotide has the structure of Formula I:

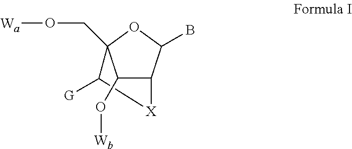

wherein B is a nucleobase;
wherein G is H, OH, NH₂, C₁-C₆ alkyl, C₂-C₆ alkenyl, C₂-C₆ alkynyl, substituted C₁-C₆ alkyl, substituted C₂-C₆ alkenyl, substituted C₂-C₆ alkynyl, acyl, substituted acyl, substituted amide, thiol, or substituted thio;
wherein X is O, S, or NR₁, wherein R₁ is H, C₁-C₆ alkyl, C₁-C₆ alkoxy, benzene or pyrene; and
wherein Wₐ and W_{b} are each independently, H, OH, a hydroxyl protecting group, a phosphorous moiety, or an internucleotide linking group attaching the nucleotide represented by Formula I to another nucleotide or to an oligonucleotide and wherein at least one of Wₐᵣ or W_{b} is an internucleotide linking group attaching the nucleotide represented by Formula I to an oligonucleotide.

In certain embodiments of Formula I, G is H and X is NR₁, wherein R₁ is benzene or pyrene. In certain embodiments, of Formula I, G is H and X is S.

In certain embodiments of Formula I, G is H and X is O:

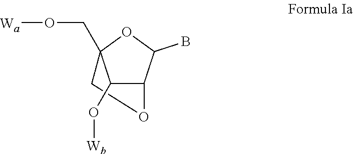

In certain embodiments of Formula I, G is H and X is NR₁, wherein R₁ is H, CH₃, or OCH₃:

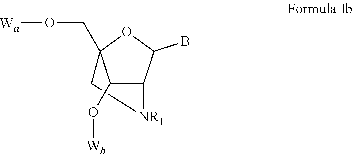

In certain embodiments of Formula I, G is OH or NH₂ and X is O.

In certain embodiments of Formula I, G is OH and X is O:

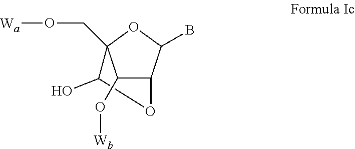

In certain embodiments of Formula I, G is NH₂ and X is O:

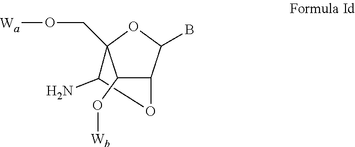

In certain embodiments, of Formula I, G is CH₃ or CH₂OCH₃ and X is O. In certain embodiments, of Formula I, G is CH₃ and X is O:

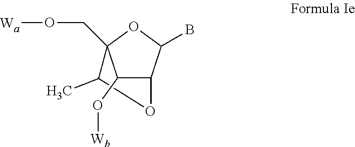

In certain embodiments, of Formula I, G is CH₂OCH₃ and X is O:

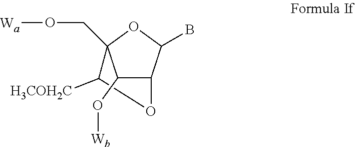

In certain embodiments, the bicyclic nucleotide has the structure of Formula II:

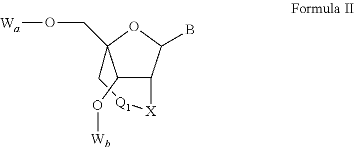

wherein B is a nucleobase;
wherein Q₁ is CH₂ or O;
wherein X is CH₂, O, S, or NR₁, wherein R₁ is H, C₁-C₆ alkyl, C₁-C₆ alkoxy, benzene or pyrene;
wherein if Q₁ is O, X is CH₂;
wherein if Q₁ is CH₂, X is CH₂, O, S, or NR₁, wherein R₁ is H, C₁-C₆ alkyl, C₁-C₆ alkoxy, benzene or pyrene;
wherein Wₐ and W_{b} are each independently, H, OH, a hydroxyl protecting group, a phosphorous moiety, or an internucleotide linking group attaching the nucleotide represented by Formula II to another nucleotide or to an oligonucleotide and wherein at least one of Wₐ or W_{b} is an internucleotide linking group attaching the nucleotide represented by Formula II to an oligonucleotide.

In certain embodiments of Formula II, Q₁ is O and X is CH₂:

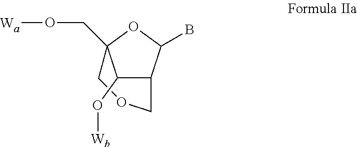

In certain embodiments of Formula II, Q₁ is CH₂ and X is O:

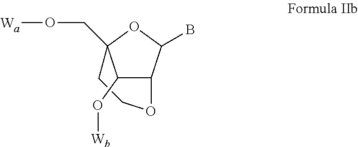

In certain embodiments of Formula II, Q₁ is CH₂ and X is NR₁, wherein R₁ is H, CH₃ or OCH₃:

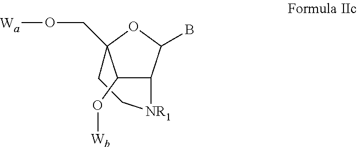

In certain embodiments of Formula II, Q₁ is CH₂ and X is NH:

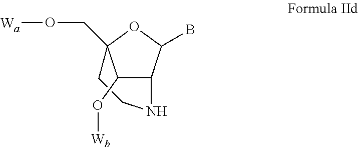

In certain embodiments, the bicyclic nucleotide has the structure of Formula III:

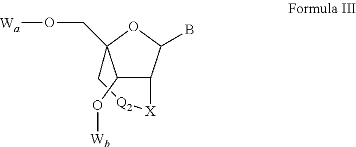

wherein B is a nucleobase;
wherein Q² is O or NR₁, wherein R₁ is H, C₁-C₆ alkyl, C₁-C₆ alkoxy, benzene or pyrene;
wherein X is CH₂, O, S, or NR₁, wherein R₁ is H, C₁-C₆ alkyl, C₁-C₆ alkoxy, benzene or pyrene;
wherein if Q₂ is O, X is NR₁;
wherein if Q₂ is NR₁, X is O or S;
wherein Wₐ and We are each independently, H, OH, a hydroxyl protecting group, a phosphorous moiety, or an internucleotide linking group attaching the nucleotide represented by Formula III to another nucleotide or to an oligonucleotide and wherein at least one of Wₐ or W_{b} is an internucleotide linking group attaching the nucleotide represented by Formula III to an oligonucleotide.

In certain embodiments of Formula III, Q₂ is O and X is NR₁. In certain embodiments of Formula III, Q₂ is O and X is NR₁, wherein R₁ is C₁-C₆ alkyl. In certain embodiments of Formula III, Q₂ is O and X is NR₁ and R₁ is H or CH₃.

In certain embodiments of Formula III, Q₂ is O and X is NR₁ and R₁ is CH₃:

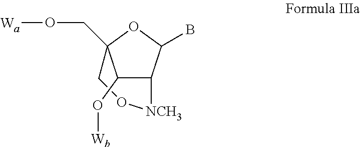

In certain embodiments of Formula III, Q₂ is NR₁ and X is O. In certain embodiments of Formula II, Q₂ is NR₁, wherein R₁ is C₁-C₆ alkyl and X is O.

In certain embodiments of Formula III, Q₂ is NCH₃ and X is O:

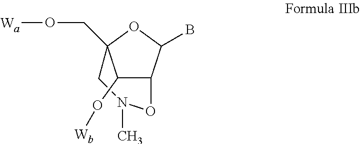

In certain embodiments, the bicyclic nucleotide has the structure of Formula IV:

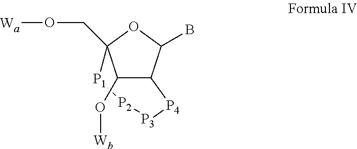

wherein B is a nucleobase;
wherein P₁ and P₃ are CH₂, P₂ is CH₂ or O and P₄ is O; and
wherein Wₐ and W_{b} are each independently, H, OH, a hydroxyl protecting group, a phosphorous moiety, or an internucleotide linking group attaching the nucleotide represented by Formula IV to another nucleotide or to an oligonucleotide and wherein at least one of Wₐ or W_{b} is an internucleotide linking group attaching the nucleotide represented by Formula IV to an oligonucleotide.

In certain embodiments of Formula IV, P₁, P₂, and P₃ are CH₂, and P₄ is O:

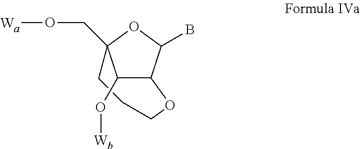

In certain embodiments of Formula IV, P₁ and P₃ are CH₂, P₂ is O and P₄ is O:

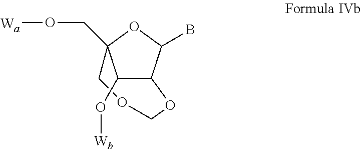

In certain embodiments, the bicyclic nucleotide has the structure of Formula Va or Vb:

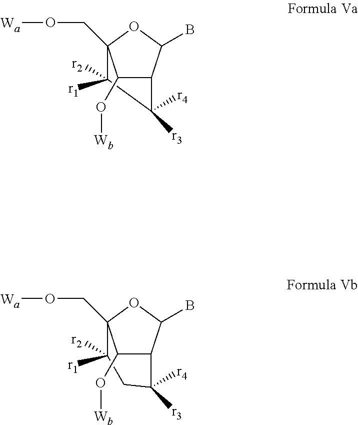

wherein B is a nucleobase;
wherein r1, r2, r3, and r4 are each independently H, halogen, C₁-C₁₂ alkyl, substituted C₁-C₁₂ alkyl, C₂-C₁₂ alkenyl, substituted C₂-C₁₂ alkenyl, C₂-C₁₂ alkynyl; substituted C₂-C₁₂ alkynyl; C₁-C₁₂ alkoxy; substituted C₂-C₁₂ alkoxy, OT₁, ST₁, SOT₁, SO₂T₁, NT₁T₂, N₃, CN, C(═O)OT₁, C(═O)NT₁T₂, C(═O)T₁, O—C(═O)NT₁T₂, N(H)C(═NH) NTIT2, N(H)C(═O)NTIT₂ or N(H)C(═S)NTIT₂, wherein each of T1 and T2 is independently H, C₁-C₆ alkyl, or substituted C₁-C₁₆ alkyl; or
r1 and r2 or r3 and r4 together are ═C(r5)(r6), wherein r5 and r6 are each independently H, halogen, C₁-C₁₂ alkyl, or substituted C₁-C₁₂ alkyl; and
wherein Wₐ and We are each independently, H, OH, a hydroxyl protecting group, a phosphorous moiety, or an internucleotide linking group attaching the nucleotide represented by Formula V to another nucleotide or to an oligonucleotide and wherein at least one of Wₐ or W_{b} is an internucleotide linking group attaching the nucleotide represented by Formula V to an oligonucleotide.

In certain embodiments, the bicyclic sugar moiety is a substituted furanosyl comprising a bridge that connects the 2′-carbon and the 4′-carbon of the furanosyl to form the second ring, wherein the bridge that connects the 2′-carbon and the 4′-carbon of the furanosyl includes, but is not limited to:
a) 4′-CH₂—O—N(R)-2′ and 4′-CH₂—N(R)—O-2′, wherein R is H, C₁-C₁₂ alkyl, or a protecting group, including, for example, 4′-CH₂—NH—O-2′ (also known as BNA^{NC}), 4′-CH₂—N(CH₃)—O-2′ (also known as BNA^{NC} [NMe]), (as described in U.S. Pat. No. 7,427,672, which is hereby incorporated by reference in its entirety);
b) 4′-CH₂-2′; 4′-(CH₂)₂-2′; 4′-(CH₂) 3-2′; 4′-(CH₂)—O-2′ (also known as LNA); 4′-(CH₂)—S-2′; 4′-(CH₂) 2-O-2′ (also known as ENA); 4′-CH(CH₃)—O-2′ (also known as cEt); and 4′-CH(CH₂OCH₃)—O-2′ (also known as cMOE), and analogs thereof (as described in U.S. Pat. No. 7,399,845, which is hereby incorporated by reference in its entirety);
c) 4′-C(CH₃)(CH₃)—O-2′ and analogs thereof (as described in U.S. Pat. No. 8,278,283, which is hereby incorporated by reference in its entirety);
d) 4′-CH₂—N(OCH₃)-2′ and analogs thereof (as described in U.S. Pat. No. 8,278,425, which is hereby incorporated by reference in its entirety);
e) 4′-CH₂—O—N(CH₃)-2′ and analogs thereof (as described in U.S. Patent Publication No. 2004/0171570, which is hereby incorporated by reference in its entirety);
f) 4′-CH₂—C(H)(CH₃)-2′ and analogs thereof (as described in Chattopadhyaya et al., J. ORG. CHEM., 2009, 74, 118-34, which is hereby incorporated by reference in its entirety); and
g) 4′-CH₂—C(═CH₂)-2′ and analogs thereof as described in U.S. Pat. No. 8,278,426, which is hereby incorporated by reference in its entirety).

In certain embodiments, the bicyclic nucleotide (BN) is one or more of the following: (a) methyleneoxy BN, (b)ethyleneoxy BN, (c) aminooxy BN; (d) oxyamino BN, (e) methyl(methyleneoxy) BN (also known as constrained ethyl or cET), (f)methylene-thio BN, (g) methylene amino BN, (h)methyl carbocyclic BN, and (i) propylene carbocyclic BN, as shown below.

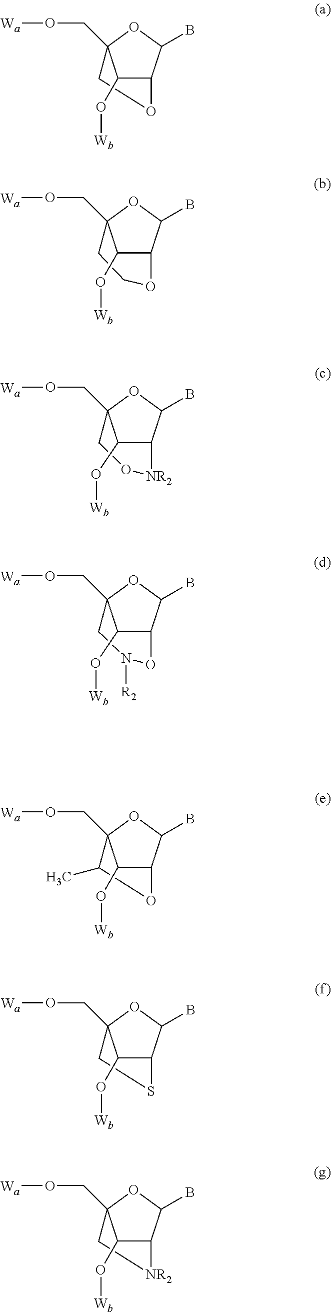

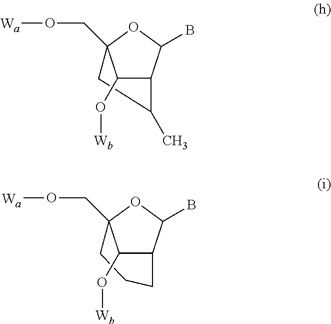

In the bicyclic nucleotides of (a) to (i) above, B is a nucleobase, R₂ is H or CH₃ and Wₐ and W_{b} are each independently, H, OH, a hydroxyl protecting group, a phosphorous moiety, or an internucleotide linking group attaching the bicyclic nucleotide to another nucleotide or to an oligonucleotide and wherein at least one of Wₐ or W_{b} is an internucleotide linking group attaching the bicyclic nucleotide to an oligonucleotide.

In one embodiment of the oxyamino BN (d), R₂ is CH₃, as follows (also known as BNA^{NC}[NMe]):

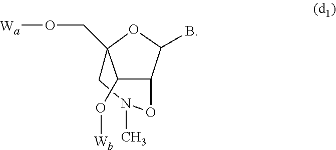

In certain embodiments, bicyclic sugar moieties and bicyclic nucleotides incorporating such bicyclic sugar moieties are further defined by isomeric configuration. In certain embodiments, the bicyclic sugar moiety or nucleotide is in the α-L configuration. In certain embodiments, the bicyclic sugar moiety or nucleotide is in the β-D configuration. For example, in certain embodiments, the bicyclic sugar moiety or nucleotide comprises a 2′0,4′-C-methylene bridge (2′-O—CH₂-4′) in the α-L configuration (α-L LNA). In certain embodiments, the bicyclic sugar moiety or nucleotide is in the R configuration. In certain embodiments, the bicyclic sugar moiety or nucleotide is in the S configuration. For example, in certain embodiments, the bicyclic sugar moiety or nucleotide comprises a 4′-CH(CH₃)—O-2′ bridge (i.e., cEt) in the S-configuration.

### Tricyclic Nucleotides

In some embodiments, the Tm-increasing nucleotide is a tricyclic nucleotide. The synthesis of tricyclic nucleotides and their incorporation into nucleic acid compounds has also been reported in the literature, including, for example, Steffens et al., J. AM. CHEM. SOC. 1997; 119:11548-549; Steffens et al., J. ORG. CHEM. 1999;121 (14): 3249-55; Renneberg et al., J. AM. CHEM. SOC. 2002; 124:5993-6002; Ittig et al., NUCLEIC ACIDS RES. 2004; 32 (1): 346-53; Scheidegger et al., CHEMISTRY 2006; 12:8014-23; Ivanova et al., OLIGONUCLEOTIDES 2007; 17:54-65; each of which is each hereby incorporated by reference in its entirety.

In certain embodiments, the tricyclic nucleotide is a tricyclo nucleotide (also called tricyclo DNA) in which the 3′-carbon and 5′-carbon centers are connected by an ethylene that is fused to a cyclopropane ring, as discussed for example in Leumann C J, BIOORG. MED. CHEM. 2002; 10:841-54 and published U.S. Applications 2015/0259681 and 2018/0162897, which are each hereby incorporated by reference. In certain embodiments, the tricyclic nucleotide comprises a substituted furanosyl ring comprising a bridge that connects the 2′-carbon and the 4′-carbon of the furanosyl to form a second ring, and a third fused ring resulting from a group connecting the 5′-carbon to the methylene group of the bridge that connects the 2′-carbon and the 4′-carbon of the furanosyl, as discussed, for example, in published U.S. Application 2015/0112055, which is hereby incorporated by reference.

### Other Tₘ-Increasing Nucleotides

In addition to bicyclic and tricyclic nucleotides, other Tₘ-increasing nucleotides can be used in the RNAi oligonucleotides described herein. For example, in certain embodiments, the Tₘ-increasing nucleotide is a G-clamp, guanidine G-clamp or analogue thereof (Wilds et al., CHEM, 2002; 114:123 and Wilds et al., CHIM ACTA 2003; 114:123), a hexitol nucleotide (Herdewijn, CHEM. BIODIVERSITY 2010; 7:1-59), or a modified nucleotide. The modified nucleotide can have a modified nucleobase, as described herein, including for example, 5-bromo-uracil, 5-iodo-uracil, 5-propynyl-modified pyrimidines, or 2-amino adenine (also called 2,6-diaminopurine)(Deleavey et al., CHEM. & BIOL. 2012; 19:937-54) or 2-thio uridine, 5 Me-thio uridine, and pseudo uridine. The modified nucleotide can also have a modified sugar moiety, as described for example, in U.S. Pat. No. 8,975,389, which is hereby incorporated by reference, or as described herein, except that the Tₘ-increasing nucleotide is not modified at the 2′-carbon of the sugar moiety with a 2′-F or a 2′-OMe.

In certain embodiments, the Tₘ-increasing nucleotide is a bicyclic nucleotide. In certain embodiments, the Tₘ-increasing nucleotide is a tricyclic nucleotide. In certain embodiments, the Tₘ-increasing nucleotide a G-clamp, guanidine G-clamp or analogue thereof. In certain embodiments, the Tₘ-increasing nucleotide is a hexitol nucleotide. In certain embodiments, the Tₘ-increasing nucleotide is a bicyclic or tricyclic nucleotide. In certain embodiments, the Tₘ-increasing nucleotide is a bicyclic nucleotide, a tricyclic nucleotide, or a G-clamp, guanidine G-clamp or analogue thereof. In certain embodiments, the Tₘ-increasing nucleotide is a bicyclic nucleotide, a tricyclic nucleotide, a G-clamp, guanidine G-clamp or analogue thereof, or a hexitol nucleotide.

In certain embodiments, the Tₘ-increasing nucleotide increases the Tₘ of the nucleic acid inhibitor molecule by at least 2° C. per incorporation. In certain embodiments, the Tₘ-increasing nucleotide increases the Tₘ of nucleic acid inhibitor molecule by at least 3° C. per incorporation. In certain embodiments, the Tₘ-increasing nucleotide increases the Tₘ of nucleic acid inhibitor molecule by at least 4° C. per incorporation. In certain embodiments, the Tₘ-increasing nucleotide increases the Tₘ of nucleic acid inhibitor molecule by at least 5° C. per incorporation.

### Targeting Ligands

In some embodiments, it is desirable to target the oligonucleotides of the disclosure (e.g., double-stranded oligonucleotides) to one or more cells or tissues of extra-hepatic tissue. In some embodiments, it is desirable to target the oligonucleotides of the disclosure (e.g., double-stranded oligonucleotides) to one or more cells of the liver (e.g., hepatocytes).

Such a strategy can help to avoid undesirable effects in other organs or avoid undue loss of the oligonucleotide to cells, tissue or organs that would not benefit from the oligonucleotide. Accordingly, in some embodiments, a double-stranded oligonucleotide disclosed herein is modified to facilitate targeting and/or delivery to a particular tissue, cell, or organ (e.g., to facilitate delivery of the conjugate to extra-hepatic tissue). In some embodiments, a double-stranded oligonucleotide comprises at least one nucleotide (e.g., 1, 2, 3, 4, 5, 6 or more nucleotides) conjugated to one or more targeting ligand(s).

In some embodiments, 1 or more (e.g., 1, 2, 3, 4, 5 or 6) nucleotides of a double-stranded oligonucleotide disclosed herein are each conjugated to a separate targeting ligand. In some embodiments, 1 nucleotide of a double-stranded oligonucleotide herein is conjugated to a separate targeting ligand. In some embodiments, 2 to 4 nucleotides of a double-stranded oligonucleotide herein are each conjugated to a separate targeting ligand. In some embodiments, targeting ligands are conjugated to 2 to 4 nucleotides at either ends of the sense or antisense strand (e.g., targeting ligands are conjugated to a 2 to 4 nucleotide overhang or extension on the 5′ or 3′ end of the sense or antisense strand) such that the targeting ligands resemble bristles of a toothbrush and the double-stranded oligonucleotide resembles a toothbrush. For example, a double-stranded oligonucleotide may comprise a stem-loop at either the 5′ or 3′ end of the sense strand and 1, 2, 3 or 4 nucleotides of the loop of the stem may be individually conjugated to a targeting ligand. In some embodiments, a double-stranded oligonucleotide provided by the disclosure comprises a stem-loop at the 3′ end of the sense strand, wherein the loop of the stem-loop comprises a triloop or a tetraloop, and wherein the 3 or 4 nucleotides comprising the triloop or tetraloop, respectfully, are individually conjugated to a targeting ligand.

GalNAc is a high affinity ligand for the ASGPR, which is primarily expressed on the sinusoidal surface of hepatocyte cells and has a major role in binding, internalizing and subsequent clearing circulating glycoproteins that contain terminal galactose or GalNAc residues (asialoglycoproteins). Conjugation (either indirect or direct) of GalNAc moieties to oligonucleotide of the instant disclosure can be used to target these oligonucleotides to the ASGPR expressed on cells. In some embodiments, an oligonucleotide of the instant disclosure is conjugated to at least one or more GalNAc moieties, wherein the GalNAc moieties target the oligonucleotide to an ASGPR expressed on human liver cells (e.g., human hepatocytes). In some embodiments, the GalNAc moiety target the oligonucleotide to the liver.

In some embodiments, an oligonucleotide of the disclosure is conjugated directly or indirectly to a monovalent GalNAc. In some embodiments, the oligonucleotide is conjugated directly or indirectly to more than one monovalent GalNAc (i.e., is conjugated to 2, 3 or 4 monovalent GalNAc moieties, and is typically conjugated to 3 or 4 monovalent GalNAc moieties). In some embodiments, an oligonucleotide is conjugated to one or more bivalent GalNAc, trivalent GalNAc or tetravalent GalNAc moieties.

In some embodiments, 1 or more (e.g., 1, 2, 3, 4, 5 or 6) nucleotides of an oligonucleotide are each conjugated to a GalNAc moiety. In some embodiments, 2 to 4 nucleotides of a tetraloop are each conjugated to a separate GalNAc. In some embodiments, 1 to 3 nucleotides of a triloop are each conjugated to a separate GalNAc. In some embodiments, targeting ligands are conjugated to 2 to 4 nucleotides at either ends of the sense or antisense strand (e.g., ligands are conjugated to a 2 to 4 nucleotide overhang or extension on the 5′ or 3′ end of the sense or antisense strand) such that the GalNAc moieties resemble bristles of a toothbrush and the oligonucleotide resembles a toothbrush. In some embodiments, GalNAc moieties are conjugated to a nucleotide of the sense strand. For example, four (4) GalNAc moieties can be conjugated to nucleotides in the tetraloop of the sense strand where each GalNAc moiety is conjugated to 1 nucleotide.

In some embodiments, the tetraloop is any combination of adenine and guanine nucleotides. In some embodiments, the tetraloop is any combination of adenine, guanine, cytosine, and uridine nucleotides.

In some embodiments, the tetraloop (L) has a monovalent GalNAc moiety attached to any one or more guanine nucleotides of the tetraloop via any linker described herein, as depicted below (X=heteroatom):

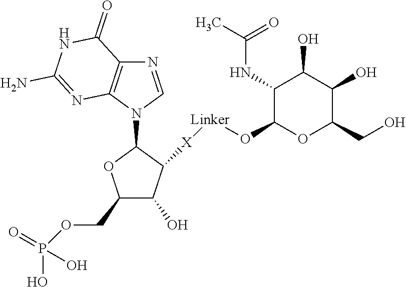

In some embodiments, the tetraloop (L) has a monovalent GalNAc attached to any one or more adenine nucleotides of the tetraloop via any linker described herein, as depicted below (X=heteroatom):

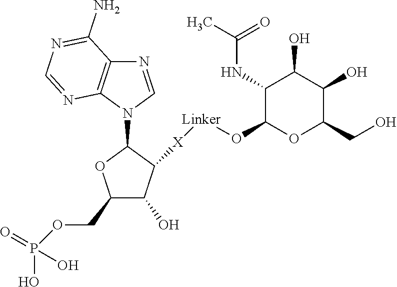

In some embodiments, a double-stranded oligonucleotide herein comprises a monovalent GalNAc attached to a guanine nucleotide referred to as [ademG-GalNAc] or 2′-aminodiethoxymethanol-Guanine-GalNAc, as depicted below:

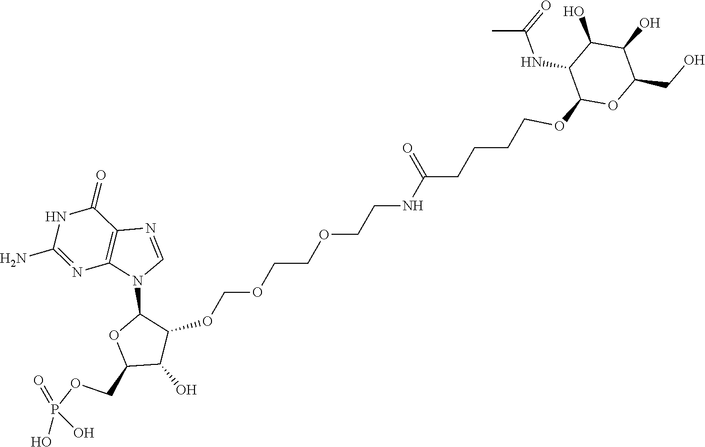 [SMILES: CC(=O)N[C@H]1[C@H](OCCCCC(=O)NCCOCCOCO[C@@H]2[C@H](O)[C@@H](COP(=O)(O)O)O[C@H]2n2cnc3c(=O)[nH]c(N)nc32)O[C@H](CO)[C@H](O)[C@@H]1O]

In some embodiments, a double-stranded oligonucleotide herein comprises a monovalent GalNAc attached to an adenine nucleotide, referred to as [ademA-GalNAc] or 2′-aminodiethoxymethanol-Adenine-GalNAc, as depicted below:

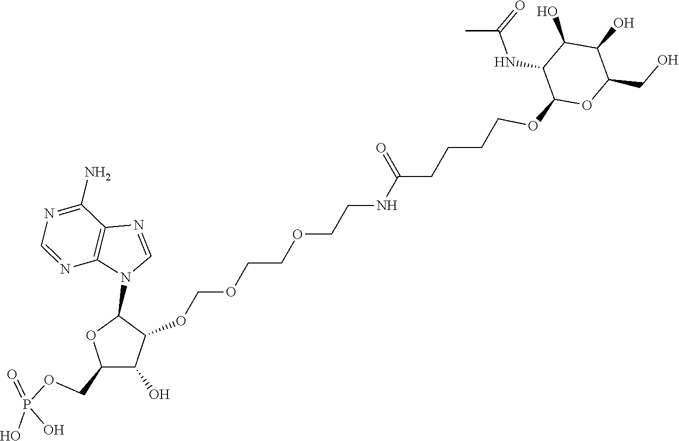 [SMILES: CC(=O)N[C@H]1[C@H](OCCCCC(=O)NCCOCCOCO[C@@H]2[C@H](O)[C@@H](COP(=O)(O)O)O[C@H]2n2cnc3c(N)ncnc32)O[C@H](CO)[C@H](O)[C@@H]1O]

An example of such conjugation is shown below for a loop comprising from 5′ to 3′ the nucleotide sequence GAAA (L=linker, X=heteroatom). Such a loop may be present, for example, at positions 27-30 of the sense strand. In the chemical formula,

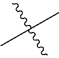

is used to describe an attachment point to the oligonucleotide strand.

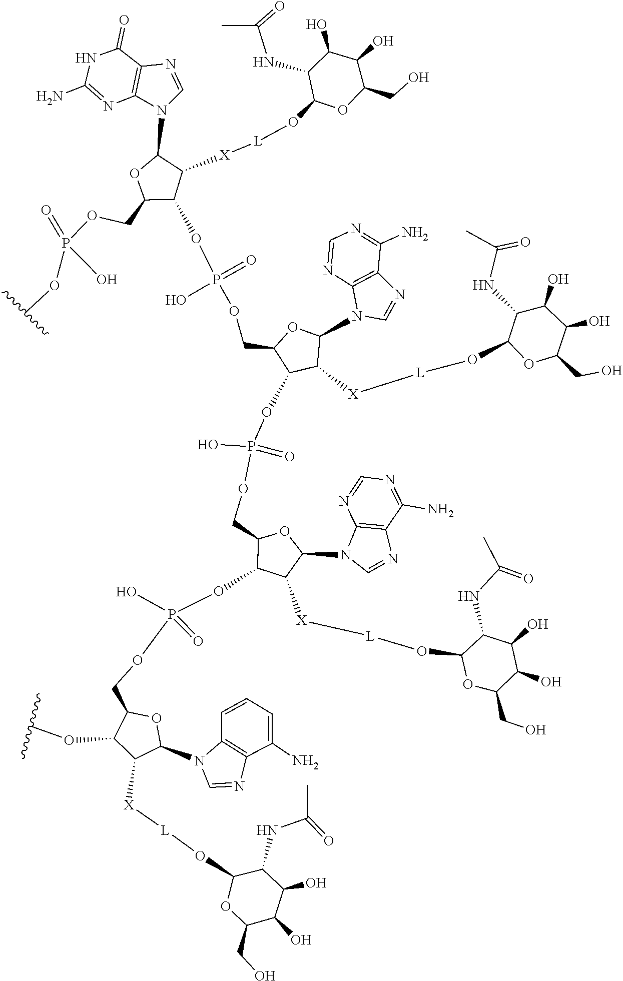

Appropriate methods or chemistry (e.g., click chemistry) can be used to link a targeting ligand to a nucleotide. In some embodiments, a targeting ligand is conjugated to a nucleotide using a click linker. In some embodiments, an acetal-based linker is used to conjugate a targeting ligand to a nucleotide of any one of the oligonucleotides described herein. Acetal-based linkers are disclosed, for example, in Intl. Patent Application Publication No. WO 2016/100401. In some embodiments, the linker is a labile linker. However, in other embodiments, the linker is stable. Examples are shown below for a loop comprising from 5′ to 3′ the nucleotides GAAA, in which GalNAc moieties are attached to nucleotides of the loop using an acetal linker. Such a loop may be present, for example, at positions 23-26 of the sense strand. In the chemical formula,

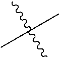

is an attachment point to the oligonucleotide strand.

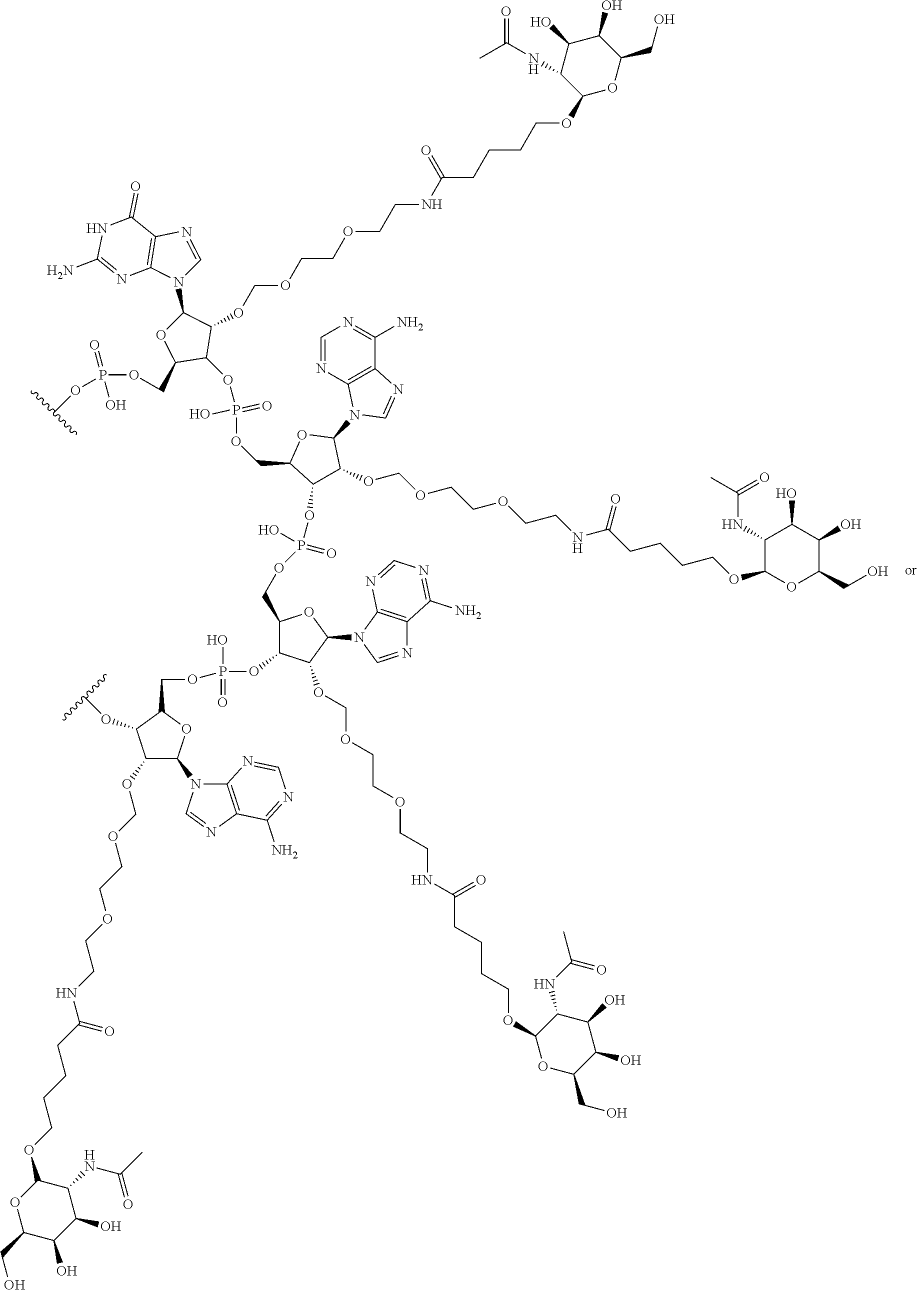

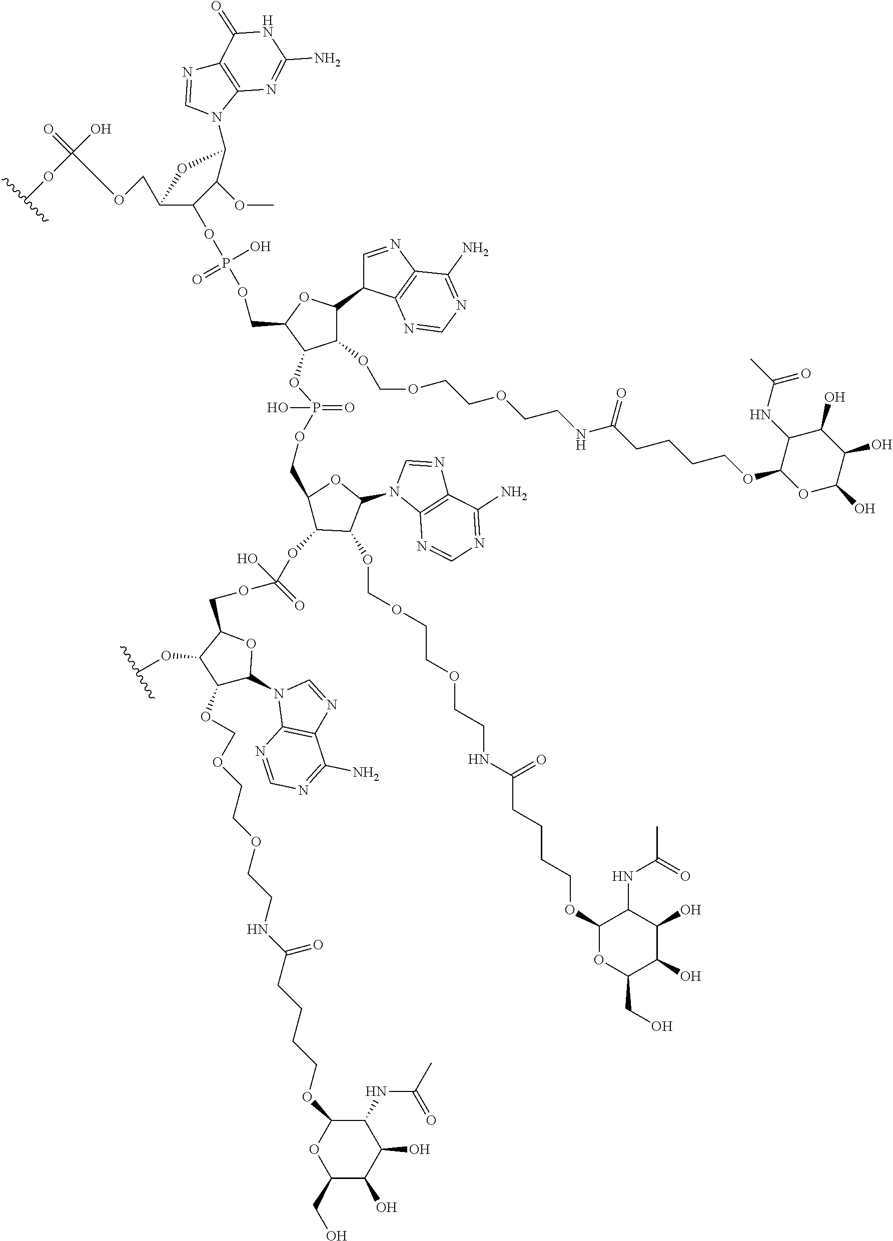

As mentioned, various appropriate methods or chemistry synthetic techniques (e.g., click chemistry) can be used to link a targeting ligand to a nucleotide. In some embodiments, a targeting ligand is conjugated to a nucleotide using a click linker. In some embodiments, an acetal-based linker is used to conjugate a targeting ligand to a nucleotide of any one of the oligonucleotides described herein. Acetal-based linkers are disclosed, for example, in Intl. Patent Application Publication No. WO 2016/100401. In some embodiments, the linker is a labile linker. However, in other embodiments, the linker is a stable linker.

In some embodiments, a duplex extension (e.g., of up to 3, 4, 5 or 6 bp in length) is provided between a targeting ligand (e.g., a GalNAc moiety) and a double-stranded oligonucleotide. In some embodiments, a double-stranded oligonucleotide herein does not have a GalNAc conjugated thereto.

### Lipid Conjugates

In some embodiments, any of the lipid moieties described herein are conjugated to a nucleotide of the sense strand of the oligonucleotide. In some embodiments, a lipid moiety is conjugated to a terminal position of the oligonucleotide. In some embodiments, the lipid moiety is conjugated to the 5′ terminal nucleotide of the sense strand. In some embodiments, the lipid moiety is conjugated to the 3′ terminal nucleotide of the sense strand.

In some embodiments, the lipid moiety is conjugated to an internal nucleotide on the sense strand. An internal position is any nucleotide position other than the two terminal positions from each end of the sense strand. In some embodiments, the lipid moiety is conjugated to one or more internal positions of the sense strand. In some embodiments, the lipid moiety is conjugated to position 1, position 2, position 3, position 4, position 5, position 6, position 7, position 8, position 9, position 10, position 11, position 12, position 13, position 14, position 15, position 16, position 17, position 18, position 19, position 20, position 21, position 22, position 23, position 24, position 25, position 26, position 27, position 28, position 29, position 30, position 31, position 32, position 33, position 34, position 35, position 36, position 37 or position 38 of a sense strand. In some embodiments, the lipid moiety is conjugated to position 1 of the sense strand. In some embodiments, the lipid moiety is conjugated to position 2 of the sense strand. In some embodiments, the lipid moiety is conjugated to position 4 of the sense strand. In some embodiments, the lipid moiety is conjugated to position 6 of the sense strand. In some embodiments, the lipid moiety is conjugated to position 8 of the sense strand. In some embodiments, the lipid moiety is conjugated to position 15 of the sense strand. In some embodiments, the lipid moiety is conjugated to position 28 of the sense strand. In some embodiments, the lipid moiety is conjugated to position 38 of the sense strand.

In some embodiments, the lipid moiety is conjugated to a nucleotide of the sense strand that forms a base pair with a nucleotide at position 18, position 17, position 16, position 15, position 14, position 13, or position 12 of the antisense strand. In some embodiments, the lipid moiety is conjugated to a nucleotide of the sense strand that forms a base pair with a nucleotide at position 18 of the antisense strand. In some embodiments, the lipid moiety is conjugated to a nucleotide of the sense strand that forms a base pair with a nucleotide at position 17 of the antisense strand. In some embodiments, the lipid moiety is conjugated to a nucleotide of the sense strand that forms a base pair with a nucleotide at position 16 of the antisense strand. In some embodiments, the lipid moiety is conjugated to a nucleotide of the sense strand that forms a base pair with a nucleotide at position 15 of the antisense strand. In some embodiments, the lipid moiety is conjugated to a nucleotide of the sense strand that forms a base pair with a nucleotide at position 14 of the antisense strand. In some embodiments, the lipid moiety is conjugated to a nucleotide of the sense strand that forms a base pair with a nucleotide at position 13 of the antisense strand. In some embodiments, the lipid moiety is conjugated to a nucleotide of the sense strand that forms a base pair with a nucleotide at position 12 of the antisense strand.

In some embodiments, a double-stranded oligonucleotide described herein comprises at least one nucleotide conjugated with one or more lipid moieties. In some embodiments, the one or more lipid moieties are conjugated to the same nucleotide. In some embodiments, the one or more lipid moieties are conjugated to different nucleotides. In some embodiments, one, two, three, four, five, or six lipid moieties are conjugated to the oligonucleotide. In some embodiments, one or more lipid moieties are conjugated to an adenine nucleotide. In some embodiments, one or more lipid moieties are conjugated to a guanine nucleotide. In some embodiments, one or more lipid moieties are conjugated to a cytosine nucleotide. In some embodiments, one or more lipid moieties are conjugated to a thymine nucleotide. In some embodiments, one or more lipid moieties are conjugated to a uracil nucleotide.

In some embodiments, the lipid moiety is a hydrocarbon chain. In some embodiments, the hydrocarbon chain is saturated. In some embodiments, the hydrocarbon chain is unsaturated. In some embodiments, the hydrocarbon chain is branched. In some embodiments, the hydrocarbon chain is straight. In some embodiments, the lipid moiety is a C8-C30 hydrocarbon chain. In some embodiments, the lipid moiety is a C8:0, C10:0, C11:0, C12:0, C14:0, C16:0, C17:0, C18:0, C18:1, C18:2, C22:5, C22:0, C24:0, C26:0, C22:6, C24:1, diacyl C16:0 or diacyl C18:1.

In some embodiments, the lipid moiety is a C16 hydrocarbon chain.

In some embodiments, the lipid moiety is conjugated to the oligonucleotide via a linker. In some embodiments, a nucleotide of the lipid-conjugated oligonucleotide is represented by formula II-b or II-c:

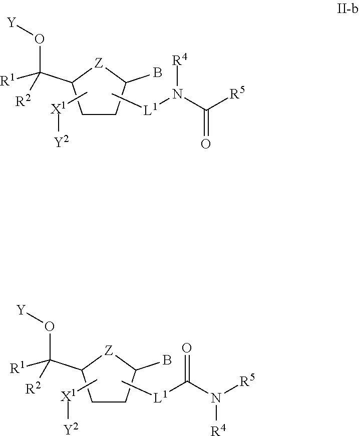

### II-c

or a pharmaceutically acceptable salt thereof, wherein:
L′ is a covalent bond, a monovalent or a bivalent saturated or unsaturated, straight, or branched C₁₋₅₀ hydrocarbon chain, wherein 0-10 methylene units of the hydrocarbon chain are independently replaced by -Cy-, —O—, —C(O)NR—, —NR—, —S—, —C(O)—, —C(O)O—, —S(O)—, —S(O)₂—, —P(O)OR—, —P(S)OR—, or

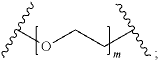

R⁴ is hydrogen, R^{A}, or a suitable amine protection group; and
R⁵ is adamantyl, or a saturated or unsaturated, straight, or branched C₁₋₅₀ hydrocarbon chain, wherein 0-10 methylene units of the hydrocarbon chain are independently replaced by —O—, —C(O)NR—, —NR—, —S—, —C(O)—, —C(O)O—, —S(O)—, —S(O)₂—, —P(O)OR—, or —P(S)OR.

In some embodiments of the double-stranded oligonucleotide, R$ is selected from

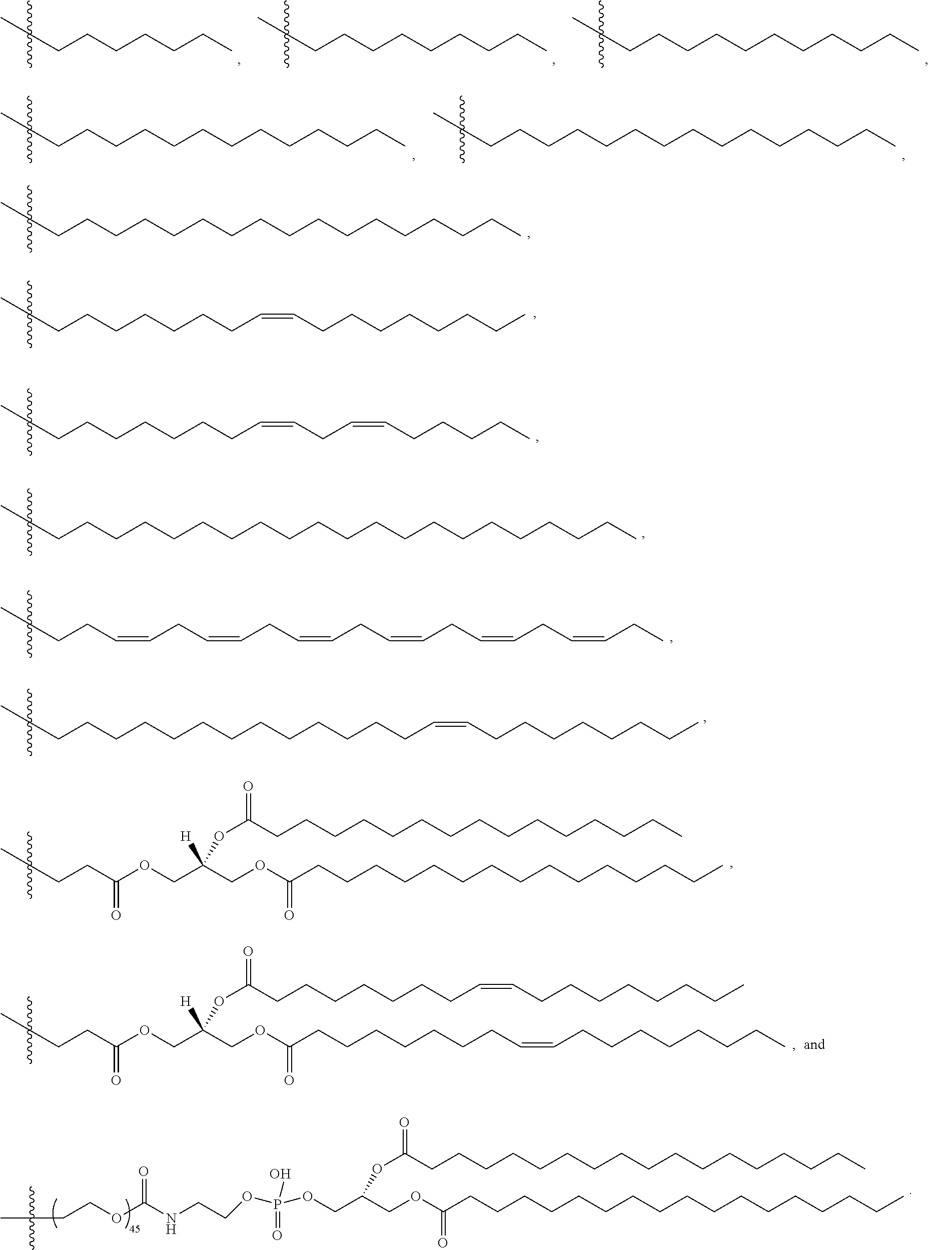

In certain embodiments of the double-stranded oligonucleotide,
R⁵ is selected from

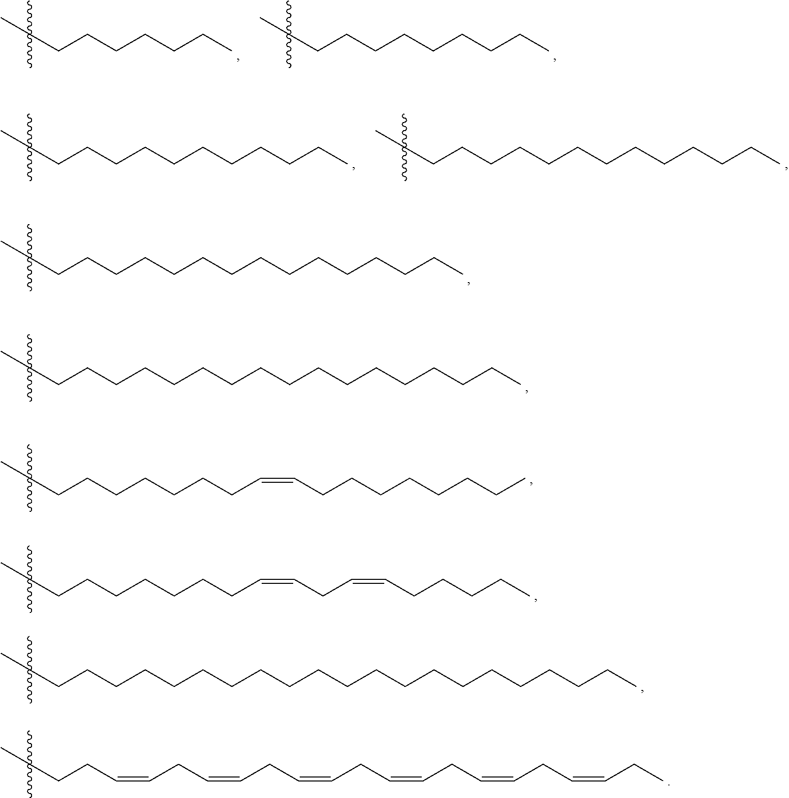

In some embodiments, R⁵ is

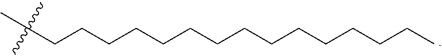 [SMILES: *CCCCCCCCCCCCCCC]

In some embodiments, R⁵ is

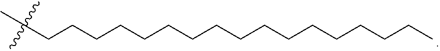 [SMILES: *CCCCCCCCCCCCCCCCC]

In some embodiments, R⁵ is

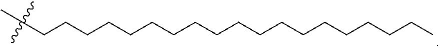 [SMILES: *CCCCCCCCCCCCCCCCCCC]

In some embodiments, a nucleotide of the double-stranded oligonucleotide is represented by formula II-Ib or II-Ic:

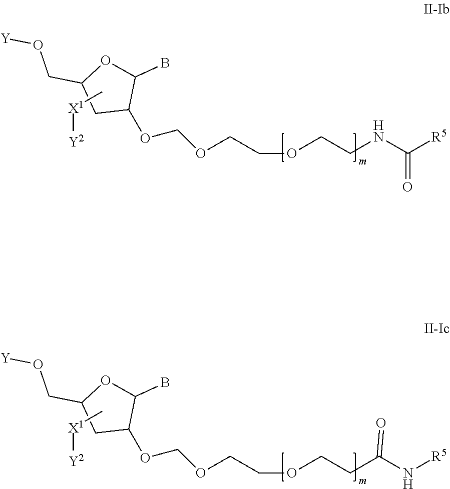

or a pharmaceutically acceptable salt thereof; wherein
B is a nucleobase or hydrogen;
m is 1-50;
X₁ is —O—, or —S—;
Y is hydrogen,

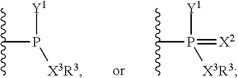

R³ is hydrogen, or a suitable protecting group;
X₂ is O, or S;
X₃ is —O—, —S—, or a covalent bond;
Y¹ is a linking group attaching to the 2′- or 3′-terminal of a nucleoside, a nucleotide, or an oligonucleotide;
Y² is hydrogen, a phosphoramidite analogue, an internucleotide linking group attaching to the 5′-terminal of a nucleoside, a nucleotide, or an oligonucleotide, or a linking group attaching to a solid support;
R⁵ is adamantyl, or a saturated or unsaturated, straight, or branched C₁₋₅₀ hydrocarbon chain, wherein 0-10 methylene units of the hydrocarbon chain are independently replaced by —O—, —C(O)NR—, —NR—, —S—, —C(O)—, —C(O)O—, —S(O)—, —S(O)₂—, —P(O)OR—, or —P(S)OR—; and
R is hydrogen, a suitable protecting group, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur.

In some embodiments, the lipid is

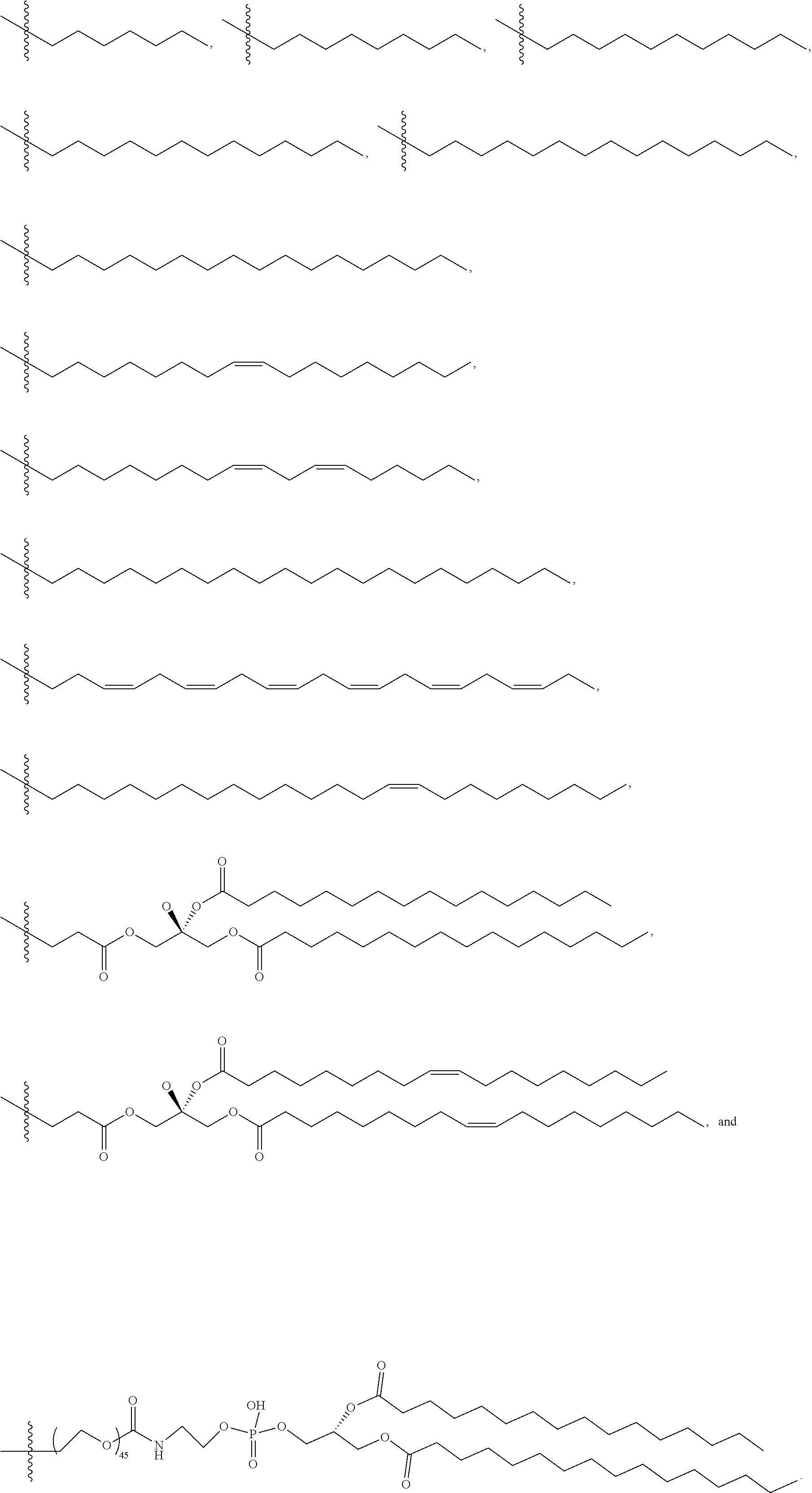

In some embodiments, the double stranded oligonucleotide comprises a stem loop. In some embodiments, the stem loop is set forth as S1-L-S2, wherein S1 is complementary to S2, and wherein L forms a loop between S1 and S2. In some embodiments, the ligand is conjugated to any of the nucleotides in the loop of the stem loop. In some embodiments, the ligand is conjugated to any of the nucleotides in the stem of the stem loop. In some embodiments, the ligand is conjugated to the first nucleotide from 5′ to 3′ in the loop. In some embodiments, the ligand is conjugated to the second nucleotide from 5′ to 3′ in the loop. In some embodiments, the ligand is conjugated to the third nucleotide from 5′ to 3′ in the loop. In some embodiments, the ligand is conjugated to the fourth nucleotide from 5′ to 3′ in the loop. In some embodiments, the ligand is conjugated to one, two, three, or four of the nucleotides in the loop. In some embodiments, the ligand is conjugated to three of the nucleotides in the stem loop.

In some embodiments, the stem loop is 16 nucleotides in length. In some embodiments, the ligand is conjugated to the seventh nucleotide from 5′ to 3′ in the stem loop. In some embodiments, the ligand is conjugated to the eighth nucleotide from 5′ to 3′ in the stem loop. In some embodiments, the ligand is conjugated to the ninth nucleotide from 5′ to 3′ in the stem loop. In some embodiments, the ligand is conjugated to the tenth nucleotide from 5′ to 3′ in the stem loop.

In some embodiments, the stem loop is 10 nucleotides in length. In some embodiments, the ligand is conjugated to fourth nucleotide from 5′ to 3′ in the stem loop. In some embodiments, the ligand is conjugated to fifth nucleotide from 5′ to 3′ in the stem loop. In some embodiments, the ligand is conjugated to sixth nucleotide from 5′ to 3′ in the stem loop. In some embodiments, the ligand is conjugated to seventh nucleotide from 5′ to 3′ in the stem loop.

### Exemplary Oligonucleotides

In some embodiments, the double-stranded oligonucleotide comprises a nucleotide conjugated with a fatty acid. In some embodiments, the fatty acid is a saturated fatty acid. In some embodiments, the fatty acid is an unsaturated fatty acid. In some embodiments, double-stranded oligonucleotide comprises a nucleotide conjugated with a lipid. In some embodiments, the lipid is a carbon chain. In some embodiments, the carbon chain is saturated. In some embodiments, the carbon chain is unsaturated. In some embodiments, the double-stranded oligonucleotide comprises a nucleotide conjugated with a 16-carbon (C16) lipid. In some embodiments, the C16 lipid comprises at least one double bond. In some embodiments, the double-stranded oligonucleotide comprises a nucleotide conjugated with a 22-carbon (C22) lipid.

In some embodiments, the oligonucleotide of the double-stranded oligonucleotide is conjugated to a C16 lipid as shown in:

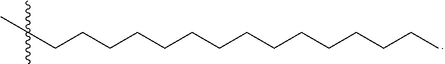 [SMILES: *CCCCCCCCCCCCCCC]

In some embodiments, the oligonucleotide of the double-stranded oligonucleotide is conjugated to a C22 lipid as shown in:

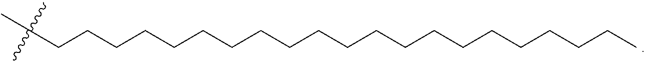 [SMILES: *CCCCCCCCCCCCCCCCCCCCC]

In some embodiments, the 3′ end of the sense strand is a blunt end. In some embodiments, the 5′ end of the antisense strand is a blunt end. In some embodiments, the 3′ end of the antisense strand comprises an overhang. In some embodiments, the 5′ end of the antisense strand comprises an overhang. In some embodiments, the 5′ and 3′ ends of the antisense strand each comprise an overhang.

In some embodiments, the double-stranded oligonucleotide comprises one or more 2′ modifications. In some embodiments, the 2′ modifications are selected from 2′-fluoro and 2′-methyl.

In some embodiments, the disclosure provides a double-stranded oligonucleotide comprising an antisense strand of 15-30 nucleotides in length and a sense strand of about 9-50 nucleotides in length, wherein
(i) the antisense and sense strands form a duplex region of about 9-26 base pairs,
(ii) the antisense strand comprises an orientation of 5′ to 3′,
(iii) the antisense strand comprises a 3′ overhang of at least four nucleotides,
(iv) the antisense strand comprises a region of complementarity to an mRNA target sequence in a target mRNA, and
(v) the antisense strand comprises at least one 2′-F modified nucleotide in the 3′ overhang, provided the at least one 2′-F modified nucleotide is not one of the two 3′ terminal nucleotides of the antisense strand.

In some embodiments, the disclosure provides a double-stranded oligonucleotide comprising an antisense strand of 15-30 nucleotides in length and a sense strand of about 9-50 nucleotides in length, wherein
(i) the antisense and sense strands form a duplex region of about 9-26 base pairs,
(ii) the antisense strand comprises an orientation of 5′ to 3′,
(iii) the antisense strand comprises a 3′ overhang of at least four nucleotides,
(iv) the sense strand comprises a lipid moiety conjugated to a nucleotide of the sense strand;
(v) the antisense strand comprises a region of complementarity to an mRNA target sequence in a target mRNA, and
(vi) the antisense strand comprises at least one 2′-F modified nucleotide in the 3′ overhang, provided the at least one 2′-F modified nucleotide is not one of the two 3′ terminal nucleotides of the antisense strand.

In some embodiments, the disclosure provides a double-stranded oligonucleotide comprising an antisense strand of 15-30 nucleotides in length and a sense strand of about 9-50 nucleotides in length, wherein
(i) the antisense and sense strands form a duplex region of about 9-26 base pairs,
(ii) the antisense strand comprises an orientation of 5′ to 3′,
(iii) the antisense strand comprises a 3′ overhang of at least four nucleotides,
(iv) the antisense strand comprises a region of complementarity to an mRNA target sequence in a target mRNA,
(v) the 3′ overhang comprises a sequence motif that reduces inhibition of the target mRNA by the double-stranded oligonucleotide in a cell of the liver, and
(vi) the sequence motif comprises at least one 2′-F modified nucleotide, provided the at least one 2′-F modified nucleotide is not one of the two 3′terminal nucleotides of the antisense strand.

In some embodiments, the disclosure provides a double-stranded oligonucleotide comprising an antisense strand of 15-30 nucleotides in length and a sense strand of about 9-50 nucleotides in length, wherein
(i) the antisense and sense strands form a duplex region of about 9-26 base pairs,
(ii) the antisense strand comprises an orientation of 5′ to 3′,
(iii) the antisense strand comprises a 3′ overhang of at least four nucleotides,
(iv) the antisense strand comprises a region of complementarity to an mRNA target sequence in a target mRNA,
(v) the sense strand comprises a lipid moiety conjugated to a nucleotide of the sense strand;
(v) the 3′ overhang comprises a sequence motif that reduces inhibition of the target mRNA by the double-stranded oligonucleotide in a cell of the liver, and
(vi) the sequence motif comprises at least one 2′-F modified nucleotide, provided the at least one 2′-F modified nucleotide is not one of the two 3′terminal nucleotides of the antisense strand.

In some embodiments, the disclosure provides a double-stranded oligonucleotide comprising an antisense strand of 15-30 nucleotides in length and a sense strand of about 9-50 nucleotides in length, wherein
(i) the antisense and sense strands form a duplex region of about 9-26 base pairs,
(ii) the antisense strand comprises an orientation of 5′ to 3′,
(iii) the antisense strand comprises a 3′ overhang of at least four nucleotides,
(iv) the antisense strand comprises a region of complementarity to an mRNA target sequence in a target mRNA,
(v) the 3′ overhang comprises a sequence motif that reduces inhibition of the target mRNA by the double-stranded oligonucleotide in a cell of the liver, and
(vi) the sequence motif comprises at least one 2′-F modified nucleotide, provided the at least one 2′-F modified nucleotide is not one of the two 3′terminal nucleotides of the antisense strand,
wherein the sequence motif comprises: 3′-P₁P₂[N]_{y}X₁-5′ wherein:
P₁ and P₂ are each independently a purine or a pyrimidine, and do not comprise a 2′-F modification;
X₁ is any nucleotide located immediately adjacent to the duplex, and comprises at least one phosphorothioate linkage to an adjacent nucleotide;
N is any nucleotide and y is an integer selected from 1-6, specifying N₁-N₆, wherein:
   (a) when y is 1, N₁ comprises a 2′-F modification;
   (b) when y is 2, one or more of N₁ and N₂ comprise a 2′-F modification;
   (c) when y is 3, one or more of N₁-N₃ comprise a 2′-F modification;
   (d) when y is 4, one or more of N₁-N₄ comprise a 2′-F modification;
   (e) when y is 5, one or more of N₁-N₅ comprise a 2′-F modification; or
   (f) when y is 6, one or more of N₁-N₆ comprise a 2′-F modification.

In some embodiments, the disclosure provides a double-stranded oligonucleotide comprising an antisense strand of 15-30 nucleotides in length and a sense strand of about 9-50 nucleotides in length, wherein
(i) the antisense and sense strands form a duplex region of about 9-26 base pairs,
(ii) the antisense strand comprises an orientation of 5′ to 3′,
(iii) the antisense strand comprises a 3′ overhang of at least four nucleotides,
(iv) the antisense strand comprises a region of complementarity to an mRNA target sequence in a target mRNA,
(v) the sense strand comprises a lipid moiety conjugated to a nucleotide of the sense strand;
(vi) the 3′ overhang comprises a sequence motif that reduces inhibition of the target mRNA by the double-stranded oligonucleotide in a cell of the liver, and
(vii) the sequence motif comprises at least one 2′-F modified nucleotide, provided the at least one 2′-F modified nucleotide is not one of the two 3′terminal nucleotides of the antisense strand,
wherein the sequence motif comprises: 3′-P₁P₂[N]_{y}X₁-5′ wherein:
P₁ and P₂ are each independently a purine or a pyrimidine, and do not comprise a 2′-F modification;
X₁ is any nucleotide located immediately adjacent to the duplex, and comprises at least one phosphorothioate linkage to an adjacent nucleotide;
N is any nucleotide and y is an integer selected from 1-6, specifying N₁-N₆, wherein:
   (a) when y is 1, N₁ comprises a 2′-F modification;
   (b) when y is 2, one or more of N₁ and N₂ comprise a 2′-F modification;
   (c) when y is 3, one or more of N₁-N₃ comprise a 2′-F modification;
   (d) when y is 4, one or more of N₁-N₄ comprise a 2′-F modification;
   (e) when y is 5, one or more of N₁-N₅ comprise a 2′-F modification; or
   (f) when y is 6, one or more of N₁-N₆ comprise a 2′-F modification.

In some embodiments, the disclosure provides a double-stranded oligonucleotide comprising an antisense strand of 15-30 nucleotides in length and a sense strand of about 9-50 nucleotides in length, wherein
(i) the antisense and sense strands form a duplex region of about 9-26 base pairs,
(ii) the antisense strand comprises an orientation of 5′ to 3′,
(iii) the antisense strand comprises a 3′ overhang of at least four nucleotides, (iv) the antisense strand comprises a region of complementarity to an mRNA target sequence in a target mRNA,
(v) the sense strand comprises a lipid moiety conjugated to a nucleotide of the sense strand;
(vi) the 3′ overhang comprises a sequence motif that reduces inhibition of the target mRNA by the double-stranded oligonucleotide in a cell of the liver, and
(vii) the sequence motif comprises at least one 2′-F modified nucleotide, provided the at least one 2′-F modified nucleotide is not one of the two 3′terminal nucleotides of the antisense strand,
wherein the sequence motif comprises: 3′-P₁P₂[N]_{y}X₁-5′ wherein:
P₁ and P₂ are each independently a purine, and do not comprise a 2′-F modification;
X₁ is any nucleotide located immediately adjacent to the duplex, and comprises at least one phosphorothioate linkage to an adjacent nucleotide;
N is any nucleotide and y is an integer selected from 1-6, specifying N₁-N₆, when y is 3, one or more of N₁-N₃ comprise a 2′-F modification.

In some embodiments, the disclosure provides a double-stranded oligonucleotide comprising an antisense strand of 15-30 nucleotides in length and a sense strand of about 9-50 nucleotides in length, wherein
(i) the antisense and sense strands form a duplex region of about 9-26 base pairs,
(ii) the antisense strand comprises an orientation of 5′ to 3′,
(iii) the antisense strand comprises a 3′ overhang of at least four nucleotides,
(iv) the antisense strand comprises a region of complementarity to an mRNA target sequence in a target mRNA,
(v) the sense strand comprises a lipid moiety conjugated to a nucleotide of the sense strand;
(vi) the 3′ overhang comprises a sequence motif that reduces inhibition of the target mRNA by the double-stranded oligonucleotide in a cell of the liver, and
(vii) the sequence motif comprises at least one 2′-F modified nucleotide, provided the at least one 2′-F modified nucleotide is not one of the two 3′terminal nucleotides of the antisense strand,
wherein the sequence motif comprises: 3′-P₁P₂[N]_{y}X₁-5′ wherein:
P₁ and P₂ are each independently a purine, and do not comprise a 2′-F modification;
X₁ is any nucleotide located immediately adjacent to the duplex, and comprises at least one phosphorothioate linkage to an adjacent nucleotide;
N is any nucleotide and y is an integer selected from 1-6, specifying N₁-N₆, when y is 3, N₂ comprise a 2′-F modification.

In some embodiments, the disclosure provides a double-stranded oligonucleotide comprising an antisense strand of 15-30 nucleotides in length and a sense strand of about 9-50 nucleotides in length, wherein
(i) the antisense and sense strands form a duplex region of about 9-26 base pairs,
(ii) the antisense strand comprises an orientation of 5′ to 3′,
(iii) the antisense strand comprises a 3′ overhang of at least four nucleotides and a 5′ overhang of at least two nucleotides,
(iv) the antisense strand comprises a region of complementarity to an mRNA target sequence in a target mRNA, and
(v) the antisense strand comprises at least one 2′-F modified nucleotide in the 3′ overhang, provided the at least one 2′-F modified nucleotide is not one of the two 3′ terminal nucleotides of the antisense strand.

In some embodiments, the disclosure provides a double-stranded oligonucleotide comprising an antisense strand of 15-30 nucleotides in length and a sense strand of about 9-50 nucleotides in length, wherein
(i) the antisense and sense strands form a duplex region of about 9-26 base pairs,
(ii) the antisense strand comprises an orientation of 5′ to 3′,
(iii) the antisense strand comprises a 3′ overhang of at least four nucleotides and a 5′ overhang of at least two nucleotides,
(iv) the sense strand comprises a lipid moiety conjugated to a nucleotide of the sense strand;
(v) the antisense strand comprises a region of complementarity to an mRNA target sequence in a target mRNA, and
(vi) the antisense strand comprises at least one 2′-F modified nucleotide in the 3′ overhang, provided the at least one 2′-F modified nucleotide is not one of the two 3′ terminal nucleotides of the antisense strand.

In some embodiments, the disclosure provides a double-stranded oligonucleotide comprising an antisense strand of 15-30 nucleotides in length and a sense strand of about 9-50 nucleotides in length, wherein
(i) the antisense and sense strands form a duplex region of about 9-26 base pairs,
(ii) the antisense strand comprises an orientation of 5′ to 3′,
(iii) the antisense strand comprises a 3′ overhang of at least four nucleotides and a 5′ overhang of at least two nucleotides,
(iv) the antisense strand comprises a region of complementarity to an mRNA target sequence in a target mRNA,
(v) the 3′ overhang comprises a sequence motif that reduces inhibition of the target mRNA by the double-stranded oligonucleotide in a cell of the liver, and
(vi) the sequence motif comprises at least one 2′-F modified nucleotide, provided the at least one 2′-F modified nucleotide is not one of the two 3′terminal nucleotides of the antisense strand.

In some embodiments, the disclosure provides a double-stranded oligonucleotide comprising an antisense strand of 15-30 nucleotides in length and a sense strand of about 9-50 nucleotides in length, wherein
(i) the antisense and sense strands form a duplex region of about 9-26 base pairs,
(ii) the antisense strand comprises an orientation of 5′ to 3′,
(iii) the antisense strand comprises a 3′ overhang of at least four nucleotides and a 5′ overhang of at least two nucleotides,
(iv) the antisense strand comprises a region of complementarity to an mRNA target sequence in a target mRNA,
(v) the sense strand comprises a lipid moiety conjugated to a nucleotide of the sense strand;
(v) the 3′ overhang comprises a sequence motif that reduces inhibition of the target mRNA by the double-stranded oligonucleotide in a cell of the liver, and
(vi) the sequence motif comprises at least one 2′-F modified nucleotide, provided the at least one 2′-F modified nucleotide is not one of the two 3′terminal nucleotides of the antisense strand.

In some embodiments, the disclosure provides a double-stranded oligonucleotide comprising an antisense strand of 15-30 nucleotides in length and a sense strand of about 9-50 nucleotides in length, wherein
(i) the antisense and sense strands form a duplex region of about 9-26 base pairs,
(ii) the antisense strand comprises an orientation of 5′ to 3′,
(iii) the antisense strand comprises a 3′ overhang of at least four nucleotides and a 5′ overhang of at least two nucleotides,
(iv) the antisense strand comprises a region of complementarity to an mRNA target sequence in a target mRNA,
(v) the 3′ overhang comprises a sequence motif that reduces inhibition of the target mRNA by the double-stranded oligonucleotide in a cell of the liver, and
(vi) the sequence motif comprises at least one 2′-F modified nucleotide, provided the at least one 2′-F modified nucleotide is not one of the two 3′terminal nucleotides of the antisense strand,
wherein the sequence motif comprises: 3′-P₁P₂[N]_{y}X₁-5′ wherein:
P₁ and P₂ are each independently a purine or a pyrimidine, and do not comprise a 2′-F modification;
X₁ is any nucleotide located immediately adjacent to the duplex, and comprises at least one phosphorothioate linkage to an adjacent nucleotide;
N is any nucleotide and y is an integer selected from 1-6, specifying N₁-N₆, wherein:
   (a) when y is 1, N₁ comprises a 2′-F modification;
   (b) when y is 2, one or more of N₁ and N₂ comprise a 2′-F modification;
   (c) when y is 3, one or more of N₁-N₃ comprise a 2′-F modification;
   (d) when y is 4, one or more of N₁-N₄ comprise a 2′-F modification;
   (e) when y is 5, one or more of N₁-N₅ comprise a 2′-F modification; or
   (f) when y is 6, one or more of N₁-N₆ comprise a 2′-F modification.

In some embodiments, the disclosure provides a double-stranded oligonucleotide comprising an antisense strand of 15-30 nucleotides in length and a sense strand of about 9-50 nucleotides in length, wherein
(i) the antisense and sense strands form a duplex region of about 9-26 base pairs,
(ii) the antisense strand comprises an orientation of 5′ to 3′,
(iii) the antisense strand comprises a 3′ overhang of at least four nucleotides and a 5′ overhang of at least two nucleotides,
(iv) the antisense strand comprises a region of complementarity to an mRNA target sequence in a target mRNA,
(v) the sense strand comprises a lipid moiety conjugated to a nucleotide of the sense strand;
(vi) the 3′ overhang comprises a sequence motif that reduces inhibition of the target mRNA by the double-stranded oligonucleotide in a cell of the liver, and
(vii) the sequence motif comprises at least one 2′-F modified nucleotide, provided the at least one 2′-F modified nucleotide is not one of the two 3′terminal nucleotides of the antisense strand,
wherein the sequence motif comprises: 3′-P₁P₂[N]_{y}X₁-5′ wherein:
P₁ and P₂ are each independently a purine or a pyrimidine, and do not comprise a 2′-F modification;
X₁ is any nucleotide located immediately adjacent to the duplex, and comprises at least one phosphorothioate linkage to an adjacent nucleotide;
N is any nucleotide and y is an integer selected from 1-6, specifying N₁-N₆, wherein:
   (a) when y is 1, N₁ comprises a 2′-F modification;
   (b) when y is 2, one or more of N₁ and N₂ comprise a 2′-F modification;
   (c) when y is 3, one or more of N₁-N₃ comprise a 2′-F modification;
   (d) when y is 4, one or more of N₁-N₄ comprise a 2′-F modification;
   (e) when y is 5, one or more of N₁-N₅ comprise a 2′-F modification; or
   (f) when y is 6, one or more of N₁-N₆ comprise a 2′-F modification.

In some embodiments, the disclosure provides a double-stranded oligonucleotide comprising an antisense strand of 15-30 nucleotides in length and a sense strand of about 9-50 nucleotides in length, wherein
(i) the antisense and sense strands form a duplex region of about 9-26 base pairs,
(ii) the antisense strand comprises an orientation of 5′ to 3′,
(iii) the antisense strand comprises a 3′ overhang of at least four nucleotides and a 5′ overhang of at least two nucleotides,
(iv) the antisense strand comprises a region of complementarity to an mRNA target sequence in a target mRNA,
(v) the sense strand comprises a lipid moiety conjugated to a nucleotide of the sense strand;
(vi) the 3′ overhang comprises a sequence motif that reduces inhibition of the target mRNA by the double-stranded oligonucleotide in a cell of the liver, and
(vii) the sequence motif comprises at least one 2′-F modified nucleotide, provided the at least one 2′-F modified nucleotide is not one of the two 3′terminal nucleotides of the antisense strand,
wherein the sequence motif comprises: 3′-P₁P₂[N]_{y}X₁-5′ wherein:
P₁ and P₂ are each independently a purine, and do not comprise a 2′-F modification;
X₁ is any nucleotide located immediately adjacent to the duplex, and comprises at least one phosphorothioate linkage to an adjacent nucleotide;
N is any nucleotide and y is an integer selected from 1-6, specifying N₁-N₆, when y is 3, one or more of N₁-N₃ comprise a 2′-F modification.

In some embodiments, the disclosure provides a double-stranded oligonucleotide comprising an antisense strand of 15-30 nucleotides in length and a sense strand of about 9-50 nucleotides in length, wherein
(i) the antisense and sense strands form a duplex region of about 9-26 base pairs,
(ii) the antisense strand comprises an orientation of 5′ to 3′,
(iii) the antisense strand comprises a 3′ overhang of at least four nucleotides and a 5′ overhang of at least two nucleotides,
(iv) the antisense strand comprises a region of complementarity to an mRNA target sequence in a target mRNA,
(v) the sense strand comprises a lipid moiety conjugated to a nucleotide of the sense strand;
(vi) the 3′ overhang comprises a sequence motif that reduces inhibition of the target mRNA by the double-stranded oligonucleotide in a cell of the liver, and
(vii) the sequence motif comprises at least one 2′-F modified nucleotide, provided the at least one 2′-F modified nucleotide is not one of the two 3′terminal nucleotides of the antisense strand,
wherein the sequence motif comprises: 3′-P₁P₂[N]_{y}X₁-5′ wherein:
P₁ and P₂ are each independently a purine, and do not comprise a 2′-F modification;
X₁ is any nucleotide located immediately adjacent to the duplex, and comprises at least one phosphorothioate linkage to an adjacent nucleotide;
N is any nucleotide and y is an integer selected from 1-6, specifying N₁-N₆, when y is 3, N₂ comprise a 2′-F modification.

In some embodiments, the disclosure provides a double-stranded oligonucleotide comprising an antisense strand of 22 nucleotides in length and a sense strand of about 11-50 nucleotides in length, wherein
(i) the antisense and sense strands form a duplex region of about 11-26 base pairs,
(ii) the antisense strand comprises an orientation of 5′ to 3′,
(iii) the antisense strand comprises a 3′ overhang of at least four nucleotides and a 5′ overhang of at least two nucleotides,
(iv) the antisense strand comprises a region of complementarity to an mRNA target sequence in a target mRNA,
(v) the sense strand comprises a lipid moiety conjugated to a nucleotide of the sense strand;
(vi) the 3′ overhang comprises a sequence motif that reduces inhibition of the target mRNA by the double-stranded oligonucleotide in a cell of the liver, and
(vii) the sequence motif comprises a 2′-F nucleotide at position 19, position 16, or positions 19 and 16, of the antisense strand.

In some embodiments, the disclosure provides a double-stranded oligonucleotide comprising an antisense strand of 22 nucleotides in length and a sense strand of about 11-50 nucleotides in length, wherein
(i) the antisense and sense strands form a duplex region of about 11-26 base pairs,
(ii) the antisense strand comprises an orientation of 5′ to 3′,
(iii) the antisense strand comprises a 3′ overhang of at least four nucleotides and a 5′ overhang of at least two nucleotides,
(iv) the antisense strand comprises a region of complementarity to an mRNA target sequence in a target mRNA,
(v) the sense strand comprises a lipid moiety conjugated to a nucleotide of the sense strand; and
(vi), the antisense strand comprises a 2′-F nucleotide at position 19, position 16, or positions 19 and 16.

In some embodiments, a double-stranded oligonucleotide for reducing expression of a target gene comprises the modification pattern of

Sense Strand: 5′-[+Xs][mX][fX][mX][fX][fX][X][fX]
[mX][fX][mX][mX][mX][mX][mX][mX][X][mX][X][mX]
[ademX-L][mX][mX][mX][mX][mX][mX][mX][mX]-3′

### Hybridized to:

Antisense Strand:
5′-[MePhosphonate-
4O-mXs][fXs][fXs]
[fX][fX][mX][fX]
[mX][mX][fX][mX]
[mX][mXs][fXs]
[mX][fX][mX][mX]
[fX][mXs][mXs]
[mX]-3′

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester linkages to neighboring nucleotides, [MePhosphonate-40-mX]=4′-O-monomethylphosphonate-2′-O-methyl modified nucleotide, and [ademX-L]=Lipid attached to a nucleotide, [+X]=a Tm-increasing nucleotide, optionally an LNA, and [+Xs]=a Tm-increasing nucleotide with a phosphorothioate linkage to the neighboring nucleotide, optionally an LNA . . .

In some embodiments, a double-stranded oligonucleotide for reducing expression of a target gene comprises the modification pattern of

Sense Strand: 5′-[+Xs][mX][X][mX][fX][fX][mX][fX]
[mX][X][mX][mX][mX][+X][mX][mX][mX][ademX-L][mX]
[mX][mX][mX][+X]-3′

### Hybridized to:

Antisense Strand:
5′-[MePhosphonate-4O-mXs]
[fXs][fXs][fX][fX]
[mX][fX][mX][mX][fX]
[mX][mX][mXs][fXs]
[mX][mX][mX][mX][fX]
[mXs][mXs][mX]-3′

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester increasing nucleotide, optionally an LNA, and [+Xs]=a Tm-increasing nucleotide with a phosphorothioate linkage to the neighboring nucleotide, optionally an LNA.

In some embodiments, a double-stranded oligonucleotide for reducing expression of a target gene comprises the modification pattern of

Sense Strand:
5′-[+Xs][mX][fX][mX][fX][fX][mX]
[fX][mX][fX][mX][mX][mX][+X][mX]
[mX][mX][ademX-L][mX][mX][mX][mX]
[+X]-3′

### Hybridized to:

Antisense Strand:
5′-[MePhosphonate-4O-mXs][fXs][fXs]
[fX][fX][mX][fX][mX][mX][fX][mX]
[mX][mXs][fXs][mX][fX][mX][mX][mX]
[mXs][mXs][mX]-3′

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester linkages to neighboring nucleotides, [MePhosphonate-40-mX]=4′-O-monomethylphosphonate-2′-O-methyl modified nucleotide, [ademX-L]=Lipid attached to a nucleotide, [+X]=a Tm-increasing nucleotide, optionally an LNA, and [+Xs]=a Tm-increasing nucleotide with a phosphorothioate linkage to the neighboring nucleotide, optionally an LNA.

In some embodiments, a double-stranded oligonucleotide for reducing expression of a target gene comprises the modification pattern of

Sense Strand: 5′-[+Xs][mX][fX][mX][[X][fX][X][fX]
[mX][fX][mX][mX][mX][+X][mX][mX][mX][ademX-L][mX]
[mX][mX][mX][+X]-3′

### Hybridized to:

Antisense Strand:
5′-[MePhosphonate-4O-mXs][fXs]
[fXs][fX][fX][mX][fX][mX][mX]
[fX][mX][mX][mXs][fXs][mX][fX]
[mX][mX][fX][mXs][mXs][mX]-3′

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester increasing nucleotide, optionally an LNA, and [+Xs]=a Tm-increasing nucleotide with a phosphorothioate linkage to the neighboring nucleotide, optionally an LNA.

In some embodiments, a double-stranded oligonucleotide for reducing expression of a target gene comprises the modification pattern of

Sense Strand:
5′-[+Xs][fX][fX][fX][mX][mX][mX]
[mX][mX][mX][mX][mX][mX][mX][mX]
[mX][mX][mX][mX][mX][ademX-L][mX]
[mX][mX][mX][mX][mX][mX][mX]-3′

### Hybridized to:

Antisense Strand:
5′-[MePhosphonate-4O-mXs][fXs][fXs]
[fX][fX][mX][fX][mX][mX][fX][mX]
[mX][mXs][fXs][mX][fX][mX][mX][fX]
[mXs][mXs][mX]-3′

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester linkages to neighboring nucleotides, [MePhosphonate-40-mX]=4′-O-monomethylphosphonate-2′-O-methyl modified nucleotide, [ademX-L]=Lipid attached to a nucleotide, [+X]=a Tm-increasing nucleotide, optionally an LNA, and [+Xs]=a Tm-increasing nucleotide with a phosphorothioate linkage to the neighboring nucleotide, optionally an LNA.

In some embodiments, a double-stranded oligonucleotide for reducing expression of a target gene comprises the modification pattern of

Sense Strand:
5′-[+Xs][fX][fX][fX][mX][mX][mX]
[mX][mX][mX][mX][mX][mX][+X][mX]
[mX][mX][ademX-L][mX][mX][mX][mX]
[+X]-3′

### Hybridized to:

Antisense Strand:
5′-[MePhosphonate-4O-mXs][fXs][fXs]
[fX][fX][mX][fX][mX][mX][fX][mX]
[mX][mX][fX][mX][mX][mX][mX][fX]
[mXs][mXs][mX]-3′

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester increasing nucleotide, optionally an LNA, and [+Xs]=a Tm-increasing nucleotide with a phosphorothioate linkage to the neighboring nucleotide, optionally an LNA.

In some embodiments, a double-stranded oligonucleotide for reducing expression of a target gene comprises the modification pattern of

Sense Strand: 5′-[+Xs][fX][fX][fX][mX][mX][mX]
[mX][mX][mX][mX][mX][mX][mX][mX][mX][mX][mX][mX]
[mX][ademX-L][mX][mX][mX][mX][mX][mX][mX][mX]-3′

### Hybridized to:

Antisense Strand:
5′-[MePhosphonate-4O-mXs][fXs]
[fXs][fX][fX][mX][fX][mX][mX][fX]
[mX][mX][mXs][fXs][mX][mX][mX][mX]
[fX][mXs][mXs][mX]-3′

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester linkages to neighboring nucleotides, [MePhosphonate-40-mX]=4′-O-monomethylphosphonate-2′-O-methyl modified nucleotide, [ademX-L]=Lipid attached to a nucleotide, [+X]=a Tm-increasing nucleotide, optionally an LNA, and [+Xs]=a Tm-increasing nucleotide with a phosphorothioate linkage to the neighboring nucleotide, optionally an LNA.

In some embodiments, a double-stranded oligonucleotide for reducing expression of a target gene comprises the modification pattern of

Sense Strand: 5′-[+Xs][fX][fX][fX][fX][mX][mX][mX]
[X][mX][mX][mX][mX][mX][mX][mX][mX][mX][mX][mX]
[mX][ademX-L][mX][mX][mX][mX][mX][mX][mX][mX]-3′

### Hybridized to:

Antisense Strand:
5′-[MePhosphonate-4O-mXs][fXs][fXs]
[fX][fX][mX][fX][mX][mX][fX][mX]
[mX][mX][fX][mX][mX][mX][mX][fX]
[mXs][mXs][mX]-3′

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester 2′-O-methyl modified nucleotide, and [ademX-L]=Lipid attached to a nucleotide, [+X]=a Tm-increasing nucleotide, optionally an LNA, and [+Xs]=a Tm-increasing nucleotide with a phosphorothioate linkage to the neighboring nucleotide, optionally an LNA.

In some embodiments, a double-stranded oligonucleotide for reducing expression of a target gene comprises the modification pattern of

Sense Strand: 5′-[+Xs][mX][fX][fX][X][fX][mX][mX]
[mX][mX][mX][X][mX][mX][mX][mX][mX][mX][mX][mX][mX]
[mX][ademX-L][mX][mX][mX][mX][mX][mX][mX][mX]-3′

### Hybridized to:

Antisense Strand:
5′-[MePhosphonate-4O-mXs][fXs][fXs]
[fX][fX][mX][fX][mX][mX][fX][mX]
[mX][mX][fX][mX][mX][mX][mX][fX]
[mXs][mXs][mX]-3′

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester linkages to neighboring nucleotides, [MePhosphonate-40-mX]=4′-O-monomethylphosphonate-2′-O-methyl modified nucleotide, [ademX-L]=Lipid attached to a nucleotide, [+X]=a Tm-increasing nucleotide, optionally an LNA, and [+Xs]=a Tm-increasing nucleotide with a phosphorothioate linkage to the neighboring nucleotide, optionally an LNA.

In some embodiments, a double-stranded oligonucleotide for reducing expression of a target gene comprises the modification pattern of

Sense Strand: 5′-[+Xs][mX][mX][fX][fX][fX][fX][mX]
[X][mX][mX][mX][mX][mX][mX][mX][mX][mX][mX][mX][mX]
[mX][X][ademX-L][mX][mX][mX][mX][mX][mX][mX][mX]-3′

### Hybridized to:

Antisense Strand:
5′-[MePhosphonate-4O-mXs][fXs][fXs]
[fX][fX][mX][fX][mX][mX][[fX][mX]
[mX][mX][fX][mX][mX][mX][mX][fX]
[mXs][mXs][mX]-3′

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester increasing nucleotide, optionally an LNA, and [+Xs]=a Tm-increasing nucleotide with a phosphorothioate linkage to the neighboring nucleotide, optionally an LNA.

In some embodiments, a double-stranded oligonucleotide for reducing expression of a target gene comprises the modification pattern of

Sense Strand: 5′-[ademXs-L][mX][mX][X][fX][fX][fX]
[mX][mX][mX][mX][mX][mX][mX][mX][mX][mX][mX][mX]
[mX][mX][mX][mX][mX][mX][mX][mX][mX][X][mX][mX]
[mX]-3′

### Hybridized to:

Antisense Strand:
5′-[MePhosphonate-4O-mXs][fXs][fXs]
[fX][fX][mX][fX][mX][mX][fX][mX]
[mX][mX][fX][mX][mX][mX][mX][fX]
[mXs][mXs][mX]-3′

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester linkages to neighboring nucleotides, [MePhosphonate-40-mX]=4′-O-monomethylphosphonate-2′-O-methyl modified nucleotide, [ademX-Ls]=Lipid attached to a nucleotide with a phosphorothioate linkage to the neighboring nucleotide.

In some embodiments, a double-stranded oligonucleotide for reducing expression of a target gene comprises the modification pattern of

Sense Strand: 5′-[+Xs][mX][mX][X][X][fX][fX][mX]
[mX][mX][mX][mX][mX][mXs][mXs][ademX-L]-3′

### Hybridized to:

Antisense Strand:
5′-[MePhosphonate-4O-mXs][fXs][fXs]
[fX][fX][mX][fX][mX][mX][fX][mX]
[mX][mX][fX][mX][mX][mX][mX][fX]
[mXs][mXs][mX]-3′

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester increasing nucleotide, optionally an LNA, and [+Xs]=a Tm-increasing nucleotide with a phosphorothioate linkage to the neighboring nucleotide, optionally an LNA.

In some embodiments, a double-stranded oligonucleotide for reducing expression of a target gene comprises the modification pattern of

Sense Strand: 5′-[+Xs][mX][X][fX][fX][X][fX][mX]
[mX][mX][mX][X][mX][mXs][mXs][ademX-L]-3′

### Hybridized to:

Antisense Strand:
5′-[MePhosphonate-4O-mXs][fXs][fXs]
[fX][fX][mX][fX][mX][mX][fX][mX]
[mX][mX][fX][mX][mX][mX][mX][fXs]
[mXs][mXs][mX]-3′

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester linkages to neighboring nucleotides, [MePhosphonate-40-mX]=4′-O-monomethylphosphonate-2′-O-methyl modified nucleotide, [ademX-L]=Lipid attached to a nucleotide, [+X]=a Tm-increasing nucleotide, optionally an LNA, and [+Xs]=a Tm-increasing nucleotide with a phosphorothioate linkage to the neighboring nucleotide, optionally an LNA.

In some embodiments, a double-stranded oligonucleotide for reducing expression of a target gene comprises the modification pattern of

Sense Strand: 5′-[+Xs][mX][mX][fX][fX][fX][X][mX]
[mX][mX][mX][mX][mX][mX][mX][mX][mX][mX][X][mX]
[mX][mX][mX][ademX-L][mX][mX][mX][mX][mX][mX][mX]
[mX]-3′

### Hybridized to:

Antisense Strand: 5′-[MePhosphonate-40-mXs][fXs]
[fXs][fX][fX][mX][fX][mX][mX][fX][mX][mX][mX][fX]
[mX][mX][mX][mX][fXs][mXs][mXs][mX]-3′

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester increasing nucleotide, optionally an LNA, and [+Xs]=a Tm-increasing nucleotide with a phosphorothioate linkage to the neighboring nucleotide, optionally an LNA.

In some embodiments, a double-stranded oligonucleotide for reducing expression of a target gene comprises the modification pattern of

Sense Strand: 5′-[ademXs-L][mX][mX][fX][fX][fX]
[fX][mX][X][mX][mX][mX][mX][mX][mX][mX][mX][mX]
[mX][mX][mX][mX][mX][mX][mX][mX][mX][mX][mX][mX]
[X][mX]-3′

### Hybridized to:

Antisense Strand: 5′-[MePhosphonate-40-mXs][fXs]
[fXs][fX][fX][mX][fX][X][mX][fX][mX][X][mX][fX]
[mX][mX][mX][mX][fXs][mXs][mXs][mX]-3′

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester linkages to neighboring nucleotides, [MePhosphonate-40-mX]=4′-O-monomethylphosphonate-2′-O-methyl modified nucleotide, and [ademX-L]=Lipid attached to a nucleotide.

### General Methods of Providing the Nucleic Acids and Analogues Thereof

The nucleic acids and analogues thereof comprising lipid conjugate described herein can be made using a variety of synthetic methods known in the art, including standard phosphoramidite methods. Any phosphoramidite synthesis method can be used to synthesize the provided nucleic acids of this disclosure. In certain embodiments, phosphoramidites are used in a solid phase synthesis method to yield reactive intermediate phosphite compounds, which are subsequently oxidized using known methods to produce phosphonate-modified oligonucleotides, typically with a phosphodiester or phosphorothioate internucleotide linkages. The oligonucleotide synthesis of the present disclosure can be performed in either direction: from 5′ to 3′ or from 3′ to 5′ using art known methods.

In certain embodiments, the method for synthesizing a provided nucleic acid comprises (a) attaching a nucleoside or analogue thereof to a solid support via a covalent linkage; (b) coupling a nucleoside phosphoramidite or analogue thereof to a reactive hydroxyl group on the nucleoside or analogue thereof of step (a) to form an internucleotide bond there between, wherein any uncoupled nucleoside or analogue thereof on the solid support is capped with a capping reagent; (c) oxidizing said internucleotide bond with an oxidizing agent; and (d) repeating steps (b) to (c) iteratively with subsequent nucleoside phosphoramidites or analogue thereof to form a nucleic acid or analogue thereof, wherein at least the nucleoside or analogue thereof of step (a), the nucleoside phosphoramidite or analogue thereof of step (b) or at least one of the subsequent nucleoside phosphoramidites or analogues thereof of step (d) comprises a lipid conjugate moiety as described herein. Typically, the coupling, capping/oxidizing steps and optionally, the deprotecting steps, are repeated until the oligonucleotide reaches the desired length and/or sequence, after which it is cleaved from the solid support. In certain embodiments, an oligonucleotide is prepared comprising 1-3 nucleic acid or analogues thereof comprising lipid conjugates units on a tetraloop.

In Scheme A below, where a particular protecting group, leaving group, or transformation condition is depicted, one of ordinary skill in the art will appreciate that other protecting groups, leaving groups, and transformation conditions are also suitable and are contemplated. Certain reactive functional groups (e.g., —N(H)—, —OH, etc.) envisioned in the genera in Scheme A requiring additional protection group strategies are also contemplated and is appreciated by those having ordinary skill in the art. Such groups and transformations are described in detail in MARCH'S ADVANCED ORGANIC CHEMISTRY: REACTIONS, MECHANISMS, AND STRUCTURE, M. B. Smith and J. March, 5^{th} Edition, John Wiley & Sons, 2001, COMPREHENSIVE ORGANIC TRANSFORMATIONS, (R. C. Larock, 2^{nd} Edition, John Wiley & Sons, 1999), and PROTECTING GROUPS IN ORGANIC SYNTHESIS, (T. W. Greene and P. G. M. Wuts, 3^{rd} edition, John Wiley & Sons, 1999), the entirety of each of which is hereby incorporated herein by reference.

In certain embodiments, nucleic acids, and analogues thereof of the present disclosure are generally prepared according to Scheme A, Scheme A1 and Scheme B set forth below:

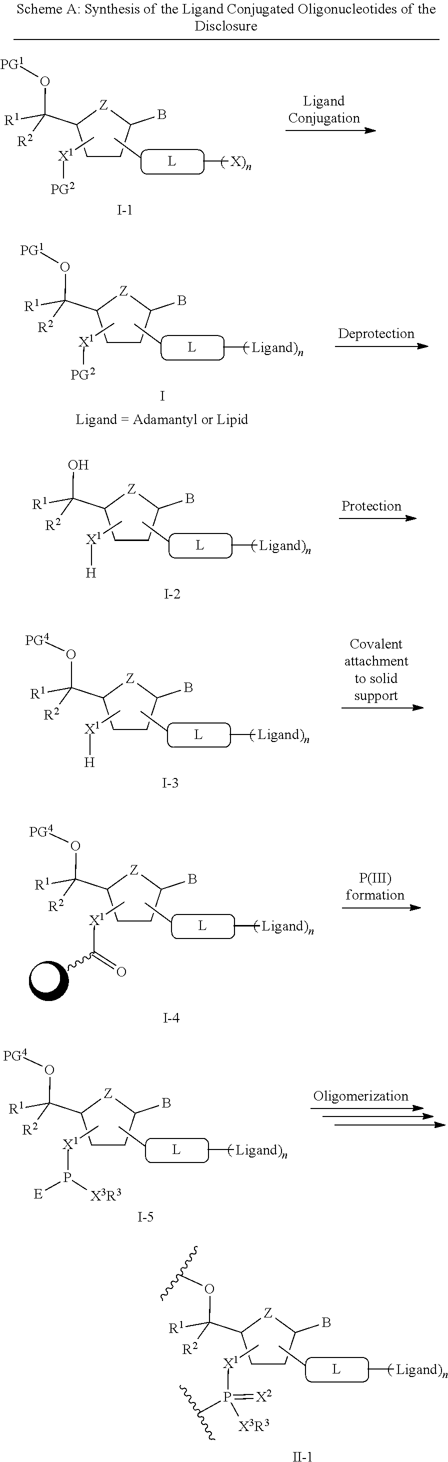

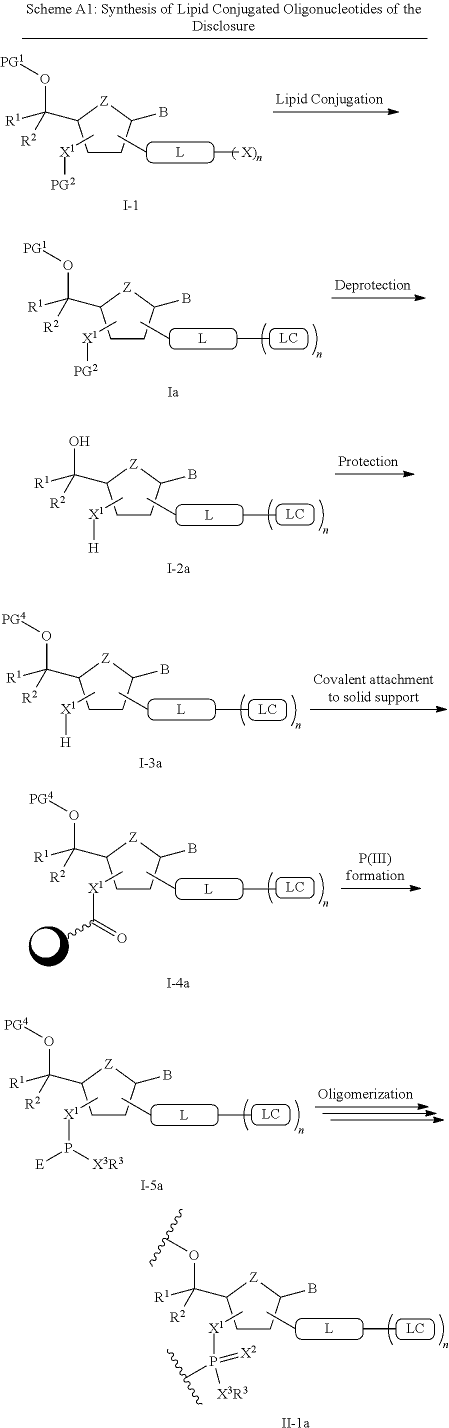

As depicted in Scheme A and Scheme A1 above, a nucleic acid or analogue thereof of formula I-1 is conjugated with one or more ligand/lipophilic compound to form a compound of formula I or Ia comprising one more ligand/lipid conjugates. Typically, conjugation is performed through an esterification or amidation reaction between a nucleic acid or analogue thereof of formula I-1 or I-1a and one or more adamantyl and/or lipophilic compound (e.g., fatty acid) in series or in parallel by known techniques in the art. Nucleic acid or analogue thereof of formula I or 1a can then be deprotected to form a compound of formula I-2 or I-2a and protected with a suitable hydroxyl protecting group (e.g., DMTr) to form a compound of formula I-3 or I-3a. In one aspect, nucleic acid-ligand conjugates of formula I-3 or I-3a can be covalently attached to a solid support (e.g., through a succinic acid linking group) to form a solid support nucleic acid-ligand conjugate or analogue thereof of formula I-4 or I-4a comprising one or more adamantyl and/or lipid conjugate. In another aspect, a nucleic acid-ligand conjugates of formula I-3 or I-3a can react with a forming reagent (e.g., 2-cyanoethyl N,N-di-P(III) isopropylchlorophosphoramidite) to form a nucleic acid or analogue thereof of formula I-5 or I-5a comprising a P(III) group. A nucleic acid-ligand conjugate or analogue thereof of formula I-5 or I-5a can then be subjected to oligomerization forming conditions preformed using known and commonly applied processes to prepare oligonucleotides in the art. For example, the compound of formula I-5 or I-5a is coupled to a solid supported nucleic acid-ligand conjugate or analogue thereof bearing a 5′-hydroxyl group. Further steps can comprise one or more deprotections, couplings, phosphite oxidation, and/or cleavage from the solid support to provide an oligonucleotide of various nucleotide lengths, including one or more lipid conjugate nucleotide units represented by a compound of formula II-1 or II-Ia. Each of B, E, L, ligand, LC, n, PG¹, PG², PG₄, R¹, R², R³, X, X¹, X², X³, and Z is as defined above and described herein.

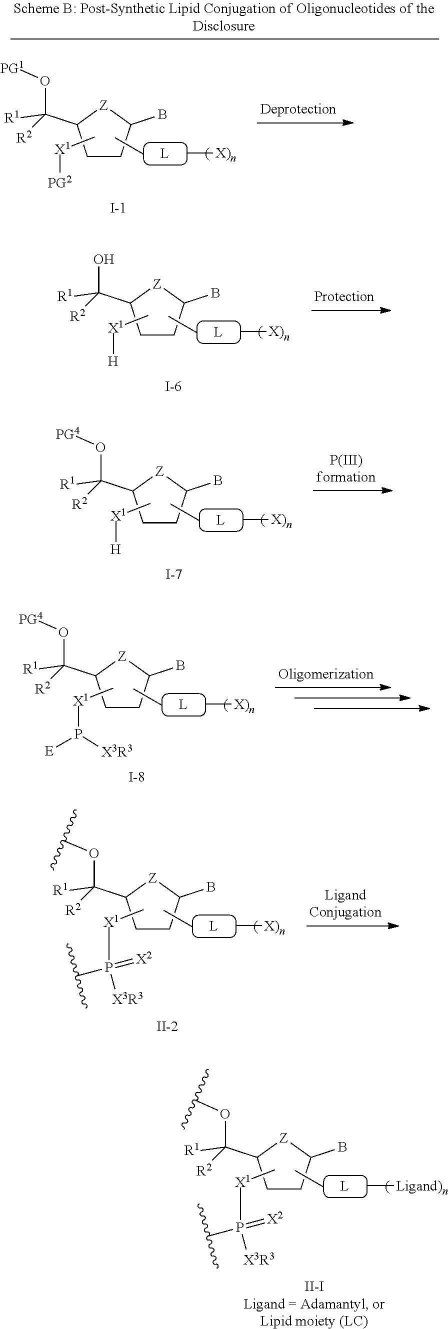

As depicted in Scheme B above, a nucleic acid or analogue thereof of formula I-1 can be deprotected to form a compound of formula I-6, protected with a suitable hydroxyl protecting group (e.g., DMTr) to form a compound of formula I-7, and reacted with a P(III) forming reagent (e.g., 2-cyanoethyl N,N-di-isopropylchlorophosphoramidite) to form a nucleic acid or analogue thereof of formula I-8 comprising a P(III) group. Next, a nucleic acid or analogue thereof of formula I-8 is subjected to oligomerization forming conditions preformed using known and commonly applied processes to prepare oligonucleotides in the art. For example, the compound of formula I-8 is coupled to a solid supported nucleic acid or analogue thereof bearing a 5′-hydroxyl group. Further steps can comprise one or more deprotections, couplings, phosphite oxidation, and/or cleavage from the solid support to provide an oligonucleotide of various nucleotide lengths represented by a compound of formula II-2. An oligonucleotide of formula II-2 can then be conjugated with one or more ligands e.g., adamantyl, or lipophilic compound (e.g., fatty acid) to form a compound of formula II-1 comprising one or more ligand conjugates. Typically, conjugation is performed through an esterification or amidation reaction between a nucleic acid or analogue thereof of formula II-2 and one or more adamantyl or fatty acid in series or in parallel by known techniques in the art. Each of B, E, L, ligand, LC, n, PG¹, PG², PG⁴, R¹, R², R³, X, X¹, X², X³, and Z is as defined above and described herein.

In certain embodiments, nucleic acids, and analogues thereof of the present disclosure are prepared according to Scheme C and Scheme D set forth below:

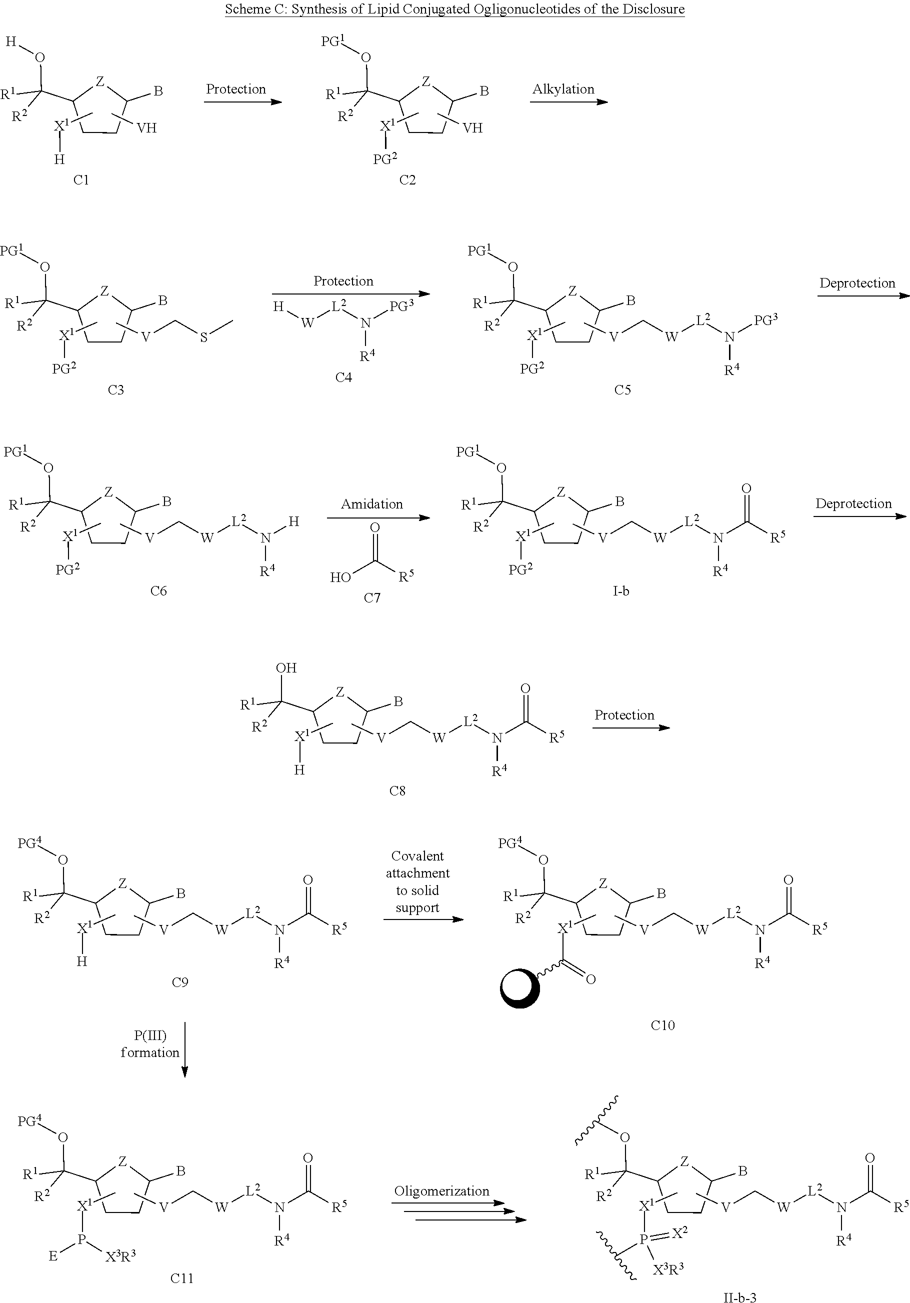

As depicted in Scheme C above, a nucleic acid or analogue thereof of formula C1 is protected to form a compound of formula C2. Nucleic acid or analogue thereof of formula C2 is then alkylated (e.g., using DMSO and acetic acid via the Pummerer rearrangement) to form a monothioacetal compound of formula C3. Next, nucleic acid or analogue thereof of formula C3 is coupled with C4 under appropriate conditions (e.g., mild oxidizing conditions) to form a nucleic acid or analogue thereof of formula C5. Nucleic acid or analogue thereof of formula C5 can then be deprotected to form a compound of formula C6 and coupled with a ligand (adamantyl or lipophilic compound (e.g., a fatty acid)) of formula C7 under appropriate amide forming conditions (e.g., HATU, DIPEA), to form a nucleic acid-ligand conjugate or analogue thereof of formula I-b comprising a lipid conjugate of the disclosure. Nucleic acid-ligand conjugate or analogue thereof of formula I-b can then be deprotected to form a compound of formula C8 and protected with a suitable hydroxyl protecting group (e.g., DMTr) to form a compound of formula C9. In one aspect, nucleic acid, or analogue thereof of formula C9 can be covalently attached to a solid support (e.g., through a succinic acid linking group) to form a solid support nucleic acid-ligand conjugate or analogue thereof of formula C10 comprising a ligand conjugate (adamantyl or lipid moiety) of the disclosure. In another aspect, a nucleic acid-ligand conjugate or analogue thereof of formula C9 can reacted with a P(III) forming reagent (e.g., 2-cyanoethyl N,N-di-isopropylchlorophosphoramidite) to form a nucleic acid-ligand conjugate or analogue thereof of formula C11 comprising a P(III) group. A nucleic acid-ligand conjugate or analogue thereof of formula C11 can then be subjected to oligomerization forming conditions preformed using known and commonly applied processes to prepare oligonucleotides in the art. For example, the compound of formula C11 is coupled to a solid supported nucleic acid-ligand conjugate or analogue thereof bearing a 5′-hydroxyl group. Further steps can comprise one or more deprotections, couplings, phosphite oxidation, and/or cleavage from the solid support to provide an oligonucleotide of various nucleotide lengths, including one or more adamantyl and/or lipid conjugate nucleotide units represented by a compound of formula II-b-3. Each of B, E, L², PG¹, PG², PG³, PG⁴, R¹, R², R³, R¹, R⁵, X¹, X², X³, V, W, and Z is as defined above and described herein.

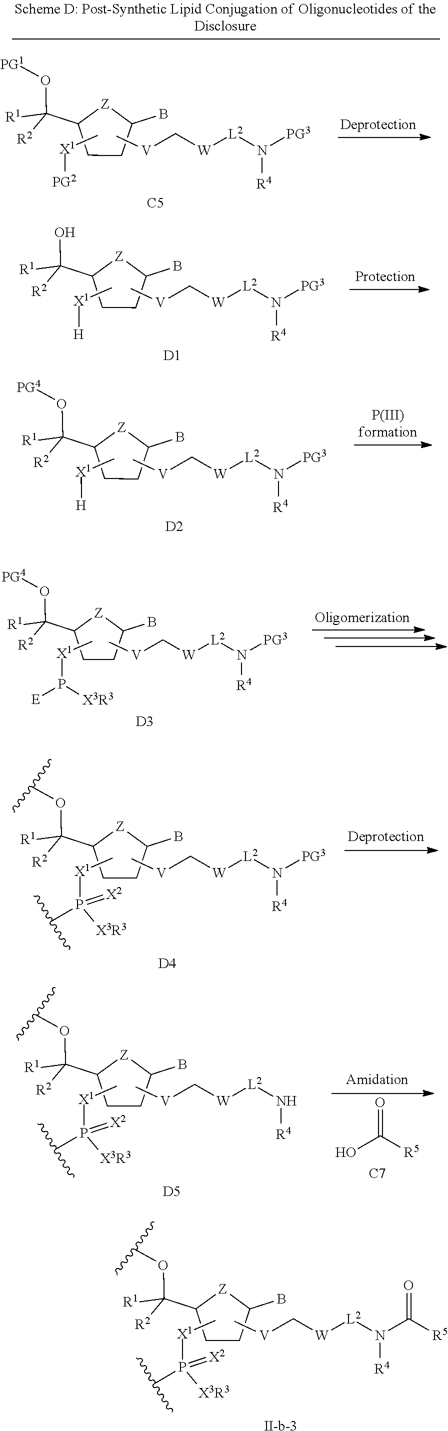

Each of B, E, L², PG¹, PG², PG³, PG⁴, R¹, R², R³, R⁴, R⁵, X¹, X², X³, V, W, and Z is as defined above and described herein. As depicted in Scheme D above, a nucleic acid or analogue thereof of formula C5 can be selectively deprotected to form a compound of formula D1, protected with a suitable hydroxyl protecting group (e.g., DMTr) to form a compound of formula D2, and reacted with a P(III) forming reagent (e.g., 2-cyanoethyl N,N-di-isopropylchlorophosphoramidite) to form a nucleic acid or analogue thereof of formula D3. Next, a nucleic acid or analogue thereof of formula D3 is subjected to oligomerization forming conditions preformed using known and commonly applied processes to prepare oligonucleotides in the art. For example, the compound of formula D3 is coupled to a solid supported nucleic acid or analogue thereof bearing a 5′-hydroxyl group. Further steps can comprise one or more deprotections, couplings, phosphite oxidation, and/or cleavage from the solid support to provide an oligonucleotide of various nucleotide lengths, represented by a compound of formula D4. An oligonucleotide of formula D4 can then be deprotected to form a compound of formula D5 and coupled with a hydrophobic ligand (e.g., adamantyl or a lipophilic moiety) to form a compound of formula C7 (e.g., adamantyl or a fatty acid) under appropriate amide forming conditions (e.g., HATU, DIPEA), to form an oligonucleotide of formula II-b-3 comprising a ligand (e.g., adamantyl or a fatty acid) conjugate of the disclosure.

One of skill in the art will appreciate that various functional groups present in the nucleic acid or analogues thereof of the disclosure such as aliphatic groups, alcohols, carboxylic acids, esters, amides, aldehydes, halogens, and nitriles can be interconverted by techniques well known in the art including, but not limited to reduction, oxidation, esterification, hydrolysis, partial oxidation, partial reduction, halogenation, dehydration, partial hydration, and hydration. See for example, “MARCH'S ADVANCED ORGANIC CHEMISTRY”, (5^{th} Ed., Ed.: Smith, M. B. and March, J., John Wiley & Sons, New York: 2001), the entirety of each of which is herein incorporated by reference. Such interconversions may require one or more of the aforementioned techniques, and certain methods for synthesizing the provided nucleic acids of the disclosure are described below in the Exemplification.

In some embodiments, the present disclosure provides a method for preparing an oligonucleotide comprising one or more lipid conjugate, said lipid conjugate unit represent by formula II-a-1:

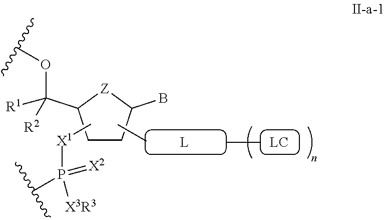

or a pharmaceutically acceptable salt thereof, comprising the steps of:
(a) providing a nucleic acid or analogue thereof of formula I-5a:

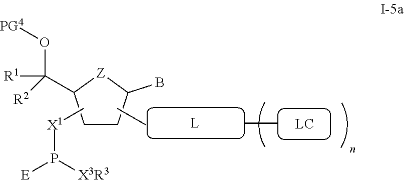

or salt thereof, and
(b) oligomerizing said compound of formula I-5a to form a compound of formula II-1a, wherein each of B, E, L, LC, n, PG⁴, R¹, R², R³, X, X₁, X₂, X₃, E, and Z is as defined above and described herein.

In step (b) above, oligomerizing refers to preforming oligomerization forming conditions using known and commonly applied processes to prepare oligonucleotides in the art. For example, the compound of formula I-5a is coupled to a solid supported nucleic acid or analogue thereof bearing a 5′-hydroxyl group. Further steps can comprise one or more deprotections, couplings, phosphite oxidation, and cleavage from the solid support to provide an oligonucleotide of various nucleotide lengths, represented by a compound of formula II-1a comprising a lipid conjugate of the disclosure.

In some embodiments, the present disclosure provides a method for preparing an oligonucleotide comprising one or more lipid conjugate, further comprising preparing a nucleic acid or analogue thereof of formula I-5a:

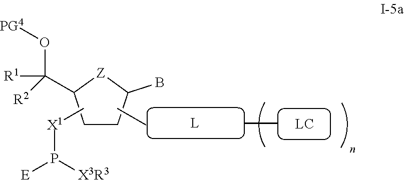

or a salt thereof, comprising the steps of:

(a) providing a nucleic acid or analogue thereof of formula Ia:

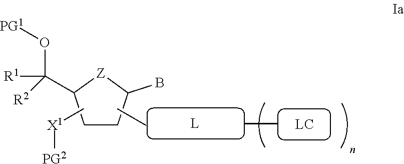

or salt thereof,
(b) deprotecting said nucleic acid or analogue thereof of formula Ia to form a compound of formula I-2a:

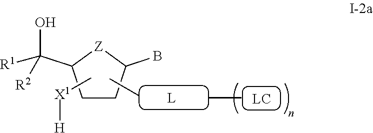

or salt thereof,
(c) protecting said nucleic acid or analogue thereof of formula I-2 to form a compound of formula I-3a:

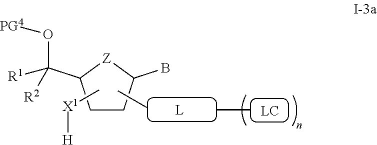

or salt thereof, and
(d) treating said nucleic acid or analogue thereof of formula I-3a with a P(III) forming reagent to form a nucleic acid or analogue thereof of formula I-5a, wherein each of B, E, L, LC, n, PG⁴, R¹, R², R³, X, X₁, X₂, X₃, E, and Z is as defined above and described herein.

In step (b) above, PG¹ and PG² of a compound of formula Ia comprise silyl ethers or cyclic silylene derivatives that can be removed under acidic conditions or with fluoride anion. Examples of reagents providing fluoride anion for the removal of silicon-based protecting groups include hydrofluoric acid, hydrogen fluoride pyridine, triethylamine trihydrofluoride, tetra-N-butylammonium fluoride, and the like.

In step (c) above, a compound of formula I-2a is protected with a suitable hydroxyl protecting group. In certain embodiments, the protecting group PG⁴ used for protection of the 5′-hydroxyl group of a compound of formula I-2a includes an acid labile protecting group such as trityl, 4-methyoxytrityl, 4,4′-dimethyoxytrityl, 4,4′,4″-trimethyoxytrityl, 9-phenyl-xanthen-9-yl, 9-(p-tolyl)-xanthen-9-yl, pixyl, 2,7-dimethylpixyl, and the like. In certain embodiments, the acid labile protecting group is suitable for deprotection during both solution-phase and solid-phase synthesis of acid-sensitive nucleic acids or analogues thereof using for example, dichloroacetic acid or trichloroacetic acid.

In step (d) above, a compound of formula I-3a is treated with a P(III) forming reagent to afford a compound of formula I-5a. In the context of the present disclosure, a P(III) forming reagent is a phosphorus reagent that is reacted to for a phosphorus (III) compound. In some embodiments, the P(III) forming reagent is 2-cyanoethyl N,N-diisopropylchlorophosphoramidite or 2-cyanoethyl phosphorodichloridate. In certain embodiments, the P(III) forming reagent is 2-cyanoethyl N,N-diisopropylchlorophosphoramidite. One of ordinary skill would recognize that the displacement of a leaving group in a P(III) forming reagent by X₁ of a compound of formula I-3a is achieved either with or without the presence of a suitable base. Such suitable bases are well known in the art and include organic and inorganic bases. In certain embodiments, the base is a tertiary amine such as triethylamine or diisopropylethylamine. In other embodiments, step (d) above is preformed using N,N-dimethylphosphoramic dichloride as a P(V) forming reagent.

In some embodiments, the present disclosure provides a method for preparing an oligonucleotide comprising one or more lipid conjugates, further comprising preparing a nucleic acid-lipid conjugate or analogue thereof of formula Ia:

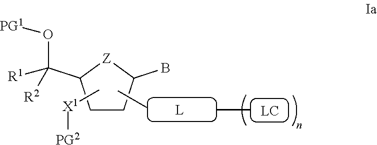

or a salt thereof, comprising the steps of:
(a) providing a nucleic acid or analogue thereof of formula I-1:

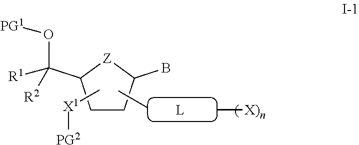

or salt thereof, and,
(b) conjugating one or more lipophilic compounds to a nucleic acid or analogue thereof of formula I-1 to form a nucleic acid or analogue thereof of formula Ia comprising one or more lipid conjugates, wherein: each of B, E, L, LC, n, PG¹, PG², R¹, R², X, X₁, and Z is as defined above and described herein.

In step (b) above, a nucleic acid or analogue thereof of formula I-la is conjugated with one or more lipophilic compounds to form a compound of formula Ia comprising one more lipid conjugates of the disclosure. Typically, conjugation is performed through an esterification or amidation reaction between a nucleic acid or analogue thereof of formula I-la and one or more fatty acids in series or in parallel by known techniques in the art. In certain embodiments, conjugation is performed under suitable amide forming conditions to afford a compound of formula I comprising one more lipid conjugates. Suitable amide forming conditions can include the use of an amide coupling reagent known in the art such as, but not limited to HATU, PyBOP, DCC, DIC, EDC, HBTU, HCTU, PyAOP, PyBrOP, BOP, BOP-CI, DEPBT, T3P, TATU, TBTU, TNTU, TOTU, TPTU, TSTU, or TDBTU. Alternatively, conjugation of a lipophilic compound can be accomplished by any one of the cross-coupling technologies described in Table A herein.

In some embodiments, the present disclosure provides a method for preparing an oligonucleotide comprising one or more lipid conjugate, said lipid conjugate unit represent by formula II-1:

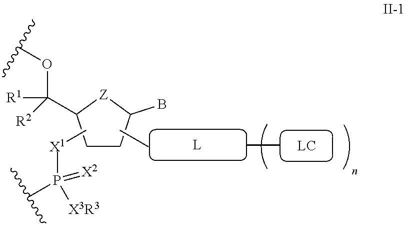

or a pharmaceutically acceptable salt thereof, comprising the steps of:
(a) providing an oligonucleotide of formula II-2:

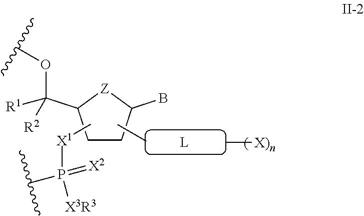

or salt thereof, and,
(b) conjugating one or more lipophilic compounds to an oligonucleotide of formula II-2 to form an oligonucleotide of formula II-1 comprising one or more lipid conjugates. In step (b) above, an oligonucleotide of formula II-2 is conjugated with one or more lipophilic compounds to form an oligonucleotide of formula II-1 comprising one more lipid conjugates of the disclosure. Typically, conjugation is performed through an esterification or amidation reaction between an oligonucleotide of formula II-2 and one or more fatty acids in series or in parallel by known techniques in the art. In certain embodiments, conjugation is performed under suitable amide forming conditions to afford an oligonucleotide of formula II-1 comprising one more lipid conjugates. Suitable amide forming conditions can include the use of an amide coupling reagent known in the art such as, but not limited to HATU, PyBOP, DCC, DIC, EDC, HBTU, HCTU, PyAOP, PyBrOP, BOP, BOP-CI, DEPBT, T3P, TATU, TBTU, TNTU, TOTU, TPTU, TSTU, or TDBTU. Alternatively, conjugation of a lipophilic compound can be accomplished by any one of the cross-coupling technologies described in Table A herein.

In some embodiments, the present disclosure provides a method for preparing an oligonucleotide comprising a unit represent by formula II-2:

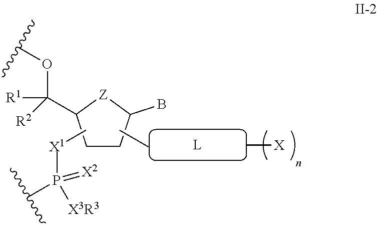

or a pharmaceutically acceptable salt thereof, comprising the steps of:
(a) providing a nucleic acid or analogue thereof of formula I-8:

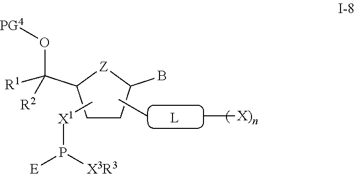

or salt thereof, and
(b) oligomerizing said compound of formula I-8 to form a compound of formula II-2.

In step (b) above, oligomerizing refers to preforming oligomerization forming conditions using known and commonly applied processes to prepare oligonucleotides in the art. For example, the compound of formula I-8 is coupled to a solid supported nucleic acid or analogue thereof bearing a 5′-hydroxyl group. Further steps can comprise one or more deprotections, couplings, phosphite oxidation, and cleavage from the solid support to provide an oligonucleotide of various nucleotide lengths, represented by a compound of formula II-2.

In some embodiments, the present disclosure provides a method for preparing a nucleic acid or analogue thereof comprising one or more lipid conjugate, further comprising preparing a nucleic acid or analogue thereof of formula I-8:

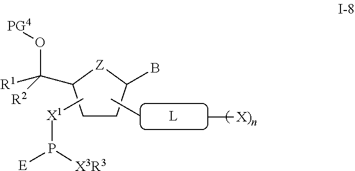

or a salt thereof, comprising the steps of:
(a) providing a nucleic acid or analogue thereof of formula I-1:

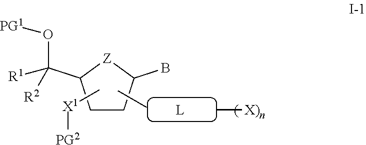

or salt thereof,
(b) deprotecting said nucleic acid or analogue thereof of formula I-1 to form a compound of formula I-6:

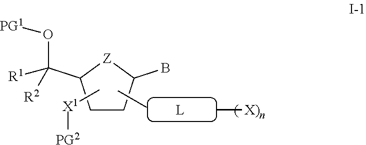

or salt thereof,
(c) protecting said nucleic acid or analogue thereof of formula I-6 to form a compound of formula I-7:

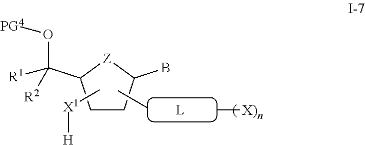

or salt thereof, and
(d) treating said nucleic acid or analogue thereof of formula I-7 with a P(III) forming reagent to form a nucleic acid or analogue thereof of formula 1-8, In step (b) above, PG¹ and PG² of a compound of formula I-1 comprise silyl ethers or cyclic silylene derivatives that can be removed under acidic conditions or with fluoride anion. Examples of reagents providing fluoride anion for the removal of silicon-based protecting groups include hydrofluoric acid, hydrogen fluoride pyridine, triethylamine trihydrofluoride, tetra-N-butylammonium fluoride, and the like.

In step (c) above, a compound of formula I-6 is protected with a suitable hydroxyl protecting group. In certain embodiments, the protecting group PG⁴ used for protection of the 5′-hydroxyl group of a compound of formula I-6 includes an acid labile protecting group such as trityl, 4-methyoxytrityl, 4,4′-dimethyoxytrityl, 4,4′,4″-trimethyoxytrityl, 9-phenyl-xanthen-9-yl, 9-(p-tolyl)-xanthen-9-yl, pixyl, 2,7-dimethylpixyl, and the like. In certain embodiments, the acid labile protecting group is suitable for deprotection during both solution-phase and solid-phase synthesis of acid-sensitive nucleic acids or analogues thereof using for example, dichloroacetic acid or trichloroacetic acid.

In step (d) above, a compound of formula I-7 is treated with a P(III) forming reagent to afford a compound of formula I-8. In the context of the present disclosure, a P(III) forming reagent is a phosphorus reagent that is reacted to for a phosphorus (III) compound. In some embodiments, the P(III) forming reagent is 2-cyanoethyl N,N-diisopropylchlorophosphoramidite or 2-cyanoethyl phosphorodichloridate. In certain embodiments, the P(III) forming reagent is 2-cyanoethyl N,N-diisopropylchlorophosphoramidite. One of ordinary skill would recognize that the displacement of a leaving group in a P(III) forming reagent by X₁ of a compound of formula I-7 is achieved either with or without the presence of a suitable base. Such suitable bases are well known in the art and include organic and inorganic bases. In certain embodiments, the base is a tertiary amine such as triethylamine or diisopropylethylamine. In other embodiments, step (d) above is preformed using N,N-dimethylphosphoramic dichloride as a P(V) forming reagent.

In some embodiments, the present disclosure provides a method for preparing an oligonucleotide-ligand conjugate comprising one or more adamantyl and/or lipid moieties, said conjugate unit represented by formula II-b-3:

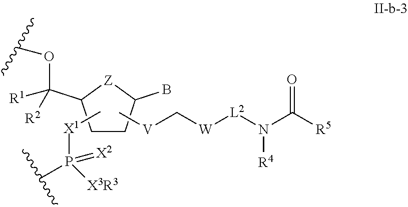

or a pharmaceutically acceptable salt thereof, comprising the steps of:
(a) providing a nucleic acid-ligand conjugate or analogue thereof of formula C11:

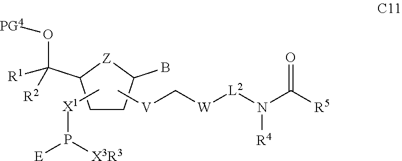

or salt thereof, and
(b) oligomerizing said compound of formula C11 to form a compound of formula II-b-3, In step (b) above, oligomerizing refers to preforming oligomerization forming conditions using known and commonly applied processes to prepare oligonucleotides in the art. For example, the compound of formula C11 is coupled to a solid supported nucleic acid or analogue thereof bearing a 5′-hydroxyl group. Further steps can comprise one or more deprotections, couplings, phosphite oxidation, and cleavage from the solid support to provide an oligonucleotide-ligand conjugate of various nucleotide lengths, with one or more nucleic acid-ligand conjugate units, wherein each unit is represented by a compound of formula II-b-3 comprising an adamantyl or lipid moiety of the disclosure.

In some embodiments, the method for preparing an oligonucleotide of formula II-b-3 comprising one or more lipid conjugate, further comprises preparing a nucleic acid-ligand conjugate or analogue thereof of formula C11:

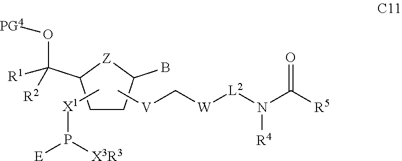

or a salt thereof, comprising the steps of:
(a) providing a nucleic acid-ligand conjugate or analogue thereof of formula I-b:

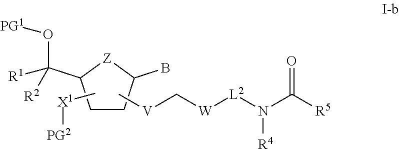

or salt thereof,
(b) deprotecting said nucleic acid-ligand conjugate or analogue thereof of formula I-b to form a compound of formula C8:

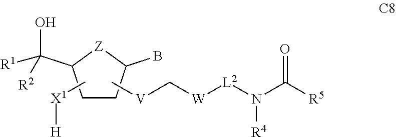

or salt thereof,
(c) protecting said nucleic acid-ligand conjugate or analogue thereof of formula C8 to form a compound of formula C9:

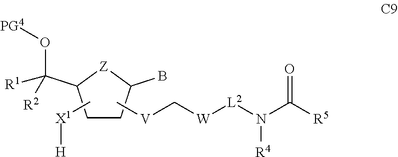

or salt thereof, and
(d) treating said nucleic acid-ligand conjugate or analogue thereof of formula C9 with a P(III) forming reagent to form a nucleic acid or analogue thereof of formula C11. In step (b) above, PG¹ and PG² of a compound of formula I-b comprise silyl ethers or cyclic silylene derivatives that can be removed under acidic conditions or with fluoride anion. Examples of reagents providing fluoride anion for the removal of silicon-based protecting groups include hydrofluoric acid, hydrogen fluoride pyridine, triethylamine trihydrofluoride, tetra-N-butylammonium fluoride, and the like.

In step (c) above, a compound of formula C8 is protected with a suitable hydroxyl protecting group. In certain embodiments, the protecting group PG⁴ used for protection of the 5′-hydroxyl group of a compound of formula C8 includes an acid labile protecting group such as trityl, 4-methyoxytrityl, 4,4′-dimethyoxytrityl, 4,4′,4″-trimethyoxytrityl, 9-phenyl-xanthen-9-yl, 9-(p-tolyl)-xanthen-9-yl, pixyl, 2,7-dimethylpixyl, and the like. In certain embodiments, the acid labile protecting group is suitable for deprotection during both solution-phase and solid-phase synthesis of acid-sensitive nucleic acids or analogues thereof using for example, dichloroacetic acid or trichloroacetic acid.

In step (d) above, a compound of formula C9 is treated with a P(III) forming reagent to afford a compound of formula C11. In the context of the present disclosure, a P(III) forming reagent is a phosphorus reagent that is reacted to for a phosphorus (III) compound. In some embodiments, the P(III) forming reagent is 2-cyanoethyl N,N-diisopropylchlorophosphoramidite or 2-cyanoethyl phosphorodichloridate. In certain embodiments, the P(III) forming reagent is 2-cyanoethyl N,N-diisopropylchlorophosphoramidite. One of ordinary skill would recognize that the displacement of a leaving group in a P(III) forming reagent by X¹ of a compound of formula C9 is achieved either with or without the presence of a suitable base. Such suitable bases are well known in the art and include organic and inorganic bases. In certain embodiments, the base is a tertiary amine such as triethylamine or diisopropylethylamine. In other embodiments, step (d) above is preformed using N,N-dimethylphosphoramic dichloride as a P(V) forming reagent.

In some embodiments, the present disclosure provides a method for preparing an oligonucleotide-ligand conjugate of formula II-b-3 comprising one or more nucleic acid-ligand conjugate units each comprising one or more adamantyl or lipid moieties, further comprising preparing a nucleic acid-ligand conjugate or analogue thereof of formula I-b:

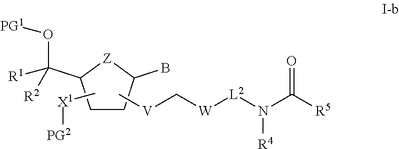

or a salt thereof, comprising the steps of:
(a) providing a nucleic acid-ligand conjugate or analogue thereof of formula C6:

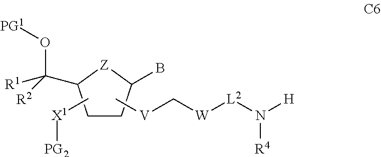

or salt thereof, and,
(b) conjugating a lipophilic compound to a nucleic acid or analogue thereof of formula C6 to form a nucleic acid-ligand conjugate or analogue thereof of formula I-b comprising one or more adamantyl and/or lipid conjugates. In step (b) above, conjugation is performed under suitable amide forming conditions to afford a compound of formula I-b comprising an adamantyl and/or lipid conjugate. Suitable amide forming conditions can include the use of an amide coupling reagent known in the art such as, but not limited to HATU, PyBOP, DCC, DIC, EDC, HBTU, HCTU, PyAOP, PyBrOP, BOP, BOP-CI, DEPBT, T3P, TATU, TBTU, TNTU, TOTU, TPTU, TSTU, or TDBTU. In certain embodiments, the amide forming conditions comprise HATU and DIPEA or TEA.

In certain embodiments, a nucleic acid-ligand conjugate or analogue thereof of formula C6 is provided in salt form (e.g., a fumarate salt) and is first converted to the free base (e.g., using sodium bicarbonate) before preforming the conjugation step.

In some embodiments, the present disclosure provides a method for preparing an oligonucleotide-ligand conjugate of formula II-b-3 comprising one or more nucleic acid-ligand conjugate units, further comprises preparing a nucleic acid-ligand conjugate or analogue thereof of formula C6:

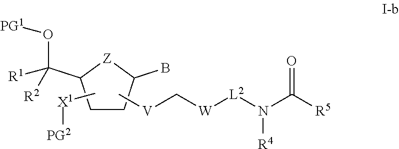

or a salt thereof, comprising the steps of:
(a) providing a nucleic acid or analogue thereof of formula C1:

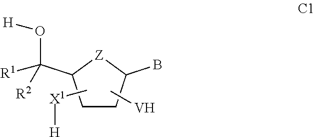

or salt thereof, and,
(b) protecting said nucleic acid or analogue thereof of formula C1 to form a compound of formula C2:

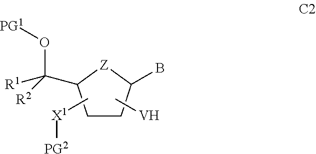

or salt thereof,
(c) alkylating said nucleic acid or analogue thereof of formula C2 to form a compound of formula C3:

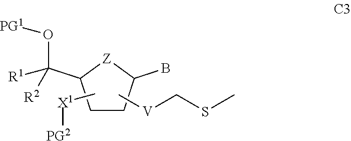

or salt thereof,
(d) substituting said nucleic acid or analogue thereof of formula C3 with a compound of formula C4:

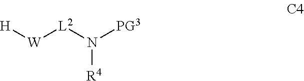

or salt thereof, to form a compound of formula C₅:

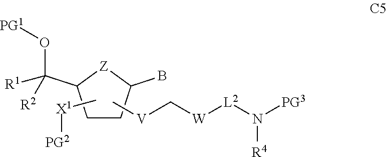

or salt thereof,
(e) deprotecting said nucleic acid or analogue thereof of formula C5 to form a nucleic acid-ligand conjugate or analogue thereof of formula C6. In step (b) above, PG¹ and PG² groups of formula C2 are taken together with their intervening atoms to form a cyclic diol protecting group, such as a cyclic acetal or ketal. Such groups include methylene, ethylidene, benzylidene, isopropylidene, cyclohexylidene, and cyclopentylidene, silylene derivatives such as di-t-butylsilylene and 1,1,3,3-tetraisopropylidisiloxanylidene, a cyclic carbonate, a cyclic boronate, and cyclic monophosphate derivatives based on cyclic adenosine monophosphate (i.e., cAMP). In certain embodiments, the cyclic diol protection group is 1,1,3,3-tetraisopropylidisiloxanylidene prepared from the reaction of a diol of formula C1 and 1,3-dichloro-1,1,3,3-tetraisopropyldisiloxane under basic conditions.

In step (c) above, a nucleic acid or analogue thereof of formula C2 is alkylated with a mixture of DMSO and acetic anhydride under acidic conditions. In certain embodiments, when-V-H is a hydroxyl group, the mixture of DMSO and acetic anhydride in the presence of acetic acid forms (methylthio)methyl acetate in situ via the Pummerer rearrangement which then reacts with the hydroxyl group of the nucleic acid or analogue thereof of formula C2 to provide a monothioacetal functionalized fragment nucleic acid or analogue thereof of formula C3.

In step (d) above, substitution of the thiomethyl group of a nucleic acid or analogue thereof of formula C3 using a nucleic acid or analogue thereof of formula C4 affords a nucleic acid or analogue thereof of formula C4. In certain embodiments, substitution occurs under mild oxidizing and/or acidic conditions. In some embodiments, V is oxygen. In some embodiments, the mild oxidation reagent includes a mixture of elemental iodine and hydrogen peroxide, urea hydrogen peroxide complex, silver nitrate/silver sulfate, sodium bromate, ammonium peroxodisulfate, tetrabutylammonium peroxydisulfate, Oxone®, Chloramine T, Selectfluor®, Selectfluor® II, sodium hypochlorite, or potassium iodate/sodium periodiate. In certain embodiments, the mild oxidizing agent includes N-iodosuccinimide, N-bromosuccinimide, N-chlorosuccinimide, 1,3-diiodo-5,5-dimethylhydantion, pyridinium tribromide, iodine monochloride or complexes thereof, etc. Acids that are typically used under mild oxidizing condition include sulfuric acid, p-toluenesulfonic acid, trifluoromethanesulfonic acid, methanesulfonic acid, and trifluoroacetic acid. In certain embodiments, the mild oxidation reagent includes a mixture of N-iodosuccinimide and trifluoromethanesulfonic acid.

In step (e) above, removal of PG³ and optionally R⁴ (when R⁴ is a suitable amine protecting group) of a nucleic acid-ligand conjugate or analogue thereof of formula C5 affords a nucleic acid-ligand conjugate or analogue thereof of formula C6 or a salt thereof. In some embodiments, PG³ and/or R⁴ comprise carbamate derivatives that can be removed under acidic or basic conditions. In certain embodiments, the protecting groups (e.g., both PG³ and R⁴ or either of PG³ or R⁴ independently) of a nucleic acid-ligand conjugate or analogue thereof of formula C5 are removed by acid hydrolysis. It will be appreciated that upon acid hydrolysis of the protecting groups of a nucleic acid-ligand conjugate or analogue thereof of formula C5, a salt of formula C6 thereof is formed. For example, when an acid-labile protecting group of a nucleic acid-ligand conjugate or analogue thereof of formula C5 is removed by treatment with an acid such as hydrochloric acid, then the resulting amine compound would be formed as its hydrochloride salt. One of ordinary skill in the art would recognize that a wide variety of acids are useful for removing amino protecting groups that are acid-labile and therefore a wide variety of salt forms of a nucleic acid or analogue thereof of formula C6 are contemplated.

In other embodiments, the protecting groups (e.g., both PG³ and R⁴ or either of PG³ or R⁴ independently) of a nucleic acid or analogue thereof of formula C5 are removed by base hydrolysis. For example, Fmoc and trifluoroacetyl protecting groups can be removed by treatment with base. One of ordinary skill in the art would recognize that a wide variety of bases are useful for removing amino protecting groups that are base-labile. In some embodiments, a base is piperidine. In some embodiments, a base is 1,8-diazabicyclo[5.4.0]undec-7-ene (DBU). In certain embodiments, a nucleic acid-ligand conjugate or analogue thereof of formula C5 is deprotected under basic conditions followed by treating with an acid to form a salt of formula C6. In certain embodiments, the acid is fumaric acid the salt of formula C6 is the fumarate.

In some embodiments, the present disclosure provides a method for preparing an oligonucleotide-ligand conjugate comprising one or more nucleic acid-ligand conjugate, said nucleic acid-ligand conjugate unit represented by formula II-b-3:

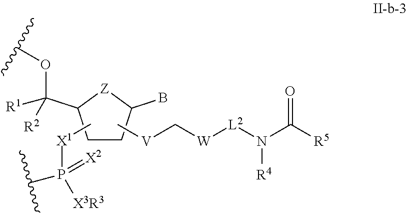

or a pharmaceutically acceptable salt thereof, comprising the steps of:
(a) providing an oligonucleotide of formula D5:

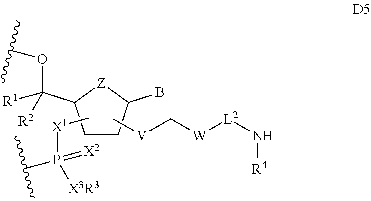

or salt thereof, and,
(b) conjugating one or more adamantyl or lipophilic compounds to an oligonucleotide of formula D5 to form an oligonucleotide-ligand conjugate of formula II-b-3 comprising one or more nucleic acid-ligand conjugate units. In step (b) above, conjugation is performed under suitable amide forming conditions to afford a compound of formula D5 comprising an adamantyl or lipid conjugate. Suitable amide forming conditions can include the use of an amide coupling reagent known in the art such as, but not limited to HATU, PyBOP, DCC, DIC, EDC, HBTU, HCTU, PyAOP, PyBrOP, BOP, BOP-CI, DEPBT, T3P, TATU, TBTU, TNTU, TOTU, TPTU, TSTU, or TDBTU. In certain embodiments, the amide forming conditions comprise HATU and DIPEA or TEA.

In some embodiments, the present disclosure provides a method for preparing an oligonucleotide-ligand conjugate comprising a unit represent by formula D5:

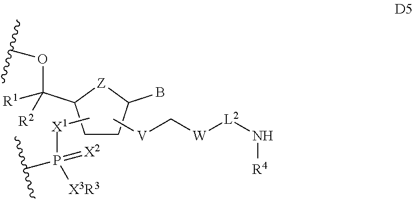

or a salt thereof, comprising the steps of:
(a) providing a nucleic acid-ligand conjugate or analogue thereof of formula D4:

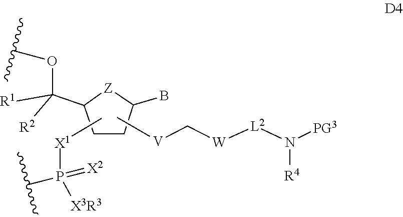

or salt thereof, and
(b) deprotecting said compound of formula D4 to form a compound of formula D5. In step (b) above, removal of PG³ and optionally R⁴ (when R⁴ is a suitable amine protecting group) of an oligonucleotide of formula D4 affords an oligonucleotide-ligand conjugate of formula D5 or a salt thereof. In some embodiments, PG³ and/or R⁴ comprise carbamate derivatives that can be removed under acidic or basic conditions. In certain embodiments, the protecting groups (e.g., both PG³ and R⁴ or either of PG³ or R⁴ independently) of an oligonucleotide-ligand conjugate of formula D4 are removed by acid hydrolysis. It will be appreciated that upon acid hydrolysis of the protecting groups of an oligonucleotide-ligand conjugate of formula D4, a salt of formula D5 thereof is formed. For example, when an acid-labile protecting group of an oligonucleotide of formula D4 is removed by treatment with an acid such as hydrochloric acid, then the resulting amine compound would be formed as its hydrochloride salt. One of ordinary skill in the art would recognize that a wide variety of acids are useful for removing amino protecting groups that are acid-labile and therefore a wide variety of salt forms of a nucleic acid-ligand conjugate unit or analogue thereof of formula D5 are contemplated.

In other embodiments, the protecting groups (e.g., both PG³ and R⁴ or either of PG³ or R⁴ independently) of an oligonucleotide-ligand conjugate of formula D4 are removed by base hydrolysis. For example, Fmoc and trifluoroacetyl protecting groups can be removed by treatment with base. One of ordinary skill in the art would recognize that a wide variety of bases are useful for removing amino protecting groups that are base-labile. In some embodiments, a base is piperidine. In some embodiments, a base is 1,8-diazabicyclo[5.4.0]undec-7-ene (DBU).

In some embodiments, the present disclosure provides a method for preparing an oligonucleotide-ligand conjugate comprising one or more nucleic acid-ligand conjugate unit with one or more adamantyl and/or lipid moiety, said conjugate unit represented by formula D4:

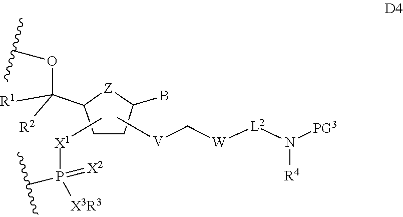

or a pharmaceutically acceptable salt thereof, comprising the steps of:
(a) providing a nucleic acid or analogue thereof of formula D3:

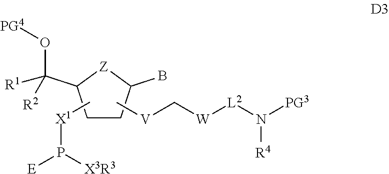

or salt thereof, and
(b) oligomerizing said compound of formula D3 to form a compound of formula D4,

In step (b) above, oligomerizing refers to preforming oligomerization forming conditions using known and commonly applied processes to prepare oligonucleotides in the art. For example, the nucleic acid or analogue thereof of formula D3 is coupled to a solid supported nucleic acid or analogue thereof bearing a 5′-hydroxyl group. Further steps can comprise one or more deprotections, couplings, phosphite oxidation, and cleavage from the solid support to provide an oligonucleotide of various nucleotide lengths, represented by a compound of formula D4 comprising an adamantyl or lipid conjugate of the disclosure.

In some embodiments, the present disclosure provides a method for preparing a nucleic acid or analogue thereof comprising one or more lipid conjugate, further comprising preparing a nucleic acid or analogue thereof of formula D3:

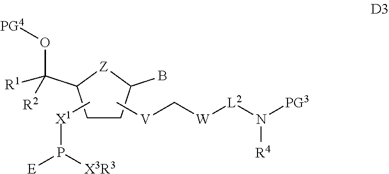

or a salt thereof, comprising the steps of:
(a) providing a nucleic acid or analogue thereof of formula C5:

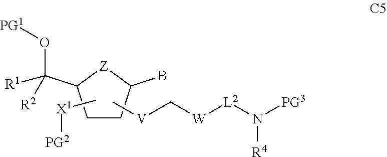

or salt thereof,
(b) deprotecting said nucleic acid or analogue thereof of formula C5 to form a compound of formula D1:

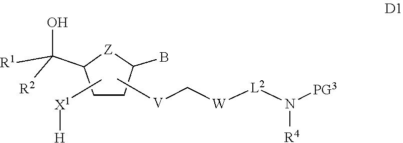

or salt thereof,
(c) protecting said nucleic acid or analogue thereof of formula D1 to form a nucleic acid or analogue thereof of formula D2:

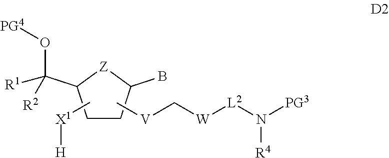

or salt thereof, and
(d) treating said nucleic acid or analogue thereof of formula D2 with a P(III) forming reagent to form a nucleic acid or analogue thereof of formula D3. In step (b) above, PG¹ and PG² of a nucleic acid or analogue thereof of formula C5 comprise silyl ethers or cyclic silylene derivatives that can be removed under acidic conditions or with fluoride anion. Examples of reagents providing fluoride anion for the removal of silicon-based protecting groups include hydrofluoric acid, hydrogen fluoride pyridine, triethylamine trihydrofluoride, tetra-N-butylammonium fluoride, and the like.

In step (c) above, a nucleic acid or analogue thereof of formula D1 is protected with a suitable hydroxyl protecting group. In certain embodiments, the protecting group PG⁴ used for protection of the 5′-hydroxyl group of a compound of formula D1 includes an acid labile protecting group such as trityl, 4-methyoxytrityl, 4,4′-dimethyoxytrityl, 4,4′,4″-trimethyoxytrityl, 9-phenyl-xanthen-9-yl, 9-(p-tolyl)-xanthen-9-yl, pixyl, 2,7-dimethylpixyl, and the like. In certain embodiments, the acid labile protecting group is suitable for deprotection during both solution-phase and solid-phase synthesis of acid-sensitive nucleic acids or analogues thereof using for example, dichloroacetic acid or trichloroacetic acid.

In step (d) above, a nucleic acid or analogue thereof of formula D2 is treated with a P(III) forming reagent to afford a compound of formula D3. In the context of the present disclosure, a P(III) forming reagent is a phosphorus reagent that is reacted to for a phosphorus (III) compound.

In some embodiments, the P(III) forming reagent is 2-cyanoethyl N,N-diisopropylchlorophosphoramidite or 2-cyanoethyl phosphorodichloridate. In certain embodiments, the P(III) forming reagent is 2-cyanoethyl N,N-diisopropylchlorophosphoramidite. One of ordinary skill would recognize that the displacement of a leaving group in a P(III) forming reagent by X¹ of a compound of formula D2 is achieved either with or without the presence of a suitable base. Such suitable bases are well known in the art and include organic and inorganic bases. In certain embodiments, the base is a tertiary amine such as triethylamine or diisopropylethylamine. In other embodiments, step (d) above is preformed using N,N-dimethylphosphoramic dichloride as a P(V) forming reagent.

### Formulations

Various formulations have been developed to facilitate oligonucleotide use. For example, oligonucleotides (e.g., double-stranded oligonucleotides) can be delivered to a subject or a cellular environment using a formulation that minimizes degradation, facilitates delivery and/or uptake, or provides another beneficial property to the oligonucleotides in the formulation. In some embodiments, provided herein are compositions comprising oligonucleotides (e.g., double-stranded oligonucleotides) reduce the expression of a target mRNA (e.g., a target mRNA expressed in extra-hepatic tissue). Such compositions can be suitably formulated such that when administered to a subject, either into the immediate environment of a target cell or systemically, a sufficient portion of the oligonucleotides enter the cell to reduce target gene expression. Any variety of suitable oligonucleotide formulations can be used to deliver oligonucleotides for the reduction of target gene expression as disclosed herein. In some embodiments, an oligonucleotide is formulated in buffer solutions such as phosphate buffered saline solutions, liposomes, micellar structures, and capsids.

In some embodiments, the formulations herein comprise an excipient. In some embodiments, an excipient confers to a composition improved stability, improved absorption, improved solubility and/or therapeutic enhancement of the active ingredient. In some embodiments, an excipient is a buffering agent (e.g., sodium citrate, sodium phosphate, a tris base, or sodium hydroxide) or a vehicle (e.g., a buffered solution, petrolatum, dimethyl sulfoxide, or mineral oil). In some embodiments, an oligonucleotide is lyophilized for extending its shelf-life and then made into a solution before use (e.g., administration to a subject). Accordingly, an excipient in a composition comprising any one of the oligonucleotides described herein may be a lyoprotectant (e.g., mannitol, lactose, polyethylene glycol or polyvinylpyrrolidone) or a collapse temperature modifier (e.g., dextran, Ficoll™ or gelatin). Likewise, the oligonucleotides herein may be provided in the form of their free acids.

In some embodiments, a pharmaceutical composition is formulated to be compatible with its intended route of administration. Examples of routes of administration include parenteral (e.g., intravenous, intramuscular, intraperitoneal, intradermal, subcutaneous, intrathecal), oral (e.g., inhalation), transdermal (e.g., topical), transmucosal and rectal administration.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL™ (BASF, Parsippany, N.J.) or phosphate buffered saline (PBS). The carrier can be a solvent or dispersion medium containing, for example, water, ethanol, polyol (e.g., glycerol, propylene glycol, and liquid polyethylene glycol, and the like), and suitable mixtures thereof. In many cases, it will be preferable to include isotonic agents, for example, sugars, polyalcohols such as mannitol, sorbitol, sodium chloride in the composition. Sterile injectable solutions can be prepared by incorporating the oligonucleotides in a required amount in a selected solvent with one or a combination of ingredients enumerated above, as required, followed by filtered sterilization.

In some embodiments, a composition may contain at least about 0.1% of the therapeutic agent (e.g., an double-stranded oligonucleotide herein) or more, although the percentage of the active ingredient(s) may be between about 1% to about 80% or more of the weight or volume of the total composition. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

### Structural Modifications

As nucleic acids are polymers of subunits or compounds, many of the modifications described below occur at a position which is repeated within a nucleic acid (e.g., a modification of a base, or a phosphate moiety, or the non-bridging oxygen of a phosphate moiety). In some cases the modification will occur at all of the subject positions in the nucleic acid but in many, and in fact in most cases it will not. By way of example, a modification may only occur at a 3′ or 5′ terminal position, may only occur in the internal unpaired region, may only occur in a terminal regions, e.g. at a position on a terminal nucleotide or in the last 2, 3, 4, 5, or 10 nucleotides of a strand. In some embodiments, a modification occurs at all of the subject positions in the nucleic acid. A modification may occur in a double strand region, a single strand region, or in both. A modification may occur only in the double strand region of an RNA agent or may only occur in a single strand region of an RNA agent. (e.g., a phosphorothioate modification at a non-bridging oxygen position may only occur at one or both termini, may only occur in a terminal regions or at a position on a terminal nucleotide or in the last 2, 3, 4, 5, or 10 nucleotides of a strand, or may occur in double strand and single strand regions, particularly at termini. The 5′ end or ends can be phosphorylated.

Many studies in the art have indicated that modified oligonucleotides and oligonucleotide analogs may be less readily internalized than their natural counterparts. As a result, the activity of many previously available RNAi trigger molecules has not been sufficient for practical therapeutic, research or diagnostic purposes.

Modifications to enhance the effectiveness of the RNAi trigger molecule oligonucleotides and overcome these problems have taken many forms. These modifications include base ring modifications, sugar moiety modifications, and sugar-phosphate backbone modifications, many exemplified herein and used in the current disclosure. Prior sugar-phosphate backbone modifications, particularly on the phosphorus atom, have affected various levels of resistance to nucleases. However, while the ability of an RNAi trigger molecule oligonucleotide to load into the RISC and direct the location of relevant mRNA sequences is fundamental to RNAi trigger molecule methodology, many modifications work at cross purposes with each other to optimize the behavior of the RNAi trigger. It is this balancing act which must be taken into account relative to the development of superior and effective RNAi molecules.

Another key factor is the stereochemical effect that arises in oligomers having P-chiral centers. In general, an oligomer with a length of n nucleosides will constitute a mixture of chirality in successive non-stereospecific chain synthesis. It has been observed that Rp and Sp homochiral chains, whose absolute configuration at all internucleotide methane phosphonate phosphorus atoms are either Rp or Sp, and non-stereoregular chains show different physicochemical properties as well as different capabilities of forming adducts with oligonucleotides of complementary sequence. In addition, phosphorothioate analogs of nucleotides have shown substantial stereoselectivity differences between Oligo-Rp and Oligo-Sp oligonucleotides in resistance to nucleases activity (Potter, BIOCHEMISTRY, 22:1369, (1983); Bryant et al., BIOCHEMISTRY, 18:2825, (1979)). Lesnikowski (NUCL. ACIDS RES., 18:2109, (1990)) observed that diastereomerically pure octathymidine methylphosphonates, in which six out of seven methylphosphonate bonds have defined configuration at the phosphorus atom when complexed with the matrix showed substantial differences in melting temperatures. According to the current disclosure chirally pure nucleotide analogs, or portions thereof, are expected to provide trigger structures with improved characteristics allowing the development of more potent and longer lasting RNAi triggers.

In some embodiments of the current disclosure it is particularly preferred to enhance stability, to include particular bases in overhangs, or to include modified nucleotides or nucleotide surrogates, in single strand overhangs, e.g., in a 5′ or 3′ overhang, or in both. Likewise, it can be desirable to include purine nucleotides in overhangs as they are more resistant to nuclease activity. In some embodiments all or some of the bases in a 3′ or 5′ overhang will be modified, with a modification described herein. Modifications can include the use of modifications at the 2′ OH group of the ribose sugar, deoxythymidine, instead of ribonucleotides, and modifications in the phosphate group, that is, phosphothioate modifications. Overhangs need not be homologous with the target sequence.

In some embodiments, the 3′ overhang comprises a sequence motif of: 3′-P₁P₂[N]_{y}X₁-5′ wherein:
P₁ and P₂ are each independently a purine or a pyrimidine, and do not comprise a 2′-F modification;
X₁ is any nucleotide located immediately adjacent to the duplex, and comprises at least one phosphorothioate linkage to an adjacent nucleotide;
N is any nucleotide and y is an integer selected from 1-6, specifying N₁-N₆, wherein:
   (a) when y is 1, N₁ comprises a 2′-F modification;
   (b) when y is 2, one or more of N₁ and N₂ comprise a 2′-F modification;
   (c) when y is 3, one or more of N₁-N₃ comprise a 2′-F modification;
   (d) when y is 4, one or more of N₁-N₄ comprise a 2′-F modification;
   (e) when y is 5, one or more of N₁-N₅ comprise a 2′-F modification; or
   (f) when y is 6, one or more of N₁-No comprise a 2′-F modification.

In some embodiments, the 3′ overhang comprises a sequence motif of: 3′-P₁P₂[N]_{y}X₁-5′ wherein:
P₁ and P₂ are each independently a purine, and do not comprise a 2′-F modification;
X₁ is any nucleotide located immediately adjacent to the duplex, and comprises at least one phosphorothioate linkage to an adjacent nucleotide;
N is any nucleotide and y is an integer selected from 1-6, specifying N₁-N₆, wherein:
   y is 3 and N₂ comprises a 2′F modification.

In some embodiments, the 3′ overhang comprises a sequence motif of: 3′-P₁P₂[N]_{y}X₁-5′ wherein:
P₁ and P₂ are each independently a purine, and do not comprise a 2′-F modification;
X₁ is any nucleotide located immediately adjacent to the duplex, and comprises at least one phosphorothioate linkage to an adjacent nucleotide;
N is any nucleotide and y is an integer selected from 1-6, specifying N₁-N₆, wherein:
   y is 3 and N₂ comprises a 2′-F modification and X₁ does not comprise a 2′-F modification.

In some embodiments, the 3′ overhang comprises a sequence motif of: 3′-P₁P₂[N]_{y}X₁-5′ wherein:
P₁ and P₂ are each independently a purine, and do not comprise a 2′-F modification;
X₁ is any nucleotide located immediately adjacent to the duplex, and comprises at least one phosphorothioate linkage to an adjacent nucleotide;
N is any nucleotide and y is an integer selected from 1-6, specifying N₁-N₆, wherein:
   y is 6 and N₂ comprise a 2′-F modification.

In some embodiments, the 3′overhang comprises a sequence motif of: 3′-P₁P₂[N] yX₁-5′ wherein:
P₁ and P₂ are each independently a purine, and do not comprise a 2′-F modification;
X₁ is any nucleotide located immediately adjacent to the duplex, and comprises at least one phosphorothioate linkage to an adjacent nucleotide;
N is any nucleotide and y is an integer selected from 1-6, specifying N₁-N₆, wherein:
   y is 6 and N₂ comprise a 2′-F modification and X₁ comprises a 2′-F modification.

In some embodiments, the 3′overhang comprises a sequence motif of: 3′-P₁P₂[N]_{y}X₁-5′ wherein:

P₁ and P₂ are each independently a purine, and do not comprise a 2′-F modification;
X₁ is any nucleotide located immediately adjacent to the duplex, and comprises at least one phosphorothioate linkage to an adjacent nucleotide;
N is any nucleotide and y is an integer selected from 1-6, specifying N₁-N₆, wherein:
   (a) y is 6 and N₂ comprise a 2′-F modification, and
   (b) N₁, N₃, N₄. N₅, and No each comprise a 2′-OMe modification.

In some embodiments, the 3′overhang comprises a sequence motif of: 3′-P₁P₂[N]_{y}X₁-5′ wherein:
P₁ and P₂ are each independently a purine, and do not comprise a 2′-F modification;
X₁ is any nucleotide located immediately adjacent to the duplex, and comprises at least one phosphorothioate linkage to an adjacent nucleotide;
N is any nucleotide and y is an integer selected from 1-6, specifying N₁-N₆, wherein:
   (a) y is 6 and N₂ comprise a 2′-F modification,
   (b) X₁ comprises a 2′-F modification, and
   (c) N₁, N₃, N₄. N₅, and N₆ each comprise a 2′-OMe modification.

In some embodiments, the 3′overhang comprises a sequence motif of: 3′-P₁P₂[N]_{y}X₁-5′ wherein:
P₁ and P₂ are each independently a purine, and do not comprise a 2′-F modification;
X₁ is any nucleotide located immediately adjacent to the duplex, and comprises at least one phosphorothioate linkage to an adjacent nucleotide;
N is any nucleotide and y is an integer selected from 1-6, specifying N₁-N₆, wherein:
   y is 6 and N₂ and N₅ each comprise the 2′-F modification.

In some embodiments, the 3′overhang comprises a sequence motif of: 3′-P₁P₂[N]_{y}X₁-5′ wherein:
P₁ and P₂ are each independently a purine, and do not comprise a 2′-F modification;
X₁ is any nucleotide located immediately adjacent to the duplex, and comprises at least one phosphorothioate linkage to an adjacent nucleotide;
N is any nucleotide and y is an integer selected from 1-6, specifying N₁-N₆, wherein:
   (a) y is 6 and N₂ and N₅ each comprise the 2′-F modification, and
   (b) X₁ comprises a 2′-F modification.

In some embodiments, the 3′overhang comprises a sequence motif of: 3′-P₁P₂[N]_{y}X₁-5′ wherein:
P₁ and P₂ are each independently a purine, and do not comprise a 2′-F modification;
X₁ is any nucleotide located immediately adjacent to the duplex, and comprises at least one phosphorothioate linkage to an adjacent nucleotide;
N is any nucleotide and y is an integer selected from 1-6, specifying N₁-N₆, wherein:
   (a) y is 6 and N₂ and N₅ each comprise the 2′-F modification, and
   (b) N₁, N₃, N₄ and N₆ each comprise a 2′-OMe modification.

In some embodiments, the 3′overhang comprises a sequence motif of: 3′-P₁P₂[N]_{y}X₁-5′ wherein:
P₁ and P₂ are each independently a purine, and do not comprise a 2′-F modification;
X₁ is any nucleotide located immediately adjacent to the duplex, and comprises at least one phosphorothioate linkage to an adjacent nucleotide;
N is any nucleotide and y is an integer selected from 1-6, specifying N₁-N₆, wherein:
   (a) y is 6 and N₂ and N₅ each comprise the 2′-F modification,
   (b) X₁ comprises a 2′-F modification, and
   (c) N₁, N₃, N₄ and N₆ each comprise a 2′-OMe modification.

In some embodiments, the double-stranded oligonucleotide comprises an antisense strand 20-22 nucleotides in length, and position 16 is a 2′-F modified nucleotide.

In some embodiments, the double-stranded oligonucleotide comprises an antisense strand 20-22 nucleotides in length, and position 16 and position 19 are 2′-F modified nucleotides.

In some embodiments, the 2′-F modified nucleotide comprises a phosphodiester linkage.

In some embodiments, the nucleotides on either side of the 2′-F modified nucleotide do not have phosphorothioate linkages. In some embodiments, the 3′ overhand comprises at least one 2′-F modified nucleotide wherein the nucleotides on either side of the 2′-F modified nucleotide do not have phosphorothioate linkages.

### METHODS OF USE

### Reducing Target Gene Expression

In some embodiments, the disclosure provides methods for contacting or delivering to a cell or population of cells an effective amount of any of the double-stranded oligonucleotides herein to reduce expression of a target gene.

In some embodiments, expression of a target gene is reduced in extra-hepatic tissue. In some embodiments, expression of a target gene is reduced in the liver. In some embodiments, expression of a target gene is reduced in a hepatocyte. In some embodiments, expression of a target gene is reduced in adipose tissue. In some embodiments, expression of a target gene is reduced in adrenal tissue. In some embodiments, expression of a target gene is reduced in skeletal muscle tissue. In some embodiments, expression of a target gene is reduced in cardiomyocytes. In some embodiments, expression of a target gene is reduced in liver non-parenchymal cells. In some embodiments, expression of a target gene is reduced in immune cells. In some embodiments, expression of a target gene is reduced in extra-hepatic tissue more than reduction of expression of the target gene in a hepatocyte.

In some embodiments, a reduction of target gene expression is determined by measuring a reduction in the amount or level of target mRNA, protein encoded by the target mRNA, or target gene (mRNA or protein) activity in a cell. The methods include those described herein and known to one of ordinary skill in the art.

Methods provided herein are useful in any appropriate cell type. In some embodiments, a cell is any cell that expresses the target mRNA. In some embodiments, the cell is a primary cell obtained from a subject. In some embodiments, the primary cell has undergone a limited number of passages such that the cell substantially maintains is natural phenotypic properties. In some embodiments, a cell to which the oligonucleotide is delivered is ex vivo or in vitro (i.e., can be delivered to a cell in culture or to an organism in which the cell resides).

In some embodiments, the double-stranded oligonucleotides disclosed herein are delivered to a cell or population of cells using a nucleic acid delivery method known in the art including, but not limited to, injection of a solution or pharmaceutical composition containing the double-stranded oligonucleotide, bombardment by particles covered by the double-stranded oligonucleotide, exposing the cell or population of cells to a solution containing the double-stranded oligonucleotide, or electroporation of cell membranes in the presence of the double-stranded oligonucleotide. Other methods known in the art for delivering oligonucleotides to cells may be used, such as lipid-mediated carrier transport, chemical-mediated transport, and cationic liposome transfection such as calcium phosphate, and others.

In some embodiments, reduction of target gene expression is determined by an assay or technique that evaluates one or more molecules, properties or characteristics of a cell or population of cells associated with target gene expression, or by an assay or technique that evaluates molecules that are directly indicative of target gene expression in a cell or population of cells (e.g., target mRNA or protein). In some embodiments, the extent to which a double-stranded oligonucleotide provided herein reduces target gene expression in a cell is evaluated by comparing target gene expression in a cell or population of cells contacted with the double-stranded oligonucleotide to a control cell or population of cells (e.g., a cell or population of cells not contacted with the double-stranded oligonucleotide or contacted with a control double-stranded oligonucleotide). In some embodiments, a control amount or level of target gene expression in a control cell or population of cells is predetermined, such that the control amount or level need not be measured in every instance the assay or technique is performed. The predetermined level or value can take a variety of forms. In some embodiments, a predetermined level or value can be single cut-off value, such as a median or mean.

In some embodiments, contacting or delivering a double-stranded oligonucleotide described herein to a cell or a population of cells results in a reduction in expression of a target gene. In some embodiments, the reduction in target gene expression is relative to a control amount or level of target gene expression in cell or population of cells not contacted with the double-stranded oligonucleotide or contacted with a control double-stranded oligonucleotide. In some embodiments, the reduction in target gene expression is about 1% or lower, about 5% or lower, about 10% or lower, about 15% or lower, about 20% or lower, about 25% or lower, about 30% or lower, about 35% or lower, about 40% or lower, about 45% or lower, about 50% or lower, about 55% or lower, about 60% or lower, about 70% or lower, about 80% or lower, or about 90% or lower relative to a control amount or level of target gene expression. In some embodiments, the control amount or level of target gene expression is an amount or level of target mRNA and/or protein in a cell or population of cells that has not been contacted with a double-stranded oligonucleotide herein. In some embodiments, the effect of delivery of a double-stranded oligonucleotide to a cell or population of cells according to a method herein is assessed after any finite period or amount of time (e.g., minutes, hours, days, weeks, months). For example, in some embodiments, target gene expression is determined in a cell or population of cells at least about 4 hours, about 8 hours, about 12 hours, about 18 hours, about 24 hours; or at least about 1 day, about 2 days, about 3 days, about 4 days, about 5 days, about 6 days, about 7 days, about 8 days, about 9 days, about 10 days, about 11 days, about 12 days, about 13 days, about 14 days, about 21 days, about 28 days, about 35 days, about 42 days, about 49 days, about 56 days, about 63 days, about 70 days, about 77 days, or about 84 days or more after contacting or delivering the double-stranded oligonucleotide to the cell or population of cells. In some embodiments, target gene expression is determined in a cell or population of cells at least about 1 month, about 2 months, about 3 months, about 4 months, about 5 months, or about 6 months or more after contacting or delivering the double-stranded oligonucleotide to the cell or population of cells.

### Reducing Target Gene Expression in Extra-hepatic Tissue

In some embodiments, expression of a target gene is reduced in extra-hepatic tissue. In some embodiments, expression of a target gene is reduced in a cell of an extra-hepatic tissue. In some embodiments, expression of a target gene is reduced in at least one extra-hepatic tissue. In some embodiments, expression of a target gene is reduced in one or more extra-hepatic tissues. In some embodiments, extra-hepatic tissue includes, but is not limited to, skeletal muscle, adipose tissue, and adrenal tissue. In some embodiments, a cell of an extra-hepatic tissue includes but is not limited to a cardiomyocyte, a liver non-parenchymal cell, an immune cells, a cell of skeletal tissue, a cell of adrenal tissue, a cell of adipose tissue, or any combination thereof.

In some embodiments, expression of a target gene in the extra-hepatic tissue of a subject is reduced by at least about 30%, about 35%, about 40%, about 45%, about 50%, about 55%, about 60%, about 65%, about 70%, about 75%, about 80%, about 85%, about 90%, about 95%, about 99% or greater than 99% when compared to target gene expression in a control tissue.

In some embodiments, expression of a target gene in the extra-hepatic tissue of a subject is reduced by at least about 30%, about 35%, about 40%, about 45%, about 50%, about 55%, about 60%, about 65%, about 70%, about 75%, about 80%, about 85%, about 90%, about 95%, about 99% or greater than 99% when compared to target gene expression in a hepatocyte.

In some embodiments, contacting or delivering a double-stranded oligonucleotide described herein to a cell or a population of cells results in a reduction in expression of a target gene in extra-hepatic tissue. In some embodiments, reduction in expression of a target gene in extra-hepatic tissue is increased by at least 1%, at least 5%, at least 10%, at least 15%, at least 20%, at least 25%, at least 30%, at least 35%, at least 40%, at least 45%, at least 50%, at least 55%, at least 60%, at least 70%, at least 80%, or at least 90% relative to reduction in expression of the target gene in a hepatocyte.

In some embodiments, contacting or delivering a double-stranded oligonucleotide described herein to a cell or a population of cells results in a reduction in expression of target gene in skeletal muscle. In some embodiments, reduction in expression of a target gene in skeletal muscle is increased by at least 1%, at least 5%, at least 10%, at least 15%, at least 20%, at least 25%, at least 30%, at least 35%, at least 40%, at least 45%, at least 50%, at least 55%, at least 60%, at least 70%, at least 80%, or at least 90% relative to reduction in expression of the target gene in a hepatocyte.

In some embodiments, contacting or delivering a double-stranded oligonucleotide described herein to a cell or a population of cells results in a reduction in expression of target gene in adipose tissue. In some embodiments, reduction in expression of a target gene in adipose is increased by at least 1%, at least 5%, at least 10%, at least 15%, at least 20%, at least 25%, at least 30%, at least 35%, at least 40%, at least 45%, at least 50%, at least 55%, at least 60%, at least 70%, at least 80%, or at least 90% relative to reduction in expression of the target gene in a hepatocyte.

In some embodiments, contacting or delivering a double-stranded oligonucleotide described herein to a cell or a population of cells results in a reduction in expression of target gene in adrenal tissue. In some embodiments, reduction in expression of a target gene in adrenal is increased by at least 1%, at least 5%, at least 10%, at least 15%, at least 20%, at least 25%, at least 30%, at least 35%, at least 40%, at least 45%, at least 50%, at least 55%, at least 60%, at least 70%, at least 80%, or at least 90% relative to reduction in expression of the target gene in a hepatocyte.

In some embodiments, contacting or delivering a double-stranded oligonucleotide described herein to a cell or a population of cells results in a reduction in expression of target gene in a cardiomyocyte. In some embodiments, reduction in expression of a target gene in a cardiomyocyte is increased by at least 1%, at least 5%, at least 10%, at least 15%, at least 20%, at least 25%, at least 30%, at least 35%, at least 40%, at least 45%, at least 50%, at least 55%, at least 60%, at least 70%, at least 80%, or at least 90% relative to reduction in expression of the target gene in a hepatocyte.

In some embodiments, contacting or delivering a double-stranded oligonucleotide described herein to a cell or a population of cells results in a reduction in expression of target gene in an immune cell. In some embodiments, reduction in expression of a target gene in an immune cell is increased by at least 1%, at least 5%, at least 10%, at least 15%, at least 20%, at least 25%, at least 30%, at least 35%, at least 40%, at least 45%, at least 50%, at least 55%, at least 60%, at least 70%, at least 80%, or at least 90% relative to reduction in expression of the target gene in a hepatocyte.

In some embodiments, contacting or delivering a double-stranded oligonucleotide described herein to a cell or a population of cells results in a reduction in expression of target gene in a liver non-parenchymal cell. In some embodiments, reduction in expression of a target gene in a liver non-parenchymal cell is increased by at least 1%, at least 5%, at least 10%, at least 15%, at least 20%, at least 25%, at least 30%, at least 35%, at least 40%, at least 45%, at least 50%, at least 55%, at least 60%, at least 70%, at least 80%, or at least 90% relative to reduction in expression of the target gene in a hepatocyte.

In some embodiments, contacting or delivering an oligonucleotide described herein to a cell or a population of cells results in a reduction in expression of a target gene in extra-hepatic tissue.

In some embodiments, differences in target mRNA expression between cell types or tissue types is measured using methods known in the art. In some embodiments, differences in target mRNA expression between cell types or tissue types measures the reduction of the target mRNA in a first cell/tissue type compared to the reduction of target mRNA in a second cell/tissue type. For example, differences in target mRNA expression between cell types or tissue types is measured using polymerase chain reaction methods (e.g., RT-qPCR) comparing relative expression between different tissue or cell types. In some embodiments, differences in target mRNA expression between cell types or tissue types is measured using Northern blot analysis, in situ hybridization, RT-qPCR, RNA sequencing, or other methods known in the art. In some embodiments, a relative amount of target mRNA expression is compared between cell or tissue types. In some embodiments, an absolute amount of target mRNA expression is compared between cell or tissue types.

### Treatment Methods

In some embodiments, the disclosure provides methods for treating a disease, disorder, or condition associated with expression of a target gene in extra-hepatic tissue. In some embodiments, the disclosure provides methods for treating a disease, disorder, or condition associated with expression of a target gene in a hepatocyte. In some embodiments, the disclosure provides methods for treating a disease, disorder, or condition associated with expression of a target gene in adipose tissue. In some embodiments, the disclosure provides methods for treating a disease, disorder, or condition associated with expression of a target gene in adrenal tissue. In some embodiments, the disclosure provides methods for treating a disease, disorder, or condition associated with expression of a target gene in skeletal muscle tissue. In some embodiments, the disclosure provides methods for treating a disease, disorder, or condition associated with expression of a target gene in cardiomyocytes. In some embodiments, the disclosure provides methods for treating a disease, disorder, or condition associated with expression of a target gene in immune cells. In some embodiments, the disclosure provides methods for treating a disease, disorder, or condition associated with expression of a target gene in liver non-parenchymal cells.

Methods described herein are typically involve administering to a subject a therapeutically effective amount of a double-stranded oligonucleotide herein, that is, an amount capable of producing a desirable therapeutic result. A therapeutically acceptable amount may be an amount that can therapeutically treat a disease or disorder. The appropriate dosage for any one subject will depend on certain factors, including the subject's size, body surface area, age, the composition to be administered, the active ingredient(s) in the composition, time and route of administration, general health, and other drugs being administered concurrently.

In some embodiments, a subject is administered any one of the compositions herein either enterally (e.g., orally, by gastric feeding tube, by duodenal feeding tube, via gastrostomy or rectally), parenterally (e.g., subcutaneous injection, intravenous injection or infusion, intra-arterial injection or infusion, intraosseous infusion, intramuscular injection, intracerebral injection, intracerebroventricular injection, intrathecal), topically (e.g., epicutaneous, inhalational, via eye drops, or through a mucous membrane), or by direct injection into a target organ (e.g., the brain of a subject).

In some embodiments, a double-stranded oligonucleotide herein, or a composition thereof, is administered once every year, once every 6 months, once every 4 months, quarterly (once every three months), bi-monthly (once every two months), monthly or weekly. In some embodiments, a double-stranded oligonucleotide herein, or a composition thereof, is administered every week or at intervals of two, or three weeks. In some embodiments, a double-stranded oligonucleotide herein, or a composition thereof, is administered daily. In some embodiments, a subject is administered one or more loading doses of a double-stranded oligonucleotide herein, or a composition thereof, followed by one or more maintenance doses of the double-stranded oligonucleotide, or a composition thereof.

In some embodiments, the subject to be treated is a human or non-human primate or other mammalian subject. Other exemplary subjects include domesticated animals such as dogs and cats; livestock such as horses, cattle, pigs, sheep, goats, and chickens; and animals such as mice, rats, guinea pigs, and hamsters.

### Treatment Methods in Extra-Hepatic Tissue

The disclosure provides oligonucleotides for use as a medicament, in particular for use in a method for the treatment of diseases, disorders, and conditions associated with extra-hepatic tissue. The disclosure also provides double-stranded oligonucleotides for use, or adaptable for use, to treat a subject (e.g., a human) having a disease, disorder or condition associated with expression of a target gene that would benefit from reducing expression of the target gene. In some embodiments, the disclosure provides double-stranded oligonucleotides for use, or adapted for use, to treat a subject having a disease, disorder or condition associated with expression of a target gene in extra-hepatic tissue. The disclosure also provides double-stranded oligonucleotides for use, or adaptable for use, in the manufacture of a medicament or pharmaceutical composition for treating a disease, disorder or condition associated with expression of a target gene in extra-hepatic tissue. In some embodiments, the double-stranded oligonucleotides for use, or adaptable for use, target mRNA and reduce expression of a target gene in extra-hepatic tissue (e.g., via the RNAi pathway). In some embodiments, the double-stranded oligonucleotides for use, or adaptable for use, target mRNA and reduce the amount or level of target mRNA, protein and/or activity.

In some embodiments, the extra-hepatic tissue is skeletal muscle, adrenal tissue, adipose tissue, cardiomyocytes, immune cells, liver non-parenchymal cells, or any combination thereof.

In addition, in some embodiments of the methods herein, a subject having a disease, disorder or condition associated with expression of a target gene in extra-hepatic tissue or is predisposed to the same is selected for treatment with a double-stranded oligonucleotide herein. In some embodiments, the method comprises selecting an individual having a marker (e.g., a biomarker) for a disease, disorder or condition associated with expression of a target gene in extra-hepatic tissue, or predisposed to the same, such as, but not limited to, target mRNA, protein, or a combination thereof. Likewise, and as detailed below, some embodiments of the methods provided by the disclosure include steps such as measuring or obtaining a baseline value for a marker of expression of a target gene in extra-hepatic tissue, and then comparing such obtained value to one or more other baseline values or values obtained after the subject is administered the double-stranded oligonucleotide to assess the effectiveness of treatment.

The disclosure also provides methods of treating a subject having, suspected of having, or at risk of developing a disease, disorder or condition associated with expression of a target gene in extra-hepatic tissue with a double-stranded oligonucleotide provided herein. In some embodiments, the disclosure provides methods of treating or attenuating the onset or progression of a disease, disorder or condition associated with expression of a target gene in extra-hepatic tissue using the double-stranded oligonucleotides provided herein. In some embodiments, the disclosure provides methods of treating or attenuating the onset or progression of a disease, disorder or condition associated with expression of a target gene in extra-hepatic tissue but not expression in a hepatocyte, using the double-stranded oligonucleotides provided herein. In some embodiments, the disclosure provides methods to achieve one or more therapeutic benefits in a subject having a disease, disorder or condition associated with expression of a target gene in extra-hepatic tissue using the double-stranded oligonucleotides provided herein.

In some embodiments of the methods herein, the subject is treated by administering a therapeutically effective amount of any one or more of the double-stranded oligonucleotides provided herein. In some embodiments, treatment comprises reducing expression of a target gene in extra-hepatic tissue. In some embodiments, the subject is treated therapeutically. In some embodiments, the subject is treated prophylactically.

In some embodiments of the methods herein, a double-stranded oligonucleotide provided herein, or a pharmaceutical composition comprising the double-stranded oligonucleotide, is administered to a subject having a disease, disorder or condition associated with expression of a target gene in extra-hepatic tissue such that target gene expression is reduced in the subject, thereby treating the subject. In some embodiments, an amount or level of target mRNA is reduced in the subject. In some embodiments, an amount or level of protein encoded by the target mRNA is reduced in the subject.

In some embodiments of the methods herein, a double-stranded oligonucleotide provided herein, or a pharmaceutical composition comprising the double-stranded oligonucleotide, is administered to a subject having a disease, disorder or condition associated with expression of a target gene in extra-hepatic tissue such that target gene expression is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to target gene expression prior to administration of the double-stranded oligonucleotide or pharmaceutical composition. In some embodiments, expression of a target gene in extra-hepatic tissue is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to target gene expression in a subject (e.g., a reference or control subject) not receiving the double-stranded oligonucleotide or pharmaceutical composition or receiving a control double-stranded oligonucleotide, pharmaceutical composition or treatment.

In some embodiments of the methods herein, a double-stranded oligonucleotide herein, or a pharmaceutical composition comprising the double-stranded oligonucleotide, is administered to a subject having a disease, disorder or condition associated with expression of a target gene in the extra-hepatic tissue such that an amount or level of target mRNA is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the amount or level of target mRNA prior to administration of the double-stranded oligonucleotide or pharmaceutical composition. In some embodiments, an amount or level of target mRNA is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to an amount or level of target mRNA in a subject (e.g., a reference or control subject) not receiving the double-stranded oligonucleotide or pharmaceutical composition or receiving a control double-stranded oligonucleotide, pharmaceutical composition or treatment.

In some embodiments of the methods herein, a double-stranded oligonucleotide herein, or a pharmaceutical composition comprising the double-stranded oligonucleotide, is administered to a subject having a disease, disorder or condition associated with expression of a target gene in extra-hepatic tissue such that an amount or level of protein encoded by the target gene is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the amount or level of protein encoded by the target gene prior to administration of the double-stranded oligonucleotide or pharmaceutical composition. In some embodiments, an amount or level of protein encoded by a target gene in extra-hepatic tissue is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to an amount or level of protein encoded by the target gene in a subject (e.g., a reference or control subject) not receiving the double-stranded oligonucleotide or pharmaceutical composition or receiving a control double-stranded oligonucleotide, pharmaceutical composition or treatment.

In some embodiments of the methods herein, a double-stranded oligonucleotide herein, or a pharmaceutical composition comprising the double-stranded oligonucleotide, is administered to a subject having a disease, disorder or condition associated with expression of a target gene in extra-hepatic tissue such that an amount or level of target gene activity is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the amount or level of target gene activity prior to administration of the double-stranded oligonucleotide or pharmaceutical composition. In some embodiments, an amount or level of target gene activity is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to an amount or level of target gene activity in a subject (e.g., a reference or control subject) not receiving the double-stranded oligonucleotide or pharmaceutical composition or receiving a control double-stranded oligonucleotide, pharmaceutical composition or treatment.

Suitable methods for determining target gene expression, an amount or level of target mRNA, an amount or level of protein encoded by the target gene, and/or an amount or level of target gene activity, in the subject, or in a sample from the subject, are known in the art. Further, the Examples set forth herein illustrate exemplary methods for determining target gene expression.

In some embodiments, target gene expression, an amount or level of target gene mRNA, an amount or level of protein encoded by a target gene, an amount or level of target gene activity, or any combination thereof, is reduced in a cell, a population or a group of cells (e.g., an organoid), an organ, blood or a fraction thereof (e.g., plasma), a tissue (e.g., skeletal muscle), a sample (e.g., a biopsy sample), or any other biological material obtained or isolated from the subject. In some embodiments, expression of a target gene in extra-hepatic tissue, an amount or level of target gene mRNA, an amount or level of protein encoded by the target gene, an amount or level of target gene activity, or any combination thereof, is reduced in more than one type of cell, more than one groups of cells, more than one organ (e.g., skeletal muscle and one or more other organ(s)), more than one fraction of blood (e.g., plasma and one or more other blood fraction(s)), more than one type of tissue (e.g., skeletal tissue and one or more other type(s) of tissue), more than one type of sample (e.g., a skeletal biopsy sample and one or more other type(s) of biopsy sample) obtained or isolated from the subject.

In some embodiments, expression of a target mRNA is reduced in one or more of skeletal muscle, adipose tissue, adrenal tissue, cardiomyocytes, immune cells, and liver non-parenchymal cells. In some embodiments, expression of a target mRNA is reduced in one or more of skeletal muscle, adipose tissue, and adrenal tissue. In some embodiments, expression of a target mRNA is reduced in skeletal muscle. In some embodiments, expression of a target mRNA is reduced in adipose tissue. In some embodiments, expression of a target mRNA is reduced in adrenal tissue.

In some embodiments, the extra-hepatic target gene may be a target gene from any mammal, such as a human. Any extra-hepatic gene may be silenced according to the method described herein.

In some embodiments, an oligonucleotide herein, or a composition thereof, is administered via subcutaneous or intravenous administration.

### Kits

In some embodiments, the disclosure provides a kit comprising a double-stranded oligonucleotide herein, or a composition thereof, described herein, and instructions for use. In some embodiments, the kit comprises a double-stranded oligonucleotide herein, or a composition thereof, described herein, and a package insert containing instructions for use of the kit and/or any component thereof. In some embodiments, the kit comprises, in a suitable container, a double-stranded oligonucleotide herein, or a composition thereof, described herein, one or more controls, and various buffers, reagents, enzymes and other standard ingredients well known in the art. In some embodiments, the container comprises at least one vial, well, test tube, flask, bottle, syringe, or other container means, into which the double-stranded oligonucleotide herein, or a composition thereof, is placed, and in some instances, suitably aliquoted. In some embodiments where an additional component is provided, the kit contains additional containers into which this component is placed. The kits can also include a means for containing a double-stranded oligonucleotide herein, or a composition thereof, and any other reagent in close confinement for commercial sale. Such containers may include injection or blow-molded plastic containers into which the desired vials are retained. Containers and/or kits can include labeling with instructions for use and/or warnings.

In some embodiments, a kit comprises a double-stranded oligonucleotide herein, or a composition thereof, described herein, and a pharmaceutically acceptable carrier, or a pharmaceutical composition comprising the double-stranded oligonucleotide and instructions for treating or delaying progression of a disease, disorder or condition associated with expression of a target gene expressed in extra-hepatic tissue in a subject in need thereof.

In some embodiments, a kit comprises a double-stranded oligonucleotide herein, or a composition thereof, described herein, and a pharmaceutically acceptable carrier, or a pharmaceutical composition comprising the double-stranded oligonucleotide and instructions for treating or delaying progression of a disease, disorder or condition associated with expression of a target gene expressed in skeletal muscle, adipose tissue, adrenal tissue, liver non-parenchymal cells, cardiomyocytes, or immune cells in a subject in need thereof.

In some embodiments, a kit comprises a double-stranded oligonucleotide herein, or a composition thereof, described herein, and a pharmaceutically acceptable carrier, or a pharmaceutical composition comprising the double-stranded oligonucleotide and instructions for treating or delaying progression of a disease, disorder or condition associated with expression of a target gene expressed in skeletal muscle in a subject in need thereof.

In some embodiments, a kit comprises a double-stranded oligonucleotide herein, or a composition thereof, described herein, and a pharmaceutically acceptable carrier, or a pharmaceutical composition comprising the double-stranded oligonucleotide and instructions for treating or delaying progression of a disease, disorder or condition associated with expression of a target gene expressed in adipose tissue in a subject in need thereof.

In some embodiments, a kit comprises a double-stranded oligonucleotide herein, or a composition thereof, described herein, and a pharmaceutically acceptable carrier, or a pharmaceutical composition comprising the double-stranded oligonucleotide and instructions for treating or delaying progression of a disease, disorder or condition associated with expression of a target gene expressed in adrenal tissue in a subject in need thereof.

In some embodiments, a kit comprises a double-stranded oligonucleotide herein, or a composition thereof, described herein, and a pharmaceutically acceptable carrier, or a pharmaceutical composition comprising the double-stranded oligonucleotide and instructions for treating or delaying progression of a disease, disorder or condition associated with expression of a target gene expressed in immune cells in a subject in need thereof.

In some embodiments, a kit comprises a double-stranded oligonucleotide herein, or a composition thereof, described herein, and a pharmaceutically acceptable carrier, or a pharmaceutical composition comprising the double-stranded oligonucleotide and instructions for treating or delaying progression of a disease, disorder or condition associated with expression of a target gene expressed in cardiomyocytes in a subject in need thereof.

In some embodiments, a kit comprises a double-stranded oligonucleotide herein, or a composition thereof, described herein, and a pharmaceutically acceptable carrier, or a pharmaceutical composition comprising the double-stranded oligonucleotide and instructions for treating or delaying progression of a disease, disorder or condition associated with expression of a target gene expressed in liver non-parenchymal cells in a subject in need thereof.

### Definitions

As used herein, the term “and/or” includes any and all combinations of one or more of the associated listed items. Further, the singular forms and the articles “a”, “an” and “the” are intended to include the plural forms as well, unless expressly stated otherwise. It will be further understood that the terms: includes, comprises, including and/or comprising, when used in this specification, specify the presence of stated features, integers, steps, operations, elements, and/or components, but do not preclude the presence or addition of one or more other features, integers, steps, operations, elements, components, and/or groups thereof. Further, it will be understood that when an element, including component or subsystem, is referred to and/or shown as being connected or coupled to another element, it can be directly connected or coupled to the other element or intervening elements may be present.

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, exemplary methods, and materials are described herein.

General texts which describe molecular biological techniques useful herein, including the use of vectors, promoters and many other relevant topics, include Berger and Kimmel, GUIDE TO MOLECULAR CLONING TECHNIQUES, METHODS IN ENZYMOLOGY, volume 152, (Academic Press, Inc., San Diego, Calif.)(“Berger”); Sambrook et al., MOLECULAR CLONING—A LABORATORY MANUAL, 2d ed., Vol. 1-3, Cold Spring Harbor Laboratory, Cold Spring Harbor, 1989 (“Sambrook”) and CURRENT PROTOCOLS IN MOLECULAR BIOLOGY, F. M. Ausubel et al., eds., CURRENT PROTOCOLS, A JOINT VENTURE BETWEEN GREENE PUBLISHING ASSOCIATES, INC. AND JOHN WILEY AND SONS, INC., (supplemented through 1999)(“Ausubel”). Examples of protocols sufficient to direct persons of skill through in vitro amplification methods, including the polymerase chain reaction (PCR), the ligase chain reaction (LCR), Q.beta.-replicase amplification and other RNA polymerase mediated techniques (e.g., NASBA), e.g., for the production of the homologous nucleic acids of the disclosure are found in Berger, Sambrook, and Ausubel, as well as in Mullis et al., (1987) U.S. Pat. No. 4,683,202; Innis et al., eds. (1990); PCR PROTOCOLS: A GUIDE TO METHODS AND APPLICATIONS (Academic Press Inc. San Diego, Calif.) (“Innis”); Arnheim and Levinson (Oct. 1, 1990) CandEN 36-47; J.NIH RES. (1991) 3:81-94; Kwoh et al., (1989) PROC. NATL. ACAD. SCI. USA 86:1173; Guatelliet al (1990) PROC. NAT'L. ACAD. SCI. USA 87:1874; Lomell et al., (1989) J. CLIN. CHEM 35:1826; Landegren et al., (1988) SCIENCE 241:1077-80; Van Brunt (1990) BIOTECHNOLOGY 8:291-94; Wu and Wallace (1989) GENE 4:560; Barringer et al., (1990) GENE 89:117; and, Sooknanan and Malek (1995) BIOTECHNOLOGY 13:563-564. Improved methods for cloning in vitro amplified nucleic acids are described in Wallace et al., U.S. Pat. No. 5,426,039. Improved methods for amplifying large nucleic acids by PCR are summarized in Cheng et al., (1994) NATURE 369:684-85 and the references cited therein, in which PCR amplicons of up to 40 kb are generated.

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one value, and/or to “about” another value. When such a range is expressed, another embodiment includes from the one value and/or to the other value. Similarly, when values are expressed as approximations, by use of the antecedent “about,” it will be understood that the value forms another embodiment. It will be further understood that the endpoints of each of the ranges are significant both in relation to the other endpoint, and independently of the other endpoint. It is also understood that there are several values disclosed herein, and that each value is also herein disclosed as “about” that value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in several different formats, and that this data, represents endpoints and starting points, and ranges for any combination of the data points. For example, if a particular datapoint “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

As used here, the term “amount” refers to an absolute amount (e.g., an absolute amount of mRNA or protein), a relative amount (e.g., a relative amount of target mRNA as measured by PCR assay or protein), or a concentration (e.g. a concentration of double-stranded RNA in a composition), whether the amount referred to in a given instance refers to an absolute amount, concentration, or both, will be clear to the skilled artisan based on the context provided herein.

As used herein, “bicyclic nucleotide” refers to a nucleotide comprising a bicyclic sugar moiety.

As used herein “bicyclic sugar moiety” refers to a modified sugar moiety comprising a 4 to 7 membered ring (including but not limited to a furanosyl) comprising a bridge connecting two atoms of the 4 to 7 membered ring to form a second ring, resulting in a bicyclic structure. Typically, the 4 to 7 membered ring is a sugar. In some embodiments, the 4-to-7-member ring is a furanosyl. In certain embodiments, the bridge connects the 2′-carbon and the 4′-carbon of the furanosyl.

As used herein, “complementary” refers to a structural relationship between two nucleotides (e.g., on two opposing nucleic acids or on opposing regions of a single nucleic acid strand) that permits the two nucleotides to form base pairs with one another. For example, a purine nucleotide of one nucleic acid that is complementary to a pyrimidine nucleotide of an opposing nucleic acid may base pair together by forming hydrogen bonds with one another. In some embodiments, complementary nucleotides can base pair in the Watson-Crick manner or in any other manner that allows for the formation of stable duplexes. In some embodiments, two nucleic acids may have regions of multiple nucleotides that are complementary with each other to form regions of complementarity, as described herein.

As used herein, “deoxyribonucleotide” refers to a nucleotide having a hydrogen in place of a hydroxyl at the 2′ position of its pentose sugar when compared with a ribonucleotide. A modified deoxyribonucleotide is a deoxyribonucleotide having one or more modifications or substitutions of atoms other than at the 2′ position, including modifications or substitutions in or of the sugar, phosphate group or base.

As used herein, “double-stranded RNA” or “dsRNA” refers to an RNA oligonucleotide that is substantially in a duplex form. In some embodiments, the complementary base-pairing of duplex region(s) of a dsRNA oligonucleotide is formed between antiparallel sequences of nucleotides of covalently separate nucleic acid strands. In some embodiments, complementary base-pairing of duplex region(s) of a dsRNA formed between antiparallel sequences of nucleotides of nucleic acid strands that are covalently linked. In some embodiments, complementary base-pairing of duplex region(s) of a dsRNA is formed from single nucleic acid strand that is folded (e.g., via a hairpin) to provide complementary antiparallel sequences of nucleotides that base pair together. In some embodiments, a dsRNA comprises two covalently separate nucleic acid strands that are fully duplexed with one another. However, in some embodiments, a dsRNA comprises two covalently separate nucleic acid strands that are partially duplexed (e.g., having overhangs at one or both ends). In some embodiments, a dsRNA comprises antiparallel sequence of nucleotides that are partially complementary, and thus, may have one or more mismatches, which may include internal mismatches or end mismatches.

As used herein, “duplex,” in reference to nucleic acids (e.g., oligonucleotides), refers to a structure formed through complementary base pairing of two antiparallel sequences of nucleotides.

As used herein, “excipient” refers to a non-therapeutic agent that may be included in a composition, for example, to provide or contribute to a desired consistency or stabilizing effect. As used herein, “loop” refers to an unpaired region of a nucleic acid (e.g., oligonucleotide) that is flanked by two antiparallel regions of the nucleic acid that are sufficiently complementary to one another, such that under appropriate hybridization conditions (e.g., in a phosphate buffer, in a cells), the two antiparallel regions, which flank the unpaired region, hybridize to form a duplex (referred to as a “stem”).

As used herein, “hepatic” or “liver” are used interchangeably and refer to tissue or cells within a liver of a subject. Cells of the liver include hepatocytes.

As used herein, “extra-hepatic” refers to a tissue or cell that is not the liver or is not a hepatocyte.

As used herein, “melting temperature” or “Tm” means the temperature at which the two strands of a duplex nucleic acid separate. Tₘ is often used as a measure of duplex stability or the binding affinity of two strands of complementary nucleic acids or portions thereof. Tₘ can be measured by using the UV spectrum to determine the formation and breakdown (melting) of hybridization. Base stacking, which occurs during hybridization, is accompanied by a reduction in UV absorption (hypochromicity). Consequently, a reduction in UV absorption indicates a higher Tₘ.

As used herein, “modified internucleotide linkage” refers to an internucleotide linkage having one or more chemical modifications when compared with a reference internucleotide linkage comprising a phosphodiester bond. In some embodiments, a modified nucleotide is a non-naturally occurring linkage. Typically, a modified internucleotide linkage confers one or more desirable properties to a nucleic acid in which the modified internucleotide linkage is present. For example, a modified nucleotide may improve thermal stability, resistance to degradation, nuclease resistance, solubility, bioavailability, bioactivity, reduced immunogenicity, etc.

As used herein, “modified nucleotide” refers to a nucleotide having one or more chemical modifications when compared with a corresponding reference nucleotide selected from: adenine ribonucleotide, guanine ribonucleotide, cytosine ribonucleotide, uracil ribonucleotide, adenine deoxyribonucleotide, guanine deoxyribonucleotide, cytosine deoxyribonucleotide and thymidine deoxyribonucleotide. In some embodiments, a modified nucleotide is a non-naturally occurring nucleotide. In some embodiments, a modified nucleotide has one or more chemical modification in its sugar, nucleobase and/or phosphate group. In some embodiments, a modified nucleotide has one or more chemical moieties conjugated to a corresponding reference nucleotide. Typically, a modified nucleotide confers one or more desirable properties to a nucleic acid in which the modified nucleotide is present. For example, a modified nucleotide may improve thermal stability, resistance to degradation, nuclease resistance, solubility, bioavailability, bioactivity, reduced immunogenicity, etc.

As used herein, “nicked tetraloop structure” refers to a structure of a RNAi oligonucleotide that is characterized by separate sense (passenger) and antisense (guide) strands, in which the sense strand has a region of complementarity with the antisense strand, and in which at least one of the strands, generally the sense strand, has a tetraloop configured to stabilize an adjacent stem region formed within the at least one strand.

As used herein, “oligonucleotide” refers to a short nucleic acid (e.g., less than about 100 nucleotides in length). An oligonucleotide may be single stranded (ss) or ds. An oligonucleotide may or may not have duplex regions. As a set of non-limiting examples, an oligonucleotide may be, but is not limited to, a small interfering RNA (siRNA), microRNA (miRNA), short hairpin RNA (shRNA), dicer substrate interfering RNA (dsiRNA), antisense oligonucleotide, short siRNA or ss siRNA. In some embodiments, a double-stranded (dsRNA) is an RNAi oligonucleotide.

The terms “lipid-conjugated oligonucleotide” and “oligonucleotide-ligand conjugate” are used interchangeably and refer to an oligonucleotide comprising one or more nucleotides conjugated with one or more targeting ligands (e.g., lipid).

As used herein, “overhang” refers to terminal non-base pairing nucleotide(s) resulting from one strand or region extending beyond the terminus of a complementary strand with which the one strand or region forms a duplex. In some embodiments, an overhang comprises one or more unpaired nucleotides extending from a duplex region at the 5′ terminus or 3′ terminus of a dsRNA. In some embodiments, the overhang is a 3′ or 5′ overhang on the antisense strand or sense strand of a dsRNA.

As used herein, “phosphate analog” refers to a chemical moiety that mimics the electrostatic and/or steric properties of a phosphate group. In some embodiments, a phosphate analog is positioned at the 5′ terminal nucleotide of an oligonucleotide in place of a 5′-phosphate, which is often susceptible to enzymatic removal. In some embodiments, a 5′ phosphate analog contains a phosphatase-resistant linkage. Examples of phosphate analogs include, but are not limited to, 5′ phosphonates, such as 5′ methylene phosphonate (5′-MP) and 5′-(E)-vinylphosphonate (5′-VP). In some embodiments, an oligonucleotide has a phosphate analog at a 4′-carbon position of the sugar (referred to as a “4′-phosphate analog”) at a 5′-terminal nucleotide. An example of a 4′-phosphate analog is oxymethyl phosphonate, in which the oxygen atom of the oxymethyl group is bound to the sugar moiety (e.g., at its 4′-carbon) or analog thereof. See, e.g., U.S. Provisional Patent Application Nos. 62/383,207 (filed on 2 Sep. 2016) and 62/393,401 (filed on 12 Sep. 2016). Other modifications have been developed for the 5′ end of oligonucleotides (see, e.g., Intl. Patent Application No. WO 2011/133871; U.S. Pat. No. 8,927,513; and Prakash et al., (2015) NUCLEIC ACIDS RES. 43:2993-3011).

As used herein, “reduced expression” of a target gene refers to a decrease in the amount or level of RNA transcript (e.g., target mRNA) or protein encoded by the target gene and/or a decrease in the amount or level of activity of the gene in a cell, a population of cells, a sample, or a subject, when compared to an appropriate reference (e.g., a reference cell, population of cells, sample, or subject). For example, the act of contacting a cell with an oligonucleotide or conjugate herein (e.g., an lipid-conjugated RNAi oligonucleotide comprising an antisense strand having a nucleotide sequence that is complementary to a nucleotide sequence comprising a target mRNA) may result in a decrease in the amount or level of target mRNA, protein encoded by a target gene, and/or target gene activity (e.g., via inactivation and/or degradation of target mRNA by the RNAi pathway) when compared to a cell that is not treated with the double-stranded oligonucleotide. Similarly, and as used herein, “reducing expression” refers to an act that results in reduced expression of a target gene.

As used herein, “region of complementarity” refers to a sequence of nucleotides of a nucleic acid (e.g., a dsRNA) that is sufficiently complementary to an antiparallel sequence of nucleotides to permit hybridization between the two sequences of nucleotides under appropriate hybridization conditions (e.g., in a phosphate buffer, in a cell, etc.). In some embodiments, an oligonucleotide herein comprises a targeting sequence having a region of complementary to a mRNA target sequence.

As used herein, “ribonucleotide” refers to a nucleotide having a ribose as its pentose sugar, which contains a hydroxyl group at its 2′ position. A modified ribonucleotide is a ribonucleotide having one or more modifications or substitutions of atoms other than at the 2′ position, including modifications or substitutions in or of the ribose, phosphate group or base.

As used herein, “RNAi oligonucleotide” refers to either (a) a dsRNA having a sense strand (passenger) and antisense strand (guide), in which the antisense strand or part of the antisense strand is used by the Argonaute 2 (Ago2) endonuclease in the cleavage of a target mRNA or (b) a ss oligonucleotide having a single antisense strand, where that antisense strand (or part of that antisense strand) is used by the Ago2 endonuclease in the cleavage of a target mRNA.
As used herein, “strand” refers to a single, contiguous sequence of nucleotides linked together through internucleotide linkages (e.g., phosphodiester linkages or phosphorothioate linkages). In some embodiments, a strand has two free ends (e.g., a 5′ end and a 3′ end).

As used herein, “subject” means any mammal, including mice, rabbits, and humans. In one embodiment, the subject is a human or NHP. Moreover, “individual” or “patient” may be used interchangeably with “subject.”

As used herein, “synthetic” refers to a nucleic acid or other molecule that is artificially synthesized (e.g., using a machine (e.g., a solid-state nucleic acid synthesizer)) or that is otherwise not derived from a natural source (e.g., a cell or organism) that normally produces the molecule.

As used herein, “targeting ligand” refers to a molecule or “moiety” (e.g., a carbohydrate, amino sugar, cholesterol, polypeptide, or lipid) that selectively binds to a cognate molecule (e.g., a receptor) of a tissue or cell of interest and/or that is conjugatable to another substance for purposes of targeting the other substance to the tissue or cell of interest. For example, in some embodiments, a targeting ligand may be conjugated to an oligonucleotide for purposes of targeting the oligonucleotide to a specific tissue or cell of interest. In some embodiments, a targeting ligand selectively binds to a cell surface receptor. Accordingly, in some embodiments, a targeting ligand when conjugated to an oligonucleotide facilitates delivery of the oligonucleotide into a particular cell through selective binding to a receptor expressed on the surface of the cell and endosomal internalization by the cell of the complex comprising the oligonucleotide, targeting ligand and receptor. In some embodiments, a targeting ligand is conjugated to an oligonucleotide via a linker that is cleaved following or during cellular internalization such that the oligonucleotide is released from the targeting ligand in the cell.

As used herein, “tetraloop” refers to a loop that increases stability of an adjacent duplex formed by hybridization of flanking sequences of nucleotides. The increase in stability is detectable as an increase in melting temperature (Tₘ) of an adjacent stem duplex that is higher than the Tₘ of the adjacent stem duplex expected, on average, from a set of loops of comparable length consisting of randomly selected sequences of nucleotides. For example, a tetraloop can confer a Tₘ of at least about 50° C., at least about 55° C., at least about 56° C., at least about 58° C., at least about 60° C., at least about 65° C. or at least about 75° C. in 10 mM NaHPO₄ to a hairpin comprising a duplex of at least 2 base pairs (bp) in length. In some embodiments, a tetraloop may stabilize a bp in an adjacent stem duplex by stacking interactions. In addition, interactions among the nucleotides in a tetraloop include, but are not limited to, non-Watson-Crick base pairing, stacking interactions, hydrogen bonding and contact interactions (Cheong et al., (1990) NATURE 346:680-82; Heus and Pardi (1991) SCIENCE 253:191-94). In some embodiments, a tetraloop comprises or consists of 3 to 6 nucleotides and is typically 4 to 5 nucleotides. In some embodiments, a tetraloop comprises or consists of 3, 4, 5 or 6 nucleotides, which may or may not be modified (e.g., which may or may not be conjugated to a targeting moiety). In one embodiment, a tetraloop consists of 4 nucleotides. Any nucleotide may be used in the tetraloop and standard IUPAC-IUB symbols for such nucleotides may be used as described in Cornish-Bowden ((1985) NUCLEIC ACIDS RES. 13:3021-3030). For example, the letter “N” may be used to mean that any base may be in that position, the letter “R” may be used to show that A (adenine) or G (guanine) may be in that position, and “B” may be used to show that C (cytosine), G (guanine), or T (thymine) may be in that position. Examples of tetraloops include the UNCG family of tetraloops (e.g., UUCG), the GNRA family of tetraloops (e.g., GAAA), and the CUUG tetraloop (Woese et al., (1990) PROC. NATL. ACAD. SCI. USA 87:8467-71; Antao et al., (1991) NUCLEIC ACIDS RES. 19:5901-05). Examples of DNA tetraloops include the d (GNNA) family of tetraloops (e.g., d (GTTA), the d (GNRA)) family of tetraloops, the d (GNAB) family of tetraloops, the d (CNNG) family of tetraloops, and the d (TNCG) family of tetraloops (e.g., d (TTCG)). (See, e.g., Nakano et al., (2002) BIOCHEM. 41:4281-92; Shinji et al., (2000) NIPPON KAGAKKAI KOEN YOKOSHU 78:731). In some embodiments, the tetraloop is contained within a nicked tetraloop structure.

As used herein, “Tₘ-increasing nucleotide” refers to a nucleotide that increases the melting temperature (Tₘ) of an oligonucleotide duplex as compared to the oligonucleotide duplex without the Tₘ-increasing nucleotide. Tₘ-increasing nucleotides include, but are not limited to, bicyclic nucleotides, tricyclic nucleotides, a G-clamp, and analogues thereof, and hexitol nucleotides. Certain modified nucleotides having a modified sugar moiety, or a modified nucleobase can also be used to increase the Tₘ of an oligonucleotide duplex. As used herein, the term “Tₘ-increasing nucleotide” specifically excludes nucleotides modified at the 2′-position of the sugar moiety with 2′-OMe or 2′-F.

As used herein, “treat” or “treating” refers to the act of providing care to a subject in need thereof, for example, by administering a therapeutic agent (e.g., an oligonucleotide herein) to the subject, for purposes of improving the health and/or well-being of the subject with respect to an existing condition (e.g., a disease, disorder) or to prevent or decrease the likelihood of the occurrence of a condition. In some embodiments, treatment involves reducing the frequency or severity of at least one sign, symptom or contributing factor of a condition (e.g., disease, disorder) experienced by a subject.

### EXAMPLES

### Example 1: Preparation of Double-Stranded RNAi Oligonucleotides

### Oligonucleotide Synthesis and Purification

The oligonucleotides (RNAi oligonucleotides) described in the Examples were chemically synthesized using methods described herein. Generally, RNAi oligonucleotides were synthesized using solid phase oligonucleotide synthesis methods as described for 19-36 mer RNAi oligonucleotides (see, e.g., Scaringe et al. (1990) NUCLEIC ACIDS RES. 18:5433-41 and Usman et al. (1987) J. AM. CHEM. SOC. 109:7845-45; see also, U.S. Pat. Nos. 5,804,683; 5,831,071; 5,998,203; 6,008,400; 6,111,086; 6,117,657; 6,353,098; 6,362,323; 6,437,117 and 6,469,158) in addition to using known phosphoramidite synthesis methodologies (see, e.g., Hughes and Ellington (2017) COLD SPRING HARB PERSPECT BIOL. 9 (1): a023812; Beaucage S.L., Caruthers M.H. STUDIES ON NUCLEOTIDE CHEMISTRY V: Deoxynucleoside Phosphoramidites A New Class of Key Intermediates for Deoxypolynucleotide Synthesis, TETRAHEDRON LETT. 1981; 22:1859-62. doi: 10.1016/S0040-4039 (01) 90461-7); PCT application No. PCT/US2021/42469 (each of which is incorporated herein by this reference)).

Individual RNA strands were synthesized and HPLC purified according to standard methods For example, RNA oligonucleotides were synthesized using solid phase phosphoramidite chemistry, deprotected and using the Amicon® Ultra-15 Centrifugal Filter 3K (MilliporeSigma) with UltraPure™ DNase/RNase-Free Distilled Water (Thermo Scientific) using standard techniques (Damha & Olgivie (1993) METHODS MOL. BIOL. 20:81-114; Wincott et al. (1995) NUCLEIC ACIDS RES. 23:2677-84) and the phosphoramidite synthesis as shown below:

### Synthesis of 2-(2-((((6aR,8R,9R,9aR)-8-(6-benzamido-9H-purin-9-yl)-2,2,4,4-tetraisopropyltetrahydro-6H-furo[3,2-f][1,3,5,2,4] trioxadisilocin-9-yl)oxy)methoxy)ethoxy) ethan-1-ammonium formate (1-6)

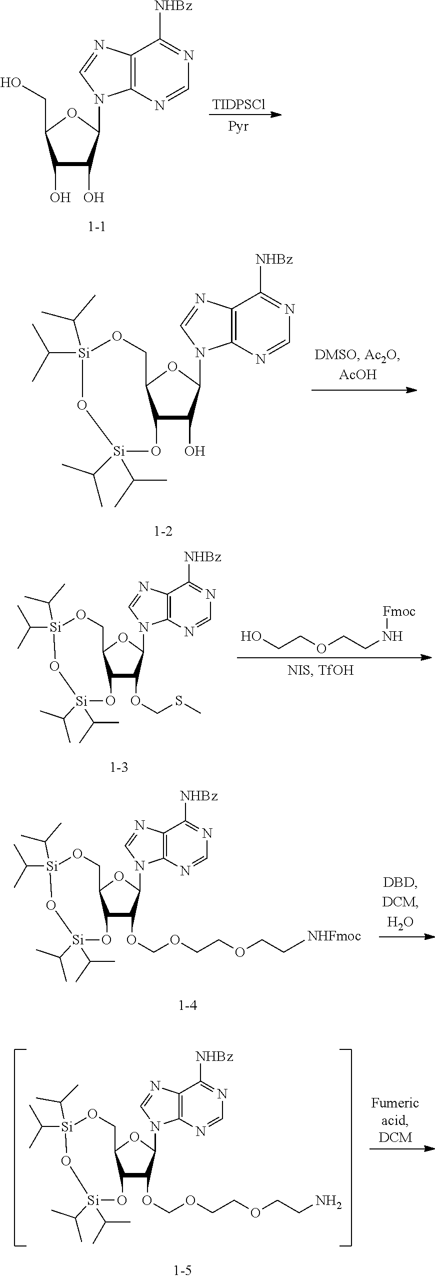

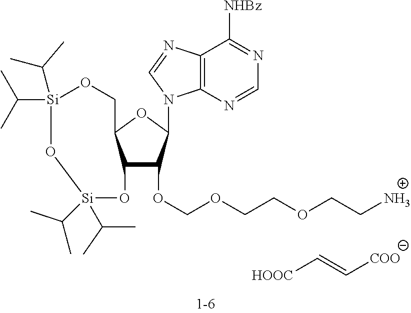

A solution of compound 1-1 (25.00 g, 67.38 mmol) in 20 mL of DMF was treated with pyridine (11 mL, 134.67 mmol) and tetraisopropyldisiloxane dichloride (22.63 mL, 70.75 mmol) at 10° C. The resulting mixture was stirred at 25° C. for 3 h and quenched with 20% citric acid (50 mL). The aqueous layer was extracted with EtOAc (3×50 mL) and the combined organic layers were concentrated in vacuo. The crude residue was recrystallized from a mixture of MTBE and n-heptane (1:15, 320 mL) to afford compound 1-2 (37.20 g, 90%) as a white oily solid.

A solution of compound 1-2 (37.00 g, 60.33 mmol) in 20 mL of DMSO was treated with AcOH (20 mL, 317.20 mmol) and Ac20 (15 mL, 156.68 mmol). The mixture was stirred at 25° C. for 15 h. The reaction was diluted with EtOAc (100 mL) and quenched with sat. K2CO₃ (50 mL). The aqueous layer was extracted with EtOAc (3×50 mL). The combined organic layers were concentrated and recrystallized with ACN (30 mL) to afford compound 1-3 (15.65 g, 38.4%) as a white solid.

A solution of compound 1-3 (20.00 g, 29.72 mmol) in 120 mL of DCM was treated with Fmoc-amino-ethoxy ethanol (11.67 g, 35.66 mmol) at 25° C. The mixture was stirred to afford a clear solution and then treated with 4 Å molecular sieves (20.0 g), N-iodosuccinimide (8.02 g, 35.66 mmol), and TfOH (5.25 mL, 59.44 mmol). The mixture was stirred at 30° C. until the HPLC analysis indicated >95% consumption of compound 1-3. The reaction was quenched with TEA (6 mL) and filtered. The filtrate was diluted with EtOAc, washed with sat. NaHCO₃ (2×100 mL), sat. Na₂SO₃ (2×100 mL), and water (2×100 mL) and concentrated in vacuo to afford crude compound 1-4 (26.34 g, 93.9%) as a yellow solid, which was used directly for the next step without further purification.

A solution of compound 1-4 (26.34 g, 27.62 mmol) in a mixture of DCM/water (10:7, 170 mL) was treated with DBU (7.00 mL, 45.08 mmol) at 5° C. The mixture was stirred at 5-25° C. for 1 h. The organic layer was then separated, washed with water (100 mL), and diluted with DCM (130 mL). The solution was treated with fumaric acid (7.05 g, 60.76 mmol) and 4 Å molecular sieves (26.34 g) in four portions. The mixture was stirred for 1 h, concentrated, and recrystallized from a mixture of MTBE and DCM (5:1) to afford compound 1-6 (14.74 g, 62.9%) as a white solid: ¹H NMR (400 MHZ, d₆-DMSO) 8.73 (s, 1H), 8.58 (s, 1H), 8.15-8.02 (m, 2H), 7.65-7.60 (m, 1H), 7.59-7.51 (m, 2H), 6.52 (s, 2H), 6.15 (s, 1H), 5.08-4.90 (m, 3H), 4.83-4.78 (m, 1H), 4.15-3.90 (m, 3H), 3.79-3.65 (m, 2H), 2.98-2.85 (m, 6H), 1.20-0.95 (m, 28H).

### Synthesis of (2R, 3R, 4R,5R)-5-(6-benzamido-9H-purin-9-yl)-2-((bis(4-methoxyphenyl)(phenyl)methoxy)methyl)-4-((2-(2-[lipid]-amidoethoxy)ethoxy)methoxy) tetrahydrofuran-3-yl (2-cyanoethyl) diisopropylphosphoramidite (2-4a to 2-4e)

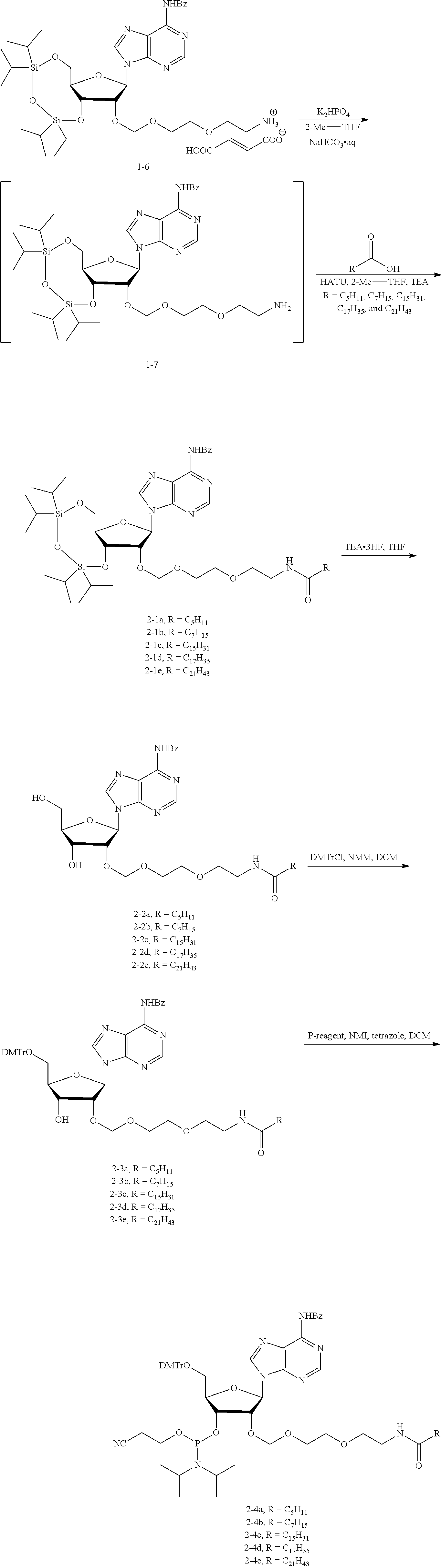

A solution of compound 1-6 (50.00 g, 59.01 mmol) in 150 mL of 2-methyltetrahydrofuran was washed with ice cold aqueous K₂HPO₄ (6%, 100 mL) and brine (20%, 2λ100 mL). The organic layer was separated and treated with hexanoic acid (10.33 mL, 82.61 mmol), HATU (33.66 g, 88.52 mmol), and DMAP (10.81 g, 147.52 mmol) at 0° C. The resulting mixture was warmed to 25° C. and stirred for 1 h. The solution was washed with water (2×100 mL), brine (100 mL), and concentrated in vacuo to afford a crude residue. Flash chromatography on silica gel (1:1 hexanes/acetone) gave compound 2-1a (34.95 g, 71.5%) as a white solid.

A mixture of compound 2-1a (34.95 g, 42.19 mmol) and TEA (9.28 mL, 126.58 mmol) in 80 mL of THF was treated with triethylamine trihydrofluoride (20.61 mL, 126.58 mmol) dropwise at 10° C. The mixture was warmed to 25° C. and stirred for 2 h. The reaction was concentrated, dissolved in DCM (100 mL), and washed with sat. NaHCO₃ (5×20 mL) and brine (50 mL). The organic layer was concentrated in vacuo to afford crude compound 2-2a (24.72 g, 99%), which was used directly for the next step without further purification.

A solution of compound 2-2a (24.72 g, 42.18 mmol) in 50 mL of DCM was treated with N-methylmorpholine (18.54 mL, 168.67 mmol) and DMTr-C₁ (15.69 g, 46.38 mmol). The mixture was stirred at 25° C. for 2 h and quenched with sat. NaHCO₃ (50 mL). The organic layer was separated, washed with water, concentrated to afford a slurry crude. Flash chromatography on silica gel (1:1 hexanes/acetone) gave compound 2-3a (30.05 g, 33.8 mmol, 79.9%) as a white solid.

A solution of compound 2-3a (25.00 g, 28.17 mmol) in 50 mL of DCM was treated with N-methylmorpholine (3.10 mL, 28.17 mmol) and tetrazole (0.67 mL, 14.09 mmol) under nitrogen atmosphere. Bis (diisopropylamino) chlorophosphine (9.02 g, 33.80 mmol) was added to the solution dropwise and the resulting mixture was stirred at 25° C. for 4 h. The reaction was quenched with water (15 mL), and the aqueous layer was extracted with DCM (3×50 mL). The combined organic layers were washed with sat. NaHCO₃ (50 mL), concentrated to afford a crude solid that was recrystallized from a mixture of DCM/MTBE/n-hexane (1:4:40) to afford compound 2-4a (25.52 g, 83.4%) as a white solid: ¹H NMR (400 MHZ, d₆-DMSO) 11.25 (s, 1H), 8.65-8.60 (m, 2H), 8.09-8.02 (m, 2H), 7.71 (s, 1H), 7.67-7.60 (m, 1H), 7.59-7.51 (m, 2H), 7.38-7.34 (m, 2H), 7.30-7.25 (m, 7H), 6.85-6.79 (m, 4H), 6.23-6.20 (m, 1H), 5.23-5.14 (m, 1H), 4. 80-4.69 (m, 3H), 4.33-4.23 (m, 2H), 3.90-3.78 (m, 1H), 3.75 (s, 6H), 3.74-3.52 (m, 3H), 3.50-3.20 (m, 6H), 3.14-3.09 (m, 2H), 3.09 (s, 1H), 2.82-2.80 (m, 1H), 2.65-2.60 (m, 1H), 2.05-1.96 (m, 2H), 1.50-1.39 (m, 2H), 1.31-1.10 (m, 14H), 1.08-1.05 (m, 2H), 0.85-0.79 (m, 3H); 31P NMR (162 MHz, d₆-DMSO) 149.43, 149.18.

Compound 2-4b, 2-4c, 2-4d, and 2-4e were prepared using similar procedures described above for compound 2-4a. Compound 2-4b was obtained (25.50 g, 85.4%) as a white solid: 1H NMR (400 MHz, d₆-DMSO) 11.23 (s, 1H), 8.65-8.60 (m, 2H), 8.05-8.02 (m, 2H), 7.73-7.70 (m, 1H), 7.67-7.60 (m, 1H), 7.59-7.51 (m, 2H), 7.38-7.34 (m, 2H), 7.30-7.25 (m, 7H), 6.89-6.80 (m, 4H), 6.21-6.15 (m, 1H), 5.23-5.17 (m, 1H), 4. 80-4.69 (m, 3H), 4.40-4.21 (m, 2H), 3.91-3.80 (m, 1H), 3.74 (s, 6H), 3.74-3.52 (m, 3H), 3.50-3.20 (m, 6H), 3.14-3.09 (m, 2H), 3.09 (s, 1H), 2.83-2.79 (m, 1H), 2.68-2.62 (m, 1H), 2.05-1.97 (m, 2H), 1.50-1.38 (m, 2H), 1.31-1.10 (m, 18H), 1.08-1.05 (m, 2H), 0.85-0.78 (m, 3H); 31P NMR (162 MHz, d₆-DMSO) 149.43, 149.19.

Compound 2-4c was obtained (36.60 g, 66.3%) as an off-white solid: ¹H NMR (400 MHz, d₆-DMSO) 11.22 (s, 1H), 8.64-8.59 (m, 2H), 8.05-8.00 (m, 2H), 7.73-7.70 (m, 1H), 7.67-7.60 (m, 1H), 7.59-7.51 (m, 2H), 7.38-7.34 (m, 2H), 7.30-7.25 (m, 7H), 6.89-6.80 (m, 4H), 6.21-6.15 (m, 1H), 5.25-5.17 (m, 1H), 4.80-4.69 (m, 3H), 4.40-4.21 (m, 2H), 3.91-3.80 (m, 1H), 3.74 (s, 6H), 3.74-3.50 (m, 3H), 3.50-3.20 (m, 6H), 3.14-3.09 (m, 2H), 3.09 (s, 1H), 2.83-2.79 (m, 1H), 2.68-2.62 (m, 1H), 2.05-1.99 (m, 2H), 1.50-1.38 (m, 2H), 1.33-1.12 (m, 38H), 1.08-1.05 (m, 2H), 0.86-0.80 (m, 3H); 31P NMR (162 MHz, d₆-DMSO) 149.42, 149.17.

Compound 2-4d was obtained (26.60 g, 72.9%) as an off-white solid: ¹H NMR (400 MHz, d₆-DMSO) 11.22 (s, 1H), 8.64-8.59 (m, 2H), 8.05-8.00 (m, 2H), 7.73-7.70 (m, 1H), 7.67-7.60 (m, 1H), 7.59-7.51 (m, 2H), 7.38-7.33 (m, 2H), 7.30-7.25 (m, 7H), 6.89-6.80 (m, 4H), 6.21-6.15 (m, 1H), 5.22-5.17 (m, 1H), 4.80-4.69 (m, 3H), 4.40-4.21 (m, 2H), 3.91-3.80 (m, 1H), 3.74 (s, 6H), 3.74-3.52 (m, 3H), 3.50-3.20 (m, 6H), 3.14-3.09 (m, 2H), 3.09 (s, 1H), 2.83-2.79 (m, 1H), 2.68-2.62 (m, 1H), 2.05-1.99 (m, 2H), 1.50-1.38 (m, 2H), 1.35-1.08 (m, 38H), 1.08-1.05 (m, 2H), 0.85-0.79 (m, 3H); 31P NMR (162 MHz, d₆-DMSO) 149.47, 149.22.

Compound 2-4e was obtained (38.10 g, 54.0%) as a white solid: ¹H NMR (400 MHZ, d₆-DMSO) 11.21 (s, 1H), 8.64-8.59 (m, 2H), 8.05-8.00 (m, 2H), 7.73-7.70 (m, 1H), 7.67-7.60 (m, 1H), 7.59-7.51 (m, 2H), 7.38-7.34 (m, 2H), 7.30-7.25 (m, 7H), 6.89-6.80 (m, 4H), 6.21-6.15 (m, 1H), 5.23-5.17 (m, 1H), 4.80-4.69 (m, 3H), 4.40-4.21 (m, 2H), 3.91-3.80 (m, 1H), 3.73 (s, 6H), 3.74-3.52 (m, 3H), 3.47-3.22 (m, 6H), 3.14-3.09 (m, 2H), 3.09 (s, 1H), 2.83-2.79 (m, 1H), 2.68-2.62 (m, 1H), 2.05-1.99 (m, 2H), 1.50-1.38 (m, 2H), 1.35-1.06 (m, 46H), 1.08-1.06 (m, 2H), 0.85-0.77 (m, 3H); 31P NMR (162 MHz, d₆-DMSO) 149.41, 149.15.

The oligomers were purified using either ion-exchange high performance liquid chromatography (IE-HPLC), or ion-pairing reversed phase high performance illiquid chromatography (IP-RP) Ion-exchange chromatography was performed on an Amersham Source 15Q column (1.0 cm×25 cm; Amersham Pharmacia Biotech) using a 15 min step-linear gradient the gradient varied from 90:10 Buffers A:B to 52:48 Buffers A:B, where Buffer A is 100 mM Tris pH 8.5 and Buffer B is 100 mM Tris pH 8.5, 1 M NaCl. IP-RP chromatography was performed on XBridge® Prep C18 5 um OBD™ 30×250 mm Column using a 30 min step-linear gradient Mobile Phase A (1% HFIP, 0.5% DIPEA, in Water), Mobile Phase B (1% HFIP, 0.5% DIPEA, in MeOH). Samples were monitored at 260 nm and peaks corresponding to the full-length oligonucleotide species were collected, pooled, desalted using the Amicon® Ultra-15 Centrifugal Filter 3K (MilliporeSigma) with UltraPure™ DNase/RNase-Free Distilled Water (Thermo Scientific), and lyophilized.

The purity of each oligomer was determined by analytical SAX chromatography. Oligoribonucleotides were obtained that were at least 90% pure as assessed by SAX for use in experiments described below. Compound identity was verified by matrix-assisted laser desorption ionization time-of-flight (MALDI-TOF) mass spectroscopy on a Waters Synapt G2-S Mass Spectrometer (Waters Corporation, Milford, MA, USA) following the manufacturer's recommended protocol. Relative molecular masses of all oligomers were obtained, often within 0.2% of expected molecular mass.

### Preparation of Duplexes

Single strand RNA oligomers were resuspended (e.g., at 100 μM concentration) in water. Complementary sense and antisense strands were mixed in equal molar amounts to yield a final solution of, for example, 50 μM duplex. Samples were heated to 95° C. for 5′ in UltraPure™ DNase/RNase-Free Distilled Water (Thermo Scientific) and were allowed to cool to room temperature before use. The RNAi oligonucleotides were stored at −20° C. Single strand RNA oligomers were stored lyophilized or in nuclease-free water at −80° C.

The synthesis methods described herein are used to generate the lipid-conjugated oligonucleotides described in the Examples below.

### Example 2: Exposed 2′-F Modified Nucleotides Affect Tissue Selectivity

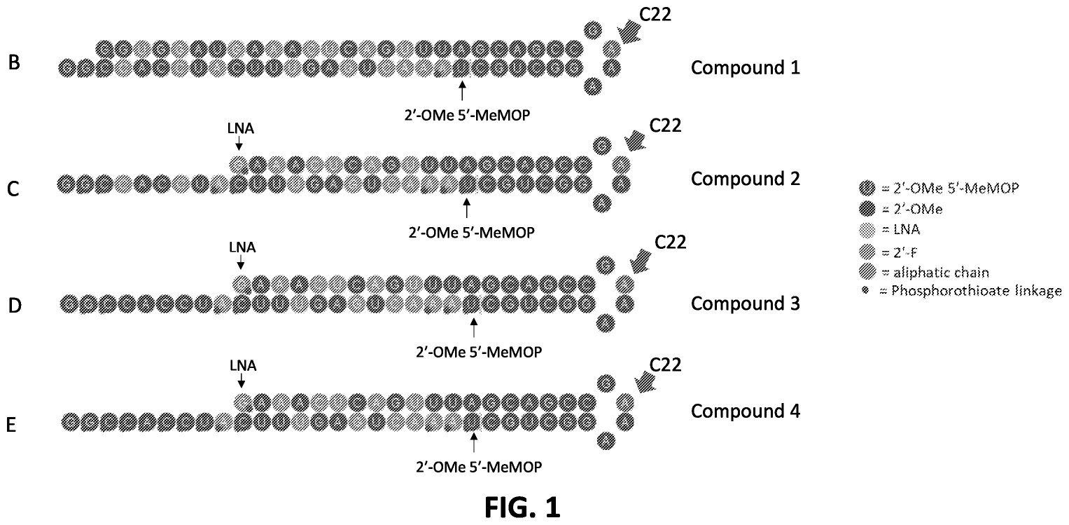

To identify an RNAi oligonucleotide-lipid conjugate capable of selectively inhibiting target mRNA in various tissues, RNAi oligonucleotides targeting ALDH2 mRNA and having a C22 acyl chain were generated by the methods described in Example 1. Aldehyde dehydrogenase 2 (ALDH2) is a ubiquitously expressed enzyme involved in oxidation. Unless otherwise indicated, all RNAi oligonucleotide-lipid conjugates evaluated herein target ALDH2. To evaluate the RNAi oligonucleotide-lipid conjugates, CD-1 female mice were given a single subcutaneous injection of 15 mg/kg RNAi oligonucleotide-lipid conjugate formulated in PBS. The RNAi oligonucleotide-lipid conjugates administered for each of the treatment groups (n=4) are outlined in Table 1 and depicted in FIG. 1. Group A were control mice that received PBS only.

Comparison was made to a parent oligonucleotide-lipid conjugate (Compound 1) which contains a stem-loop at the 3′terminus of the sense strand and a 2-nt overhang at the 3′terminus of the antisense strand. The antisense strand comprises a 2′-F modified nucleotide at least at positions G16 and G19 (G-Guide strand followed by #=position on the guide strand when numbered 5′-3′). The stem-loop contains a tetraloop having the nucleotide sequence 5′-GAAA-3′, a stem of 6-nt in length, and a C22 lipid conjugated at the second nucleotide of the tetraloop. RNAi oligonucleotide-lipid conjugates that were evaluated in comparison to Compound 1 had structures according to Compounds 2-4 as shown in FIG. 1. These structures contain the structural features described in Table 1.

TABLE 1
Lipid-conjugated RNAi oligonucleotides

| | | Description | Sense SEQ ID | | Antisense SEQ ID | |
|---|---|---|---|---|---|---|
| Group | Compound | (difference from | NOs | | NOs | |
| # | ID | parent Compound 1) | Unmod | Mod | Unmod | Mod |
| A | PBS | | — | — | — | — |
| B | 1 | Parent | 1 | 20 | 16 | 39 |
| C | 2 | 9-nt overhang on | 2 | 21 | 16 | 40 |
| | | 3′terminus of antisense | | | | |
| | | strand; PS linkage in | | | | |
| | | antisense between | | | | |
| | | positions 13 and 14, and | | | | |
| | | 14 and 15 | | | | |
| D | 3 | 9-nt overhang on | 2 | 21 | 16 | 41 |
| | | 3′terminus of antisense | | | | |
| | | strand; G16 and G19 | | | | |
| | | comprise 2′-OMe | | | | |
| | | modification; PS linkage | | | | |
| | | in antisense between | | | | |
| | | positions 13 and 14, and | | | | |
| | | 14 and 15 | | | | |
| E | 4 | 9-nt overhang on | 2 | 21 | 16 | 42 |
| | | 3′terminus of antisense | | | | |
| | | strand; G16 and G19 | | | | |
| | | comprise 2′-OMe | | | | |
| | | modification; PS | | | | |
| | | linkages in antisense for | | | | |
| | | all nucleotides in 9-nt | | | | |
| | | overhang | | | | |

*PS = phosphorthioate; nt = nucleotide

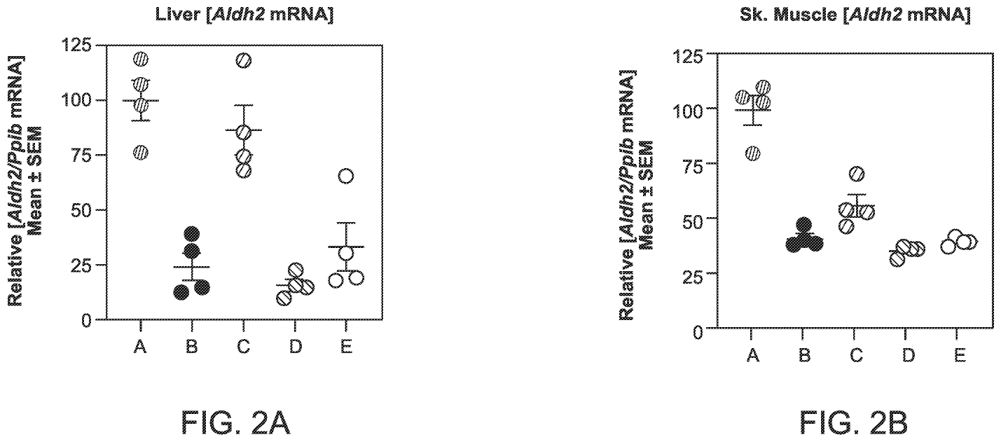

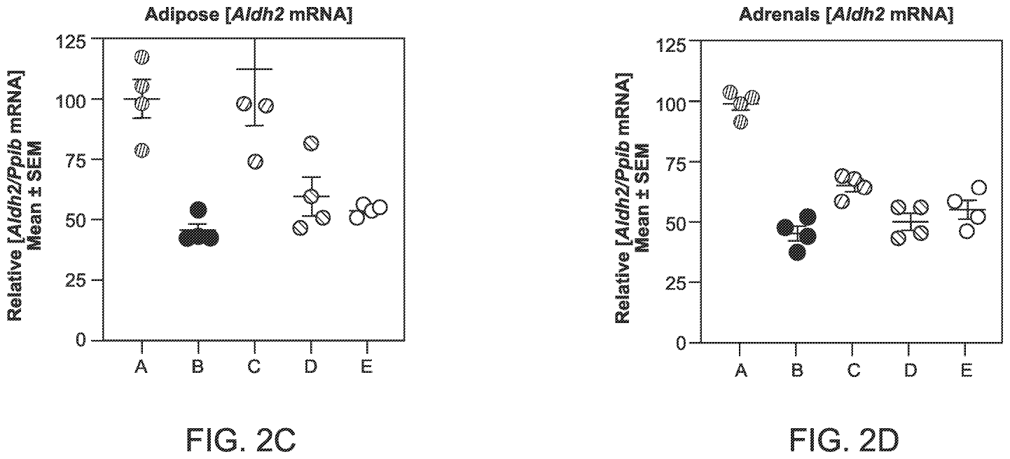

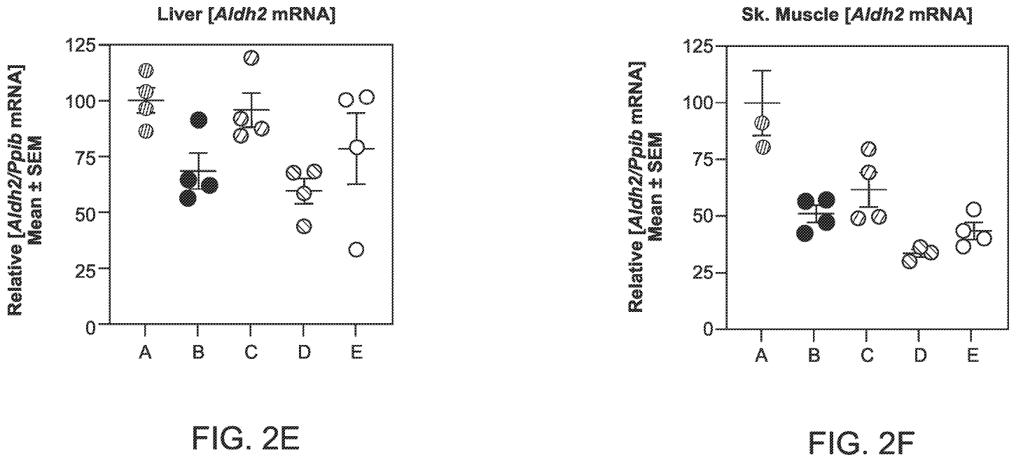

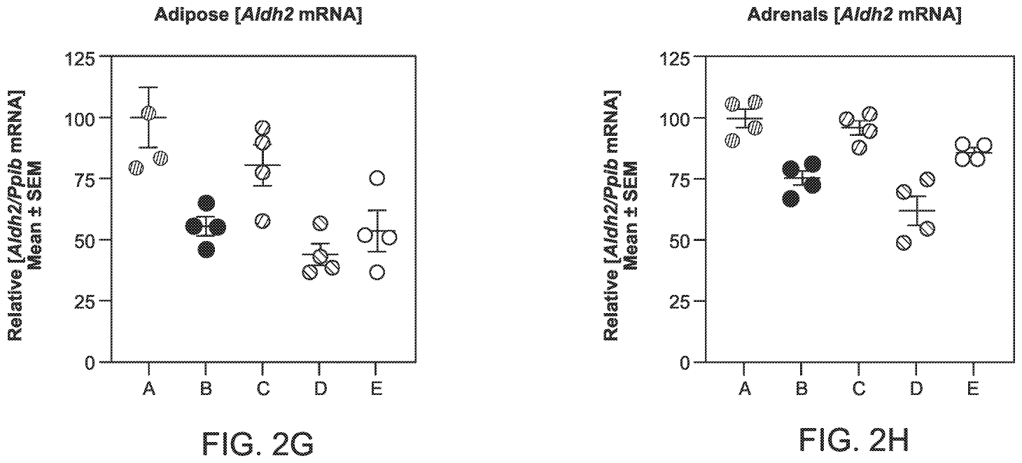

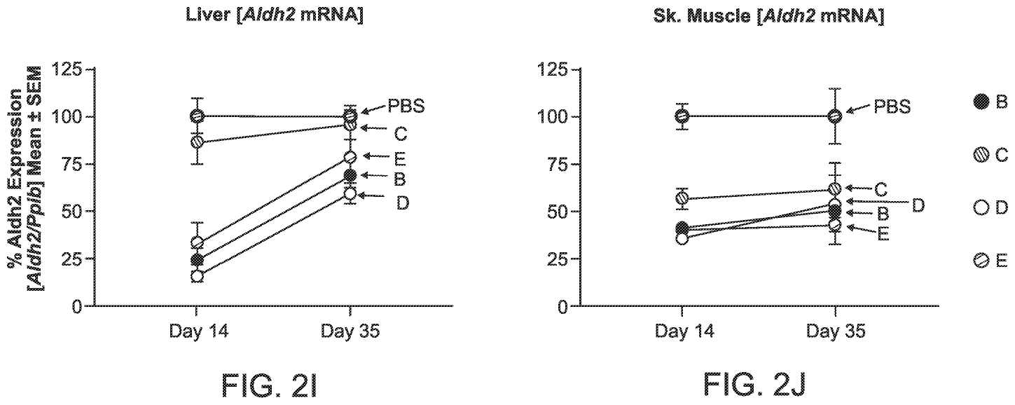

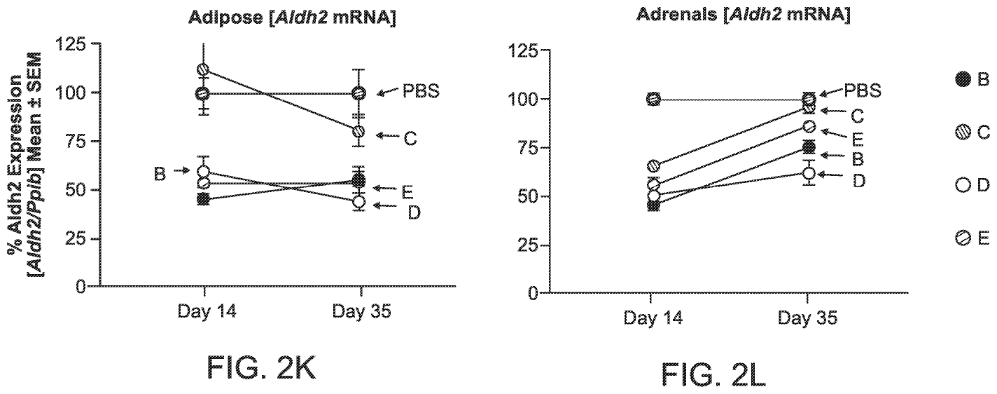

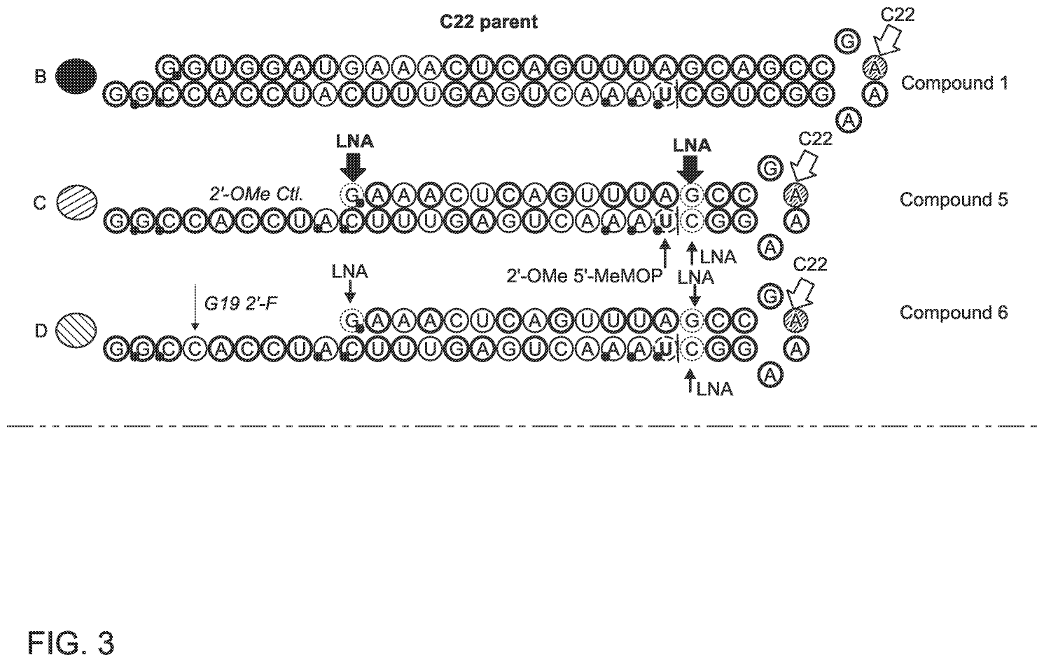

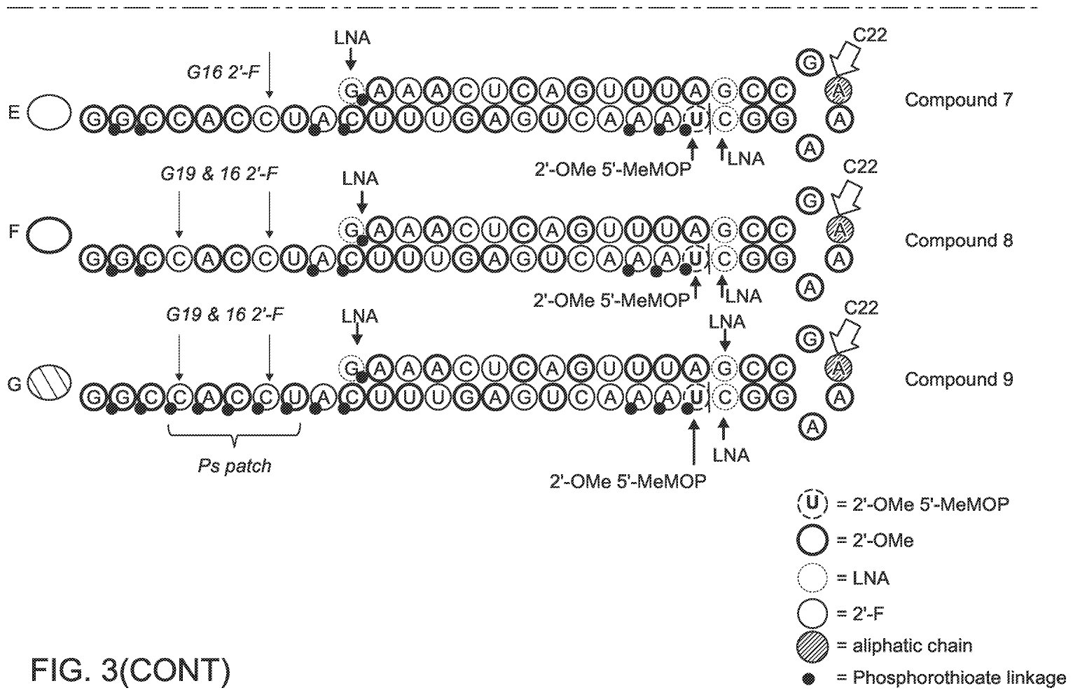

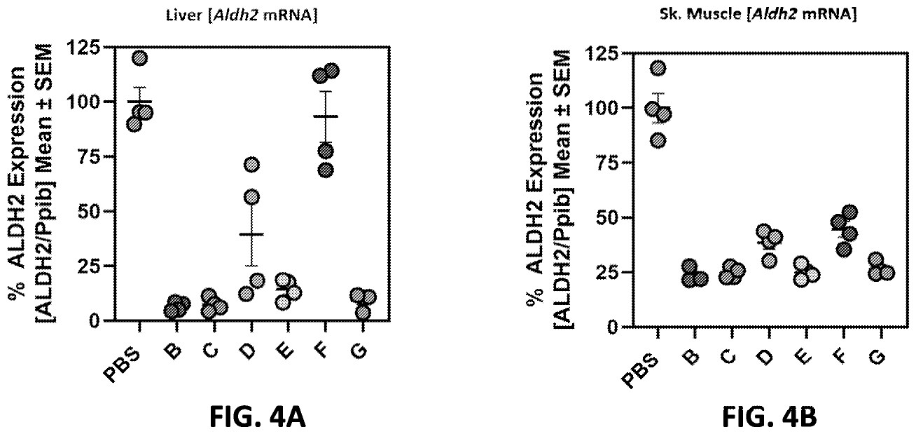

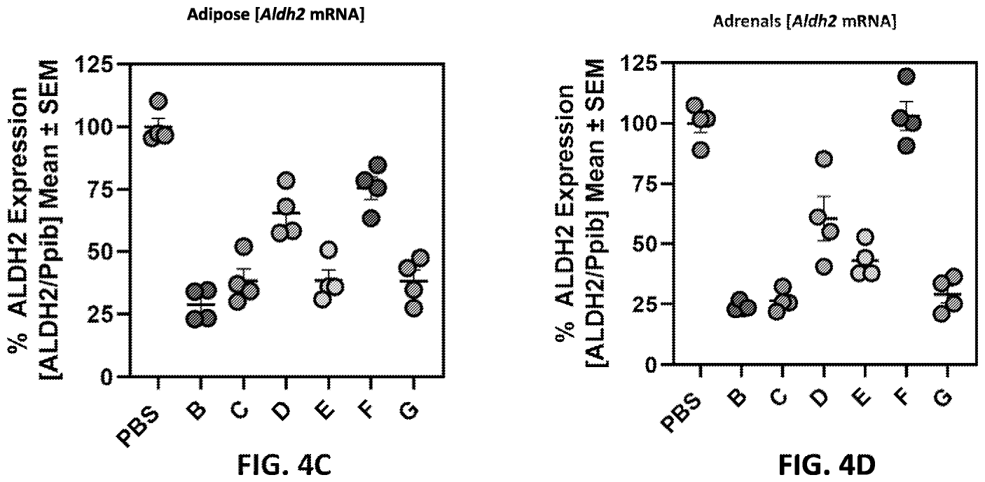

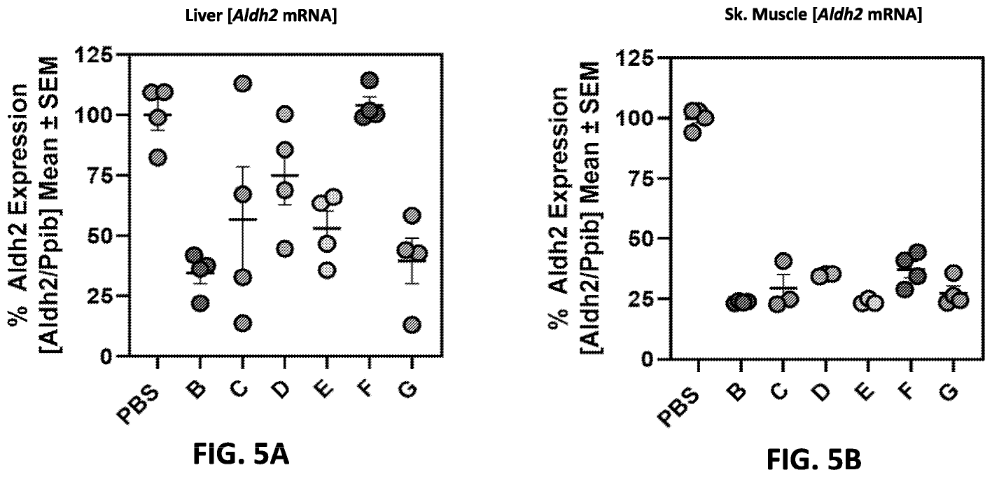

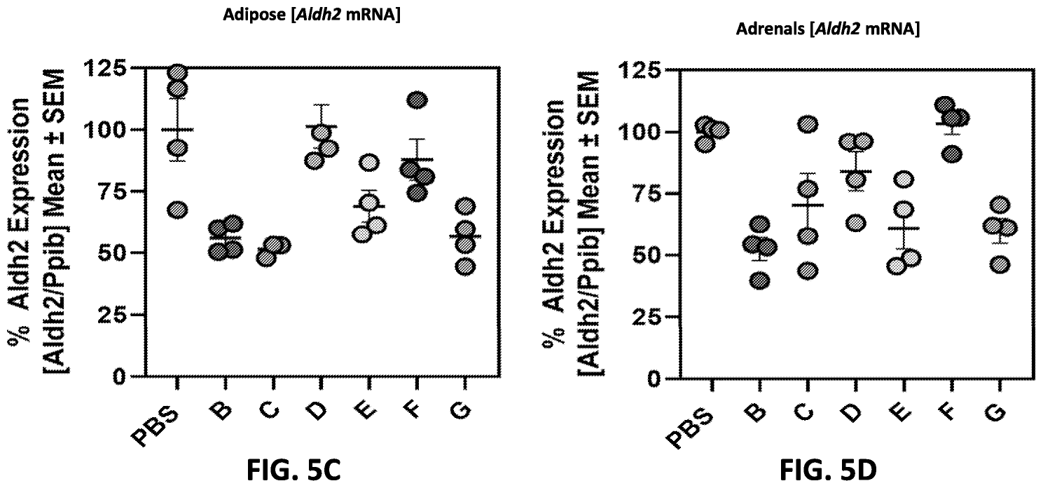

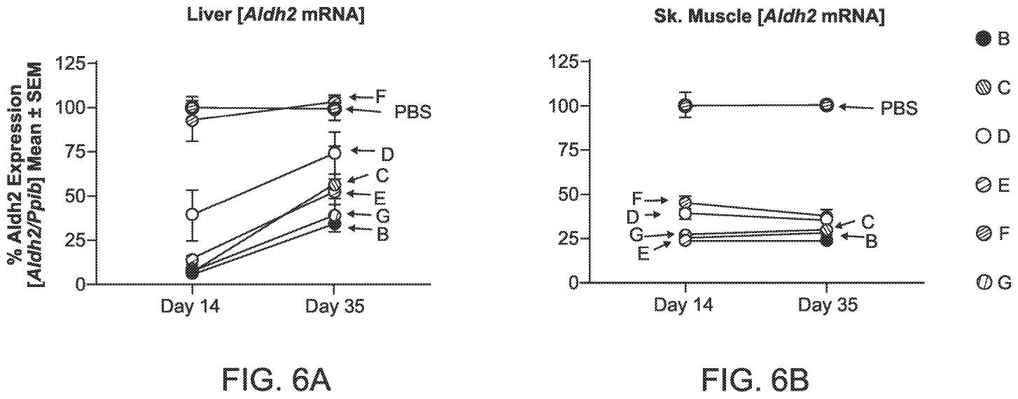

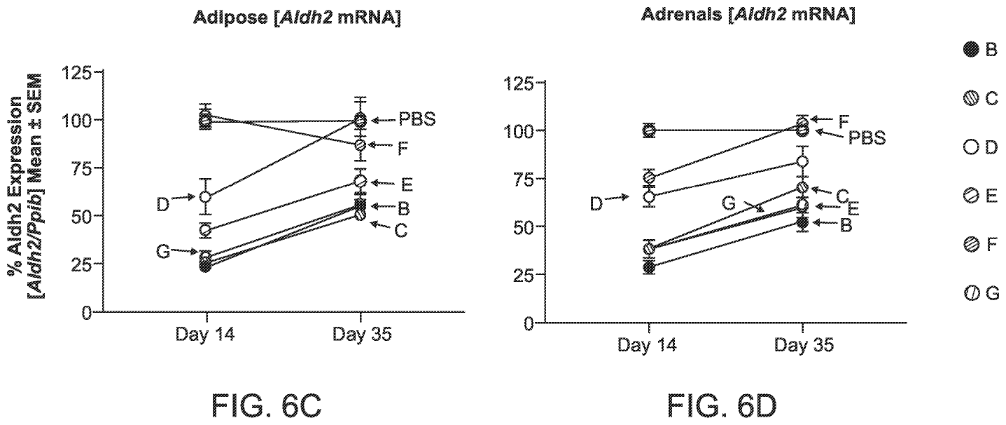

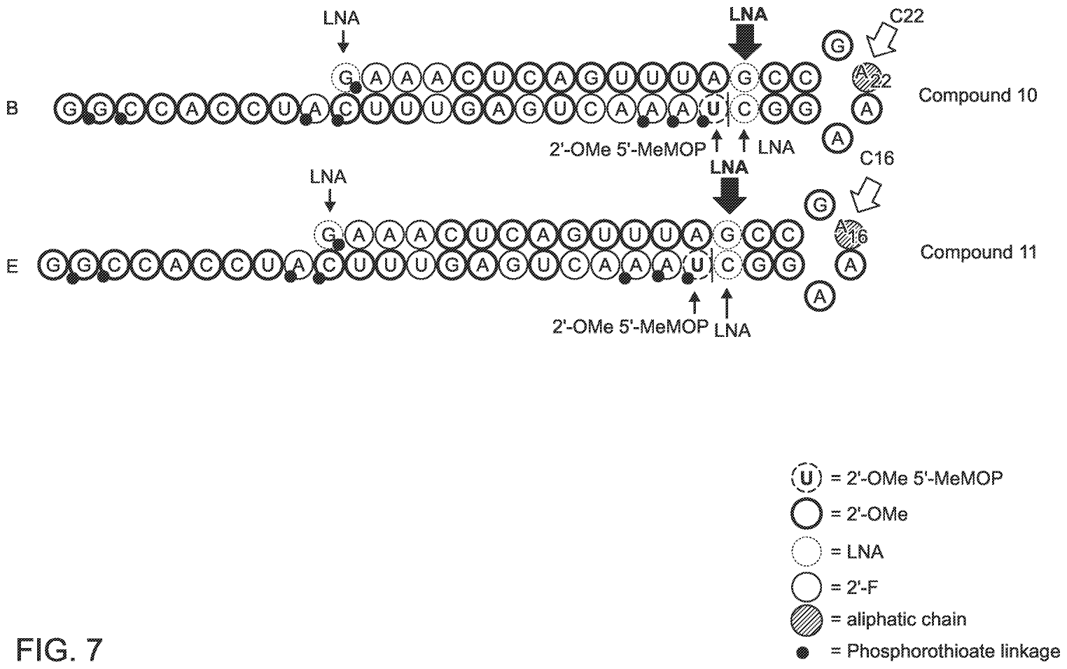

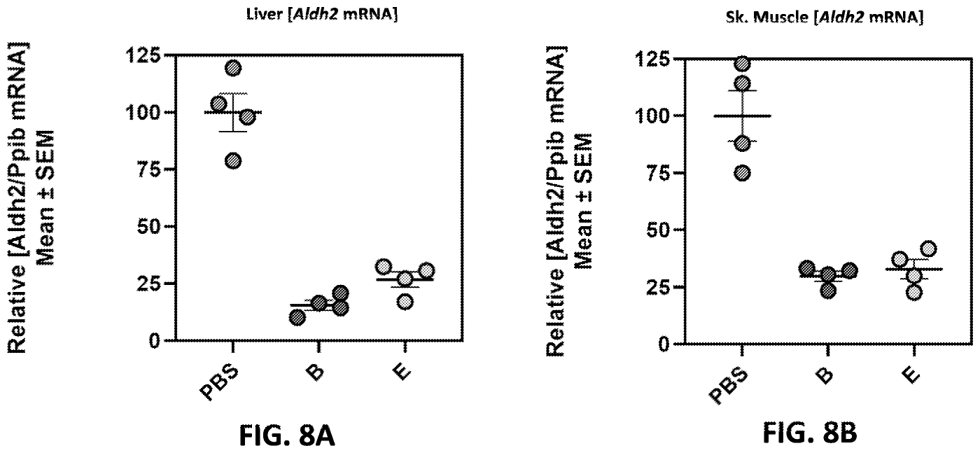

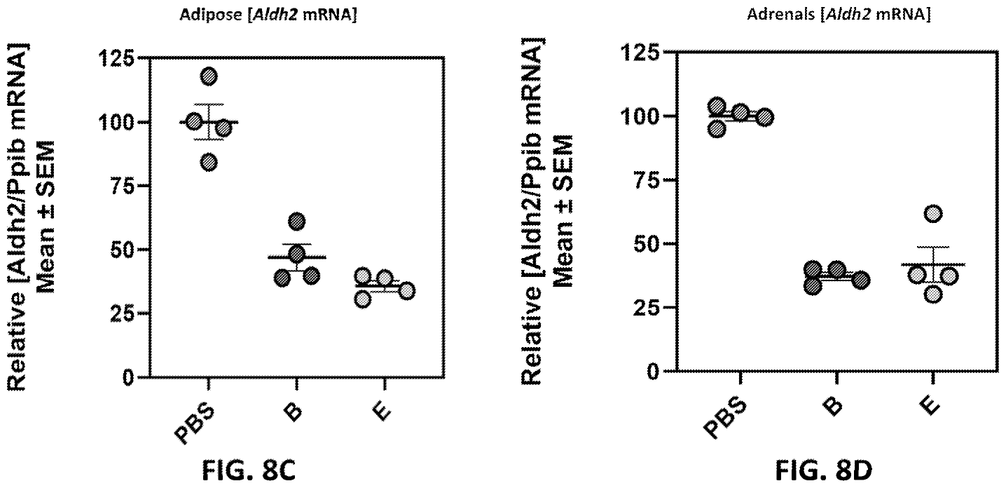

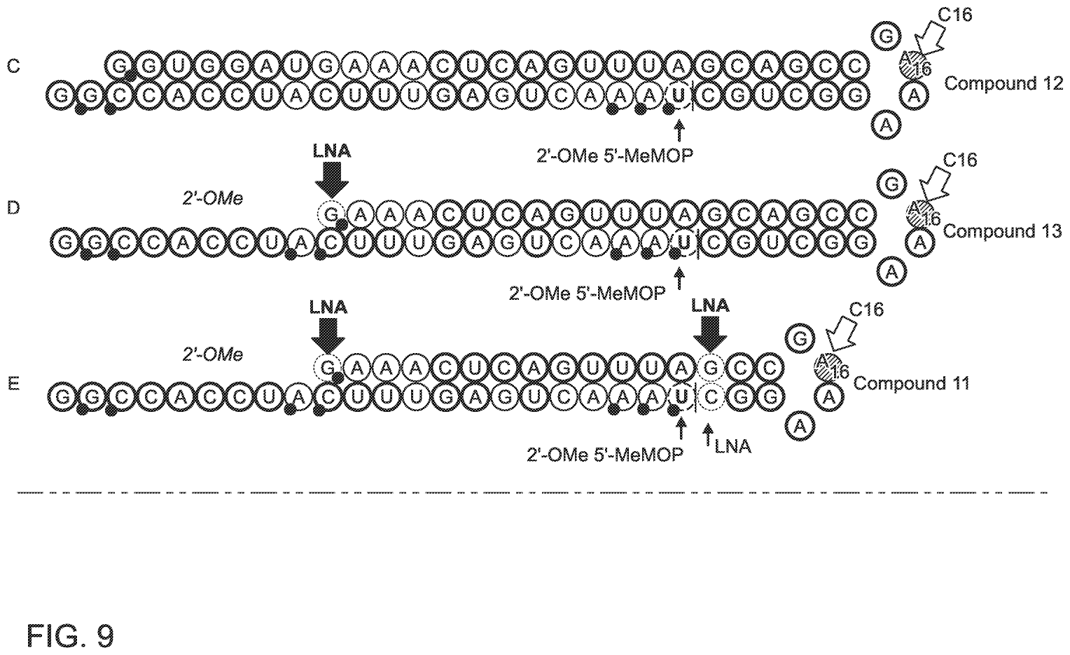

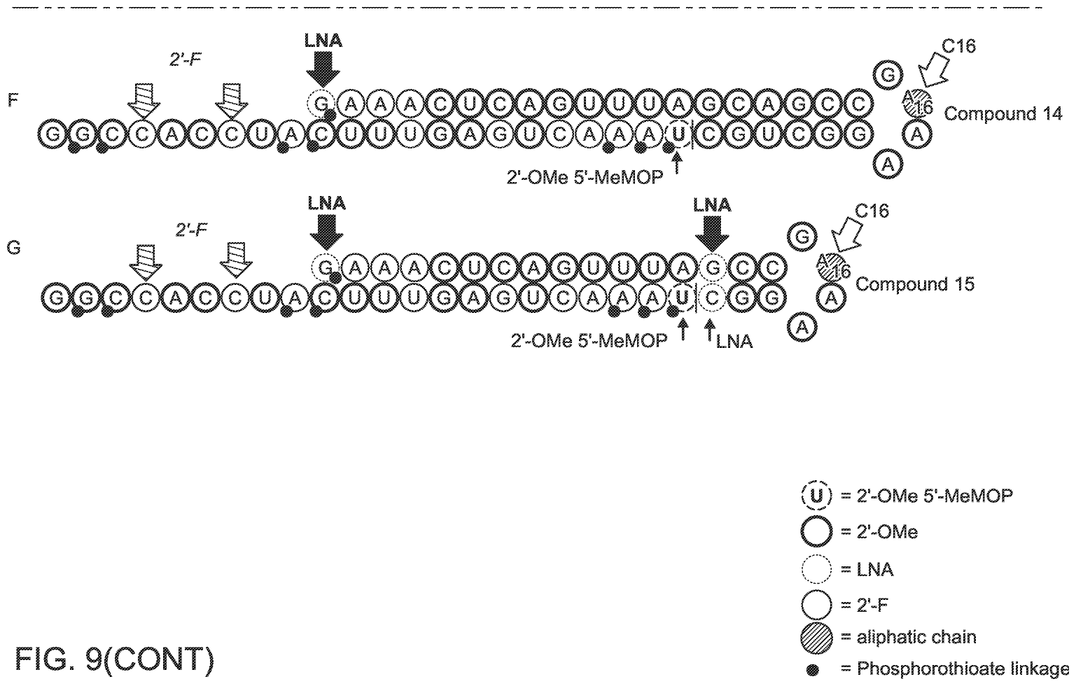

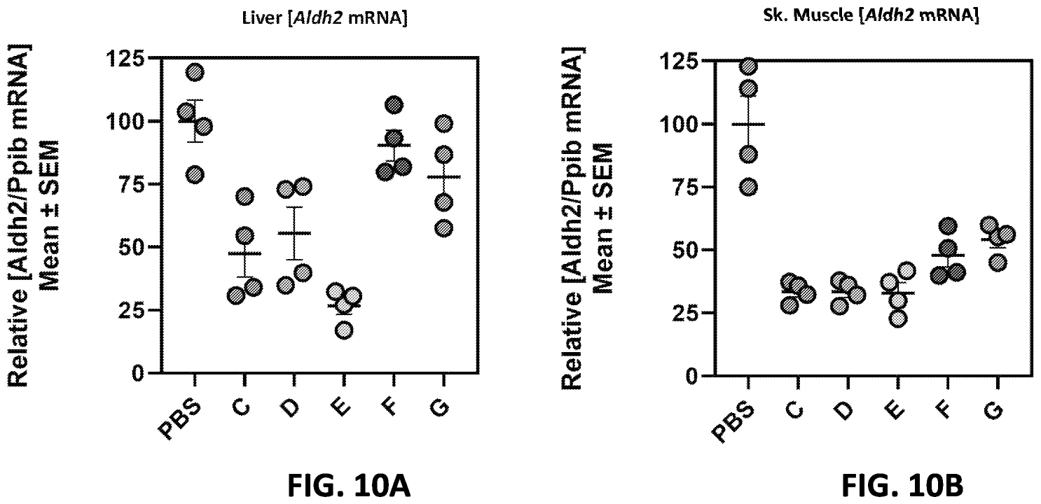

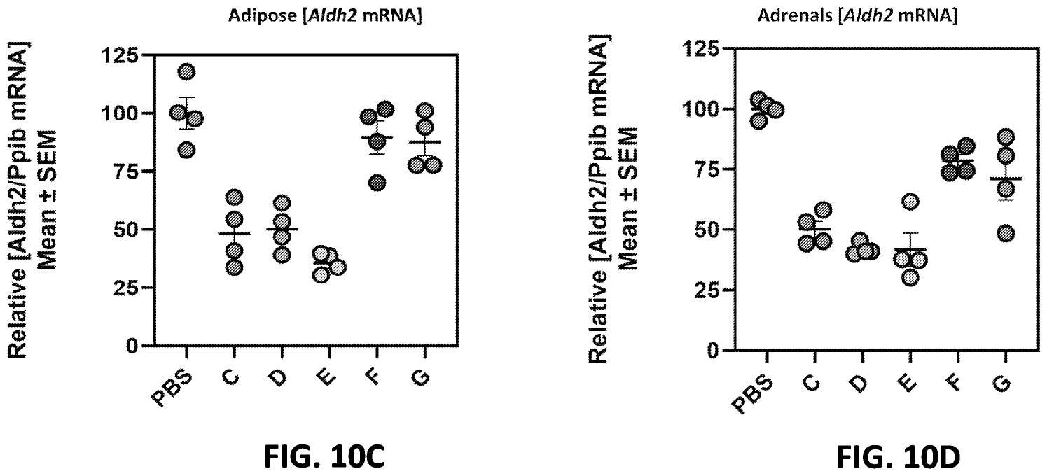

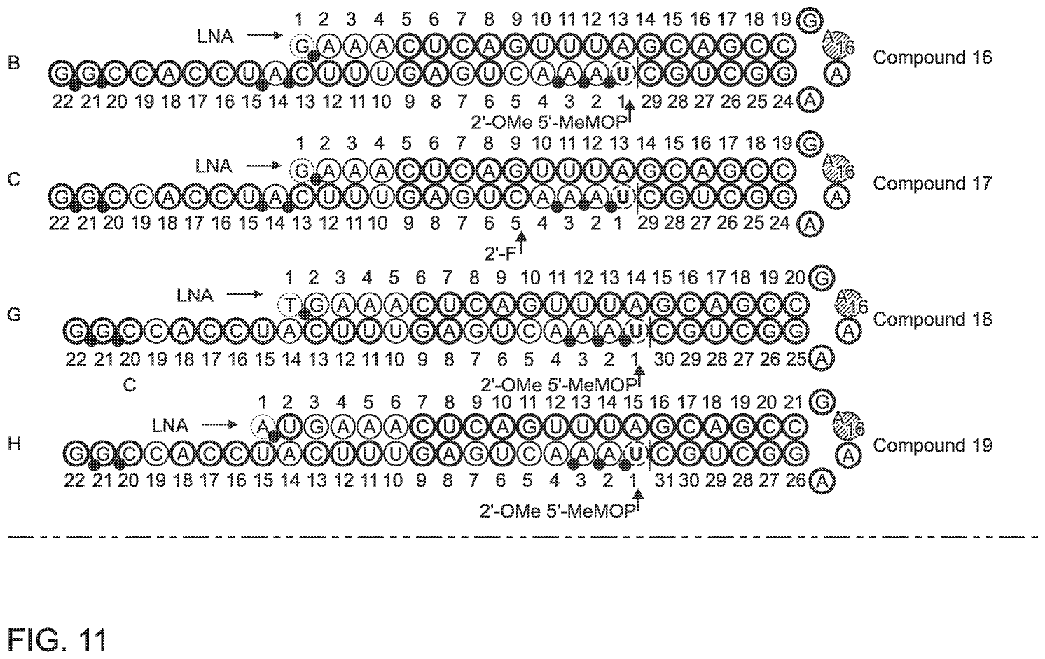

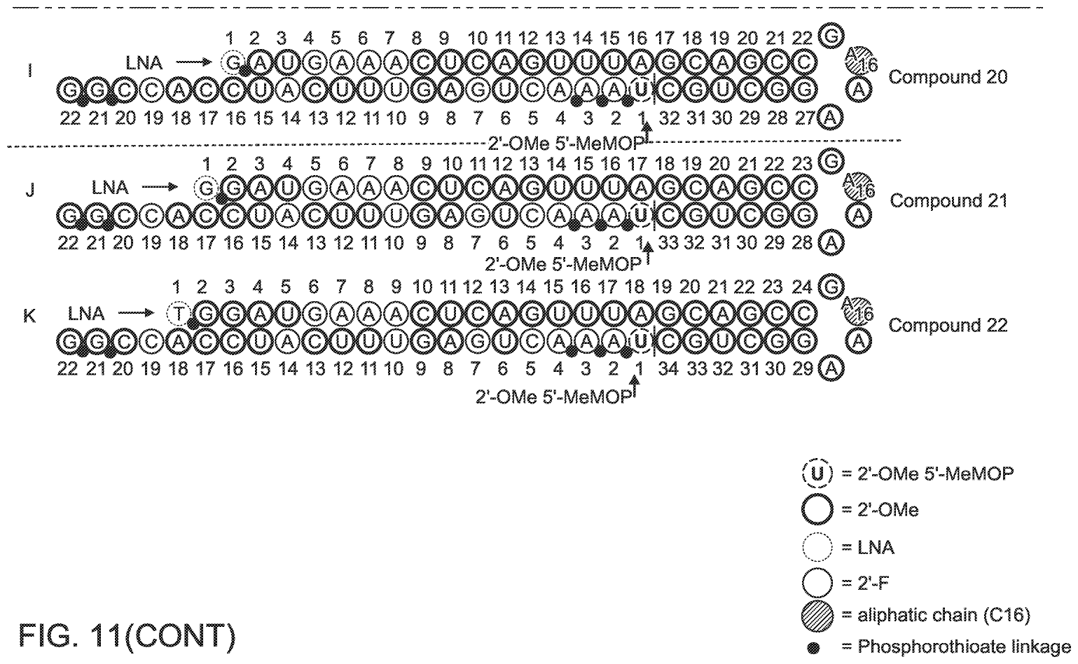

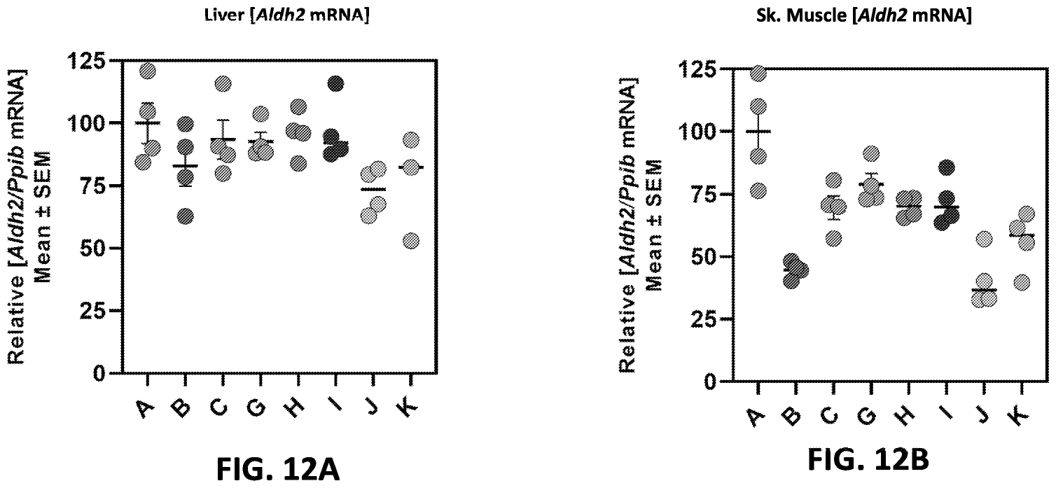

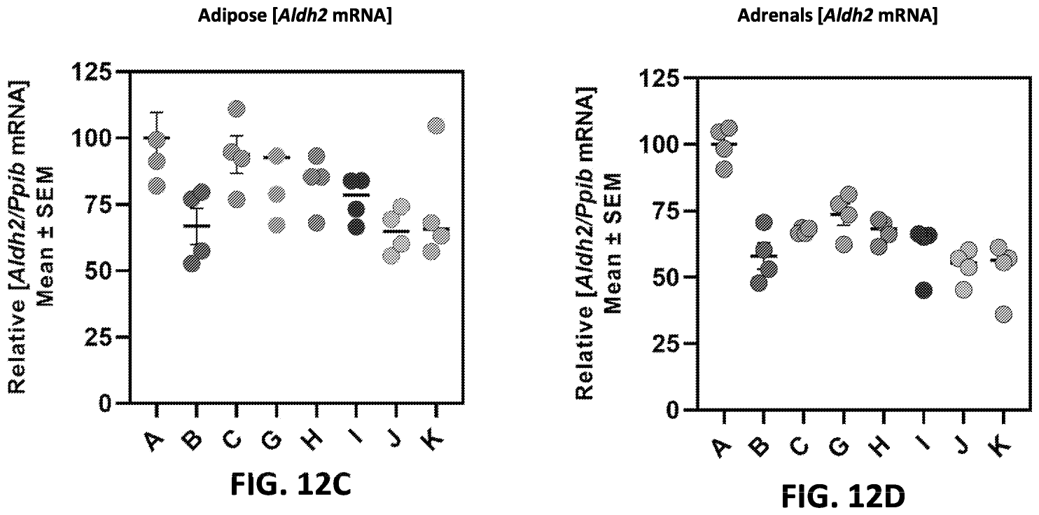

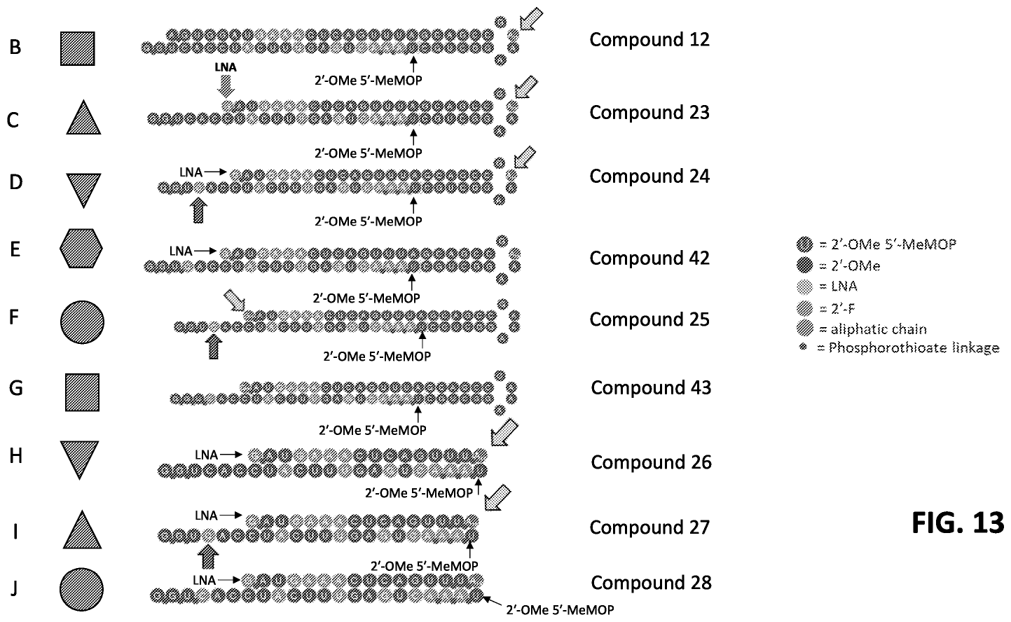

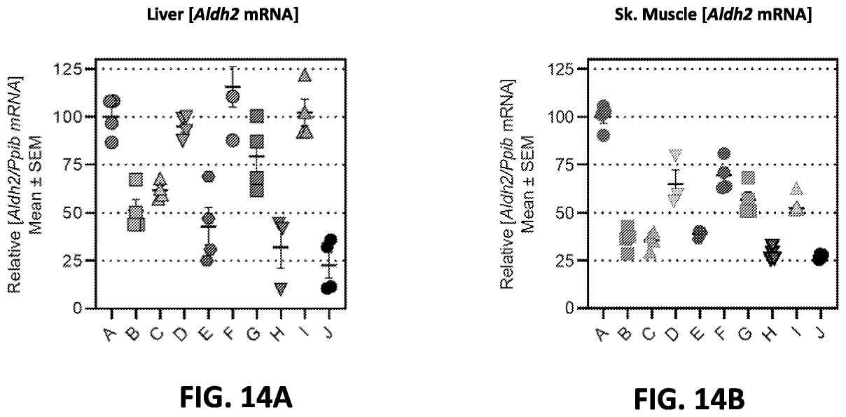

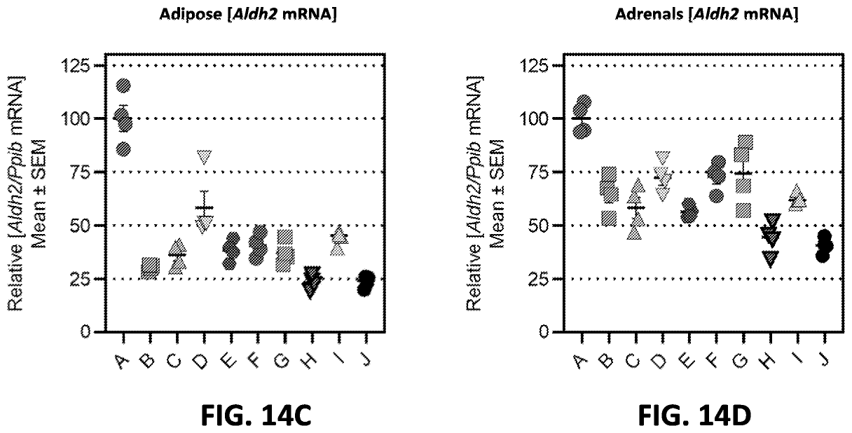

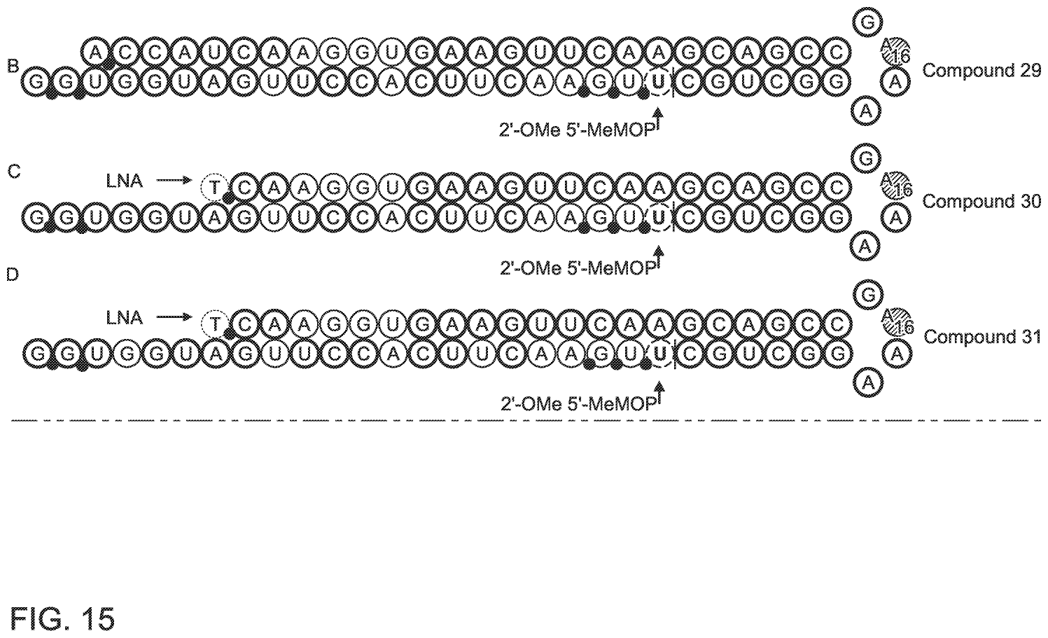

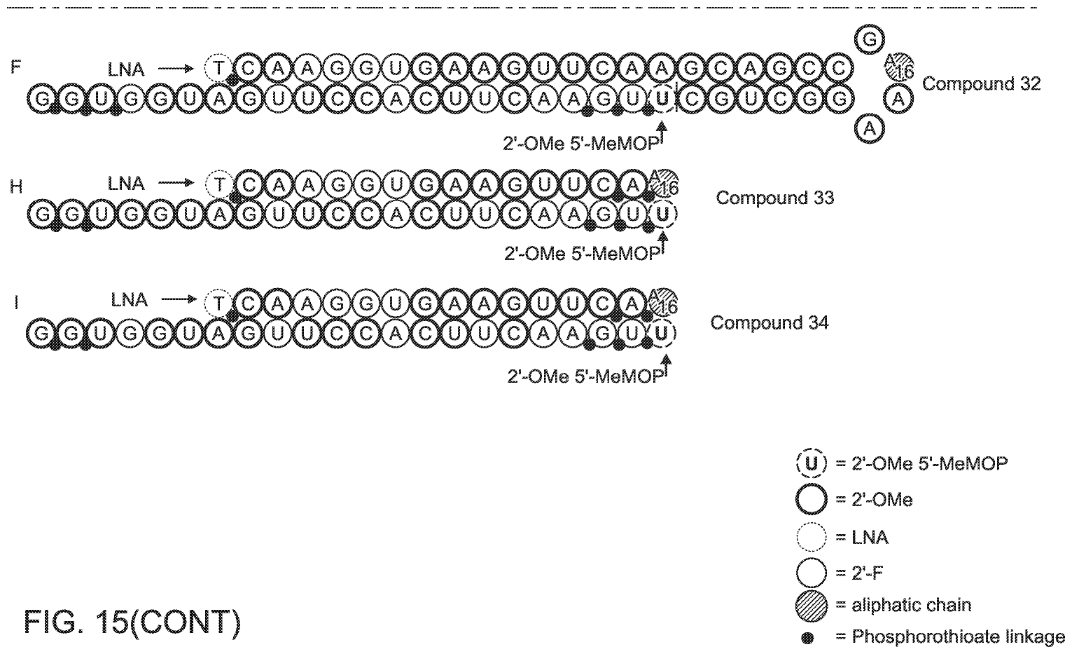

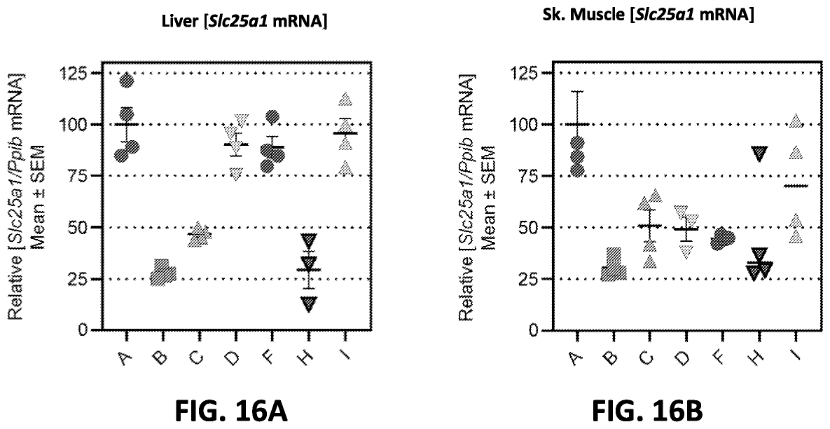

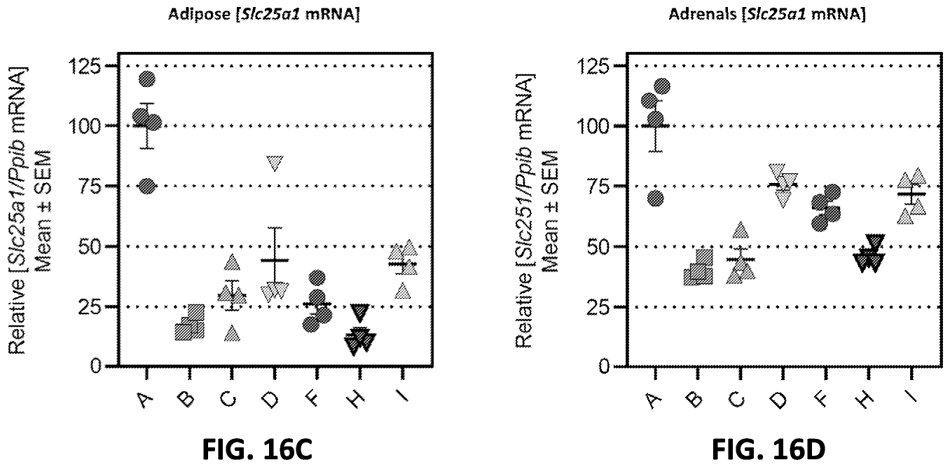

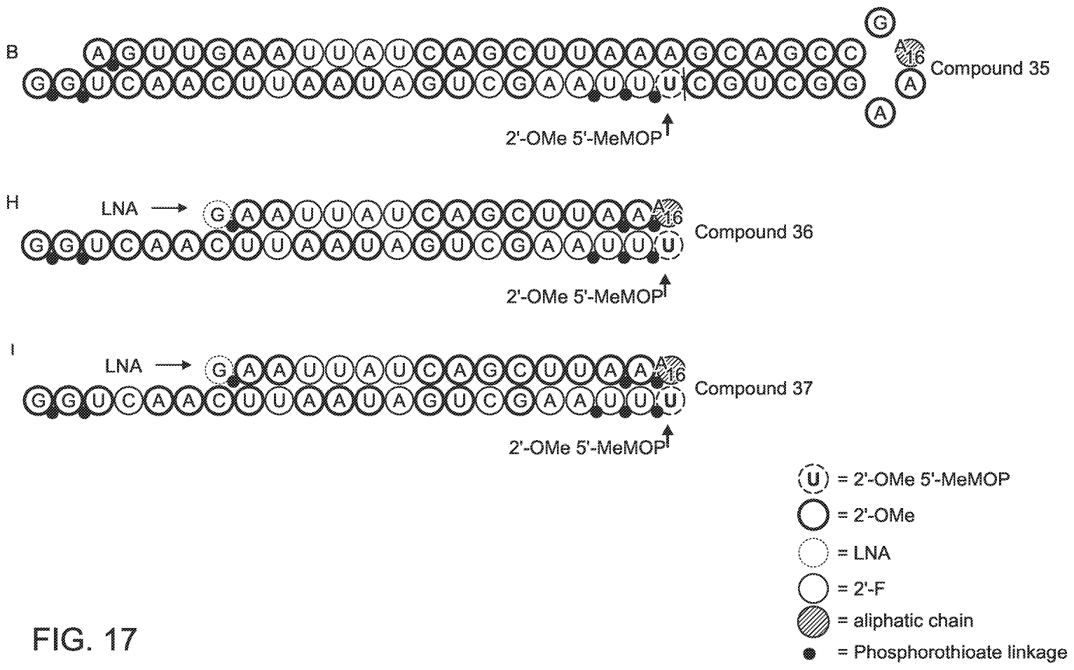

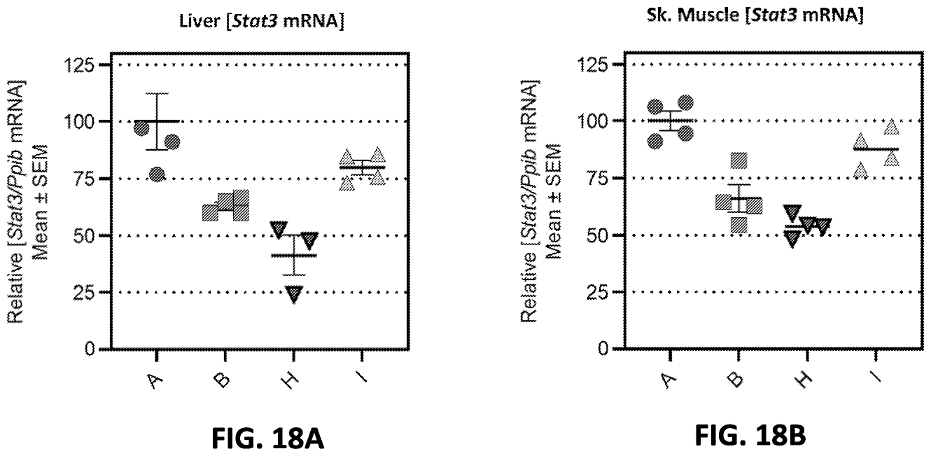

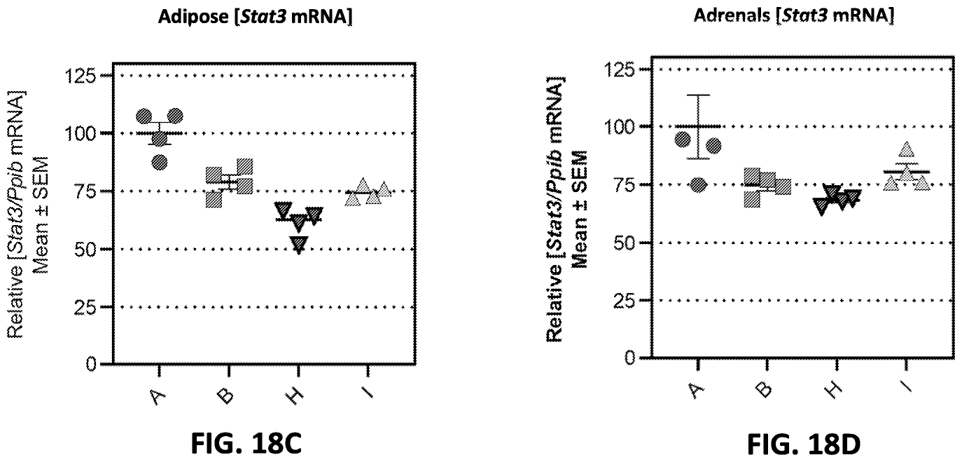

Target knockdown was assessed 14, 28 and 35 days post-injection. RNA was extracted from liver, skeletal muscle, adipose, and adrenal tissue and murine ALDH2 mRNA levels were determined by qPCR. All samples were normalized to the PBS treated control animals. Normalized ALDH2 mRNA expression in the liver, skeletal muscle, adipose, and adrenal tissue relative to control as measured for each treatment group is shown in FIGS. 2A-2D for day 14, respectively, and FIGS. 2E-2H for day 28, respectively. Comparison between day 14 and day 35 is shown in FIGS. 21-2L. Reduction of ALDH2 mRNA expression was observed for most treatment groups. Compounds with exposed 2′-F nucleotides (i.e., 2′-F modified nucleotides without phosphorothioate linkages) on the guide strand had inhibited reduction of the ALDH2 mRNA in the liver compared to compounds without 2′-F modification or with phosphorothioate linkages on the exposed guide strand. Specifically, Compound 2 which comprises a G16 and G19 (G-Guide strand followed by #=position on the guide strand when numbered 5′-3′) exposed 2′-F had reduced inhibition of ALDH2 in the liver compared to Compounds 3 and 4. Notably, reduction of ALDH2 mRNA was maintained in the extra-hepatic tissues, i.e., skeletal muscle, adipose and adrenals. Together, this data provides an exemplary chemical modification for avoiding reduction of gene expression in the liver while reducing gene expression in extra-hepatic tissue.

### Example 3: RNAi Comprising Position G19 Exposed 2′-F Modified Nucleotide Modulates Tissue Activity

To identify if the position of an exposed 2′-F modified nucleotide on the guide strand of an RNAi oligonucleotide-lipid conjugate impacted tissue specific activity, RNAi oligonucleotide-lipid conjugates were generated by the methods described in Example 1. To evaluate the RNAi oligonucleotide-lipid conjugates, CD-1 female mice were given a single subcutaneous injection of 15 mg/kg RNAi oligonucleotide-lipid conjugate formulated in PBS. The RNAi oligonucleotide-lipid conjugates administered for each of the treatment groups are outlined in Table 2 and depicted in FIG. 3.

Comparison was made to parent Compound 1 described above and short stem parent Compound 5. Compound 5 differs from Compound 1 in that it comprises a shortened 3-nucleotide stem-loop which comprises locked nucleic acids (LNA) at the first and last nucleotides of the stem loop. The sense strand comprises a LNA at the 5′terminal nucleotide. The antisense strand comprises a 2′-OMe modified nucleotide at least at positions G16 and G19. The compound further comprises a 9-nt overhand at the 3′terminus of the antisense strand. RNAi oligonucleotide-lipid conjugates that were evaluated in comparison to Compound 1 had structures according to Compounds 5-9 as shown in FIG. 3. These structures contain the structural features described in Table 2.

TABLE 2
Lipid-conjugated RNAi oligonucleotides

| | | Description | Sense SEQ ID | | Antisense SEQ ID | |
|---|---|---|---|---|---|---|
| Group | Compound | (difference from | NOs | | NOs | |
| # | ID | Compound 5) | Unmod | Mod | Unmod | Mod |
| PBS | — | | — | — | — | — |
| B | 1 | 2-nt overhang on 3′ terminus | 1 | 20 | 16 | 39 |
| | | of antisense strand; 6-nt | | | | |
| | | length stem loop; no LNAs | | | | |
| C | 5 | | 3 | 22 | 16 | 43 |
| D | 6 | G19 modified to 2′-F | 3 | 22 | 16 | 44 |
| E | 7 | G16 modified to 2′-F | 3 | 22 | 16 | 45 |
| F | 8 | G16 and G19 | 3 | 22 | 16 | 46 |
| | | modified to 2′-F | | | | |
| G | 9 | G16 and G19 | 3 | 22 | 16 | 47 |
| | | modified to 2′-F; all | | | | |
| | | nucleotides in antisense | | | | |
| | | overhang comprise PS | | | | |
| | | linkages | | | | |

; PS linkage between positions 13 and 14, and 14 and 15

Target knockdown was assessed 14 and 35-days post-injection. RNA was extracted from liver, skeletal muscle, adipose, and adrenals and murine ALDH2 mRNA levels were determined by RT-qPCR. All samples were normalized to the PBS treated control animals. Normalized ALDH2 mRNA expression in the liver, skeletal muscle, adipose, and adrenals relative to control as measured for each treatment group at day 14 are shown in FIGS. 4A-4D, respectively. Normalized ALDH2 mRNA expression in the liver, skeletal muscle, adipose, and adrenals relative to control as measured for each treatment group at day 35 are shown in FIGS. 5A-5D, respectively. FIGS. 6A-6D show inhibition of ALDH2 mRNA expression from day 14 to day 35. Reduction of ALDH2 mRNA expression was observed for most treatment groups. Compounds 6 and 8 which each comprise a 2′-F modified nucleotide at G19 had reduced inhibition of ALDH2 mRNA expression in the liver, adipose, and adrenal tissue but maintained inhibition of ALDH2 mRNA in skeletal muscle. Including an exposed 2′-F nucleotide at position G16 (Compound 8) enhanced reduction of inhibition. In contrast, a 2′-F nucleotide at position G16 alone (e.g., Compound 7) did not impact tissue specific inhibition of target mRNA. Compound 9, having a 2′-F nucleotide at position G16 and position G19 and phosphorothioate linkages in the exposed strand, rescued activity in all tissue, including the liver. Without being bound by theory, it is believed loss of activity with exposed antisense strands is a result of nuclease activity which is protected by phosphorothioate linkages. These data indicate a 2′-F nucleotide at position G19 is sufficient to reduce inhibition of target mRNA expression in tissue including the liver while maintaining inhibitory activity in extra-hepatic tissue such as skeletal muscle.

### Example 4: Acyl Chain and Stem Length do not Impact Tissue Specificity Activity of RNAi Oligonucleotide-Lipid Conjugates with Exposed 2′-F Nucleotide

To identify if the acyl chain length or length of the stem impacted the tissue specific activity of RNAi oligonucleotide-lipid conjugates comprising an exposed 2′-F modified nucleotide on the guide strand, RNAi oligonucleotide-lipid conjugates were generated by the methods described in Example 1. To evaluate the RNAi oligonucleotide-lipid conjugates, CD-1 female mice were given a single subcutaneous injection of 15 mg/kg oligonucleotide-lipid conjugate formulated in PBS. The RNAi oligonucleotide-lipid conjugates administered for each of the treatment groups are outlined in Tables 3 and 4 and depicted in FIGS. 7 and 9.

### Acyl Chain Length

To determine whether acyl chain length impacted activity, RNAi oligonucleotide-lipid conjugates having a C16 or C22 acyl chain were compared. Specifically, Compounds 10 and 11 as shown in FIG. 7 were evaluated. Compound 10 contains a stem-loop at the 3′terminus of the sense strand and a 9-nt overhang at the 3′terminus of the antisense strand. The stem-loop contains a tetraloop having the nucleotide sequence 5′-GAAA-3′, a stem of 3-nt in length, and a C22 lipid conjugated at the second nucleotide of the tetraloop. In contrast, Compound 11 has a C16 lipid conjugated to the second nucleotide of the tetraloop. These structures contain the structural features described in Table 3.

TABLE 3
Lipid-conjugated RNAi oligonucleotides

| | | | | | Antisense SEQ ID | |
|---|---|---|---|---|---|---|
| Group | Compound | | Sense SEQ ID NOS | | NOs | |
| # | ID | Description | Unmod | Mod | Unmod | Mod |
| PBS | — | | — | — | — | — |
| B | 10 | C22 lipid | 3 | 23 | 16 | 43 |
| E | 11 | C16 lipid | 3 | 24 | 16 | 43 |

Target knockdown was assessed 14 days post-injection. RNA was extracted from liver, skeletal muscle, adipose, and adrenals and murine ALDH2 mRNA levels were determined by qPCR. All samples were normalized to the PBS treated control animals. Normalized ALDH2 mRNA expression in the liver, skeletal muscle, adipose, and adrenals relative to control as measured for each treatment group at day 14 are shown in FIGS. 8A-8D, respectively. Compounds 10 and 11 which comprise C22 and C16 lipid chains on the stem-loop of the sense strand, respectively, showed no difference in ALDH2 mRNA inhibition.

### Stem Length

To determine whether stem length impacted activity, RNAi oligonucleotide-lipid conjugates having varying stem lengths were compared. Specifically, Compounds 11-15 as provided in FIG. 9 were evaluated. Compound 12 is identical to the parent Compound 1 in Example 2 except having a C16 lipid conjugated to the second nucleotide of the tetraloop. RNAi oligonucleotide-lipid conjugates that were evaluated in comparison to Compound 12 had structures according to Compounds 11, and 13-15. These structures contain the structural features described in Table 4.

TABLE 4
Lipid-conjugated RNAi oligonucleotides

| | | Description | Sense SEQ ID | | Antisense SEQ ID | |
|---|---|---|---|---|---|---|
| Group | Compound | (difference from | NOs | | NOs | |
| # | ID | Compound 12) | Unmod | Mod | Unmod | Mod |
| PBS | — | | — | — | — | — |
| C | 12 | — | 2 | 25 | 16 | 44 |
| D | 13 | 9-nt overhang on | 2 | 25 | 16 | 48 |
| | | 3′terminus of antisense | | | | |
| | | strand; LNA at 5′terminal | | | | |
| | | nucleotide of sense strand; | | | | |
| | | PS linkage in antisense | | | | |
| | | between positions 13 and | | | | |
| | | 14, and 14 and 15 | | | | |
| E | 11 | 9-nt overhang on | 3 | 24 | 16 | 43 |
| | | 3′terminus of antisense | | | | |
| | | strand; shortened 3-nt | | | | |
| | | length stem-loop; LNA at | | | | |
| | | 5′ terminal nucleotide of | | | | |
| | | sense strand and the first | | | | |
| | | and last position of the | | | | |
| | | stem-loop; PS linkage in | | | | |
| | | antisense between | | | | |
| | | positions 13 and 14, and | | | | |
| | | 14 and 15 | | | | |
| F | 14 | 9-nt overhang on | 2 | 25 | 16 | 49 |
| | | 3′terminus of antisense | | | | |
| | | strand; LNA at 5′terminal | | | | |
| | | nucleotide of sense strand; | | | | |
| | | G16 and G19 modified to | | | | |
| | | 2′-F; PS linkage in | | | | |
| | | antisense between | | | | |
| | | positions 13 and 14, and | | | | |
| | | 14 and 15 | | | | |
| G | 15 | 9-nt overhang on | 4 | 26 | 16 | 50 |
| | | 3'terminus of antisense | | | | |
| | | strand; shortened 3-nt | | | | |
| | | length stem-loop; LNA at | | | | |
| | | 5′ terminal nucleotide of | | | | |
| | | sense strand and the first | | | | |
| | | and last position of the | | | | |
| | | stem-loop; G16 and G19 | | | | |
| | | modified to 2′-F; PS | | | | |
| | | linkage in antisense | | | | |
| | | between positions 13 and | | | | |
| | | 14, and 14 and 15 | | | | |

PS = phosphorthioate; nt = nucleotide

To evaluate the impact of stem length on the tissue specificity of the RNAi oligonucleotide-lipid conjugates, C16 was utilized based on the above studies. Target knockdown was assessed 14 days post-injection. RNA was extracted from liver, skeletal muscle, adipose, and adrenals and murine ALDH2 mRNA levels were determined by qPCR. All samples were normalized to the PBS treated control animals. Normalized ALDH2 mRNA expression in the liver, skeletal muscle, adipose, and adrenals relative to control as measured for each treatment group at day 14 are shown in FIGS. 10A-10D, respectively. Compounds with shorter stem lengths (e.g., Compounds 11 and 15) provided similar levels of reduced mRNA expression across the various tissues compared to longer stem length compounds (e.g., Compounds 13 and 14) demonstrating stem length does not alter RNAi oligonucleotide-lipid conjugate activity in different tissues. The data further demonstrates the length of the stem had no observable impact on the improved performance of the RNAi oligonucleotide-lipid conjugate having exposed 2′-F nucleotides in muscle relative to liver. However, when exposed 2-F′ nucleotides were introduced at positions G16 and G19 of the anti-sense strand, activity improved in skeletal muscle compared to liver, adipose, and adrenal tissue.

### Example 5: Tissue Specific Activity of RNAi-Oligonucleotides with Exposed G19 2′-F Modified Nucleotide is Reduced with Shorter Sense Strand Truncation Length

To identify whether the length of sense strand truncation impacted the tissue specific activity of an RNAi oligonucleotide-lipid conjugate comprising an exposed 2′-F modified nucleotide on the guide strand, RNAi oligonucleotide-lipid conjugates were generated by the methods described in Example 1. To evaluate the RNAi oligonucleotide-lipid conjugates, CD-1 female mice were given a single subcutaneous injection of 15 mg/kg oligonucleotide-lipid conjugate formulated in PBS. The RNAi oligonucleotide-lipid conjugates administered for each of the treatment groups are outlined in Table 5 and depicted in FIG. 11. Group A were control mice that received PBS only.

To determine whether sense strand truncation impacted activity, RNAi oligonucleotide-lipid conjugates having varying sense strand lengths were compared. Specifically, Compounds 16-22 as provided in FIG. 11 were evaluated. Comparison was made to Compound 16 which contains a stem-loop at the 3′terminus of the sense strand and a 9-nt overhang at the 3′terminus of the antisense strand. The sense strand comprises an LNA at the 5′ terminal nucleotide. The stem-loop contains a tetraloop having the nucleotide sequence 5′-GAAA-3′, a stem of 6-nt in length, and a C16 lipid conjugated at the second nucleotide of the tetraloop. RNAi oligonucleotide-lipid conjugates that were evaluated in comparison to Compound 16 had structures according to Compounds 17-22. These structures contain the structural features described in Table 5.

TABLE 5
Lipid-conjugated RNAi oligonucleotides

| | | Description | Sense SEQ ID | | Antisense SEQ | |
|---|---|---|---|---|---|---|
| Group | Compound | (difference from | NOs | | ID NOS | |
| # | ID | Compound 16) | Unmod | Mod | Unmod | Mod |
| A | PBS | | — | — | — | — |
| B | 16 | — | 2 | 25 | 16 | 44 |
| C | 17 | G19 modified to 2'-F | 2 | 25 | 16 | 48 |
| G | 18 | 8-nt overhang on 3'terminus | 4 | 26 | 16 | 50 |
| | | of antisense strand; G19 | | | | |
| | | modified to 2'-F; no PS | | | | |
| | | linkage in antisense between | | | | |
| | | positions 13 and 14, and 14 | | | | |
| | | and 15 | | | | |
| H | 19 | 7-nt overhang on 3'terminus | 5 | 27 | 16 | 50 |
| | | of antisense strand; G19 | | | | |
| | | modified to 2'-F; no PS | | | | |
| | | linkage in antisense between | | | | |
| | | positions 13 and 14, and 14 | | | | |
| | | and 15 | | | | |
| I | 20 | 6-nt overhang on 3'terminus | 6 | 28 | 16 | 50 |
| | | of antisense strand; G19 | | | | |
| | | modified to 2'-F; no PS | | | | |
| | | linkage in antisense between | | | | |
| | | positions 13 and 14, and 14 | | | | |
| | | and 15 | | | | |
| J | 21 | 5-nt overhang on 3'terminus | 7 | 29 | 16 | 50 |
| | | of antisense strand; G19 | | | | |
| | | modified to 2'-F; no PS | | | | |
| | | linkage in antisense between | | | | |
| | | positions 13 and 14, and 14 | | | | |
| | | and 15 | | | | |
| K | 22 | 4-nt overhang on 3'terminus | 8 | 30 | 16 | 50 |
| | | of antisense strand; G19 | | | | |
| | | modified to 2'-F; no PS | | | | |
| | | linkage in antisense between | | | | |
| | | positions 13 and 14, and 14 | | | | |
| | | and 15 | | | | |

PS = phosphorothioate; nt = nucleotide

Target knockdown was assessed 14 days post-injection. RNA was extracted from liver, skeletal muscle, adipose, and adrenals and murine ALDH2 mRNA levels were determined by qPCR. All samples were normalized to the PBS treated control animals. Normalized ALDH2 mRNA expression in the liver, skeletal muscle, adipose, and adrenals relative to control as measured for each treatment group at day 14 are shown in FIGS. 12A-12D, respectively. Notably, compounds having sense strand truncations of less than 4 bases (i.e., less than 6 total nucleotide overhang; e.g., Compounds 21 and 22), did not provide reduction of inhibition of target mRNA expression in the liver while avoiding reduction of inhibition in extra-hepatic tissue was observed with RNAi oligonucleotide-lipid conjugates having the exposed G19 2′-F. Without wishing to be bound by theory, the data suggests steric hinderance of the nucleases which cut at the exposed G19 2′-F nucleotide.

### Example 6: Acyl Chain Position and Blunt RNAi Oligonucleotides Do Not Impact Tissue Specific Activity of RNAi Oligonucleotides with Exposed G19 2′-F Modified Nucleotide

To identify if the position of the acyl chain or 3′ structure of the RNAi oligonucleotide modified tissue specific activity of RNAi oligonucleotide-lipid conjugates comprising exposed 2′-F modified nucleotide on the guide strand, RNAi oligonucleotide-lipid conjugates were generated by the methods described in Example 1. To evaluate the RNAi oligonucleotide-lipid conjugates, CD-1 female mice were given a single subcutaneous injection of 15 mg/kg oligonucleotide-lipid conjugate formulated in PBS. The RNAi oligonucleotide-lipid conjugates administered for each of the treatment groups targeted ALDH2 which are outlined in Table 6 and depicted in FIG. 13, Slc25a1 which are outlined in Table 7 and depicted in FIG. 15, and Stat3 which are outlined in Table 8 and depicted in FIG. 17. Group A were control mice that received PBS only.

### ALDH2 RNAi Oligonucleotides

Comparison was made to Compound 12 as described in Example 4. RNAi oligonucleotide-lipid conjugates that were evaluated in comparison to Compound 12 had structures according to Compounds 23-28 as shown in FIG. 13. These structures contain the structural features described in Table 6.

TABLE 6
Lipid-conjugated RNAi oligonucleotides targeting ALDH2

| | | Description | Sense SEQ ID | | Antisense SEQ | |
|---|---|---|---|---|---|---|
| Group | Compound | (difference from | NOs | | ID NOS | |
| # | ID | Compound 12) | Unmod | Mod | Unmod | Mod |
| A | — | | — | — | — | — |
| B | 12 | — | 2 | 25 | 16 | 44 |
| C | 23 | 6-nt overhang on 3′terminus | 6 | 28 | 17 | 51 |
| | | of antisense strand; LNA at | | | | |
| | | 5′terminal nucleotide of | | | | |
| | | sense strand | | | | |
| D | 24 | 6-nt overhang on 3′terminus | 6 | 28 | 17 | 52 |
| | | of antisense strand; LNA at | | | | |
| | | 5′terminal nucleotide of | | | | |
| | | sense strand; G19 modified | | | | |
| | | to 2′-F | | | | |
| E | 42 | 6-nt overhang on 3′terminus | 6 | 28 | 17 | 53 |
| | | of antisense strand; LNA at | | | | |
| | | 5′terminal nucleotide of | | | | |
| | | sense strand; G19 modified | | | | |
| | | to 2′-F; PS linkage in | | | | |
| | | antisense between positions | | | | |
| | | 19 and 20 | | | | |
| F | 25 | 6-nt overhang on 3′terminus | 6 | 31 | 17 | 52 |
| | | of antisense strand; C16 | | | | |
| | | lipid at 5′terminal | | | | |
| | | nucleotide of sense strand | | | | |
| | | rather than stem loop; G19 | | | | |
| | | modified to 2′-F | | | | |
| G | 43 | 6-nt overhang on 3′terminus | 6 | 31 | 17 | 53 |
| | | of antisense strand; C16 | | | | |
| | | lipid at 5′terminal | | | | |
| | | nucleotide of sense strand | | | | |
| | | rather than stem loop; G19 | | | | |
| | | modified to 2′-F; PS linkage | | | | |
| | | in antisense between | | | | |
| | | positions 19 and 20 | | | | |
| H | 26 | No stem loop, sense strand | 9 | 32 | 17 | 51 |
| | | comprises 3′ blunt end with | | | | |
| | | C16 lipid; LNA at 5′ | | | | |
| | | terminal nucleotide of sense | | | | |
| | | strand | | | | |
| I | 27 | No stem loop, sense strand | 9 | 32 | 17 | 52 |
| | | comprises 3′ blunt end with | | | | |
| | | C16 lipid; LNA at 5′ | | | | |
| | | terminal nucleotide of sense | | | | |
| | | strand; G19 modified to 2′- | | | | |
| | | F | | | | |
| J | 28 | No stem loop, sense strand | 9 | 32 | 17 | 53 |
| | | comprises 3′ blunt end with | | | | |
| | | C16 lipid; LNA at 5′ | | | | |
| | | terminal nucleotide of sense | | | | |
| | | strand; G19 modified to 2′- | | | | |
| | | F; PS linkage in antisense | | | | |
| | | between positions 19 and 20 | | | | |

PS = phosphorothioate; nt = nucleotide

Target knockdown was assessed 14 days post-injection. RNA was extracted from liver, skeletal muscle, adipose, and adrenals and murine ALDH2 mRNA levels were determined by qPCR. All samples were normalized to the PBS treated control animals. Normalized ALDH2 mRNA expression in the liver, skeletal muscle, adipose, and adrenals relative to control as measured for each treatment group at day 14 are shown in FIGS. 14A-14D, respectively. Similar to the ALDH2 compounds evaluated in Example 3, incorporating an exposed G19 2-F′ modified nucleotide on the antisense strand yielded extra-hepatic tissue specific activity. Specifically, there was little to no inhibition of target mRNA in the liver whereas inhibition is observed in extra-hepatic tissues such as skeletal muscle, adipose, and adrenal tissue (see Compound 24 vs. Compounds 12 and 23). Even when the acyl chain is conjugated to the 5′ terminal nucleotide of the sense strand rather than on the stem loop (Compound 25), inhibition of target mRNA is maintained in extra-hepatic tissue but the inhibitory function is nearly eliminated in the liver (see e.g., Compound 24 vs. Compound 25). Similarly, when the compound comprises a blunt end and the acyl chain is conjugated to the blunt end (Compound 27), inhibition of target mRNA is maintained in extra-hepatic tissue but the inhibitory function is nearly eliminated in the liver (see e.g., Compound 26 vs. Compound 27). Together this data suggests that the location of an acyl chain does not impact the extra-hepatic specificity of RNAi-oligonucleotide ligand conjugates comprising a G19 2′-F mutation. Without wishing to be bound by theory, the structure of the 3′ of the sense strand and the 5′ of the antisense strand does not appear to impact the activity of the exposed 2′-F nucleotide(s) on the antisense strand, allowing for further structural changes while maintaining the improved activity in extra-hepatic tissue.

### SLC25A1 RNAi Oligonucleotides

TABLE 7
Lipid-conjugated RNAi oligonucleotides targeting Slc25a1

| | | Description | | | | |
|---|---|---|---|---|---|---|
| | | (difference | | | Antisense SEQ ID | |
| Group | Compound | from | Sense SEQ ID NOS | | NOs | |
| # | ID | Compound 29) | Unmod | Mod | Unmod | Mod |
| A | — | | — | — | — | — |
| B | 29 | — | 10 | 33 | 18 | 54 |
| C | 30 | 6-nt overhang on | 11 | 34 | 18 | 54 |
| | | 3′terminus of | | | | |
| | | antisense; LNA | | | | |
| | | at 5′terminal nt | | | | |
| | | of sense strand | | | | |
| D | 31 | 6-nt overhang on | 11 | 34 | 18 | 55 |
| | | 3′terminus of | | | | |
| | | antisense; LNA | | | | |
| | | at 5′terminal nt | | | | |
| | | of sense strand; | | | | |
| | | G19 modified to | | | | |
| | | 2′-F | | | | |
| F | 32 | 6-nt overhang on | 11 | 34 | 18 | 56 |
| | | 3′terminus of | | | | |
| | | antisense; LNA | | | | |
| | | at 5′terminal nt | | | | |
| | | of sense strand; | | | | |
| | | G19 modified to | | | | |
| | | 2′-F; PS linkage | | | | |
| | | in antisense | | | | |
| | | between | | | | |
| | | positions 19 and | | | | |
| | | 20 | | | | |
| H | 33 | No stem-loop; | 12 | 35 | 18 | 54 |
| | | LNA at | | | | |
| | | 5′terminal nt of | | | | |
| | | sense strand; | | | | |
| | | C16 at 3′ | | | | |
| | | terminal nt of | | | | |
| | | sense strand | | | | |
| I | 34 | No stem-loop; | 12 | 35 | 18 | 55 |
| | | LNA at | | | | |
| | | 5′terminal nt of | | | | |
| | | sense strand; | | | | |
| | | C16 at 3′ | | | | |
| | | terminal nt of | | | | |
| | | sense strand; | | | | |
| | | G19 modified to | | | | |
| | | 2′-F | | | | |

Target knockdown was assessed 14 days post-injection. RNA was extracted from liver, skeletal muscle, adipose, and adrenals and murine Slc25a1 mRNA levels were determined by qPCR. All samples were normalized to the PBS treated control animals. Normalized Slc25a1 mRNA expression in the liver, skeletal muscle, adipose, and adrenals relative to control as measured for each treatment group at day 14 are shown in FIGS. 16A-16D, respectively. Similar to the ALDH2 compounds evaluated in Example 3, incorporating an exposed G19 2-F′ modified nucleotide on the antisense strand yielded extra-hepatic tissue specific activity. Specifically, inhibition of target mRNA in the liver is significantly reduced whereas inhibition is maintained in extra-hepatic tissues such as skeletal muscle, adipose, and adrenal tissue (see Compound 31 vs. Compounds 29 and 30). Even when the acyl chain is conjugated to the 5′ terminal nucleotide of the sense strand rather than on the stem loop (Compound 32), inhibition of target mRNA is maintained in extra-hepatic tissue but the inhibitory function is nearly eliminated in the liver (see e.g., Compound 31 vs. Compound 32). Similarly, when the compound comprises a blunt end and the acyl chain is conjugated to the blunt end (Compound 34), inhibition of target mRNA is maintained in extra-hepatic tissue but the inhibitory function is nearly eliminated in the liver (see e.g., Compound 33 vs. Compound 34). Together this data suggests that the location of an acyl chain and the structure of the 3′end of the RNAi lipid-conjugated oligonucleotide (i.e., stem-loop or blunt end) do not impact the extra-hepatic specificity of RNAi-oligonucleotide ligand conjugates comprising a G19 2′-F modification.

### STAT3 RNAi Oligonucleotides

TABLE 8
Lipid-conjugated RNAi oligonucleotides targeting STAT3

| | | Description | | | | |
|---|---|---|---|---|---|---|
| | | (difference | | | Antisense SEQ ID | |
| Group | Compound | from | Sense SEQ ID NOS | | NOs | |
| # | ID | Compound 35) | Unmod | Mod | Unmod | Mod |
| A | — | | — | — | — | — |
| B | 35 | — | 13 | 36 | 19 | 57 |
| H | 36 | no stem loop; | 14 | 37 | 19 | 57 |
| | | LNA at 5′ | | | | |
| | | terminal | | | | |
| | | nucleotide of | | | | |
| | | sense; C16 lipid | | | | |
| | | at 3′ terminal | | | | |
| | | nucleotide of | | | | |
| | | sense | | | | |
| I | 37 | no stem loop; | 14 | 37 | 19 | 58 |
| | | LNA at 5′ | | | | |
| | | terminal | | | | |
| | | nucleotide of | | | | |
| | | sense; C16 lipid | | | | |
| | | at 3′ terminal | | | | |
| | | nucleotide of | | | | |
| | | sense; G19 | | | | |
| | | modified to 2′-F | | | | |

Target knockdown was assessed 14 days post-injection. RNA was extracted from liver, skeletal muscle, adipose, and adrenals and murine STAT3 mRNA levels were determined by qPCR. All samples were normalized to the PBS treated control animals. Normalized STAT3 mRNA expression in the liver, skeletal muscle, adipose, and adrenals relative to control as measured for each treatment group at day 14 are shown in FIGS. 18A-18D, respectively. When an exposed G19 2′-F modified nucleotide is incorporated on the antisense strand of a STAT3 RNAi oligonucleotide-lipid conjugate comprising an acyl chain on a blunt-end, activity of inhibition is maintained in adrenal and adipose tissue whereas the inhibitory effect is lost in the liver. Together this data demonstrates that a blunt end RNAi-oligonucleotide comprising an acyl chain at the 3′ end of the sense strand and a 2′-F modified nucleotide at G19 of the antisense strand has extra-hepatic tissue specificity for inhibiting target mRNA.

SEQUENCE LISTING

| | | | SEQ |
|---|---|---|---|
| | | | ID |
| Compound | Description | Sequence | NO |
| ALDH- | Unmodified | GGUGGAUGAAACUCAGUUUAGCAGCCGAAAG | 1 |
| 1203 | Sense Strand | GCUGC | |
| ALDH- | Unmodified | GAAACUCAGUUUAGCAGCCGAAAGGCUGC | 2 |
| 1203 | Sense Strand | | |
| ALDH- | Unmodified | GAAACUCAGUUUAGCCGAAAGGC | 3 |
| 1203 | Sense Strand | | |
| ALDH- | Unmodified | TGAAACUCAGUUUAGCAGCCGAAAGGCUGC | 4 |
| 1203 | Sense Strand | | |
| ALDH- | Unmodified | AUGAAACUCAGUUUAGCAGCCGAAAGGCUGC | 5 |
| 1203 | Sense Strand | | |
| ALDH- | Unmodified | GAUGAAACUCAGUUUAGCAGCCGAAAGGCUGC | 6 |
| 1203 | Sense Strand | | |
| ALDH- | Unmodified | GGAUGAAACUCAGUUUAGCAGCCGAAAGGCU | 7 |
| 1203 | Sense Strand | GC | |
| ALDH- | Unmodified | TGGAUGAAACUCAGUUUAGCAGCCGAAAGGCU | 8 |
| 1203 | Sense Strand | GC | |
| ALDH- | Unmodified | GAUGAAACUCAGUUUA | 9 |
| 1203 | Sense Strand | | |
| SLC25A1- | Unmodified | ACCAUCAAGGUGAAGUUCAAGCAGCCGAAAGG | 10 |
| 0597 | Sense Strand | CUGC | |
| SLC25A1- | Unmodified | TCAAGGUGAAGUUCAAGCAGCCGAAAGGCUGC | 11 |
| 0597 | Sense Strand | | |
| SLC25A1- | Unmodified | TCAAGGUGAAGUUCAA | 12 |
| 0597 | Sense Strand | | |
| STAT3- | Unmodified | AGUUGAAUUAUCAGCUUAAAGCAGCCGAAAG | 13 |
| 1402 | Sense Strand | GCUGC | |
| STAT3- | Unmodified | GAAUUAUCAGCUUAAA | 14 |
| 1402 | Sense Strand | | |
| Stem loop | — | GCAGCCGAAAGGCUGC | 15 |
| ALDH- | Unmodified | UAAACUGAGUUUCAUCCACCGG | 16 |
| 1203 | Anti-sense | | |
| | Strand | | |
| ALDH- | Unmodified | UAAACUGAGUUUCAUCCACUGG | 17 |
| 1203 | Anti-sense | | |
| | Strand | | |
| SLC25A1- | Unmodified | UUGAACUUCACCUUGAUGGUGG | 18 |
| 0597 | Anti-sense | | |
| | Strand | | |
| STAT3- | Unmodified | UUUAAGCUGAUAAUUCAACUGG | 19 |
| 1402 | Anti-sense | | |
| | Strand | | |
| ALDH- | Modified | [mGs][mG][fU][mG][fG][mA][mU][fG] | 20 |
| 1203 | Sense strand | [mA][fA][mA][fC][fU][mC][fA][mG][fU][mU] | |
| | | [mU][mA][mG][mC][mA][mG][mC][mC][mG][ademA- | |
| | | C22][mA][mA][mG][mG][mC][mU][mG][mC] | |
| ALDH- | Modified | [+Gs][mA][fA][mA][fC][fU][mC][fA] | 21 |
| 1203 | Sense strand | [mG][fU][mU][mU][mA][mG][mC][mA][mG][mC] | |
| | | [mC][mG][ademA-C22] | |
| | | [mA][mA][mG][mG][mC][mU][mG][mC] | |
| ALDH- | Modified | [+Gs][mA][fA][mA][fC][fU][mC][fA][mG][fU][mU] | 22 |
| 1203 | Sense strand | [mU][mA][+G][mC][mC][mG][ademA- | |
| | | C22][mA][mA][mG][mG][+C] | |
| ALDH- | Modified | [+Gs][fA][fA][fA][mC][mU][mC][mA][mG][mU][mU] | 23 |
| 1203 | Sense strand | [mU][mA][+G][mC][mC][mG][ademA- | |
| | | C22][mA][mA][mG][mG][+C] | |
| ALDH- | Modified | [+Gs][fA][fA][fA][mC][mU][mC][mA][mG][mU][mU] | 24 |
| 1203 | Sense strand | [mU][mA][+G][mC][mC][mG][ademA- | |
| | | C16][mA][mA][mG][mG][+C] | |
| ALDH- | Modified | [+Gs][fA][fA][fA][mC][mU][mC][mA][mG][mU][mU] | 25 |
| 1203 | Sense strand | [mU][mA][mG][mC][mA][mG][mC][mC][mG][ademA- | |
| | | C16][mA][mA][mG][mG][mC][mU][mG][mC] | |
| ALDH- | Modified | [+Ts][fG][fA][fA][fA][mC][mU][mC][mA][mG][mU] | 26 |
| 1203 | Sense strand | [mU][mU][mA][mG][mC][mA][mG][mC][mC][mG] | |
| | | [ademA-C16][mA][mA][mG\|mG][mC\|mU][mG][mC] | |
| ALDH- | Modified | [+As][mU][fG][fA][fA][fA][mC][mU][mC][mA][mG] | 27 |
| 1203 | Sense strand | [mU][mU][mU][mA][mG][mC][mA][mG][mC][mC][mG] | |
| | | [ademA- | |
| | | C16][mA][mA][mG][mG][mC][mU][mG][mC] | |
| ALDH- | Modified | [+Gs][mA][mU][fG][fA][fA][fA][mC][mU][mC][mA] | 28 |
| 1203 | Sense strand | [mG][mU][mU][mU][mA][mG][mC][mA][mG][mC][mC] | |
| | | [mG][ademA- | |
| | | C16][mA][mA][mG][mG][mC][mU][mG][mC] | |
| ALDH- | Modified | [+Gs][mG][mA][mU][fG][fA][fA][fA][mC][mU][mC] | 29 |
| 1203 | Sense strand | [mA][mG][mU][mU][mU][mA][mG][mC][mA][mG][mC] | |
| | | [mC][mG][ademA- | |
| | | C16][mA][mA][mG][mG][mC][mU][mG][mC] | |
| ALDH- | Modified | [+Ts][mG][mG][mA][mU][fG][fA][fA][fA][mC][mU] | 30 |
| 1203 | Sense strand | [mC][mA][mG][mU][mU][mU][mA][mG][mC][mA][mG] | |
| | | [mC][mC][mG][ademA- | |
| | | C16][mA][mA][mG][mG][mC][mU][mG][mC] | |
| ALDH- | Modified | [ademGs- | 31 |
| 1203 | Sense strand | C16][mA][mU][fG][fA][fA][fA][mC][mU][mC][mA] | |
| | | [mG][mU][mU][mU][mA][mG][mC][mA][mG][mC][mC] | |
| | | [mG][mA][mA][mA][mG][mG][mC][mU][mG][mC] | |
| ALDH- | Modified | [+Gs][mA][mU][fG][fA][fA][fA][mC][mU][mC][mA] | 32 |
| 1203 | Sense strand | [mG][mU][mUs][mUs][ademA-C16] | |
| SLC25A1- | Modified | [mAs][mC][mC][mA][mU][mC][mA][fA][fG][fG][fU] | 33 |
| 0597 | Sense strand | [mG][mA][mA][mG][mU][mU][mC][mA][mA][mG] | |
| | | [mC][mA][mG][mC][mC][mG][ademA- | |
| | | C16][mA][mA][mG][mG][mC][mU][mG][mC] | |
| SLC25A1- | Modified | [+Ts][mC][mA][fA][fG][fG][fU][mG][mA][mA][mG] | 34 |
| 0597 | Sense strand | [mU][mU][mC][mA][mA][mG][mC][mA][mG][mC][mC] | |
| | | [mG][ademA- | |
| | | C16][mA][mA][mG][mG][mC][mU][mG][mC] | |
| SLC25A1- | Modified | [+Ts][mC][mA][fA][fG][fG][fU][mG][mA][mA][mG] | 35 |
| 0597 | Sense strand | [mU][mU][mCs][mAs][ademA-C16] | |
| STAT3- | Modified | [mAs][mG][mU][mU][mG][mA][mA][fU][fU][fA][fU] | 36 |
| 1402 | Sense strand | [mC][mA][mG][mC][mU][mU][mA][mA][mA][mG] | |
| | | [mC][mA][mG][mC][mC][mG][ademA- | |
| | | C16][mA][mA][mG][mG][mC][mU][mG][mC] | |
| STAT3- | Modified | [+Gs][mA][mA][fU][fU][fA][fU][mC][mA][mG][mC] | 37 |
| 1402 | Sense strand | [mU][mU][mAs][mAs][ademA-C16] | |
| Tetraloop | | GAAA | 38 |
| ALDH- | Modified | [MePhosphonate-40-mUs][fAs][fA] | 39 |
| 1203 | Anti-sense | [fA][fC][mU][fG][mA][mG][fU][mU][mU][mC][fA] | |
| | strand | [mU][fC][mC][mA ][fC][mCs][mGs][mG] | |
| ALDH- | Modified | [MePhosphonate-40-mUs][fAs][fAs] | 40 |
| 1203 | Anti-sense | [fA][fC][mU][fG][mA][mG][fU][mU][mU][mCs] | |
| | strand | [fAs][mU][fC][mC] [mA][fC][mCs][mGs][mG] | |
| ALDH- | Modified | [MePhosphonate-40-mUs][fAs][fAs] | 41 |
| 1203 | Anti-sense | [fA][fC][mU][fG][mA][mG][fU][mU][mU][mCs] | |
| | strand | [fAs][mU][mC][mC] [mA][mC][mCs][mGs][mG] | |
| ALDH- | Modified | [MePhosphonate-40-mUs][fAs][fAs] | 42 |
| 1203 | Anti-sense | [fA][fC][mU][fG][mA][mG][fU][mU][mU][mCs] | |
| | strand | [fAs][mUs][mCs][m Cs][mAs][mCs][mCs][mGs][mG] | |
| ALDH- | Modified | [MePhosphonate-40- | 43 |
| 1203 | Anti-sense | mUs][fAs][fAs][fA][fC][mU][fG][mA][mG][fU][mU] | |
| | strand | [mU][mCs][fAs][mU][mC][mC][mA][mC][mCs][mGs] | |
| | | [mG] | |
| ALDH- | Modified | [MePhosphonate-40- | 44 |
| 1203 | Anti-sense | mUs][fAs][fAs][fA][fC][mU][fG][mA][mG][fU][mU] | |
| | strand | [mU][mCs][fAs][mU][mC][mC][mA][fC][mCs][mGs] | |
| | | [mG] | |
| ALDH- | Modified | [MePhosphonate-40- | 45 |
| 1203 | Anti-sense | mUs][fAs][fAs][fA][fC][mU][fG][mA][mG][fU][mU] | |
| | strand | [mU][mCs][fAs][mU][fC][mC][mA][mC][mCs][mGs] | |
| | | [mG] | |
| ALDH- | Modified | [MePhosphonate-40- | 46 |
| 1203 | Anti-sense | mUs][fAs][fAs][fA][fC][mU][fG][mA][mG][fU][mU] | |
| | strand | [mU][mCs][fAs][mU][fC][mC][mA][fC][mCs][mGs] | |
| | | [mG] | |
| ALDH- | Modified | [MePhosphonate-40- | 47 |
| 1203 | Anti-sense | mUs][fAs][fAs][fA][fC][mU][fG][mA][mG][fU][mU] | |
| | strand | [mU][mCs][fAs][mUs][fCs][mCs][mAs][fCs][mCs] | |
| | | [mGs][mG] | |
| ALDH- | Modified | [MePhosphonate-40- | 48 |
| 1203 | Anti-sense | mUs][fAs][fAs][fA][fC][mU][fG][mA][mG][fU][mU] | |
| | strand | [mU][mCs][fAs][mU][mC][mC][mAs][fCs][mCs][mGs] | |
| | | [mG] | |
| ALDH- | Modified | [MePhosphonate-40- | 49 |
| 1203 | Anti-sense | mUs][fAs][fAs][fA][fC][mU][fG][mA][mG][fU][mU] | |
| | strand | [mU][mCs][fAs][mU][mC][mC][mAs][fC][mCs][mGs] | |
| | | [mG] | |
| ALDH- | Modified | [MePhosphonate-40- | 50 |
| 1203 | Anti-sense | mUs][fAs][fAs][fA][fC][mU][fG][mA][mG][fU][mU] | |
| | strand | [mU][mC][fA][mU][mC][mC][mA][fC][mCs][mGs] | |
| | | [mG] | |
| ALDH- | Modified | [MePhosphonate-40- | 51 |
| 1203 | Anti-sense | mUs ][fAs][fAs][fA][fC][mU][fG][mA][mG][fU][mU] | |
| | strand | [mU][mC][fA][mU][mC][mC][mA][mC][mUs][mGs] | |
| | | [mG] | |
| ALDH- | Modified | [MePhosphonate-40- | 52 |
| 1203 | Anti-sense | mUs][fAs][fAs][fA][fC][mU][fG][mA][mG][fU][mU] | |
| | strand | [mU][mC][fA][mU][mC][mC][mA][fC][mUs][mGs] | |
| | | [mG] | |
| ALDH- | Modified | [MePhosphonate-40- | 53 |
| 1203 | Anti-sense | mUs][fAs][fAs][fA][fC][mU][fG][mA][mG][fU][mU] | |
| | strand | [mU][mC][fA][mU][mC][mC][mA][fCs][mUs][mGs] | |
| | | [mG] | |
| SLC25A1- | Modified | [MePhosphonate-40- | 54 |
| 0597 | Anti-sense | mUs][fUs][fGs][fA][fA][mC][fU][mU][mC][fA][mC] | |
| | strand | [mC][mU][fU][mG][mA][mU][mG][mG][mUs][mGs] | |
| | | [mG] | |
| SLC25A1- | Modified | [MePhosphonate-40- | 55 |
| 0597 | Anti-sense | mUs][fUs][fGs][fA][fA][mC][fU][mU][mC][fA][mC] | |
| | strand | [mC][mU][fU][mG][mA][mU][mG][fG][mUs][mGs] | |
| | | [mG] | |
| SLC25A1- | Modified | [MePhosphonate-40- | 56 |
| 0597 | Anti-sense | mUs][fUs][fGs][fA][fA][mC][fU][mU][mC][fA][mC] | |
| | strand | [mC][mU][fU][mG][mA][mU][mG][fGs][mUs][mGs] | |
| | | [mG] | |
| STAT3- | Modified | [MePhosphonate-40- | 57 |
| 1402 | Anti-sense | mUs][fUs][fUs][fA][fA][mG][fC][mU][mG][fA][mU] | |
| | strand | [mA][mA][fU][mU][mC][mA][mA][mC][mUs][mGs] | |
| | | [mG] | |
| STAT3- | Modified | [MePhosphonate-40- | 58 |
| 1402 | Anti-sense | mUs][fUs][fUs][fA][fA][mG][fC][mU][mG][fA][mU] | |
| | strand | [mA][mA][fU][mU][mC][mA][mA][fC][mUs][mGs] | |
| | | [mG] | |

1. A double-stranded oligonucleotide comprising an antisense strand of 15-30 nucleotides in length and a sense strand of about 9-50 nucleotides in length, wherein (i) the antisense and sense strands form a duplex region of about 9-26 base pairs, (ii) the antisense strand comprises an orientation of 5′ to 3′, (iii) the antisense strand comprises a 3′ overhang of at least four nucleotides, (iv) the antisense strand comprises a region of complementarity to an mRNA target sequence in a target mRNA, (v) the 3′ overhang comprises a sequence motif that reduces inhibition of the target mRNA by the double-stranded oligonucleotide in a cell of the liver, and (vi) the sequence motif comprises at least one 2′-F modified nucleotide, provided the at least one 2′-F modified nucleotide is not one of the two 3′terminal nucleotides of the antisense strand.

2. The double-stranded oligonucleotide of claim 1, wherein inhibition of the target mRNA is reduced compared to inhibition of the target mRNA by a double-stranded oligonucleotide not having the sequence motif.

3. The double-stranded oligonucleotide of claim 1 or 2, wherein the cell of the liver is a hepatocyte.

4. The double-stranded oligonucleotide of any one of claims 1-3, wherein the sequence motif comprises: 3′-P₁P₂[N]_{y}X₁-5′ wherein:
P₁ and P₂ are each independently a purine or a pyrimidine, and do not comprise a 2′-F modification;
X₁ is any nucleotide located immediately adjacent to the duplex, and comprises at least one phosphorothioate linkage to an adjacent nucleotide;
N is any nucleotide and y is an integer selected from 1-6, specifying N₁-N₆, wherein:
(a) when y is 1, N₁ comprises a 2′-F modification;
(b) when y is 2, one or more of N₁ and N₂ comprise a 2′-F modification;
(c) when y is 3, one or more of N₁-N₃ comprise a 2′-F modification;
(d) when y is 4, one or more of N₁-N₄ comprise a 2′-F modification;
(e) when y is 5, one or more of N₁-N₅ comprise a 2′-F modification; or
(f) when y is 6, one or more of N₁-N₆ comprise a 2′-F modification.

5. A double-stranded oligonucleotide for inhibiting a target mRNA in a cell of an extra-hepatic tissue comprising an antisense strand of 15-30 nucleotides in length and a sense strand of about 9-50 nucleotides in length, wherein (i) the antisense and sense strands form a duplex region of about 9-26 base pairs, (ii) the antisense strand comprises an orientation of 5′ to 3′, (iii) the antisense strand comprises a 3′ overhang of at least 4 nucleotides, (iv) the antisense strand comprises a region of complementarity to a mRNA target sequence in a target mRNA, and (v) the antisense strand comprises at least one 2′-F modified nucleotide in the 3′ overhang, provided the at least one 2′-F modified nucleotide is not one of the two 3′ terminal nucleotides of the antisense strand.

6. A double-stranded oligonucleotide for increasing inhibition of a target mRNA in a cell of an extra-hepatic tissue relative to inhibition of the target mRNA in a cell of liver tissue, comprising an antisense strand of 15-30 nucleotides in length and a sense strand of about 9-50 nucleotides in length, wherein (i) the antisense and sense strands form a duplex region of about 9-26 base pairs, (ii) the antisense strand comprises an orientation of 5′ to 3′, (iii) the antisense strand comprises a 3′ overhang of at least 4 nucleotides, (iv) the antisense strand comprises a region of complementarity to a mRNA target sequence in a target mRNA, and (v) the antisense strand comprises at least one 2′-F modified nucleotide in the 3′ overhang, provided the at least one 2′-F modified nucleotide is not one of the two 3′ terminal nucleotides of the antisense strand.

7. The double-stranded oligonucleotide of claim 5 or 6, wherein the extra-hepatic tissue is selected from skeletal muscle, adipose tissue, adrenal tissue, and any combination thereof.

8. The double-stranded oligonucleotide of claim 5 or 6, wherein the cell of the cell of the extra-hepatic tissue is selected from a cardiomyocyte, an immune cell, a liver non-parenchymal cell, a cell of skeletal muscle, a cell of adipose tissue, a cell of adrenal tissue, and any combination thereof.

9. The double-stranded oligonucleotide of any one of claims 5-8, wherein the 3′overhang comprises a sequence motif of: 3′-P₁P₂[N]_{y}X₁-5′
wherein:
P₁ and P₂ are each independently a purine or a pyrimidine, and do not comprise a 2′-F modification;
X₁ is any nucleotide located immediately adjacent to the duplex, and comprises at least one phosphorothioate linkage to an adjacent nucleotide;
N is any nucleotide and y is an integer selected from 1-6, specifying N₁-N₆, wherein:
(a) when y is 1, N₁ comprises a 2′-F modification;
(b) when y is 2, one or more of N₁ and N₂ comprise a 2′-F modification;
(c) when y is 3, one or more of N₁-N₃ comprise a 2′-F modification;
(d) when y is 4, one or more of N₁-N₄ comprise a 2′-F modification;
(e) when y is 5, one or more of N₁-N₅ comprise a 2′-F modification; or
(f) when y is 6, one or more of N₁-N₆ comprise a 2′-F modification.

10. The double-stranded oligonucleotide of claim 4 or 9, wherein when y is 3, N₂ comprises the 2′-F modification.

11. The double-stranded oligonucleotide of claim 10, wherein X₁ does not comprise a 2′-F modification.

12. The double-stranded oligonucleotide of claim 10 or 11, wherein N₁, N₃, and N₄ each comprise a 2′-OMe modification.

13. The double-stranded oligonucleotide of claim 4 or 9, wherein when y is 6, N₂ comprises the 2′-F modification.

14. The double-stranded oligonucleotide of claim 13, wherein X₁ comprises a 2′-F modification.

15. The double-stranded oligonucleotide of claim 13 or 14, wherein N₁, N₃, N₄, N₅, and N₆ each comprise a 2′-OMe modification.

16. The double-stranded oligonucleotide of claim 4 or 9, wherein when y is 6, N₂ and N₅ each comprise the 2′-F modification.

17. The double-stranded oligonucleotide of claim 16, wherein X₁ comprises a 2′-F modification.

18. The double-stranded oligonucleotide of claim 16 or 17, wherein N₁, N₃, N₄ and N₆ each comprise a 2′-OMe modification.

19. The double-stranded oligonucleotide of any one of claims 4 and 9-18, wherein P₁ and P₂ are each independently a purine.

20. The double-stranded oligonucleotide of claim 19, wherein P₁ and P₂ are each independently selected from adenosine and guanine.

21. The double-stranded oligonucleotide of claim 19, wherein P₁ and P₂ are each guanine.

22. The double-stranded oligonucleotide of nay one of claims 1-4 and 9-21, wherein the double-stranded oligonucleotide reduces expression of the target mRNA in an extra-hepatic cell, provided the double-stranded oligonucleotide does not reduce expression of the mRNA target in a cell of the liver.

23. The double-stranded oligonucleotide of any one of claims 1-3 and 5-8, wherein the antisense strand is 20-22 nucleotides, and the 2′-F modified nucleotide is at position 19.

24. The double-stranded oligonucleotide of any one of claims 1-3 and 5-8, wherein the antisense strand is 20-22 nucleotides, and the 2′-F modified nucleotide is at position 16.

25. The double-stranded oligonucleotide of any one of claims 1-3 and 5-8, wherein the antisense strand is 20-22 nucleotides, and the 2′-F modified nucleotide is at position 16 and position 19.

26. The double-stranded oligonucleotide of any one of claims 23-25, wherein the antisense strand is 22 nucleotides.

27. The double-stranded oligonucleotide of any one of claims 1-26, wherein:
(i) the sense strand is 29 nucleotides, the antisense strand is 22 nucleotides, and the duplex region is 13 nucleotides;
(ii) the sense strand is 23 nucleotides, the antisense strand is 22 nucleotides, and the duplex region is 13 nucleotides;
(iii) the sense strand is 30 nucleotides, the antisense strand is 22 nucleotides, and the duplex region is 14 nucleotides;
(iv) the sense strand is 31 nucleotides, the antisense strand is 22 nucleotides, and the duplex region is 15 nucleotides;
(v) the sense strand is 32 nucleotides, the antisense strand is 22 nucleotides, and the duplex region is 16 nucleotides; or
(vi) the sense strand is 16 nucleotides, the antisense strand is 22 nucleotides, and the duplex region is 16 nucleotides.

28. The double-stranded oligonucleotide of any one of claims 1-27, wherein the 2′-F modified nucleotide comprises a phosphorothioate linkage.

29. The double-stranded oligonucleotide of any one of claims 1-28, wherein the nucleotides adjacent to the 2′-F modified nucleotide do not have phosphorothioate linkages.

30. The double-stranded oligonucleotide of any one of claims 1-29, wherein the sense strand comprises at least one lipid moiety conjugated to a nucleotide on the sense strand.

31. The double-stranded oligonucleotide of claim 30, wherein the lipid moiety is selected from:

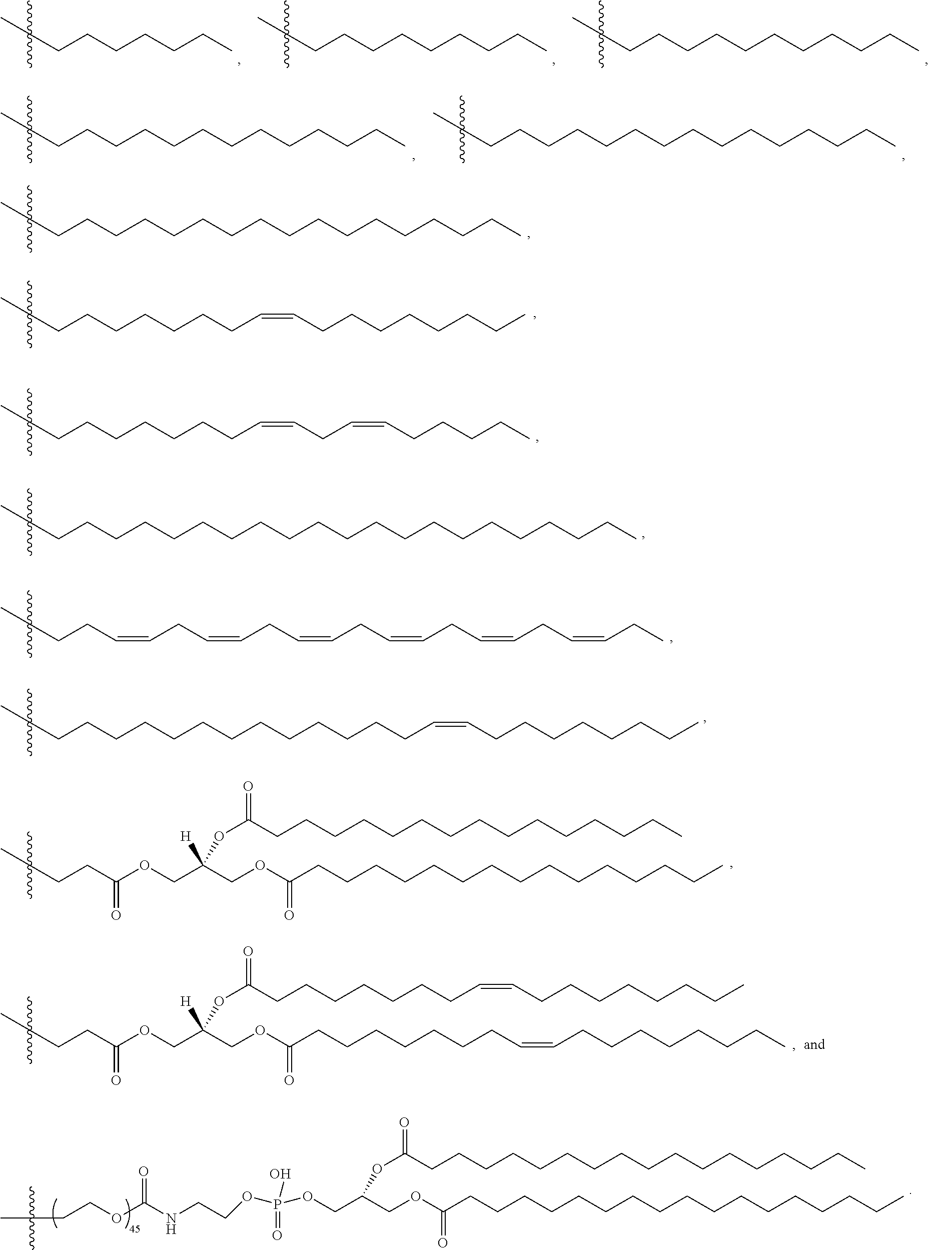

32. The double-stranded oligonucleotide of claim 30 or 31, wherein the lipid moiety is a hydrocarbon chain.

33. The double-stranded oligonucleotide of claim 32, wherein the hydrocarbon chain is a C8-C30 hydrocarbon chain.

34. The double-stranded oligonucleotide of claim 33, wherein the hydrocarbon chain is a C16 hydrocarbon chain.

35. The double-stranded oligonucleotide of claim 34, wherein the C16 hydrocarbon chain is represented by

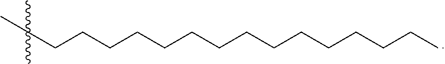 [SMILES: *CCCCCCCCCCCCCCC]

36. The double-stranded oligonucleotide of claim 33, wherein the hydrocarbon chain is a C22 hydrocarbon chain.

37. The double-stranded oligonucleotide of claim 36, wherein the C22 hydrocarbon chain is represented by

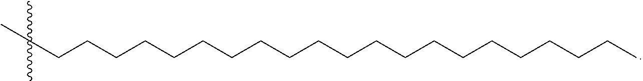 [SMILES: *CCCCCCCCCCCCCCCCCCCCC]

38. The double-stranded oligonucleotide of any one of claims 30-37, wherein the lipid moiety is conjugated to the 5′terminal nucleotide of the sense strand.

39. The double-stranded oligonucleotide of any one of claims 30-37, wherein the sense strand comprises a stem-loop, and wherein the lipid moiety is conjugated to a nucleotide of the stem-loop

40. The double-stranded oligonucleotide of any one of claims 30-39, wherein the lipid moiety is conjugated to the 2′ carbon of the ribose ring of the nucleotide.

41. The double-stranded oligonucleotide of any one of claims 1-38 and 40, wherein the sense strand comprises a stem-loop.

42. The double-stranded oligonucleotide of claim 39 or 41, wherein the stem-loop comprises a nucleotide sequence represented by the formula: 5′-S1-L-S2-3′, wherein S1 is complementary to S2, and wherein L forms a loop between S1 and S2.

43. The double-stranded oligonucleotide of claim 42, wherein S1 and S2 are each independently 1-20 nucleotides in length, optionally wherein S1 and S2 are the same length.

44. The double-stranded oligonucleotide of claim 42 or 43, wherein L is a triloop or a tetraloop.

45. The double-stranded oligonucleotide of claim 44, wherein the tetraloop comprises the sequence 5′-GAAA-3′.

46. The double-stranded oligonucleotide of any one of claims 42-45, wherein the stem-loop comprises the sequence 5′-GCAGCCGAAAGGCUGC-3′ (SEQ ID NO: 15).

47. The double-stranded oligonucleotide of any one of claims 1-38 and 40, comprising a blunt end.

48. The double-stranded oligonucleotide of claim 47, wherein the blunt end comprises the 3′ end of the sense strand and the 5′ end of the antisense strand.

49. The double-stranded oligonucleotide of any one of claims 1-38 and 40, comprising an overhang at the 5′ end of the antisense strand.

50. The double-stranded oligonucleotide of claim 49, wherein the overhang at the 5′ end of the antisense strand is 2-6 nucleotides in length.

51. A double-stranded oligonucleotide comprising an antisense strand of 15-30 nucleotides in length and a sense strand of about 9-50 nucleotides in length, wherein (i) the antisense and sense strands form a duplex region of about 9-26 base pairs, (ii) the antisense strand comprises an orientation of 5′ to 3′, (iii) the antisense strand comprises a 3′ overhang of at least four nucleotides and a 5′ overhang of at least 2 nucleotides, (iv) the antisense strand comprises a region of complementarity to an mRNA target sequence in a target mRNA, (v) the 3′ overhang comprises a sequence motif that reduces inhibition of the target mRNA by the double-stranded oligonucleotide in a cell of the liver, and (vi) the sequence motif comprises at least one 2′-F modified nucleotide, provided the at least one 2′-F modified nucleotide is not one of the two 3′terminal nucleotides of the antisense strand.

52. The double-stranded oligonucleotide of claim 51, wherein the sequence motif comprises: 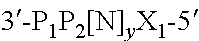
wherein:
P₁ and P₂ are each independently a purine or a pyrimidine, and do not comprise a 2′-F modification;
X₁ is any nucleotide located immediately adjacent to the duplex, and comprises at least one phosphorothioate linkage to an adjacent nucleotide;
N is any nucleotide and y is an integer selected from 1-6, specifying N₁-N₆, wherein:
(a) when y is 1, N₁ comprises a 2′-F modification;
(b) when y is 2, one or more of N₁ and N₂ comprise a 2′-F modification;
(c) when y is 3, one or more of N₁-N₃ comprise a 2′-F modification;
(d) when y is 4, one or more of N₁-Na comprise a 2′-F modification;
(e) when y is 5, one or more of N₁-N₅ comprise a 2′-F modification; or
(f) when y is 6, one or more of N₁-N₆ comprise a 2′-F modification.

53. The double-stranded oligonucleotide of claim 52, wherein when y is 3, N₂ comprises the 2′-F modification.

54. The double-stranded oligonucleotide of claim 53, wherein X₁ does not comprise a 2′-F modification.

55. The double-stranded oligonucleotide of claim 53 or 54, wherein N₁, N₃, and N₄ each comprise a 2′-OMe modification.

56. The double-stranded oligonucleotide of claim 52, wherein when y is 6, N₂ comprises the 2′-F modification.

57. The double-stranded oligonucleotide of claim 56, wherein X₁ comprises a 2′-F modification.

58. The double-stranded oligonucleotide of claim 56 or 57, wherein N₁, N₃, N₄, N₅, and N₆ each comprise a 2′-OMe modification.

59. The double-stranded oligonucleotide of claim 52, wherein when y is 6, N₂ and N₅ each comprise the 2′-F modification.

60. The double-stranded oligonucleotide of claim 59, wherein X₁ comprises a 2′-F modification.

61. The double-stranded oligonucleotide of claim 59 or 40, wherein N₁, N₃, N₄, and No each comprise a 2′-OMe modification.

62. The double-stranded oligonucleotide of any one of claims 52-61, wherein P₁ and P₂ are each independently a purine.

63. The double-stranded oligonucleotide of claim 62, wherein P₁ and P₂ are each independently selected from adenosine and guanine.

64. The double-stranded oligonucleotide of claim 62, wherein P₁ and P₂ are each guanine.

65. The double-stranded oligonucleotide of claim 52, wherein the antisense strand is 22 nucleotides, wherein the 3′ overhang is 4-9 nucleotides, and wherein the 2′-F nucleotide is located at position 19.

66. The double-stranded oligonucleotide of claim 52, wherein the antisense strand is 22 nucleotides, wherein the 3′ overhang is 6-9 nucleotides, and wherein the 2′-F nucleotide is located at position 16.

67. The double-stranded oligonucleotide of claim 52, wherein the antisense strand is 22 nucleotides, wherein the 3′ overhang is 6-9 nucleotides, and wherein the 2′-F nucleotide is located at position 16 and position 19.

68. The double-stranded oligonucleotide of any one of claims 52-67, wherein the sense strand comprises a lipid moiety conjugated to a nucleotide of the sense strand.

69. The double-stranded oligonucleotide of any one of claims 1-68, wherein the region of complementarity is fully complementary to the mRNA target sequence.

70. The double-stranded oligonucleotide of any one of claims 1-68, wherein the region of complementarity is partially complementary to the mRNA target sequence.

71. The double-stranded oligonucleotide of claim 70, wherein the region of complementarity comprises no more than four mismatches to the mRNA target sequence.

72. The double-stranded oligonucleotide of any one of claims 1-71, wherein the sense strand comprises at least one modified nucleotide.

73. The double-stranded oligonucleotide of any one of claims 1-72, wherein the antisense strand comprises at least one modified nucleotide in addition to the 2′-F modified nucleotide.

74. The double-stranded oligonucleotide of claim 72 or 73, wherein the modified nucleotide comprises a 2′-modification.

75. The double-stranded oligonucleotide of claim 74, wherein the 2′-modification is a modification selected from 2′-aminoethyl, 2′-fluoro, 2′-O-methyl, 2′-O-methoxyethyl, and 2′-deoxy-2′-fluoro-β-d-arabinonucleic acid.

76. The double-stranded oligonucleotide of any one of claims 74-75, wherein the sense strand comprises a 2′-fluoro modification at each of nucleotides forming a base pair with nucleotides at positions 10-13 of the antisense strand.

77. The double-stranded oligonucleotide of any one of claims 74-75, wherein the sense strand comprises a 2′-fluoro modification at each of nucleotides forming a base pair with nucleotides at positions 10-12 of the antisense strand.

78. The double-stranded oligonucleotide of any one of claims 74-75, wherein the sense strand comprises 16-32 nucleotides, wherein nucleotides at each of positions 3, 5, 6, 8, and 10 comprise a 2′-fluoro modification.

79. The double-stranded oligonucleotide of any one of claims 74-75, wherein the sense strand comprises 16-32 nucleotides, wherein nucleotides at each of positions 4-7 comprise a 2′-fluoro modification.

80. The double-stranded oligonucleotide of any one of claims 73-79, wherein the antisense strand comprises 22 nucleotides, and wherein each of positions 2-5, 7, 10, and 13 comprise a 2′-fluoro modification.

81. The double-stranded oligonucleotide of any one of claims 73-80, wherein the remaining nucleotides comprise a 2′-O-methyl modification, provided the nucleotide of the sense strand conjugated to the at least one lipid moiety does not comprise a 2′-O-methyl modification.

82. The double-stranded oligonucleotide of any one of claims 1-81, wherein the oligonucleotide comprises at least one modified internucleotide linkage.

83. The double-stranded oligonucleotide of claim 82, wherein the at least one modified internucleotide linkage is a phosphorothioate linkage.

84. The double-stranded oligonucleotide of claim 83, wherein the antisense strand comprises a phosphorothioate linkage (i) between positions 1 and 2, and between positions 2 and 3; or (ii) between positions 1 and 2, between positions 2 and 3, and between positions 3 and 4.

85. The double-stranded oligonucleotide of claim 83 or 84, wherein the antisense strand is 22 nucleotides in length, and wherein the antisense strand comprises a phosphorothioate linkage between positions 20 and 21 and between positions 21 and 22.

86. The double-stranded oligonucleotide of any one of claims 83-85, wherein the sense strand comprises a phosphorothioate linkage between positions 1 and 2.

87. The double-stranded oligonucleotide of any one of claims 1-86, wherein the antisense strand comprises a phosphorylated nucleotide at the 5′ terminus, wherein the phosphorylated nucleotide is selected from uridine and adenosine.

88. The double-stranded oligonucleotide of claim 87, wherein the phosphorylated nucleotide is uridine.

89. The double-stranded oligonucleotide of any one of claims 1-88, wherein the 4′-carbon of the sugar of the 5′-nucleotide of the antisense strand comprises a phosphate analog.

90. The double-stranded oligonucleotide of claim 89, wherein the phosphate analog is oxymethyl phosphonate, vinyl phosphonate or malonyl phosphonate.

91. The double-stranded oligonucleotide of any one of claims 68-70, wherein the phosphorylated nucleotide is 4′-O-monomethylphosphonate-2′-O-methyl uridine.

92. The double-stranded oligonucleotide of any one of claims 1-91, wherein the sense strand comprises at least one Tₘ-increasing nucleotide.

93. The double-stranded oligonucleotide of claim 92, wherein the sense strand comprises up to four Tₘ-increasing nucleotides.

94. The double-stranded oligonucleotide of claim 92, wherein the 5′ terminal nucleotide of the sense strand is a Tₘ-increasing nucleotide.

95. The double-stranded oligonucleotide of claim 92, wherein the sense strand comprises a stem-loop, and wherein the stem comprises at least one pair of Tₘ-increasing nucleotides.

96. The double-stranded oligonucleotide of any one of claims 92-95, wherein the Tₘ-increasing nucleotide is a bicyclic nucleotide.

97. The double-stranded oligonucleotide of any one of claims 92-95, wherein the Tₘ-increasing nucleotide is a locked nucleic acid.

98. The double-stranded oligonucleotide of any one of claims 1-97, wherein the oligonucleotide is a Dicer substrate.

99. A pharmaceutical composition comprising the double-stranded oligonucleotide of any one of claims 1-98, and a pharmaceutically acceptable carrier, delivery agent, or excipient.

100. A method of inhibiting target mRNA expression in a cell of an extra-hepatic tissue in a subject, comprising administering to the subject the double-stranded oligonucleotide of any one of claims 1-98, or the pharmaceutical composition of claim 99, thereby inhibiting target mRNA expression in the cell of the extra-hepatic tissue.

101. The method of claim 100, wherein the extra-hepatic tissue is selected from skeletal muscle, adipose tissue, adrenal tissue, and any combination thereof.

102. The method of claim 100 or 101, wherein the cell of the cell of the extra-hepatic tissue is selected from a cardiomyocyte, an immune cell, a liver non-parenchymal cell, a cell of skeletal muscle, a cell of adipose tissue, a cell of adrenal tissue, and any combination thereof.

103. The method of any one of claims 100-102, wherein reduction of the target mRNA in the cell of the extra-hepatic tissue is increased compared to reduction in a cell of the liver, optionally wherein reduction of the target mRNA is increased by at least 10%.

104. The method of claim 103, wherein reduction of the target mRNA is increased by at least 15%, at least 20%, at least 25%, at least 30%, at least 35%, at least 40%, at least 45% or at least 50%.

105. The method of claim 103 or 104, wherein the cell of the liver is a hepatocyte.

106. The double-stranded oligonucleotide of any one of claims 1-97 in the manufacture of a medicament for inhibiting target mRNA expression in a cell of an extra-hepatic tissue in a subject.

107. Use of the double-stranded oligonucleotide of any one of claims 1-97 for inhibiting target mRNA expression in a cell of an extra-hepatic tissue in a subject.

108. A kit comprising a container comprising the double-stranded oligonucleotide of any one of claims 1-97, and optionally a pharmaceutically acceptable carrier, and instructions for administering the double-stranded oligonucleotide to a subject in need thereof, wherein the double-stranded oligonucleotide inhibits target mRNA expression in a cell of an extra-hepatic tissue in the subject.

109. The double-stranded oligonucleotide of claim 106, the use of claim 107, or the kit of claim 108, wherein the extra-hepatic tissue is selected from skeletal muscle, adipose tissue, adrenal tissue, and any combination thereof.

110. The double-stranded oligonucleotide of claim 106, the use of claim 107, or the kit of claim 108, wherein the cell of the cell of the extra-hepatic tissue is selected from a cardiomyocyte, an immune cell, a liver non-parenchymal cell, a cell of skeletal muscle, a cell of adipose tissue, a cell of adrenal tissue, and any combination thereof.

111. The double-stranded oligonucleotide, use, or kit of any one of claims 106-110, wherein reduction of the target mRNA in the cell of the extra-hepatic tissue is increased compared to reduction in a cell of the liver, optionally wherein reduction of the target mRNA is increased by at least 10%.

112. The double-stranded oligonucleotide, use, or kit of claim 111, wherein reduction of the target mRNA is increased by at least 15%, at least 20%, at least 25%, at least 30%, at least 35%, at least 40%, at least 45% or at least 50%.

113. The double-stranded oligonucleotide, use, or kit of claim 111 or 112, wherein the cell of the liver is a hepatocyte.
